(19) United States
(12) Patent Application Publication
(10) Pub. No.: US 2026/0034081 A1
(43) Pub. Date: Feb. 5, 2026

(54) COMPOSITIONS AND METHODS RELATED TO INHIBITION OF ADENOMAS AND ADENOCARCINOMAS

(71) Applicant: THE TRUSTEES OF COLUMBIA UNIVERSITY IN THE CITY OF NEW YORK, New York, NY (US)

(72) Inventors: Piero D. Dalerba, Dobbs Ferry, NY (US); Sara Viragova, San Francisco, CA (US); Pierangela Palmerini, New York, NY (US)

(21) Appl. No.: 19/351,201

(22) Filed: Oct. 6, 2025

Related U.S. Application Data
(63) Continuation-in-part of application No. PCT/US2024/023164, filed on Apr. 4, 2024.
(60) Provisional application No. 63/494,178, filed on Apr. 4, 2023.

(51) Int. Cl. A61K 31/192; A61K 31/203; A61P 35/00; G01N 33/574

(52) U.S. Cl. CPC A61K 31/192; A61K 31/203; A61P 35/00; G01N 33/57492; G01N 2333/70503; G01N 2333/7055

(57) ABSTRACT

This application is directed to methods and compositions related to the treatment and diagnosis of adenocarcinomas, such as adenoid cystic carcinoma (ACC). The methods and compositions related to the use of CD49f, TP63, and/or KIT/CD117 cell-surface markers for subtyping the cancer cells. One method involves using a retinoic acid receptor/retinoid-X receptor inhibitor to inhibit the differentiation of myoepithelial-like cells into ductal-like cells. Another method involves using a retinoic acid receptor/retinoid-X receptor inhibitor to selectively reduce the viability of ductal-like cells.

### CROSS-REFERENCE TO RELATED APPLICATIONS

The present application is a Continuation-in-Part application of International Application No. PCT/US2024/23164, filed on Apr. 4, 2024, which claims priority to U.S. Provisional Patent Application 63/494,178, filed Apr. 4, 2023. The foregoing application is hereby incorporated by reference in its entirety.

### STATEMENT REGARDING FEDERALLY SPONSORED RESEARCH OR DEVELOPMENT

This invention was made with government support under TR001875 and DE020687, awarded by the National Institutes of Health. The government has certain rights in the invention.

### TECHNICAL FIELD

This invention relates to the field of targeted therapy for the treatment of cancers, such as adenoid cystic carcinoma, and related disorders.

### BACKGROUND

Adenoid cystic carcinomas (ACCs) are malignant adenocarcinomas that originate in exocrine glands, most commonly the salivary glands (SGs) [1]. ACCs display indolent growth, but their slow proliferation kinetics often belie an aggressive and relentless nature, characterized by peri-neural infiltration and early hematogenous spread [1-3]. Current treatments for ACCs are limited to surgery and radiotherapy. Because ACCs usually arise within the craniofacial district, such treatments are often destructive and, in approximately 60% of cases, unable to prevent metastatic relapse and patient death [2-5]. ACCs are usually refractory to chemotherapy, immunotherapy and various types of targeted therapies [6-9]. ACCs often associate with t(6;9) MYB-NFIB chromosomal translocations [10-13], but no actionable treatments are currently available to suppress the oncogenic signaling that results from them [14].

Histologically, ACCs are characterized by a distinctive feature: the co-existence of two populations of malignant cells, termed “ductal-like” and “myoepithelial-like”, because of their phenotypic similarity to ductal and myoepithelial lineages of normal SG epithelia [15-21]. The molecular causes of this feature are poorly understood, and remain difficult to investigate, due to the lack of experimental means to differentially isolate the two cell-types. It remains unknown, for example, whether the two populations represent distinct genetic clones, arising from the divergent accumulation of distinct repertoires of somatic mutations, or distinct developmental lineages, arising from the retention by malignant tissues of normal differentiation programs [22-25]. It also remains unclear how the two populations compare in terms of differential sensitivity to anti-tumor therapies.

### SUMMARY OF THE INVENTION

Adenoid Cystic Carcinoma (ACC) is a lethal malignancy of exocrine glands, characterized by the co-existence within tumor tissues of two distinct populations of cancer cells, phenotypically similar to the myoepithelial and ductal lineages of normal salivary epithelia. The developmental relationship linking these two cell-types, and their differential vulnerability to anti-tumor treatments, remain unknown.

Using single-cell RNA-sequencing (scRNA-seq), cell-surface markers were identified (for example, CD49f, TP63, and KIT) that enabled the differential purification of myoepithelial-like (CD49f^{high}/KIT^{neg} or TP63⁺/KIT^{neg}) and ductal-like (CD49f^{low}/KIT⁺ or TP63^{neg}/KIT⁺) cells from patient-derived xenografts (PDX) of human ACCs. Using prospective xeno-transplantation experiments, the tumor-initiating capacity of the two cell-types was compared and then tested as to whether one could differentiate into the other. Finally, signaling pathways with differential activation between the two cell-types were sought and tested for their role as lineage-specific therapeutic targets. Thus, the use of KIT/CD117, CD49f, TP63, alone or in combination to detect the presence of myoepithelial-like cells (for example, myoepithelial-like ACC cells) and the presence of ductal-like cells (for example, ductal-like ACC cells) is disclosed. In some aspects, the use of KIT/CD117, CD49f, TP63, alone or in combination, to type adenocarcinoma cells (for example, ACC cells) as myoepithelial-like or ductal-like is disclosed.

Myoepithelial-like cells displayed higher tumorigenicity than ductal-like cells and acted as their progenitors. Myoepithelial-like and ductal-like cells displayed differential expression of genes encoding for suppressors and activators of retinoic acid signaling, respectively. Agonists of retinoic acid receptor (RAR) or retinoid X receptor (RXR) signaling (ATRA, bexarotene) promoted myoepithelial-to-ductal differentiation, whereas suppression of RAR/RXR signaling with a dominant-negative RAR construct abrogated it. Inverse agonists of RAR/RXR signaling (BMS493, AGN193109) displayed selective toxicity against ductal-like cells, and in vivo anti-tumor activity against PDX models of ACC. In human ACCs, myoepithelial-like cells act as progenitors of ductal-like cells, and myoepithelial-to-ductal differentiation is promoted by RAR/RXR signaling. Suppression of RAR/RXR signaling is lethal to ductal-like cells and represents a new therapeutic approach against human ACCs.

Accordingly, disclosed herein are a method of reducing tumorigenicity and/or aggression of adenocarcinoma cells (for example, ACC cells). The method comprises administering a therapeutic agent that activates retinoic acid receptor and/or retinoid-X receptor signaling to the adenocarcinoma cells. In some aspects, the therapeutic agent is administered at a dose effective to induce myoepithelial-to-ductal differentiation the adenocarcinoma cells. In some implementations, the method further comprises detecting the expression of CD49f, TP63, and/or KIT/CD117 in the adenocarcinoma cells. Upon detection of more than 5% of the adenocarcinoma cells express TP63, less than 95% of the adenocarcinoma cells express KIT/CD117, or the adenocarcinoma cells have high expression of CD49f, the adenocarcinoma cells are administered the therapeutic agent that activates retinoic acid receptor and/or retinoid-X receptor signaling. In some implementations, the method further comprises administering a therapeutic agent that inhibits retinoic acid receptor and/or retinoid-X receptor signaling to the adenocarcinoma cells after the administration of the therapeutic agent that activates retinoic acid receptor and/or retinoid-X receptor signaling.

Also disclosed herein is a method of reducing viability of adenocarcinoma cells (for example, ACC cells). The method comprises detecting the expression of CD49f, TP63, and/or KIT/CD117 in the adenocarcinoma cells; and administering a therapeutic agent that inhibits retinoic acid receptor and/or retinoid-X receptor signaling to the adenocarcinoma cells. Upon detection of less than 5% of the adenocarcinoma cells express TP63, more than 95% of the adenocarcinoma cells express KIT/CD117, or the adenocarcinoma cells have low expression of CD49f, the adenocarcinoma cells are administered the therapeutic agent that inhibits retinoic acid receptor and/or retinoid-X receptor signaling. In some implementations, upon the detection more than 5% of the adenocarcinoma cells express TP63, less than 95% of the adenocarcinoma cells express KIT/CD117, or the adenocarcinoma cells have high expression of CD49f, the method further comprises administering a therapeutic agent that activates retinoic acid receptor and/or retinoid-X receptor signaling to the adenocarcinoma cells prior to administering the therapeutic agent that inhibits retinoic acid receptor and/or retinoid-X receptor signaling to the adenocarcinoma cells. The administration of the therapeutic agent that activates retinoic acid receptor and/or retinoid-X receptor signaling produces a population of treated adenocarcinoma cells expressing KIT/CD117 without expression of TP63 or expressing KIT/CD117 with low expression of CD49f.

In some aspects of the methods related to adenocarcinoma cells, the step of detecting the expression of CD49f, TP63, and/or KIT/CD117 in the adenocarcinoma cells comprises combining an antibody of CD49f, antibody of TP63, and/or an antibody of KIT/CD117 with the adenocarcinoma cells; and sorting the adenocarcinoma cells based on binding of the antibody of CD49f, the antibody of TP63, and/or the antibody of KIT/CD117 to the adenocarcinoma cells. In certain implementations, the antibody of CD49f, the antibody of TP63, and/or the antibody of KIT/CD117 are conjugated to a fluorescence marker, a magnetic particle, or microbubbles.

Further described herein in is a method of reducing the size of a tumor (for example an ACC tumor). In one embodiment, the method comprises providing a tumor sample from a subject; detecting the expression of at least one cell-surface marker in the tumor sample, wherein the at least one cell-surface marker is selected from the group consisting of: CD49f, TP63, and KIT/CD117; and administering a therapeutic agent that inhibits retinoic acid receptor and/or retinoid-X receptor signaling to the subject with a tumor sample comprising more than 95% of cells expressing KIT/CD117 or with a tumor sample comprising less than 5% of cells expressing TP63. In some aspects, the subject is administered the therapeutic agent that inhibits retinoic acid receptor and/or retinoid-X receptor signaling if the subject's tumor sample has low expression level of CD49f, for example.

In some implementations, the method of reducing the size of a tumor further comprises confirming the expression of at least a second cell-surface marker in the tumor sample selected from the group consisting of: ACTA2, MYH11, PDPN, ELF5, SLPI, and ANXA8. In such implementations, the therapeutic agent that inhibits retinoic acid receptor and/or retinoid-X receptor signaling is administered to the subject with a tumor sample comprising more than 95% of cells expressing KIT/CD117 and at least a second cell-surface marker selected from the group consisting of ELF5, SLPI, and ANXA8.

In certain implementations, the step of detecting the expression of CD49f, TP63, and/or KIT/CD117 in the tumor sample comprises combining an antibody of CD49f, antibody of TP63, and/or an antibody of KIT/CD117 with cells of the tumor sample; and sorting the cells of the tumor sample based on binding of the antibody of CD49f, the antibody of TP63, and/or the antibody of KIT/CD117 to the cells of the tumor sample. In some aspects, the antibody of CD49f, the antibody of TP63, and/or the antibody of KIT/CD117 are conjugated to a fluorescence marker, a magnetic particle, or microbubbles.

In another embodiment, the method of reducing the size of a tumor comprises providing a tumor sample from a subject; sorting cells from the tumor sample based on expression level of CD49f, TP63, and KIT/CD117; and administering a therapeutic agent that inhibits retinoic acid receptor and/or retinoid-X receptor signaling to the subject with a tumor sample comprising more than 95% of cells expressing KIT/CD117 or less than 5% of cells expressing TP63 or a tumor sample having low expression of CD49f. In some implementations, the method further comprises administering a therapeutic agent that activates retinoic acid receptor and/or retinoid-X receptor signaling to the subject with a tumor sample comprising more than 5% of the cells expressing TP63 or less than 95% of the cells expressing KIT/CD117 or a tumor sample having high expression of CD49f. In such implementations, the therapeutic agent that activates retinoic acid receptor and/or retinoid-X receptor signaling is administered prior to the therapeutic agent that inhibits retinoic acid receptor and/or retinoid-X receptor signaling. The administration of the therapeutic agent that activates retinoic acid receptor and/or retinoid-X receptor signaling alters the cells of the tumor to produce a population of cells expressing KIT/CD117 without expression of TP63 or expressing KIT/CD117 with low expression of CD49f.

Additionally described herein is a method of inhibiting growth of ACC in a subject. The method comprises obtaining an ACC tumor sample from the subject; sorting cells of the tumor sample based on the expression of CD49f and KIT/CD117 in the ACC tumor sample; and administering a therapeutic agent to the subject that inhibits retinoic acid receptor and/or retinoid-X receptor signaling upon the indication of the presence of ductal-like ACC cells in the sample. The presence of cells positive for KIT/CD117 with low expression of CD49f indicates the presence of ductal-like ACC cells. The presence of cells negative for KIT/CD117 with high expression of CD49f indicates the presence of myoepithelial-like ACC cells. In some implementations, where the sorting step indicates the tumor sample comprises myoepithelial-like ACC cells, the method further comprising administering to the subject a therapeutic agent that activates retinoic acid receptor and/or retinoid-X receptor signaling before administering the therapeutic agent that inhibits retinoic acid receptor and/or retinoid-X receptor, thereby inducing the differentiation of myoepithelial-like tumor cells into ductal-like tumor cells.

For the methods described herein, therapeutic agent that activates retinoic acid receptor and/or retinoid-X receptor signaling is selected from the group consisting of: all-trans retinoic acid (ATRA), bexarotene, or a combination thereof.

The use of a dominant-negative version of RARα (DNRARα) for reducing viability of ductal adenoid cystic carcinoma is additionally described. The DNRARα is expressed in a gene construct.

For the methods and uses described herein, the therapeutic agent that inhibits retinoic acid receptor/retinoid-X receptor signaling is selected from the group consisting of: BMS493, AGN193109, or a combination thereof. In another aspects, the therapeutic agent that inhibits retinoic acid receptor/retinoid-X receptor signaling is a gene construct encoding a dominant-negative version of RARα (DNRARα). In some embodiments, the DNRARα is a retinoic acid receptor alpha lacking its C-terminal transcriptional activation domain, for example, the DNRARα is a retinoic acid receptor alpha truncated at amino acid residue 403. In some implementations, the gene construct encoding DNRARα comprises DNhRARα subcloned into a lentivirus backbone. In some aspects, the lentivirus backbone is based on the pLL3.7 backbone.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J show the identification of surface markers for the differential purification of myoepithelial-like and ductal-like cell populations from human ACCs. FIG. 1A shows histological analysis of the ACCX22 human PDX line, confirming retention of a cribriform histology with pseudo-cyst formation, characteristic of well-differentiated (Grade 1) ACCs. FIG. 1B shows magnification of the tissue area outlined in panel A (dashed box), demonstrating the presence of: 1) ductal-like cells, characterized by abundant eosinophilic cytoplasm and arranged in ring-like structures (arrows); and 2) myoepithelial-like cells (arrow-heads), characterized by spindle-shaped morphology and arranged to line pseudo-cysts. FIG. 1C shows visualization by Uniform Manifold Approximation and Projection (UMAP) of scRNA-seq data obtained from a purified preparation of human malignant cells (EpCAM⁺) sorted by FACS from the ACCX22 human PDX line. In the UMAP scatter-plot, the three cell clusters identified as representing the most robust clustering solution (i.e., as displaying the highest mean silhouette score following clustering based on the Leiden algorithm) were labeled and displayed clear visual separation. Based on differentially expressed genes (FIGS. 11A-11C), the three clusters were annotated as follows: Cluster 1=myoepithelial-like cells; Cluster 2=ductal-like cells; Cluster 3=proliferating ductal-like cells. FIG. 1D shows the list of genes identified as displaying a statistically significant difference in mean expression levels between Cluster 1 (myoepithelial-like) and Cluster 2 (ductal-like), based on a Student t-test (two-tailed) adjusted for multiple comparisons (FDR<0.001; Benjamini-Hochberg method). Among the differentially expressed genes are those encoding for two surface markers: ITGA6 (CD49f) and KIT (CD117). FIGS. 1E-1G show UMAP plots displaying gene-expression levels for ITGA6 (E), KIT (F) and the proliferation marker MKI67 (G); q-values are based on a Student t-test (two-tailed), corrected for multiple comparisons (Benjamini-Hochberg method), as described in FIG. 11. FIGS. 1H-1I show the violin plots displaying the distribution of gene-expression levels for ITGA6 (H), KIT (I) and MKI67 (J) across the three cell clusters identified by scRNA-seq; p-values are based on a Kruskal-Wallis H-test.

FIGS. 2A-2D show differential purification by f^{low} cytometry of myoepithelial-like (CD49f^{high}/KIT^{neg}) and ductal-like (CD49f^{low}/KIT⁺) cells. FIG. 2A shows analysis by f^{low} cytometry of CD49f and KIT surface expression in five human PDX lines representative of bi-phenotypic ACCs, enabling visual discrimination of two distinct populations of human malignant cells: CD49f^{high}/KIT^{neg} (black gates) and CD49f^{low}/KIT⁺ (gray gates). FIG. 2B shows analysis by immunohistochemistry (IHC) of corresponding tumors, confirming the mutually exclusive expression of TP63 (a myoepithelial marker) and KIT (a ductal marker). Scale bar: 50 μm. FIG. 2C shows Principal component analysis (PCA) of RNA-seq data obtained from five autologous pairs of CD49f^{high}/KIT^{neg} (red) and CD49f^{low}/KIT⁺ (green) cells, purified in parallel from five bi-phenotypic PDX lines of human ACCs (ACCX5M1, ACCX6, ACCX14, ACCX22, SGTX6). PCA was performed using the top 500 genes displaying the highest level of variance across the full 10-sample dataset. The 10 samples segregated into two distinct clusters, corresponding to their surface marker phenotype (CD49f^{high}/KIT^{neg} vs. CD49f^{low}/KIT⁺) and separating along the first principal component (PC1). FIG. 2D shows the heatmap of the top 100 genes identified as differentially expressed between CD49f^{high}/KIT^{neg} and CD49f^{low}/KIT⁺ cells, after mean-centering of gene-expression levels and hierarchical clustering of both genes and samples. Differentially expressed genes were defined as those with a >2-fold difference in mean expression levels between the two populations (log₂ fold-change >1) that was considered to be statistically robust based on a Wald test corrected for multiple comparisons (FDR<0.05; Benjamini-Hochberg method). Differentially expressed genes were ranked based on the p-value from the Wald test. Previously validated ductal and myoepithelial cell markers are highlighted in green and red, respectively.

FIGS. 3A-3Q show the tumorigenic properties of myoepithelial-like (CD49f^{high}/KIT^{neg}) and ductal-like (CD49f^{low}/KIT⁺) cells. FIG. 3A shows the predicted outcomes of cell transplantation experiments under a “clonal” model, whereby different cell types give rise to distinct progenies, each retaining the phenotypic properties of the parent cells. FIG. 3B shows the predicted outcomes of cell transplantation experiments under a “differentiation” model, whereby one or more cell types can serve as a progenitors of others, in a plastic and dynamic fashion. FIG. 3C shows the experimental workflow of prospective xeno-transplantation experiments aimed at comparing the tumor-initiating capacity of CD49f^{high}/KIT^{neg} and CD49f^{low}/KIT⁺ cells. The two populations were purified in parallel by FACS, starting from the same tumor lesion, double-sorted to achieve high purity (>95%) and injected subcutaneously, side-by-side, into the opposite flanks of the same animal. FIGS. 3D and 3E show the Extreme limiting dilution analysis (ELDA) of xeno-transplantation experiments using paired sets of CD49f^{high}/KIT^{neg} and CD49f^{low}/KIT⁺ cells sorted from ACCX5M1 (FIG. 3D) and SGTX6 (FIG. 3E) PDX lines. In both models, the frequency of tumor-initiating cells was higher in CD49f^{high}/KIT^{neg} as compared to CD49f^{low}/KIT⁺ cells. FIGS. 3F-3Q show the analysis by FACS and IHC of the cell composition of tumors originated from the xeno-transplantation of purified preparations of either CD49f^{high}/KIT^{neg} or CD49f^{low}/KIT⁺ cells, sorted from either ACCX5M1 (F-K) or SGTX6 (L-Q) PDX lines. Analysis by FACS (FIGS. 3F, 3G, 3I, 3J, 3L, 3M, 3O, and 3P) showed that tumors originated from sorted cells contained both CD49f^{high}/KIT^{neg} and CD49f^{low}/KIT⁺ populations, irrespective of the original phenotype of sorted cells. In tumors originated from CD49f^{high}/KIT^{neg} cells, the percentage of CD49f^{high}/KIT^{neg} cancer cells did not appear increased as compared to that observed in parent tumors but was lower than that observed in the purified preparations (FIGS. 3G and 3M), indicating spontaneous in vivo differentiation (n.s.: not significant; *p<0.05; Mann-Whitney U-test, one-tailed). A symmetric scenario was observed in tumors originated from CD49f^{low}/KIT⁺ cells (FIGS. 3J and 3P). Analysis by IHC of tumors originated from sorted cells (FIGS. 3H, 3K, 3N, and 3Q) confirmed the reconstitution of a cribriform histology and of a bi-phenotypic cell composition, defined by the co-existence of two distinct subsets of cancer cells with mutually exclusive expression of myoepithelial-specific (TP63) and ductal-specific (KIT) biomarkers. Scale bars: 50 μm.

FIGS. 4A-4M show the role of retinoic acid (RA) signaling in controlling the cell composition of human ACC organoids. FIG. 4A shows the schematic modeling of the RA signaling pathway. FIG. 4B shows the comparison of the gene-expression levels for known mediators of RA signaling in CD49f^{high}/KIT^{neg} (filled circle) and CD49f^{low}/KIT⁺ (filled square) cells, as measured by RNA-seq on autologous pairs from bi-phenotypic ACCs. Genes identified as attenuators of RA signaling displayed preferential expression in CD49f^{high}/KIT^{neg} cells (CD49f), while genes identified as potentiators of RA signaling displayed preferential expression in CD49f^{low}/KIT⁺ cells (KIT). Error bars: mean+/−standard deviation (n.s.: not-significant, *p<0.10, *p<0.05, **p<0.01; Student's t-test, paired samples). FIG. 4C shows the heatmap displaying mean-centered z-scores for the average expression levels of modulators of RA signaling in CD49f^{high}/KIT^{neg} and CD49f^{low}/KIT⁺ cells. FIGS. 4D-4G show the analysis by microscopy and IHC of 3D organoids established from human ACCs. Organoids consisted in large adenoid structures (FIGS. 4D and 4E) that recapitulated key elements of the histological architecture of primary tumors, such as the co-existence of two cell-types with mutually exclusive expression of TP63 (FIG. 4F) and KIT (FIG. 4G). FIGS. 4H-4I show the analysis by flow cytometry of ACCX5M1 organoids treated for 1 week with either agonists (ATRA, 10 μM; bexarotene, 10 μM) or inhibitors (BMS493, 10 μM; AGN193109, 10 μM) of RAR/RXR signaling. Treatment with agonists induced an increase in the percentage of CD49f^{low}/KIT⁺ cells, while treatment with inhibitors resulted in their reduction. FIGS. 4J-4M show the dose-response studies of the effects of agonists and inhibitors of RAR/RXR signaling on the cell composition of human ACC organoids. Treatment with increasing concentrations of ATRA (0.1-100 μM) resulted in a progressive increase of the percentage of CD49f^{low}/KIT⁺ cells (ACCX5M1, FIGS. 4J and 4K). The effects of ATRA were already detectable at low concentrations (0.1-1 μM; ACCX5M1, FIG. 4J). Treatment with inhibitors of RAR/RXR signaling (BMS493, AGN193109) resulted in a profound reduction of the percentage of CD49f^{low}/KIT⁺ cells, even at low pharmacological doses (1 μM; ACCX5M1, FIG. 4L; SGTX6, FIG. 4M). Changes in the percentage of CD49f^{low}/KIT⁺ cells were evaluated by FACS and tested for statistical significance using Welch's one-way ANOVA followed by Dunnett's T3 test (n.s.: not-significant, *p<0.05, **p<0.01, ***p<0.001) assuming a normal distribution (FIGS. 15A-15F). NT: untreated.

FIGS. 5A-5S show the pharmacological perturbation of RAR/RXR signaling across different PDX models and analysis of its effects. FIGS. 5A-5F show the analysis and quantification by f^{low} cytometry of the relative percentage of CD49f^{low}/KIT⁺ cells in ACC organoids established from three independent PDX lines (ACCX5M1, SGTX6, ACCX6), following one week of treatment with either ATRA (10 μM) or BMS493 (10 μM). Treatment with ATRA was associated with an increase in the percentage of CD49f^{low}/KIT⁺ cells, while treatment with BMS493 was associated with its reduction, as compared to control organoids treated only with DMSO, the solvent used to resuspend the two drugs. Differences in the mean percentage of CD49f^{low}/KIT⁺ cells were tested for statistical significance using Welch's one-way ANOVA followed by Dunnett's T3 test (*p<0.05, **p<0.01, ***p<0.001) assuming a normal distribution (FIGS. 15A-15F). Box-plots report the results of at least two independent experiments (with a minimum of 3 replicates for each condition). FIGS. 5G-5R show the analysis by IHC of 3D organoids established from the ACCX6 PDX line and treated with DMSO, ATRA (10 μM) or BMS493 (10 μM). Treatment with ATRA resulted in a visual expansion of KIT⁺ cells (FIG. 5M) as compared to treatment with DMSO alone (FIG. 51), while treatment with BMS493 resulted in a complete loss of KIT expression (FIG. 5Q) and was associated with a dramatic change in the organoids' morphology, characterized by the appearance of amorphous, eosin-rich deposits at their center (FIG. 5O). Neither ATRA nor BMS493 appeared to upregulate MKI67 expression in either cell population (FIGS. 5H, 5L, and 5P). Scale bars: 100 μm. FIG. 5S shows the schematic modeling of the effects produced by agonism and inhibition of RAR/RXR signaling on the cell composition of human ACCs, as hypothesized based on the observations conducted on whole 3D organoids: stimulation of RAR/RXR signaling induces the differentiation of myoepithelial-like cells into ductal-like cells, while inhibition of RAR/RXR causes selective death of ductal-like cells.

FIGS. 6A-6N show the effects of RAR/RXR signaling on the differentiation of myoepithelial-like cells into ductal-like cells and the survival of ductal-like cells. FIG. 6A shows the schematic workflow of experiments aimed at elucidating the population-specific effects of pharmacological manipulations of RAR/RXR signaling. Paired sets of CD49f^{high}/KIT^{neg} and CD49f^{low}/KIT⁺ cells were sorted in parallel from the same tumor (ACCX5M1) and cultured for 1 week as 2D monolayers, in the presence of either ATRA (10 μM) or BMS493 (10 μM), respectively. FIGS. 6B-6D show the evaluation of the effects of ATRA on sorted CD49f^{high}/KIT^{neg} cells. Treatment with ATRA did not affect the viability of CD49f^{high}/KIT^{neg} cells (FIG. 6B; alamarBlue assay) but caused CD49f^{high}/KIT^{neg} cells to change phenotype and become CD49f^{low}/KIT⁺ (FIGS. 6C and 6D; FACS). FIGS. 6E-6F show the evaluation of the effects of BMS493 on sorted CD49f^{low}/KIT⁺ cells. Treatment with BMS493 caused the death of the majority CD49f^{low}/KIT⁺ cells (FIG. 6E; alamarBlue assay). Upon visual inspection by conventional microscopy, CD49f^{low}/KIT⁺ cells treated with BMS493 appeared fragmented as compared to control cells treated with DMSO alone (FIG. 6F). Scale bar: 100 μm. FIG. 6G shows the schematic workflow of the experiment aimed at testing the effects of a DNhRARα construct on the capacity of CD49f^{high}/KIT^{neg} cells to undergo myoepithelial-to-ductal differentiation. CD49f^{high}/KIT^{neg} cells were sorted from ACCX5M1 tumors, cultured for 6 days as 2D monolayers, and infected with lentivirus vectors encoding for either a DNhRARα-EGFP construct or a control EGFP reporter. Infected cells were then cultured for one additional week and analyzed by FACS for CD49f and KIT expression, restricting the analysis to infected (EGFP⁺) cells. FIGS. 6H-6J show the analysis by FACS of CD49f^{high}/KIT^{neg} cells purified form ACCX5M1 tumors and infected with lentivirus vectors encoding for either a DNhRARα-EGFP construct or a control EGFP reporter. Forced DNhRARα expression completely abrogated the capacity of CD49f^{high}/KIT^{neg} cells to produce a CD49f^{low}/KIT⁺ progeny, while forced expression of EGFP alone did not. FIGS. 6K-6N show the evaluation of the role of DNhRARα as a suppressor of myoepithelial-to-ductal differentiation in a second, independent PDX model (SGTX6). Error bars: mean+/−standard deviation; p-values: Student's t-test, two-tailed (n.s.: not significant, *p<0.05, ***p<0.001).

FIGS. 7A-7K show the transcriptional profile and drug sensitivity of ACCs with solid histology. FIGS. 7A-7B show the analysis by f^{low} cytometry of two PDX lines representative of human ACCs with solid histology (FIG. 7A: ACCX9; FIG. 7B: ACCX11) revealing a ductal-like, mono-phenotypic (CD49f^{low}/KIT⁺) cell composition. FIGS. 7C-7F show the analysis by IHC of KIT and TP63 expression in ACCX9 and ACCX11 tumors, showing ubiquitous expression of the ductal-specific marker KIT (FIGS. 7C and 7E) and complete loss of the myoepithelial-specific marker TP63 (FIGS. 7D and 7F). Scale bars: 50 μm. FIG. 7G shows Principal component analysis (PCA) of RNA-seq data from human ACCs, in which data from the two PDX lines with solid histology (ACCX9, ACCX11) are combined with those from the 5 autologous pairs of CD49f^{high}/KIT^{neg} and CD49f^{low}/KIT⁺ cells isolated from bi-phenotypic PDX lines (ACCX5M1, ACCX6, ACCX14, ACCX22, SGTX6). PCA was performed using the top 500 genes displaying the highest level of variance across the full 12-sample dataset. FIG. 7H shows hierarchical clustering of RNA-seq data from human ACCs, based on the expression levels of the same list of 100 genes identified as differentially expressed between CD49f^{high}/KIT^{neg} and CD49f^{low}/KIT⁺ cells and reported in FIG. 2D. Solid ACCs clustered with CD49f^{low}/KIT⁺ cells from bi-phenotypic tumors, irrespective of the method used to analyze their transcriptional profile. FIG. 7I-7J show that upon visual inspection by conventional microscopy, ACCX9 organoids cultured for one week in the presence of BMS493 (10 μM) displayed widespread cell fragmentation, in contrast to organoids cultured with DMSO alone. FIG. 7K shows the quantification of organoid viability using the alamarBlue assay, confirming the cytotoxic activity of BMS493 (10 μM) against PDX lines with solid histology (ACCX9, ACCX11). Error bars: mean+/−standard deviation; p-values: Student's t-test (two-tailed; **p<0.01).

FIGS. 8A-8H show in vivo anti-tumor activity of BMS493. FIG. 8A shows the schematic description of the BMS493 dosing regimen utilized for the in vivo treatment of solid ACC models (40 mg/kg doses, i.p., 3 times/week×3 weeks). FIGS. 8B-8E show the comparison of tumor growth kinetics between mice treated with BMS493 and mice treated with the drug's vehicle alone (DMSO), following subcutaneous engraftment of two solid ACC models (ACCX9: FIGS. 8B and 8C; ACCX11: FIGS. 8D and 8E). FIG. 8F shows the schematic of the BMS493 dosing regimen utilized for the in vivo treatment of the bi-phenotypic ACC model (40 mg/kg doses, i.p., 4 times/week×3 weeks). FIGS. 8G-8H show the comparison of tumor growth kinetics between mice treated with BMS493 and mice treated with the drug's vehicle alone (DMSO), following subcutaneous engraftment of a bi-phenotypic ACC model (ACCX5M1). Differences in tumor growth kinetics were quantified by comparing either mean fold-increases in tumor volume (FIGS. 8B, 8D, and 8G) or mean growth rates (FIGS. 8C, 8E, and 8H). Differences in mean fold-increases in tumor volume (FIGS. 8B, 8D, and 8G) were tested for statistical significance using two approaches: 1) at each time-point, using a two-tailed Student's t-test (*p<0.05, **p<0.01, ***p<0.001); and 2) across the full experimental dataset, using a two-way ANOVA for repeated measures (RM), where measurements performed on the same mouse at different time-points were treated as repeated measures (p_{interaction}=time×treatment). Growth rates were calculated assuming exponential kinetics. Differences between mean growth rates (FIGS. 8C, 8E, and 8H) were tested for statistical significance using a two-tailed Welch's t-test. Error bars: mean+/−standard deviation. Schematic descriptions of dosing regimens were created using BioRender.com.

FIGS. 9A-9D show the workflow of the single-cell RNA-sequencing (scRNA-seq) experiment performed to analyze the cell composition of the human ACCX22 patient-derived xenograft (PDX) line. FIG. 9A shows that the solid tumor tissues were harvested from mice, minced into small fragments using scissors and dissociated into a single-cell suspension by enzymatic digestion (DNase-I, collagenase-III, hyaluronidase). FIG. 9B shows that single-cell suspensions were stained with monoclonal antibodies and analyzed using a fluorescence-activated cell sorter (BD FACSAria). FIG. 9C shows the gating strategy used to isolate single, live (DAPIneg), human (mouse Cd45neg, mouse H-2Kdneg), epithelial (EpCAM+) cells from ACCX22 tumors. FIG. 9D shows the overview of the experimental pipeline used for the technical execution and computational analysis of the scRNA-seq experiment. Single-cell libraries were prepared using the 10× Chromium system (Single Cell 3′ v3 chemistry) and sequenced using the NovaSeq-6000 platform (Illumina). Sequencing data were filtered to exclude cells expressing.

FIGS. 10A-10E show the computational analysis of single-cell RNA-sequencing (scRNA-seq) data obtained from the patient-derived xenograft (PDX) line ACCX22. FIG. 10A shows the spectral distribution (histogram) of the Wishart matrix for 3,533 cells identified as sequenced at sufficient depth (>500 expressed genes), after elimination of sparsity-induced signal. After fitting the Marchenko-Pastur (MP) distribution to the data (curve), 47 eigenvalues (1.3%; n=47/3,533) are identified as lying outside the MP distribution, and thus as corresponding to eigenvectors that carry informative signal. FIG. 10B shows the mathematical properties of the data distribution. FIG. 10C shows the study of the chi-squared test for the variance (normalized sample variance) of each gene's projection into noise and signal eigenvectors. The black distribution (curve with dashed line) was generated based on the 47 signal-like eigenvectors, the dark gray distribution (curve with dotted line) based on the eigenvectors corresponding to the highest 47 eigenvalues within the MP distribution, and the light gray distribution (curve with solid line) based on the eigenvectors corresponding to the smallest 47 eigenvalues within the MP distribution. FIG. 10D shows the distribution of the number of genes identified as mostly responsible for the signal (dashed and dotted line) and of their false discovery rate (FDR; dashed line) as a function of the normalized sample variance. The FDR is calculated as the ratio of the black and dark gray distributions in FIG. 10C. Approximately, 5,500 genes are found responsible for the signal when adopting an FDR threshold of <0.001 (horizontal solid line). FIG. 10E shows the relationship between mean silhouette score, number of candidate cell clusters and level of resolution imposed through the Leiden clustering algorithm (Wolf et al., Genome Biology, 19:1-5, 2018), after removal of signals attributable to noise using Randomly. The optimal clustering solution (i.e., the clustering solution with the highest mean silhouette score) corresponds to three clusters (arrow). A complete description of this computational pipeline was previously published (Aparicio et al., Patterns, 1:100035, 2020).

FIGS. 11A-11C show the distribution of expression levels for myoepithelial-specific, ductal-specific and proliferation-specific biomarkers in scRNA-seq data from the patient-derived xenograft (PDX) line ACCX22. FIG. 11A shows the visualization using UMAP scatter-plots and violin plots of the distribution of the expression levels of three genes encoding for reference myoepithelial markers: smooth muscle actin alpha 2 (ACTA2), calponin (CNN1) and tumor protein p63 (TP63). All three genes are over-expressed in cells belonging to Cluster 1. FIG. 11B shows the visualization using UMAP scatter-plots and violin plots of the distribution of the expression levels of three genes encoding for markers of ductal/luminal cells in exocrine glands: keratin 7 (KRT7), keratin 18 (KRT18) and E74-like ETS transcription factor 5 (ELF5). All three genes are over-expressed in cells belonging to Cluster 2 and Cluster 3. FIG. 11C shows the visualization using UMAP scatter-plots and violin plots of the distribution of the expression levels of three genes encoding for established proliferation markers: DNA topoisomerase II alpha (TOP2A), cyclin-dependent kinase 1 (CDK1) and proliferating cell nuclear antigen (PCNA). All three genes are enriched in cells belonging to Cluster 3. The q-values reported within UMAP scatter-plots correspond to the false-discovery rates (FDRs) associated with each gene, calculated using the Benjami-ni-Hochberg method to correct for multiple comparisons, starting from the p-values for the difference in the mean expression level of each gene, computed between the cluster that preferentially expresses it and all other cell clusters (Student's t-test, two-tailed). The p-values associated with violin-plots correspond to the results of a Kruskal-Wallis H-test (performed as a confirmatory test for heterogeneous expression across the three clusters).

FIGS. 12A-12E show the analysis of MYB-NFIB fusion transcripts in myoepithelial-like (CD49f^{high}/KIT^{neg}) and ductal-like (CD49f^{low}/KIT⁺) cells. FIG. 12A shows the schematic illustration of MYB-NFIB fusion genes and resulting chimeric mRNAs. MYB-NFIB mRNAs undergo alternative splicing, usually in their NFIB portion, yielding distinct isoforms. In RNA-seq data sets, chimeric mRNAs can be identified either as chimeric reads (reads encompassing two genes) or as spanning reads (paired reads mapping to different genes in paired-end sequencing). FIGS. 12B-12E show the analysis of MYB, NFIB and MYB-NFIB expression in RNA-seq data from 5 bi-phenotypic PDX lines (ACCX5M1, ACCX6, ACCX14, ACCX22, SGTX6). MYB and NFIB were expressed in both CD49f^{high}/KIT^{neg} and CD49f^{low}/KIT⁺ cells. Expression levels were higher in CD49f^{high}/KIT^{neg} cells, but differences were not statistically significant (Student's t-test, paired samples, 2-tailed). In the 3 PDX lines with MYB-NFIB translocations predicted to yield fusion transcripts (ACCX5M1, ACCX14, SGTX6), analysis with the STAR-fusion software detected chimeric mRNAs in both CD49f^{high}/KIT^{neg} and CD49f^{low}/KIT⁺ cells and did not reveal statistically significant differences in expression levels (Student's t-test, paired samples, 2-tailed). In the same three PDX lines, the repertoire of splice variants identified by the STAR-fusion software did not display systematic differences between CD49f^{high}/KIT^{neg} and CD49f^{low}/KIT⁺ cells. Autologous pairs shared identical MYB breakpoints, indicating shared origin from a common cellular ancestor. Exons and genomic DNA breakpoints were mapped to the GRCh37 human reference genome.

FIGS. 13A-13H show the comparison of tumorigenic capacity and cell cycle distribution of CD49f^{high}/KIT^{neg} and CD49f^{low}/KIT⁺ cells from human ACCs. FIGS. 13A and 13B depict Extreme Limiting Dilution Analysis (ELDA) of tumorigenicity data from CD49f^{high}/KIT^{neg} and CD49f^{low}/KIT⁺ cells isolated by f^{low} cytometry from the ACCX5M1. FIGS. 13A and 13B show SGTX6 and PDX lines, respectively, reported using a Log-fraction plot. The slope of fitted lines (dark gray: CD49f^{high}/KIT^{neg}; light gray: CD49f^{low}/KIT⁺) represents the log-active cell fraction (shaded areas: 95% confidence interval). FIGS. 13C and 13D show the comparison of tumor volumes measured at euthanasia in animals engrafted with CD49f^{high}/KIT^{neg} (black dots) and CD49f^{low}/KIT⁺ (gray dots) cells purified by FACS from ACCX5M1 (FIG. 13C) and SGTX6 (FIG. 13D) PDX lines. Tumors originated from CD49f^{high}/KIT^{neg} cells reached higher volumes than those originated from CD49f^{low}/KIT⁺ cells (Welch's t-test, two-tailed). FIGS. 13E and 13F show the growth curves of individual tumors originated from in vivo injection of CD49f^{high}/KIT^{neg} (dark gray) and CD49f^{low}/KIT⁺ (gray) cells, isolated by FACS from ACCX5M1 (FIG. 13E) and SGTX6 (FIG. 13F) PDX lines. Tumors appeared between 150-300 days (ACCX5M1) and 250-450 days (SGTX6) post-engraftment. FIG. 13G shows analysis of cell-cycle distribution in CD49f^{high}/KIT^{neg} and CD49f^{low}/KIT⁺ cells from five bi-phenotypic PDX lines. FIG. 13H shows the percentage of cells in the G2/M phase of the cell cycle (DAPI^{high}) was lower in CD49f^{high}/KIT^{neg} as compared to CD49f^{low}/KIT⁺ cells (p=0.038; Mann-Whitney U-test, two-tailed).

FIGS. 14A-14H. show the comparative histo-morphological analysis of solid tumor tissues and three-dimensional (3D) organoid cultures established from the same human Adenoid Cystic Carcinoma (ACC) PDX line (ACCX5M1). FIGS. 14A and 14B respectively show the analysis by immuno-histochemistry (IHC) of TP63 and KIT expression in a solid tumor lesion established by sub-cutaneous transplantation t in immuno-deficient NOD/SCID/IL2Rγ^{-/-} (NSG) mice of a bi-phenotypic PDX line (ACCX5M1). The tumor tissues display a classical “cribriform” architecture, characterized by pseudo-cysts surrounded by myoepithelial-like (TP63⁺) cells (FIG. 14A; arrowheads), and ring/tubular-like structures formed by ductal-like (KIT⁺) cells (FIG. 14B; arrows). FIGS. 14C-14H show the analysis by immuno-histochemistry (IHC) of TP63 (FIGS. 14C-14E) and KIT (FIGS. 14F-14H) expression in 3D organoids established from the ACCX5M1 PDX line. In all three cases, the organoids display a heterogeneous cell composition, characterized by the co-existence of two populations with mutually exclusive expression of TP63 (FIGS. 14C-14E; arrowheads) and KIT (FIGS. 14F-14H; arrows). In 3D organoids, myoepithelial-like cells (TP63⁺) are positioned at the outer surface, where they interface with the Matrigel scaffolding that acts as the 3D support for the organoids' growth (and which contains basement membrane proteins and proteo-glycans similar to those found in the pseudo-cysts of primary ACCs), while ductal-like cells (KIT⁺) are positioned at the center, where they arrange to form ring/tubular-like structures, reminiscent of those observed in primary tumors. Scale bars: 25 μm.

FIGS. 15A-15F show the statistical modeling of the distribution of data consisting in the percentage of cells displaying a myoepithelial phenotype (CD49f^{high}/KIT⁺), as measured sequentially in independent tumors. FIG. 15A shows the visualization by histogram of the distribution of the percentage of cancer cells displaying a myoepithelial-like phenotype (CD49f^{high}/KIT^{neg}) across a series of tumors (n=17) established by sub-cutaneous engraftment in immune-deficient NOD/SCID/IL2Rγ^{-/-} (NSG) mice of the same patient-derived xenograft (PDX) line (SGTX6), representative of a biphenotypic Adenoid Cystic Carcinoma (ACC). Overlayed to the histogram are the curves representing the continuous density distributions of the primary data (solid line) and of a normal distribution (dashed line) and a β-distribution (dotted line) that share the same mean and standard deviation of the primary data. FIG. 15B shows the distribution of the primary data was tested for deviation from normality using a Shapiro-Wilk test (p=0.9973) and then visualized for similarity to the normal distribution using a quantile-to-quantile (QQ) plot, which displayed tight adherence to the line of equality (gray). FIGS. 15C-15F show the visualization using histograms and QQ-plots of the distribution of the mean values for the same percentage data, as estimated using a bootstrapping approach to perform serial re-samplings (n=1,000), each consisting of either 3 (FIGS. 15C and 15D) or 5 (FIGS. 15E and 15F) experimental replicates, picked randomly and with replacements. All analyses were conducted using the “R” software (v4.1.2).

FIGS. 16A-16S show that the perturbation of retinoic acid (RA) signaling does not induce proliferation in ACC organoids. FIGS. 16A-160 show the analysis of organoid morphology and histology, following treatment with either activators (ATRA; direct agonist) or inhibitors (BMS493; inverse agonist) of RAR/RXR signaling. Organoids were established from a PDX line with bi-phenotypic histology (ACCX5M1) and treated for one week with either DMSO (FIGS. 16A-16E), 10 μM ATRA (FIGS. 16F-16J) or 10 μM BMS493 (FIGS. 16K-16O). Scale bars=100 μm. Treatment with ATRA did not change organoid morphology (FIG. 16F) but increased the number of KIT⁺ cells (FIG. 16I), as visualized by immunohistochemistry (IHC). Treatment with BMS493 caused a dramatic change in organoid morphology, characterized by the appearance of dense areas in the organoid centers, when observed using bright-field microscopy (FIG. 16K, arrowheads). When organoids were stained with hematoxylin and eosin (FIG. 16H and FIG. 16E), these areas consisted of an eosin-rich material, with apoptotic nuclei (FIG. 16L, arrowheads). FIGS. 16P-16S show the analysis by f^{low} cytometry of cell-cycle distribution in organoids established from ACCX5M1 (FIG. 16P) and ACCX6 (FIG. 16Q) PDX lines, following 1 week of treatment with either DMSO, ATRA (10 μM) or BMS493 (10 μM). Treatment with either ATRA or BMS493 did not increase the percentage of cells in the G2/M phase of the cell-cycle in ACCX5M1 (FIG. 16R) and ACCX6 (FIG. 16S) organoids. Experiments included at least three replicates (n=3 wells/condition). Error bars: mean+/−standard deviation. Organoid cultures were tested for the presence of a statistically significant increase in the percentage of cells in the G2/M phase following treatment, using a one-way Mann-Whitney U-test (n.s.=non-significant).

FIGS. 17A-17I show the in vivo anti-tumor activity and toxicity of BMS493. FIG. 17A shows the individual growth curves of ACCX9 tumors treated with DMSO (gray) or BMS493 (black). Crosses (+) identify two animals who were sacrificed early, due to health deterioration. FIG. 17B shows growth rates of ACCX9 tumors treated with either DMSO (n=6) or BMS493 (n=7). Two treatment cohorts are identified by different symbols (circles=cohort 1; triangles=cohort 2). Differences in mean growth rates were statistically significant (Welch's t-test; **p<0.01). Black symbols identify the two animals who were sacrificed because of health deterioration. FIG. 17C shows animal weight over the course of in vivo treatment with BMS493 (†: animals sacrificed due to health deterioration). FIG. 17D shows individual growth curves of ACCX11 tumors treated with DMSO (gray) or BMS493 (black). FIG. 17E shows the growth rates of ACCX11 tumors treated with either DMSO (n=4) or BMS493 (n=5). Differences in mean growth rates were statistically significant (Welch's t-test; **p<0.01). FIG. 17F shows the animal weight over the course of in vivo treatment with BMS493. FIG. 17G shows the individual growth curves of ACCX5M1 tumors treated with DMSO (gray) or BMS493 (black). Crosses (†) identify two animals who either were sacrificed early due to health deterioration or who died at the final time point. FIG. 17H shows the growth rates of ACCX5M1 tumors treated with either DMSO (n=6) or BMS493 (n=6). Two treatment cohorts are identified by different symbols (circles=cohort 1; triangles=cohort 2). Differences in mean growth rates were statistically significant (Welch's t-test; *p<0.05). Black symbols denote the two animals who either were sacrificed early due to health deterioration or died at the final time point. FIG. 17I shows animal weights over the course of treatment (†: animals sacrificed or found dead).

### DETAILED DESCRIPTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

As used herein, the terms “low” and “high” when used in the context of the expression of a cell-surface marker refer to relative expression level as determined using f^{low} cytometry methods. Flow cytometry quantifies expression levels as a relative increase in fluorescence as compared to a baseline level of fluorescence. As a general rule, the baseline level of fluorescence is established during each experiment and corresponds to the lower range of auto-fluorescence of the same preparation of cells (i.e., the fluorescence displayed by the same preparation of cells in the absence of labeling with fluorescent antibodies that are specifically directed against the antigens being measured). The cytometry instrument detectors are adjusted so that unlabeled cells distribute across a range of fluorescence that does not exceed 10e3 (1,000-fold) of the baseline autofluorescence. The discrimination between “low” expression and “high” expression levels is typically associated with the visual assessment of a bimodal distribution of expression levels in the positive space (i.e., range of fluorescence that exceed 10e3 (1,000-fold) of the baseline autofluorescence). Optimization of f^{low} cytometry methods for assessing cell surface marker expression level is well-established in the prior art (see, for example, Herzenberg et al., Nature Immunology, 2006, 7:681-685).

As used herein, the terms “positive”, “pos”, or “+” and the terms “negative”, “neg”, or “−” when used in the context of the expression of a cell-surface marker from flow cytometry results refer to a fluorescence level of that is superior to 10e3 (>1,000-fold) the baseline autofluorescence for indication of positive expression and a fluorescence level that is inferior to 10e3 (<1,000-fold) the baseline autofluorescence for indication of negative expression. The terms are also applicable to the assessment of the expression level of a cell-surface marker using immunohistochemistry (IHC) methods (which an established methodology, see, for example, Meyerholz and Beck, Laboratory Investigation, 2018, 98:844-855). Being able to detect the presence of the cell surface marker using IHC indicate positive expression, while inability to detect the presence of the cell surface marker indicates negative expression.

As used herein, the term “tumor aggression” or the term “aggression” used in the describing a trait of a tumor refers to rapid growth and/or rapid spread (for example, rapidly progressing through the initial stages of metastasis).

As used herein, the term “treating” or “treatment” has the same meaning in the present context as commonly understood to one of ordinary skill in the art. Specifically, “treating” a disease or condition means providing any form of relief to the patient from the disease or condition or its recurrence, including without limitation, reducing severity, reducing expected further development, or reducing the expected duration, of the disease or condition or any symptoms or recurrence thereof, or otherwise providing relief to the patient from normally-expected development, severity, duration, or any lasting consequences of the disease or condition or any of its symptoms. In some aspects, “treating” or “treatment of” adenocarcinoma refers to reducing further advancement of the adenocarcinoma, for example, by killing tumor cells, inhibiting, or slowing the growth of tumor cells, and/or inhibiting metastasis.

The abbreviations used herein are defined as follows:
2D: Two-dimensional
3D: Three-dimensional
ACC: Adenoid Cystic Carcinoma
ATRA: All-trans Retinoic Acid
dbGAP: Database of Genotypes and Phenotypes
DNhRARa: Dominant Negative human Retinoic Acid Receptor alpha
DMSO: Dimethyl-sulfoxide
ED₅₀: Effective dose 50%
EGFP: Enhanced Green Fluorescent Protein
ELDA: Extreme Limiting Dilution Analysis
FACS: Fluorescence Activated Cell Sorting.
FDR: False Discovery Rate
IACUC: Institutional Animal Care and Use Committee
IHC: Immunohistochemistry
NSG: NOD·Cg-Prkdc^{scid} Il2rg^{tmlWj1}/SzJ.
PCA: Principal Component Analysis
PC1: First Principal Component
PDX: Patient-Derived Xenograft
RA: Retinoic Acid
RAR: Retinoic Acid Receptor.
RMT: Random Matrix Theory
RXR: Retinoid-X Receptor
scRNA-seq: single-cell RNA-sequencing
SG: Salivary Gland

Disclosed herein are methods and compositions related to the diagnosis and treatment of adenocarcinomas, which may originate from salivary gland, lung, breast tissue, colon, kidney, pancreas, ovary, and prostate. In particular embodiments, methods and compositions related to the diagnosis and treatment of adenoid cystic carcinoma (ACC), lung cancer, breast cancer, pancreatic cancer, and prostate cancer are described. In one aspect, a method of reducing tumorigenicity and/or aggression of adenocarcinoma cells, such as those from ACC, breast cancer, pancreatic cancer, or prostate cancer, is disclosed. In another aspect, a method of reducing viability of adenocarcinoma cells is disclosed. In yet another aspect, a method of reducing the size of a tumor is disclosed, wherein the tumor is of an adenocarcinoma such as ACC, lung cancer, breast cancer, pancreatic cancer, or prostate cancer. In still another aspect, a method of inhibiting growth of ACC, lung cancer, breast cancer, pancreatic cancer, and prostate cancer is disclosed. Uses of cell surface markers for subtyping adenocarcinoma cells are also described herein. Further described herein are therapeutic agents useful for the treatment of leukemia, non-small cell lung cancer, colon cancer, brain cancer, melanoma, sarcoma, ovarian cancer, renal cancer, prostate cancer, breast cancer, pancreatic cancer, and ACC.

Adenoid cystic carcinoma (ACC) is a lethal form of cancer for which there are currently no approved drug treatments. ACCs usually originate in secretory glands of the cranio-facial district (i.e. salivary glands, lacrimal glands) and preferentially affect young and middle-aged adults. These malignancies are characterized by a high propensity towards local invasion by peri-neural infiltration (i.e. towards the invasion of surrounding tissues by dissemination along nerve sheaths) and a high propensity towards distant-site metastasis (i.e. towards the dissemination to other organs through the blood circulation). There are currently no FDA-approved systemic or targeted therapies for the medical treatment of human ACCs.

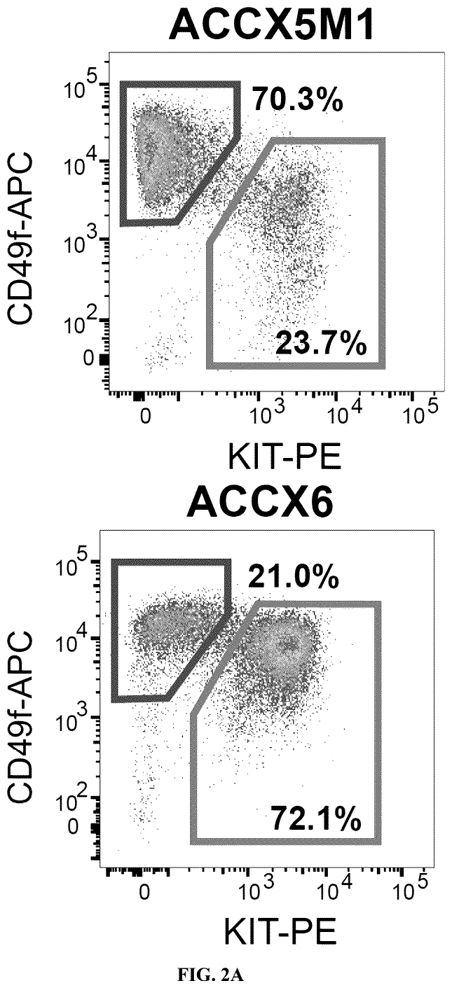

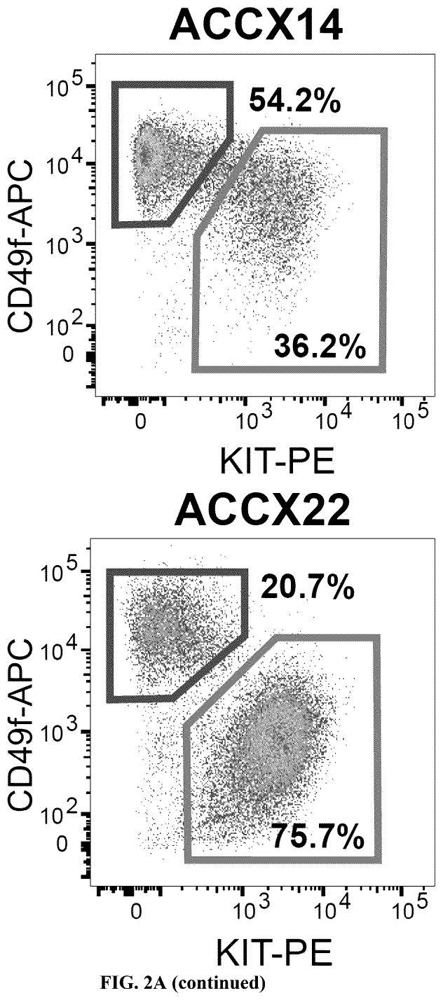

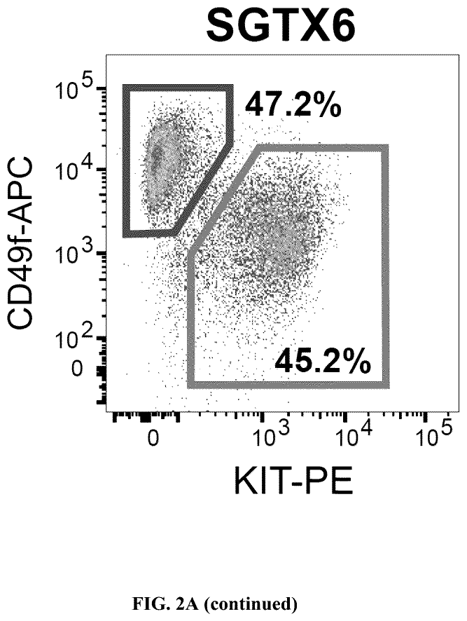

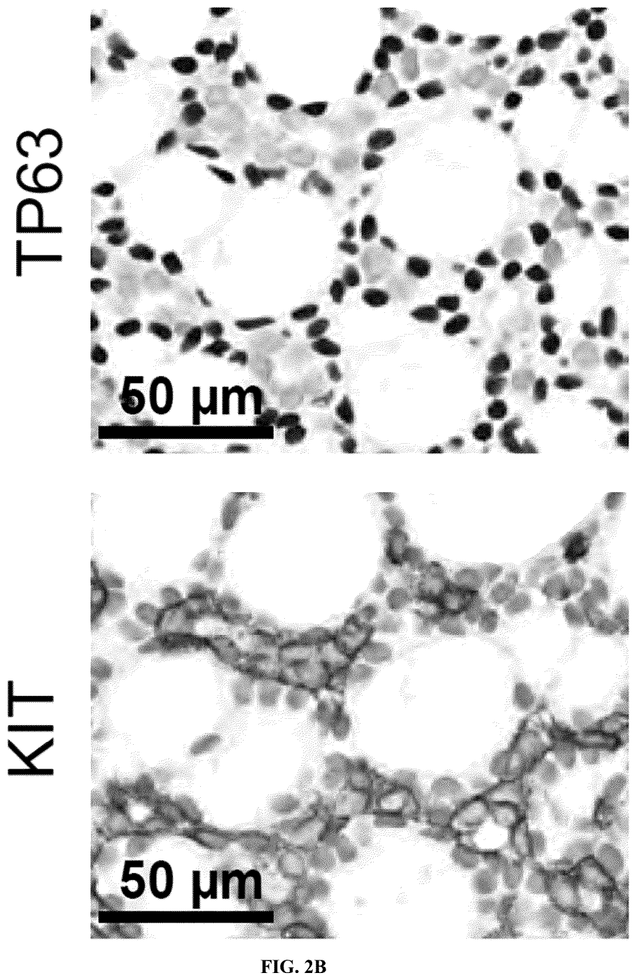

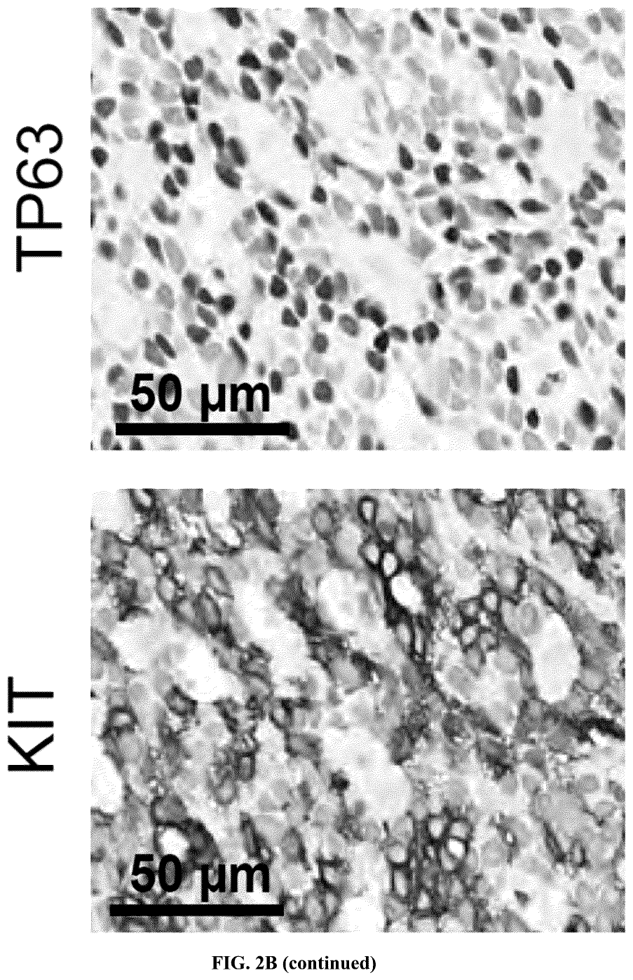

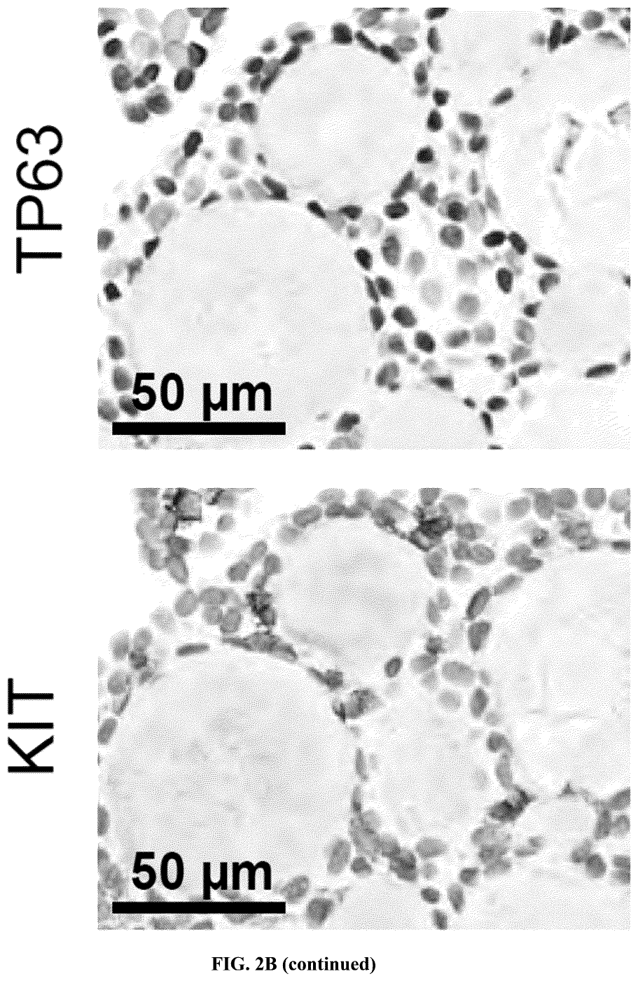

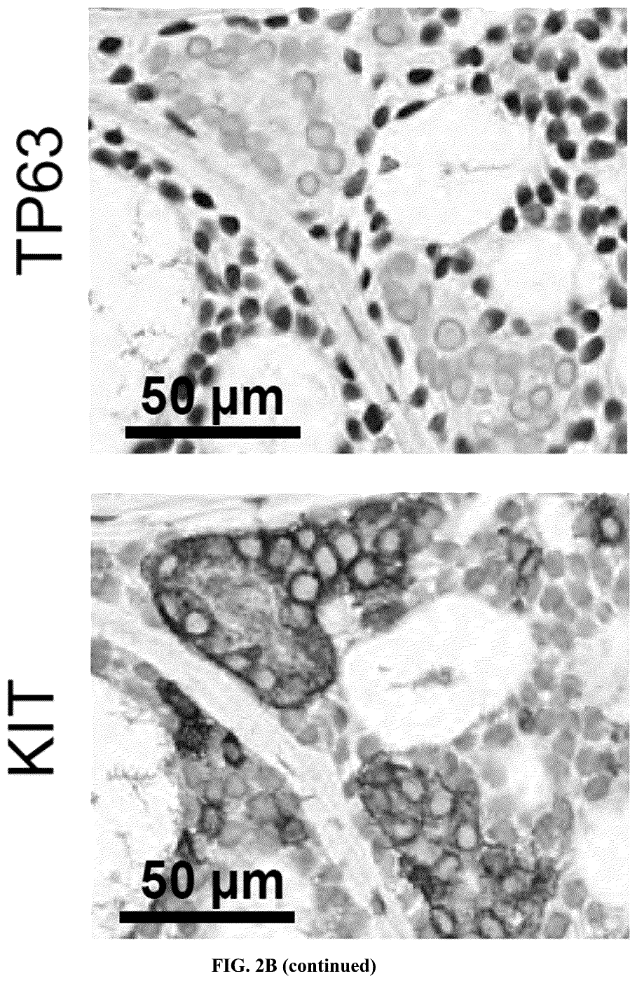

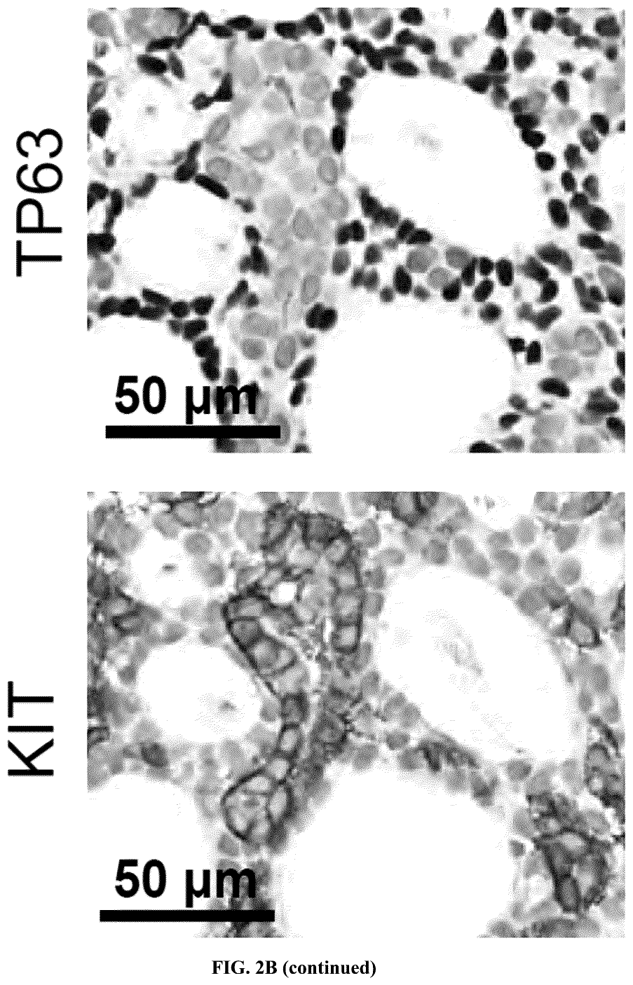

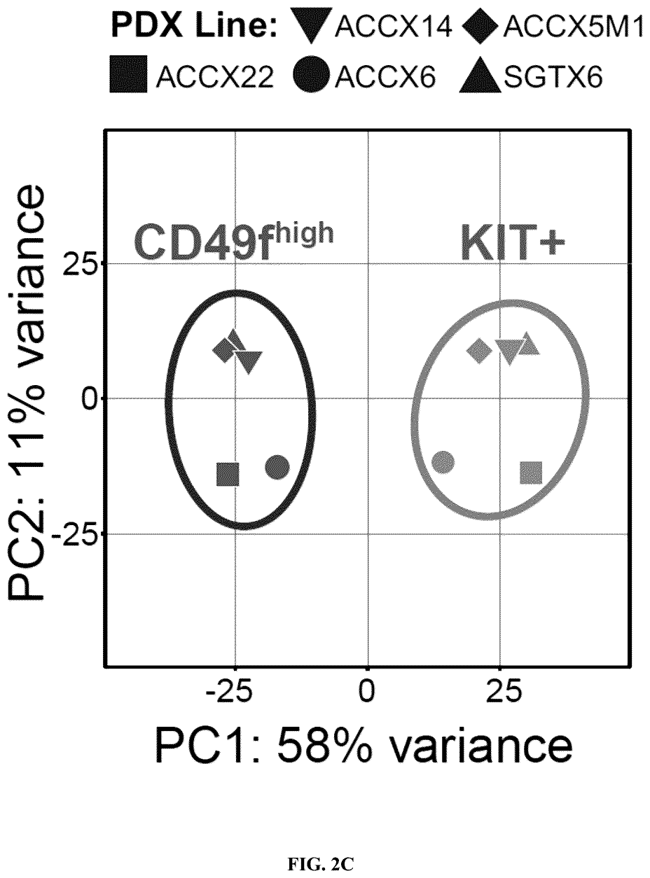

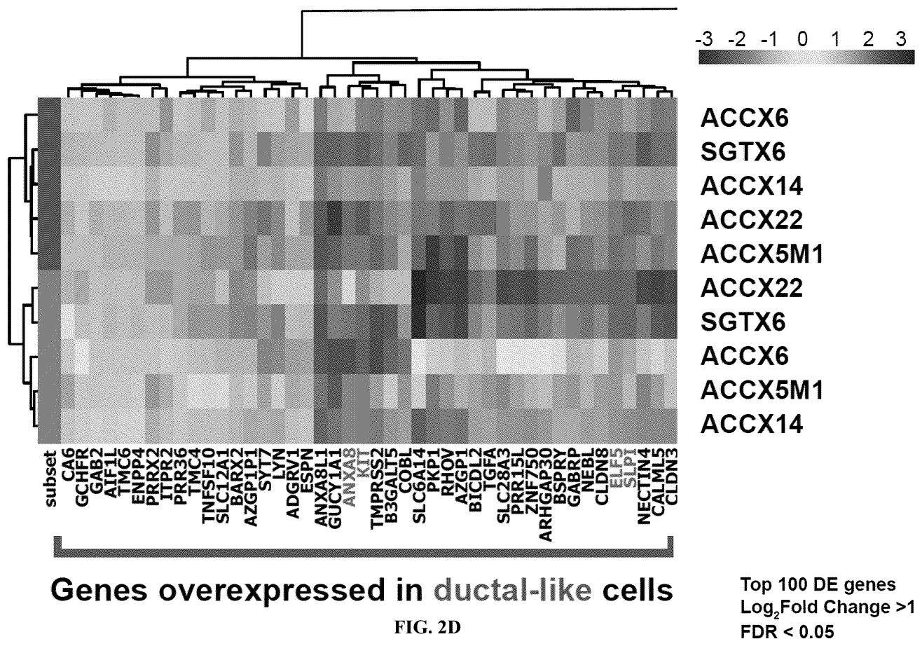

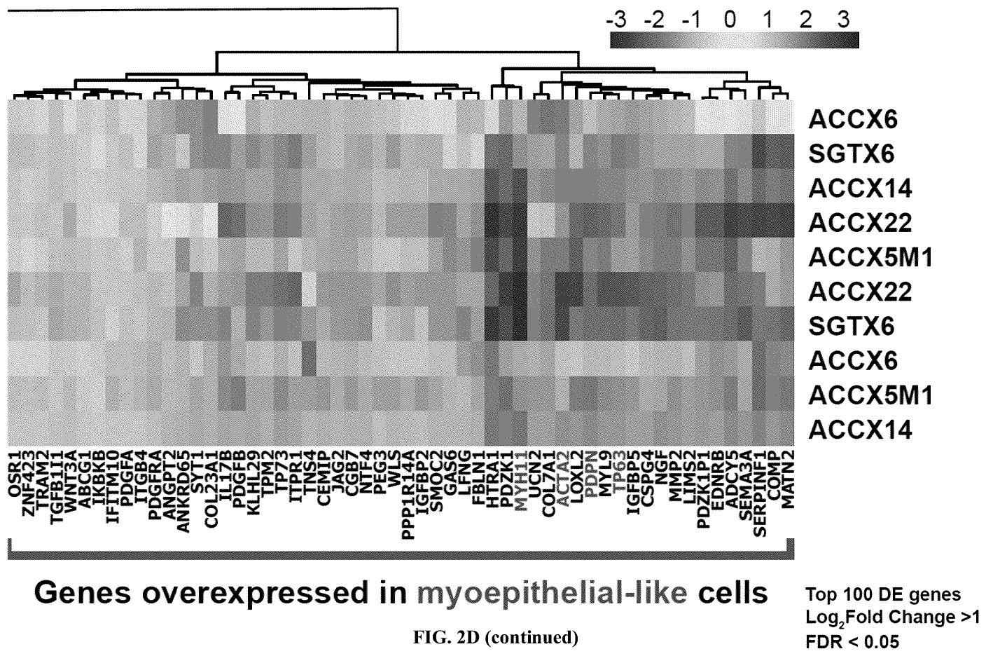

From a histological point of view, ACCs are usually characterized by a “bi-phasic differentiation” in that the malignant tissues contain two distinct populations of cancer cells, which are commonly referred to as “myoepithelial-like” and “ductal-like” cells. As disclosed herein, cell-surface markers (for example, CD49f, TP63, KIT) that enable the differential purification and comparative study of the two sub-types of malignant cells (myoepithelial-like and ductal-like) known to co-exist in human ACCs were identified for the first time. As such, CD49f and KIT/CD117 cell surface markers enable differential purification and quantification of the two populations of ACC through sorting mechanisms, such as fluorescence activated cell sorting (FACS). On the other hand, the combination of TP63 and KIT/CD117 enable detecting the presence of myoepithelial-like and ductal-like cells via immunohistochemistry methods. Within human ACCs, TP63 and KIT/CD117 are expressed in a mutually exclusive manner. TP63 is a marker of myoepithelial-like cells (TP63⁺/KIT^{neg}), because TP63 is not expressed in ductal-like cells, which are (TP63^{neg}/KIT⁺), as shown in FIG. 2B. Myoepithelial-like and ductal-like ACC cells can also be distinguished by their expression of KIT/CD117.

The data in the examples reveal that the two cell-types do not represent distinct genetic clones, but distinct developmental lineages (i.e., distinct cell-types that originate as a result of multi-lineage differentiation processes, akin to those that enable stem-cell populations to sustain the homeostatic turnover of normal tissues). With the ability to separate these two cell populations, it was discovered that myoepithelial-like cells (CD49f^{high}, TP63⁺, KIT^{neg}) are associated with more aggressive biological properties as compared to ductal-like cells (CD49f^{low}, TP63^{neg}, KIT⁺), when tested for their tumorigenic capacity (i.e. their capacity to sustain the formation of a new tumor upon xenotransplantation in immuno-deficient mice). Myoepithelial-like cells are highly tumorigenic upon xeno-transplantation in immune-deficient animals, despite their low proliferation rates. In tumors originated from exocrine glands (e.g., breast cancer), myoepithelial-like cells are often considered tumor-suppressive [65, 66]. The findings caution against this interpretation in ACCs, and indicate that, in order to be curative, treatment strategies will need to eradicate myoepithelial-like components. Furthermore, the data show that, in ACCs, myoepithelial-like cells act as progenitors of ductal-like cells and that myoepithelial-to-ductal differentiation is promoted by RAR/RXR signaling. These findings provide a mechanistic explanation for the conflicting results that have been recently obtained in studies that tested ATRA's anti-tumor activity in human ACCs. ATRA displayed marked anti-proliferative activity against PDX models [55, 56], but appeared to provide limited benefit when administered to patients [67]. It is now hypothesized that, in ACC patients, the therapeutic benefit of ATRA might be short-lived because of the cytostatic nature of its effects, which consist in a transient perturbation of the tumor tissues' cell composition.

Also as shown in Examples, direct agonists of either retinoic acid receptor (RAR) or retinoid x receptor (RXR) signaling (such as all-trans retinoic acid (ATRA) and bexarotene) can modify the cell composition of human ACCs, inducing the differentiation of myoepithelial-like cells into ductal-like cells, thus changing their relative representation in malignant tissues. For example, administration of direct agonists of either retinoic acid receptor (RAR) or retinoid x receptor (RXR) signaling to ACC cell reduces the percentage of myoepithelial-like cells and increases the percentage of ductal-like cells. It should be noted that that suppression of RAR/RXR signaling induces selective death of ductal-like cells. This finding provides an opportunity for the selective pharmacological targeting of ACCs, especially of cases with solid histology, which are characterized by mono-phenotypic expansions of ductal-like cells. These tumors often originate during the natural progression of ACCs, following the acquisition of NOTCH1 activating mutations, in a scenario that is reminiscent of the “blast crisis” observed in chronic myelogenous leukemias (CMLs), whereby a population of more differentiated, yet highly proliferative cells becomes dominant, due to mutations that aberrantly activate self-renewal [68-70]. The data indicate that, in solid ACCs, treatment with an inverse agonist of RAR/RXR signaling (BMS493) have robust anti-tumor activity. While agonists of RAR/RXR signaling have been extensively explored as anti-tumor agents in humans [71-75], inverse agonists, have not. As disclosed herein, treatment with an inverse agonist of RAR/RXR signaling (for example, BMS-493 and AGN-193109) selectively kills ductal-like (CD49f^{low}, KIT⁺) adenocarcinoma cells. Thus, RAR/RXR signaling is not only required for the differentiation of myoepithelial-like cells into ductal-like cells but also for the continuing survival of ductal-like cells. Accordingly, modulating RAR/RXR signaling is a promising therapeutic strategy in the treatment of adenocarcinomas, such as those from ACC, breast cancer, pancreatic cancer, and prostate cancer. Inverse agonist of RAR/RXR signaling may also be useful for treating melanoma and sarcomas, which also have altered RAR/RXR signaling. For example, melanomas express high levels of ALDH1A3, the enzyme that synthesizes retinoic acid.

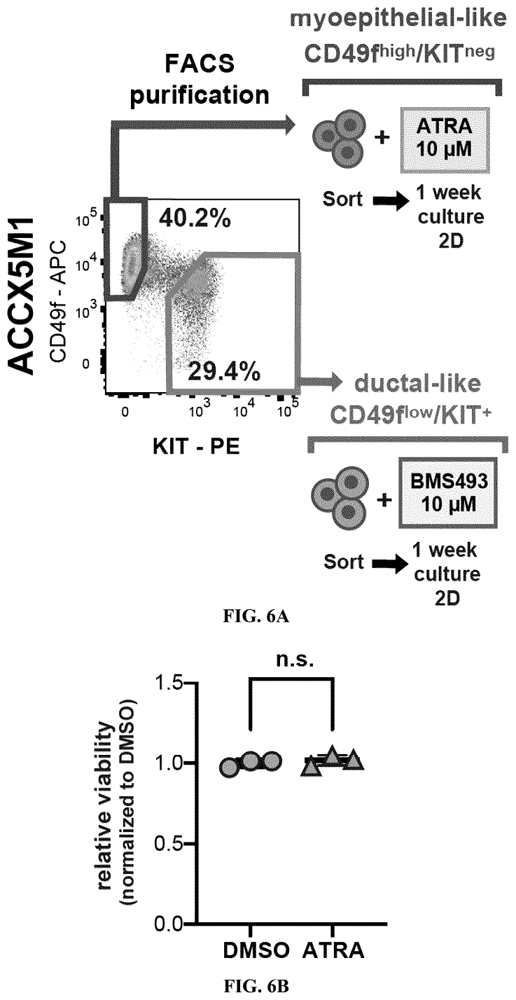

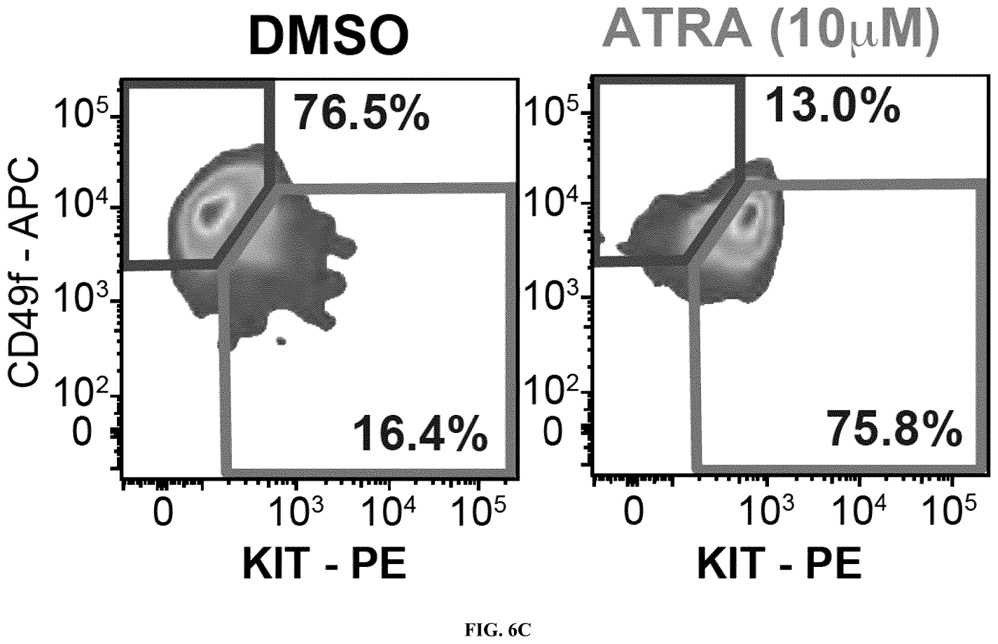

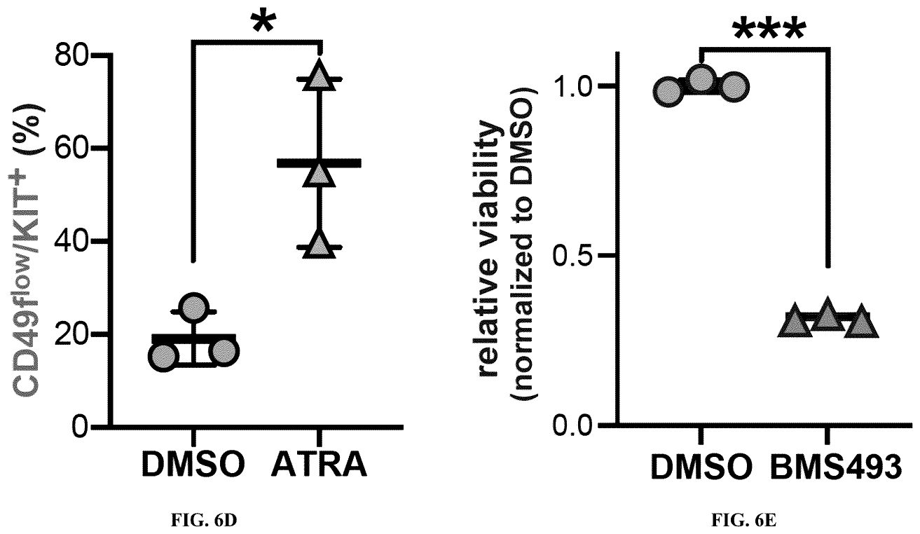

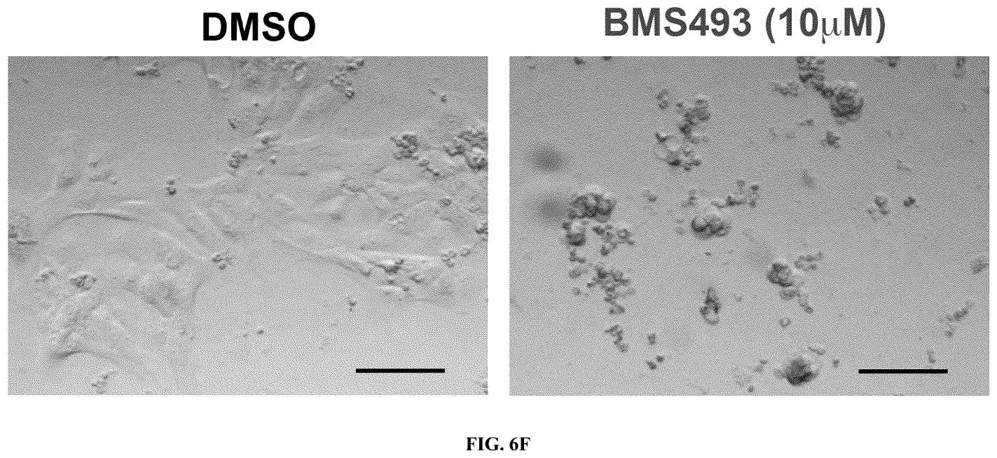

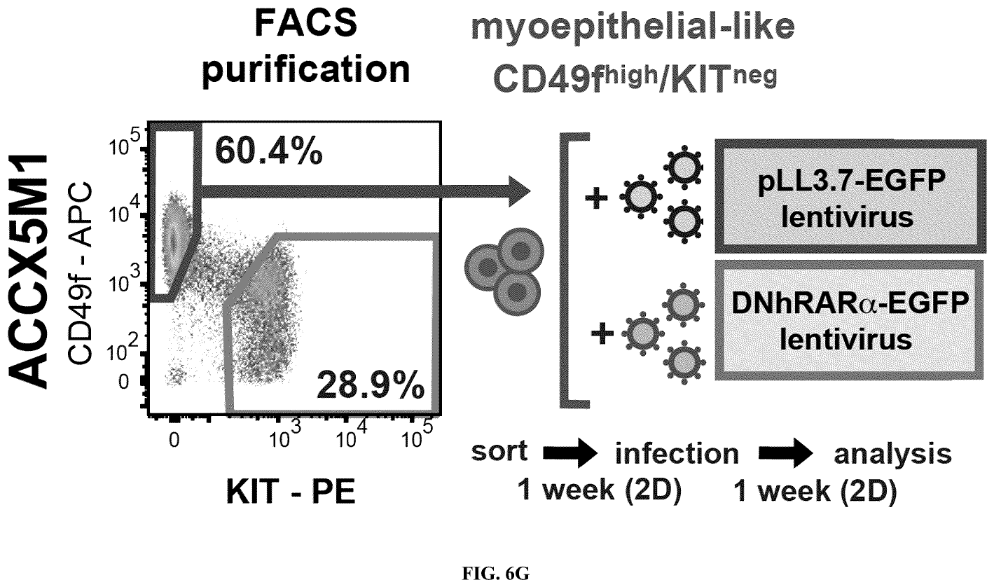

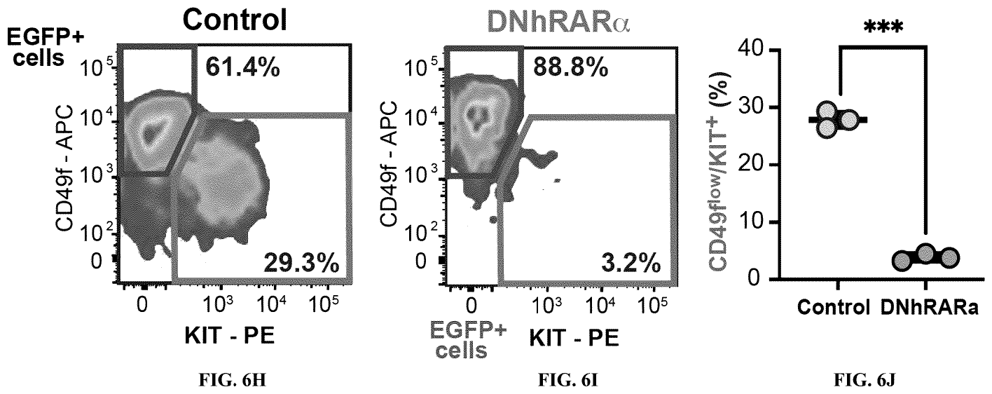

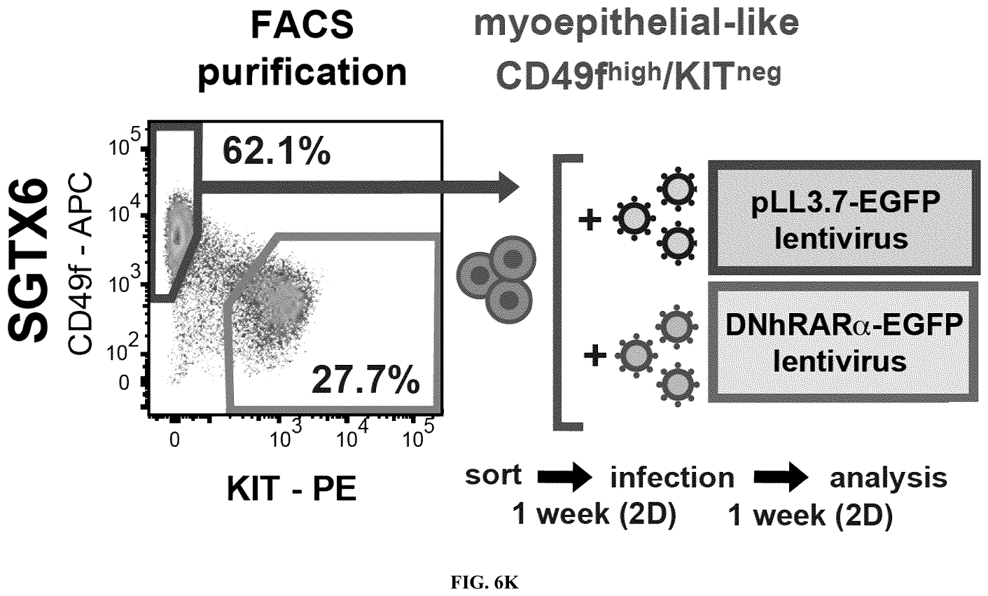

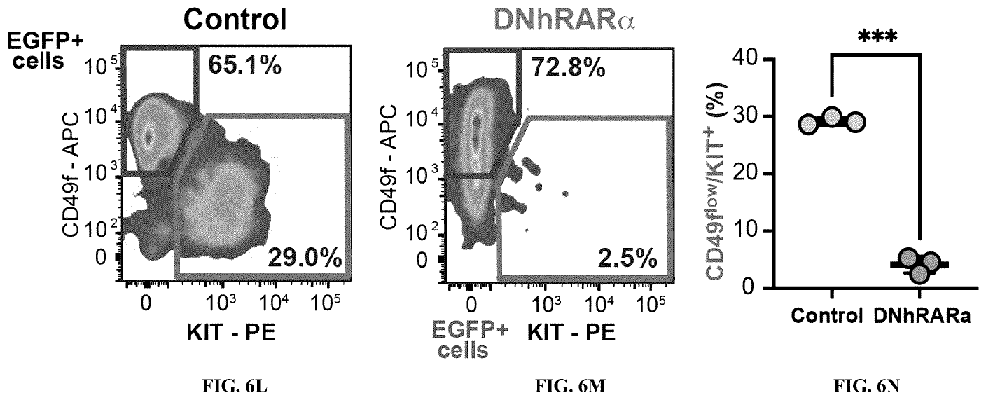

In addition, the use of that infection of myoepithelial-like (CD49f^{high}, KIT^{neg}) cells with a lentivirus encoding a “dominant-negative” version of the RAR-alpha receptor (DN-hRAR-alpha) can fully abrogate their differentiation into ductal-like cells, thus phenocopying the effects of the pharmacological inhibitors of RAR/RXR signaling (e.g, the inverse agonists BMS-493 and AGN-193109). This observation, show in FIGS. 6G-6N, is very important, because it shows that: 1) the therapeutic activity observed following administration of inverse agonists of RAR/RXR signaling (BMS-493, AGN-193109) is unlikely to be caused by “off-target” effects (i.e., is not attributable to an unknown pharmacological activity of BMS-493 and AGN-193109 on receptors other than RAR/RXRs); and 2) it is conceivable that other agents capable of suppressing RAR/RXR signaling might be leveraged for the therapeutic management of ACCs, irrespective of their chemical nature (e.g., recombinant cDNAs encoding DN-hRAR-alpha constructs, delivered using viral vectors for gene therapy).

In one aspect, the method of reducing tumorigenicity and/or aggression of adenocarcinoma cells (for example, those from ACC, breast cancer, pancreatic cancer, or prostate cancer) comprises administering a therapeutic agent that activates retinoic acid receptor and/or retinoid-X receptor signaling to the adenocarcinoma cells. In some aspects, the therapeutic agent is administered at a dose effective to induce myoepithelial-to-ductal differentiation the adenocarcinoma cells. In some implementations, the method further comprises detecting the expression of CD49f, TP63, and/or KIT/CD117 in the adenocarcinoma cells. Upon detection of more than 5% of the adenocarcinoma cells express TP63, less than 95% of the adenocarcinoma cells express KIT/CD117, or the adenocarcinoma cells have high expression of CD49f, the adenocarcinoma cells are administered the therapeutic agent that activates retinoic acid receptor and/or retinoid-X receptor signaling. In some implementations, the method further comprises administering a therapeutic agent that inhibits retinoic acid receptor and/or retinoid-X receptor signaling to the adenocarcinoma cells after the administration of the therapeutic agent that activates retinoic acid receptor and/or retinoid-X receptor signaling.

In some aspects, the expression levels of CD49f and KIT/CD117 are detected using f^{low} cytometry methods, for example, fluorescence-activated cells sorting. In other aspects, the expression levels of TP63 and KIT/CD117 are detected using immunohistochemistry methods. Accordingly, the step of the detecting the expression of CD49f, TP63, and/or KIT/CD117 in the adenocarcinoma cells comprises combining an antibody of CD49f and/or an antibody of KIT/CD117 with the adenocarcinoma cells. In certain implementations, the antibody of CD49f and/or the antibody of KIT/CD11 are conjugated to a fluorescence marker, a magnetic particle, or microbubbles. In some implementations, the method further comprises sorting the adenocarcinoma cells based on binding of the antibody of CD49f, the antibody of TP63, and/or the antibody of KIT/CD117 to the adenocarcinoma cells.

In another aspect, the method of method of reducing viability of adenocarcinoma cells comprises detecting the expression of CD49f, TP63, and/or KIT/CD117 in the adenocarcinoma cells; and administering a therapeutic agent that inhibits retinoic acid receptor and/or retinoid-X receptor signaling to the adenocarcinoma cells. Upon detection of less than 5% of the adenocarcinoma cells express TP63, more than 95% of the adenocarcinoma cells express KIT/CD117, or the adenocarcinoma cells have low expression of CD49f, the adenocarcinoma cells are administered the therapeutic agent that inhibits retinoic acid receptor and/or retinoid-X receptor signaling. In some implementations, upon the detection of more than 5% of the adenocarcinoma cells express TP63, less than 95% of the adenocarcinoma cells express KIT/CD117, or the adenocarcinoma cells have high expression of CD49f, the method further comprises administering a therapeutic agent that activates retinoic acid receptor and/or retinoid-X receptor signaling to the adenocarcinoma cells prior to administering the therapeutic agent that inhibits retinoic acid receptor and/or retinoid-X receptor signaling to the adenocarcinoma cells. The administration of the therapeutic agent that activates retinoic acid receptor and/or retinoid-X receptor signaling produces a population of treated adenocarcinoma cells expressing KIT/CD117 without expression of TP63 or expressing KIT/CD117 with low expression of CD49f. In some aspects, the adenocarcinoma cells are from ACC, breast cancer, pancreatic cancer, or prostate cancer.

In such methods, the step of the combining an antibody of CD49f and/or an antibody of KIT/CD117 with the adenocarcinoma cells. In certain implementations, the antibody of CD49f and/or the antibody of KIT/CD11 are conjugated to a fluorescence marker, a magnetic particle, or microbubbles. In some implementations, the method further comprises sorting the adenocarcinoma cells based on binding of the antibody of CD49f, the antibody of TP63, and/or the antibody of KIT/CD117 to the adenocarcinoma cells. In some aspects, the expression levels of CD49f and KIT/CD117 are detected using flow cytometry methods, for example, fluorescence-activated cells sorting. In other aspects, the expression levels of TP63 and KIT/CD117 are detected using immunohistochemistry methods.

In yet another aspect, the method of reducing the size of a tumor comprises providing a tumor sample from a subject; detecting the expression of at least one cell-surface marker (selected from the group consisting of: CD49f, TP63, and KIT/CD117) in the tumor sample; and administering a therapeutic agent that inhibits retinoic acid receptor and/or retinoid-X receptor signaling to the subject with a tumor sample comprising more than 95% of cells expressing KIT/CD117 or with a tumor sample comprising less than 5% of cells expressing TP63. In some implementations, the tumor sample of the sample administered the therapeutic agent that inhibits retinoic acid receptor and/or retinoid-X receptor signaling has low expression level of CD49f. In some implementations, the tumor is from the salivary gland, lung, breast tissue, colon, kidney, pancreas, ovary, or prostate. In some embodiments, the tumor sample is provided from a subject with leukemia, non-small cell lung cancer, colon cancer, brain cancer, melanoma, sarcoma, ovarian cancer, renal cancer, prostate cancer, breast cancer, pancreatic cancer, or ACC.

In some implementations, the method of reducing the size of a tumor further comprises confirming the expression of at least one cell-surface marker in the tumor sample selected from the group consisting of: ACTA2, MYH11, PDPN, ELF5, SLPI, and ANXA8. In further implementations, the method of reducing the size of a tumor also comprises a therapeutic agent that inhibits retinoic acid receptor and/or retinoid-X receptor signaling and is administered to the subject with a tumor sample comprising more than 95% of cells expressing KIT/CD117 and at least a second cell-surface marker selected from the group consisting of ELF5, SLPI, and ANXA8. In some implementations, the expression levels of cell surface markers, such as CD49f and KIT/CD117, are detected using flow cytometry methods, for example, fluorescence-activated cells sorting. In other aspects, the expression levels of cell surface markers, such as TP63, KIT/CD117, ACTA2, MYH11, PDPN, ELF5, SLPI, and ANXA8, are detected using immunohistochemistry methods. Accordingly, the step of the detecting the expression of the cell surface markers in the adenocarcinoma cells comprises combining antibodies of the cell surface markers with the adenocarcinoma cells. In certain implementations, the antibodies are conjugated to a fluorescence marker, a magnetic particle, or microbubbles. In some implementations, the method further comprises sorting the adenocarcinoma cells based on binding of the antibodies of the cell surface markers to the adenocarcinoma cells. Cell sorting may be achieve using conventional methods, including fluorescence-activated cell sorting.

In another aspect, the method of reducing the size of a tumor comprises providing a tumor sample from a subject; sorting cells from the tumor sample based on expression level of CD49f, TP63, and KIT/CD117; and administering a therapeutic agent that inhibits retinoic acid receptor and/or retinoid-X receptor signaling to the subject with a tumor sample comprising more than 95% of cells expressing KIT/CD117 or less than 5% of cells expressing TP63 or a tumor sample having low expression of CD49f. In some implementations, the method further comprises administering a therapeutic agent that activates retinoic acid receptor and/or retinoid-X receptor signaling to the subject with a tumor sample more than 5% of the cells expressing TP63 or less than 95% of the cells expressing KIT/CD117 or a tumor sample having high expression of CD49f. In such implementations, the therapeutic agent that activates retinoic acid receptor and/or retinoid-X receptor signaling is administered prior to the therapeutic agent that inhibits retinoic acid receptor and/or retinoid-X receptor signaling. The administration of the therapeutic agent that activates retinoic acid receptor and/or retinoid-X receptor signaling alters the cells of the tumor to produce a population of cells expressing KIT/CD117 without expression of TP63 or expressing KIT/CD117 with low expression of CD49f.

In still another aspect, the method of inhibiting growth of ACC in a subject comprises obtaining an ACC tumor sample from the subject; sorting cells of the tumor sample based on the expression of CD49f and KIT/CD117 in the ACC tumor sample (for example, through flow cytometry); and administering a therapeutic agent to the subject that inhibits retinoic acid receptor and/or retinoid-X receptor signaling upon the indication of the presence of ductal-like ACC cells in the sample. The presence of CD49f^{low}/KIT⁺ cells indicates the presence of ductal-like ACC cells. The presence of CD49f^{high}/KIT^{neg} cells indicates the presence of myoepithelial-like ACC cells. In some implementations, where the sorting step indicates the tumor sample comprises myoepithelial-like ACC cells, the method further comprising administering to the subject a therapeutic agent that activates retinoic acid receptor and/or retinoid-X receptor signaling before administering the therapeutic agent that inhibits retinoic acid receptor and/or retinoid-X receptor, thereby inducing the differentiation of myoepithelial-like tumor cells into ductal-like tumor cells.

In some implementations, the therapeutic agent that inhibits retinoic acid receptor/retinoid-X receptor signaling is selected from the group consisting of: BMS493, AGN193109, or a combination thereof.

In some implementations, the therapeutic agent that inhibits retinoic acid receptor/retinoid-X receptor signaling is a gene construct encoding a dominant-negative version of RARα (DNRARα). In some aspects, the DNRARα is a retinoic acid receptor alpha lacking its C-terminal transcriptional activation domain. For example, the DNRARα is a retinoic acid receptor alpha truncated at amino acid residue 403. In some implementations, the gene construct encoding DNRARα comprises DNhRARα subcloned into a lentivirus backbone, and in further implementations, the lentivirus backbone is based on the pLL3.7 backbone.

In another aspect, the use of CD49f to detect the presence of myoepithelial-like adenoma cells or adenocarcinoma cells is disclosed. In another aspect, the use of KIT/CD117 to detect the presence of ductal-like adenoma cells or adenocarcinoma cells is disclosed. In a further aspect, the use of CD49f and KIT/CD117 to type adenocarcinoma cells as myoepithelial-like or ductal-like is disclosed. In some implementations, the adenocarcinoma cells being detected or typed are non-small cell lung cancer cells, colon cancer cells, ovarian cancer cells, renal cancer cells, prostate cancer cells, breast cancer cells, pancreatic cancer cells, or adenoid cystic carcinoma (ACC) cells.

In another aspect, a therapeutic agent is disclosed that inhibits retinoic acid receptor/retinoid-X receptor signaling for use in the inhibiting growth of ductal adenocarcinoma. The therapeutic agent that inhibits retinoic acid receptor/retinoid-X receptor signaling is selected from BMS493, AGN193109, or a gene construct encoding a dominant-negative version of RARα (DNRARα). In yet another aspect, a therapeutic agent is disclosed that inhibits retinoic acid receptor/retinoid-X receptor signaling for use in inhibiting myoepithelial-to-ductal differentiation in adenoma cells or adenocarcinoma cells. The therapeutic agent that inhibits retinoic acid receptor/retinoid-X receptor signaling is selected from BMS493, AGN193109, or a gene construct encoding a dominant-negative version of RARα (DNRARα).

In some embodiments, the DNRARα is a retinoic acid receptor alpha lacking its C-terminal transcriptional activation domain. In further embodiments, the DNRARα is a retinoic acid receptor alpha truncated at amino acid residue 403. In further embodiments, the gene construct encoding DNRARα comprises a nucleic acid encoding DNhRARα subcloned into a lentivirus backbone. In still further embodiments, the lentivirus backbone is based on the pLL3.7 backbone.

In another aspect, the use of a dominant-negative version of RARα (DNRARα) expressed in a gene construct for reducing viability of adenocarcinoma cells is disclosed. In some implementations, wherein the DNRARα is a retinoic acid receptor alpha lacking its C-terminal transcriptional activation domain. In further implementations, the DNRARα is a retinoic acid receptor alpha truncated at amino acid residue 403. In some implementations, the gene construct comprises a nucleic acid encoding DNhRARα subcloned into a lentivirus backbone, and in further implementations, the lentivirus backbone is based on the pLL3.7 backbone.

In another aspect, a method of inhibiting growth of adenoma cells or adenocarcinoma cells in a subject is disclosed. In some aspects, the adenoma cells or adenocarcinoma cells are from salivary gland, lung, breast tissue, colon, kidney, pancreas, ovary, or prostate. The method comprises obtaining a tumor sample from the subject, sorting cells of the tumor sample based on the expression of CD49f and/or KIT/CD117 in the tumor sample, and administering a therapeutic agent to the subject that inhibits retinoic acid receptor and/or retinoid-X receptor signaling upon the indication of the presence of ductal-like tumor cells in the sample. The presence of cells positive for KIT/CD117 (optionally with low expression of CD49f) indicates the presence of ductal-like tumor cells. The presence of cells negative for KIT/CD117 with high expression of CD49f indicates the presence myoepithelial-like tumor cells. In some implementations, where the sorting step indicates the tumor sample comprises less than 95% cells positive for KIT/CD117 (indication of ductal-like tumor cells), the method further comprises administering to the subject a therapeutic agent that activates retinoic acid receptor and/or retinoid-X receptor signaling before administering the therapeutic agent that inhibits retinoic acid receptor and/or retinoid-X receptor, thereby inducing the differentiation of myoepithelial-like tumor cells into ductal-like tumor cells. In certain implementation, the tumor sample is from a subject diagnosed with or suspected of having ACC.

In another implementation, the method of inhibiting growth of adenoma cells or adenocarcinoma cells in a subject comprises obtaining a tumor sample from the subject, determining the expression of TP63 and/or KIT/CD117 in cells of the tumor sample using immunohistochemistry, and administering a therapeutic agent to the subject that inhibits retinoic acid receptor and/or retinoid-X receptor signaling upon the indication of the presence of ductal-like tumor cells in the sample. The presence of cells positive for KIT/CD117 and negative for TP63 indicates the presence of ductal-like tumor cells. The presence of cells negative for KIT/CD117 and positive for TP63 indicates the presence of myoepithelial-like tumor cells. In some implementations, where the tumor sample is identified to comprise more than 5% of the cells positive for TP63 (indication of myoepithelial-like tumor cells), the method further comprises administering to the subject a therapeutic agent that activates retinoic acid receptor and/or retinoid-X receptor signaling before administering the therapeutic agent that inhibits retinoic acid receptor and/or retinoid-X receptor, thereby inducing the differentiation of myoepithelial-like tumor cells into ductal-like tumor cells. In certain implementation, the tumor sample is from a subject diagnosed with or suspected of having ACC.

In some implementations, the therapeutic agent that activates retinoic acid receptor and/or retinoid-X receptor signaling is selected from the group consisting of: all-trans retinoic acid (ATRA), bexarotene, or a combination thereof. In some implementations, the therapeutic agent that inhibits retinoic acid receptor/retinoid-X receptor signaling is selected from the group consisting of: BMS493, AGN193109, or a combination thereof.

In some implementations, the therapeutic agent that inhibits retinoic acid receptor/retinoid-X receptor signaling is a gene construct encoding a dominant-negative version of RARα (DNRARα). In some implementations, the DNRARα is a retinoic acid receptor alpha lacking its C-terminal transcriptional activation domain. In further implementations, the DNRARα is a retinoic acid receptor alpha truncated at amino acid residue 403. In further implementations, the gene construct encoding DNRARα comprises DNhRARα subcloned into a lentivirus backbone, and in even further implementations, the lentivirus backbone is based on the pLL3.7 backbone.

In another aspect, the use of a therapeutic agent that inhibits retinoic acid receptor and/or retinoid-X receptor signaling for the manufacture of a medicament for use in the treatment of cancer is disclosed. In some implementations, the therapeutic agent that inhibits retinoic acid receptor/retinoid-X receptor signaling is selected from the group consisting of: BMS493, AGN193109, or a combination thereof. In some implementations, the cancer is leukemia, non-small cell lung cancer, colon cancer, brain cancer, melanoma, sarcoma, ovarian cancer, renal cancer, prostate cancer, breast cancer, pancreatic cancer, or ACC. In some aspects, the therapeutic agent that inhibits retinoic acid receptor/retinoid-X receptor signaling inhibits the growth of cells from at least one cell line selected from the group consisting of: CCRF-CEM, HL-60 (TB), K-562, MOLT-4, RPMI-8226, SR, A-549/ATCC, EKVX, HOP-62, HOP-92, NCI-H226, NCI-H23, NCI-H322M, NCI-H460, NCI-H522, COLO 205, HCC-2998, HCT-116, HCT-15, HT-29, KM12, SW620, SF-268, SF-295, SF-539, SNB-19, SNB-75, U251, LOX-IMVI, MALME-3M, M14, MDA-MB-435, SK-MEL-2, SK-MEL-28, SK-MEL-5, UACC-257, UACC-62, IGROV-1, OVCAR-3, OVCAR-4, OVCAR-5, OVCAR-8, NCI/ADR-RES, SK-OV-3, 786-0, A-498, ACHN, CAKI-1, RXF 393, SN12C, TK-10, UO-31, PC-3, DU-145, MCF-7, MDA-MB-231/ATCC, HS 578T, BT-549, T-47D, and MDA-MB-468.

In another aspect, the use of a therapeutic agent that inhibits retinoic acid receptor and/or retinoid-X receptor signaling for the manufacture of a medicament for use in the treatment of cancer is disclosed. In some implementations, the therapeutic agent that inhibits retinoic acid receptor/retinoid-X receptor signaling is selected from the group consisting of: BMS493, AGN193109, or a combination thereof. In some implementations, the cancer is leukemia, non-small cell lung cancer, colon cancer, brain cancer, melanoma, sarcoma, ovarian cancer, renal cancer, prostate cancer, breast cancer, pancreatic cancer, or ACC. In particular implementations, the cancer comprises ductal-like cells.

The use of a therapeutic agent that activates retinoic acid receptor and/or retinoid-X receptor signaling for the manufacture of a medicament for use in the treatment of cancer is additionally disclosed. In some implementations, the therapeutic agent that inhibits retinoic acid receptor/retinoid-X receptor signaling is selected from the group consisting of: all-trans retinoic acid (ATRA), bexarotene, or a combination thereof. In some implementations, the cancer is leukemia, non-small cell lung cancer, colon cancer, brain cancer, melanoma, sarcoma, ovarian cancer, renal cancer, prostate cancer, breast cancer, pancreatic cancer, or ACC. In particular implementations, the cancer comprises myoepithelial-like cells.

By “hacking” the signaling pathways that control multi-lineage differentiation in epithelial tissues, it is possible to discover novel pharmacological manipulations with selective toxicity on specific cellular lineages. As such a method of screening therapeutic candidates useful for the treatment and/or management of cancer, such as leukemia, non-small cell lung cancer, colon cancer, brain cancer, melanoma, sarcoma, ovarian cancer, renal cancer, prostate cancer, breast cancer, pancreatic cancer, or ACC, is disclosed. The method comprising providing a tumor sample; sorting cells of the tumor sample based on expression of CD49f and/or KIT/CD117; and administering therapeutic candidates to the sorted cells of the tumor sample. In some implementations, the method further comprises measuring the efficacy of the therapeutic candidates in relation to tumorigenesis, cell growth, and/or cell viability. In some aspects the efficacy of the therapeutic candidates in relation to tumorigenesis, cell growth, and/or cell viability are assessed by analyzing expression of genes and/or proteins related to tumorigenesis, cell growth, and/or cell viability. In some implementations, the cells of the tumor are sorted using fluorescence-activated cell sorting. In particular implementations, the method comprises providing an ACC tumor sample; sorting cells of the ACC tumor sample based on expression of CD49f and/or KIT/CD117; and administering therapeutic candidates to the sorted cells of the ACC tumor sample. In some implementations, the method further comprises measuring the efficacy of the therapeutic candidates in relation to tumorigenesis, cell growth, and/or cell viability. In some aspects the efficacy of the therapeutic candidates in relation to tumorigenesis, cell growth, and/or cell viability are assessed by analyzing expression of genes and/or proteins related to tumorigenesis, cell growth, and/or cell viability. In some embodiments, the cells of the ACC tumor are sorted using fluorescence-activated cell sorting.

### EXAMPLES

### Example 1. Identification of Surface Markers Differentially Expressed Between Myoepithelial-Like and Ductal-Like Cells

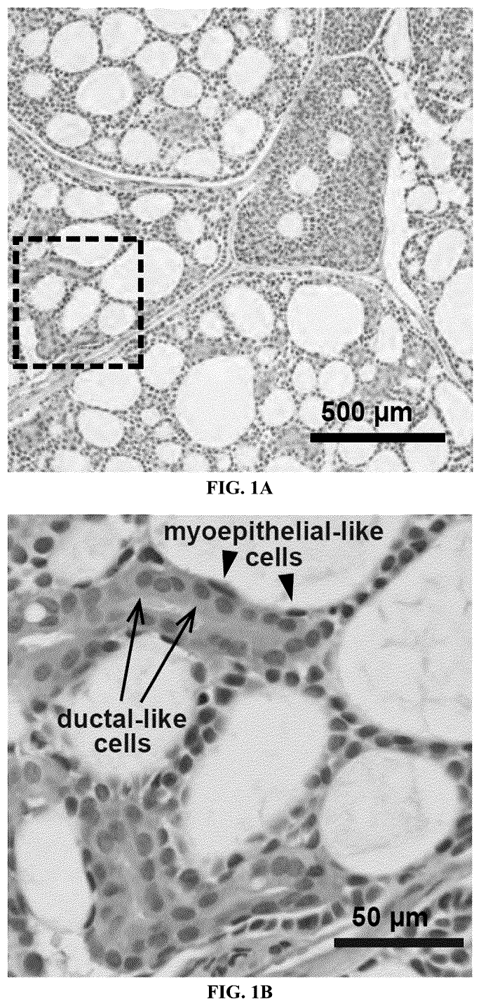

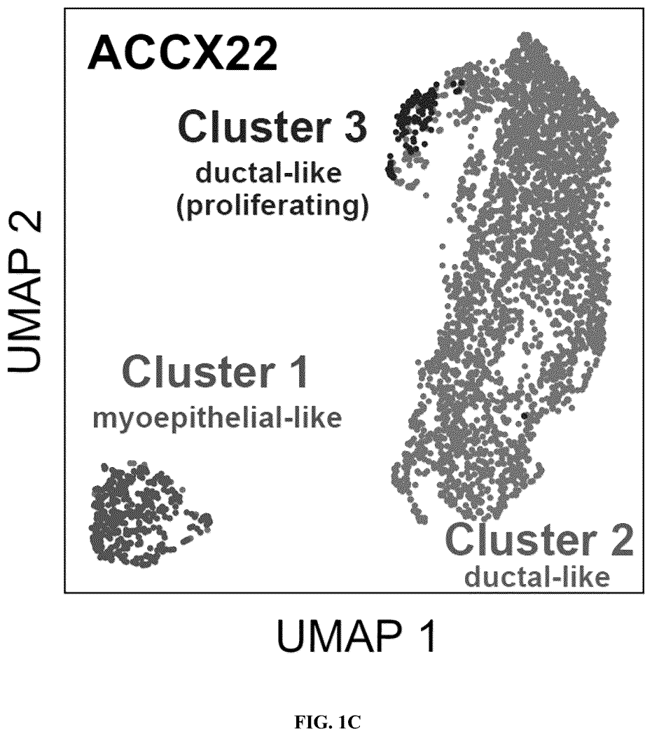

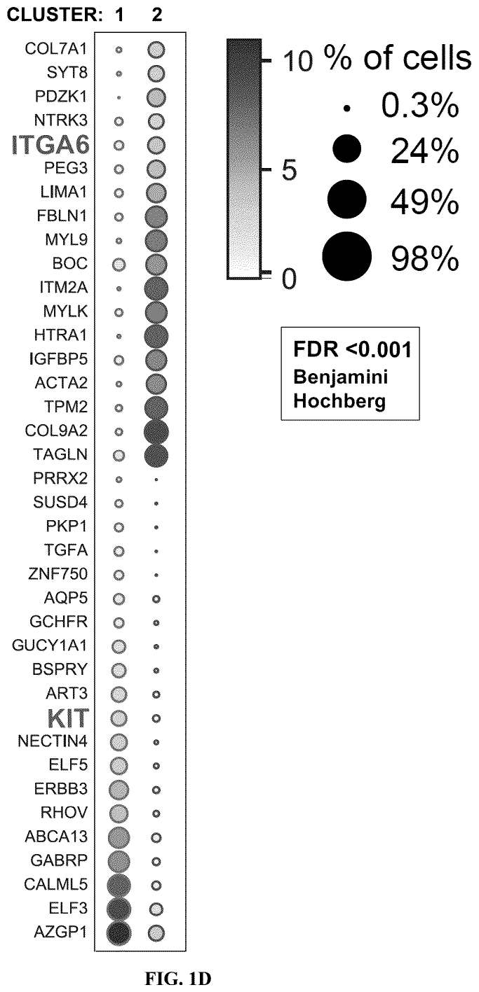

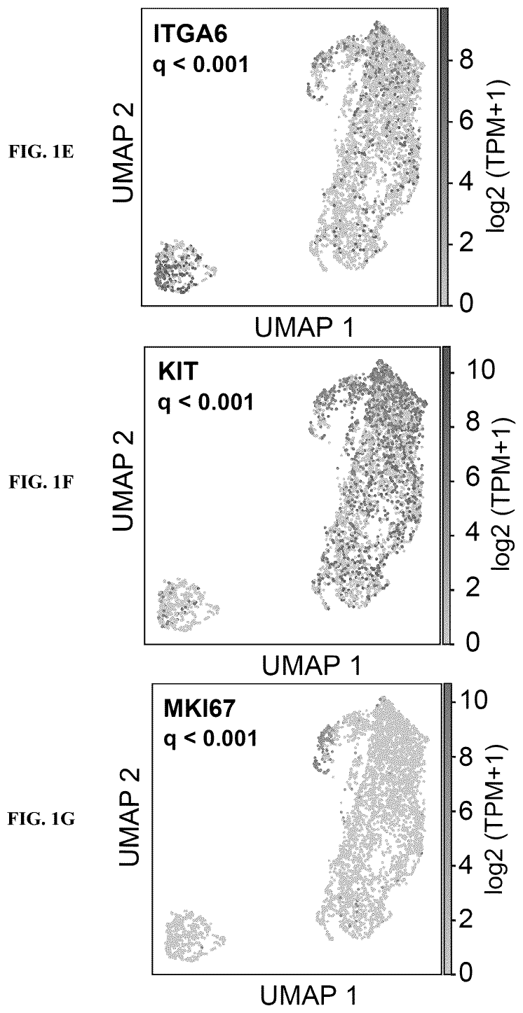

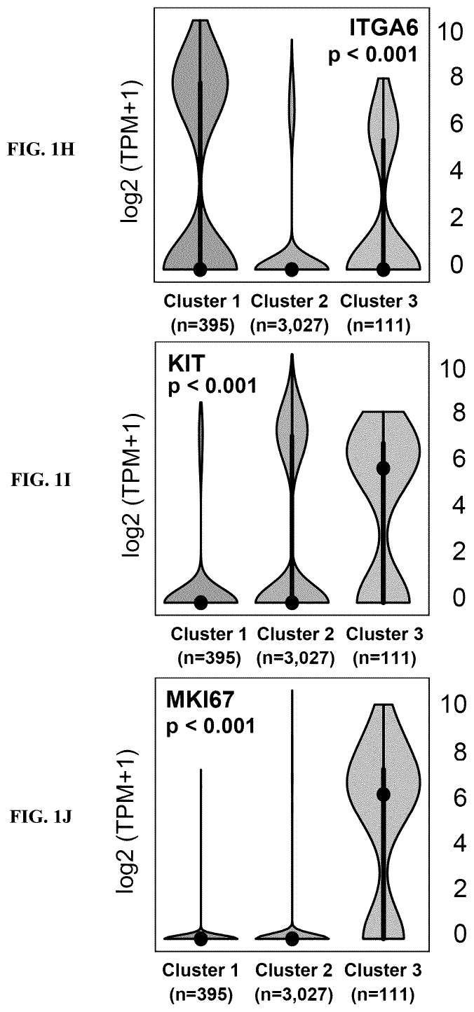

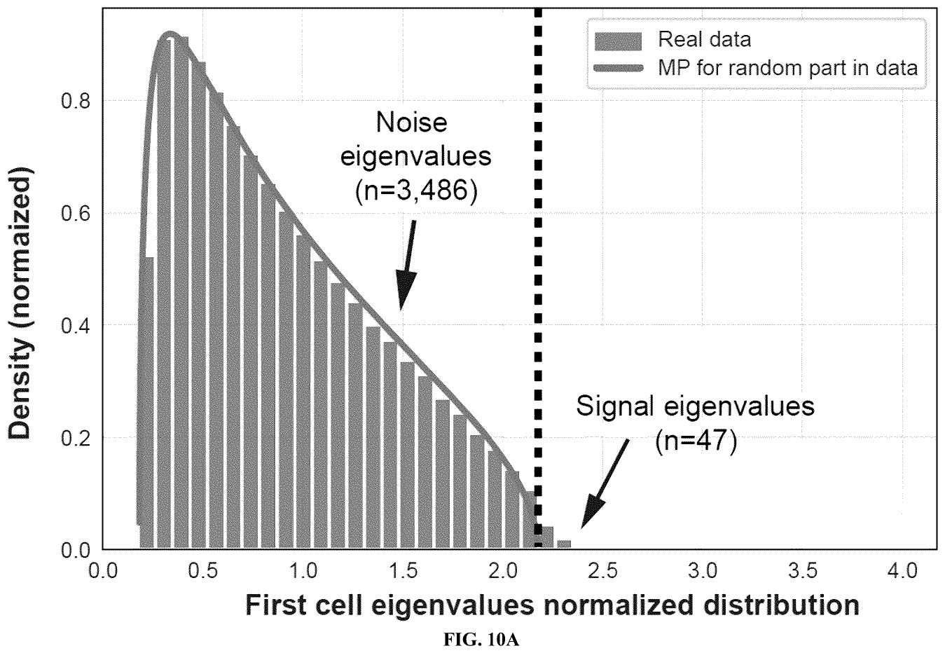

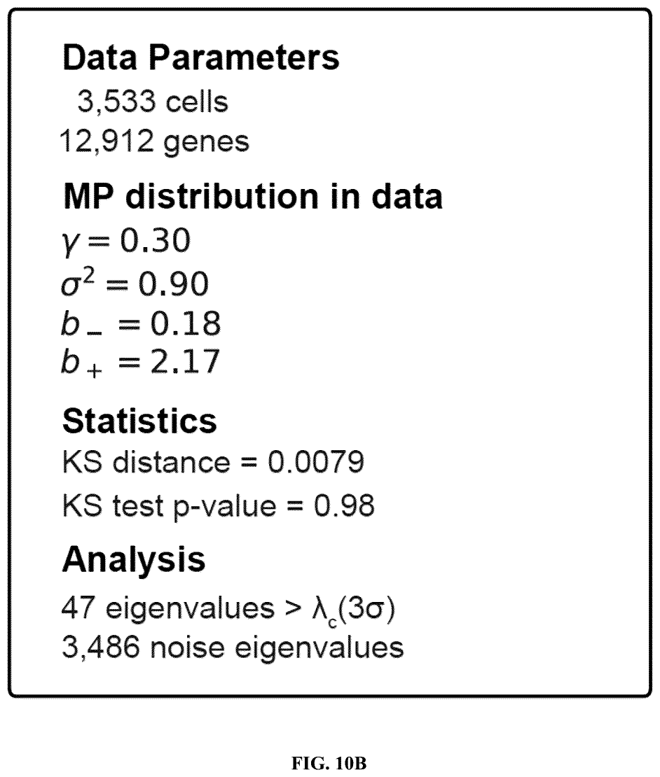

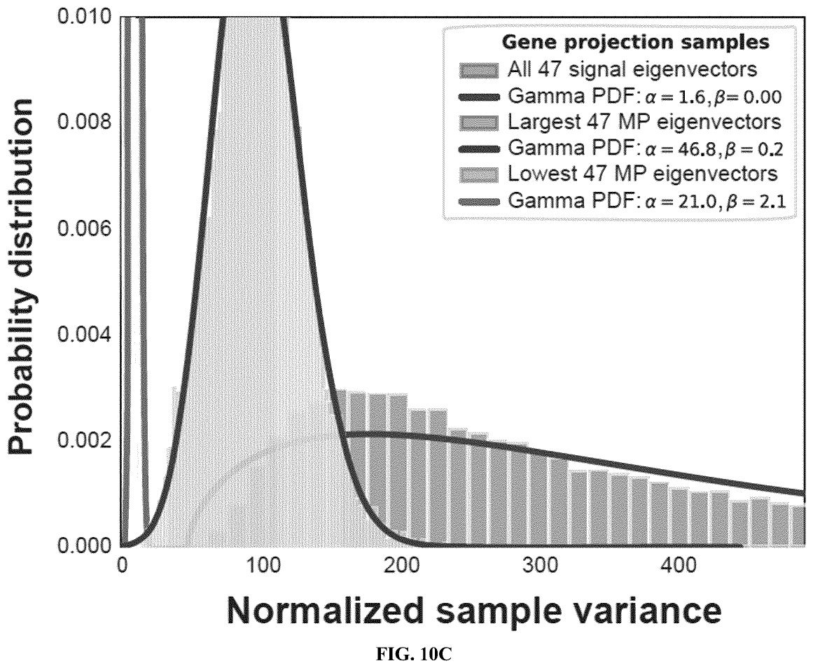

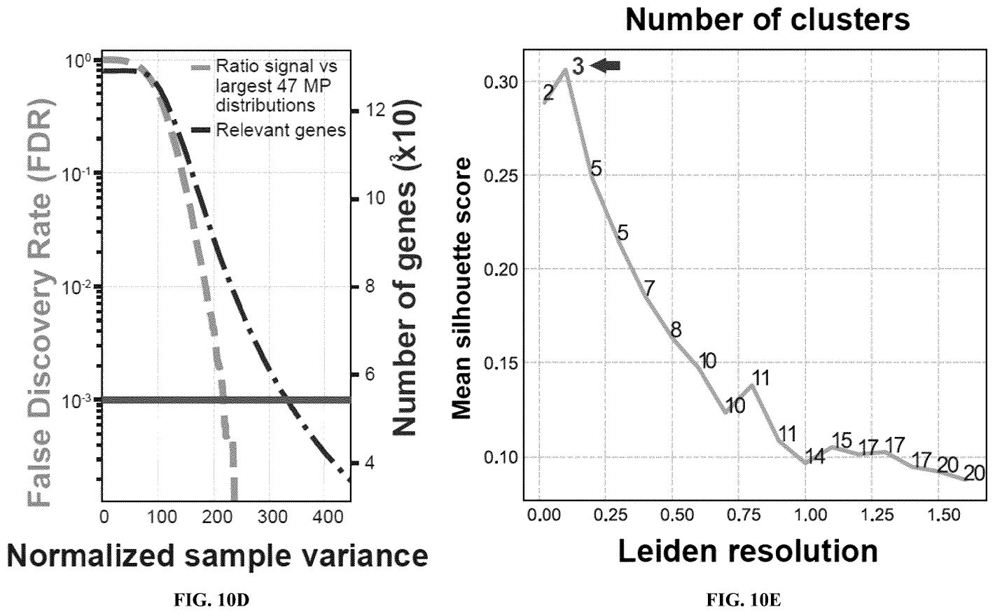

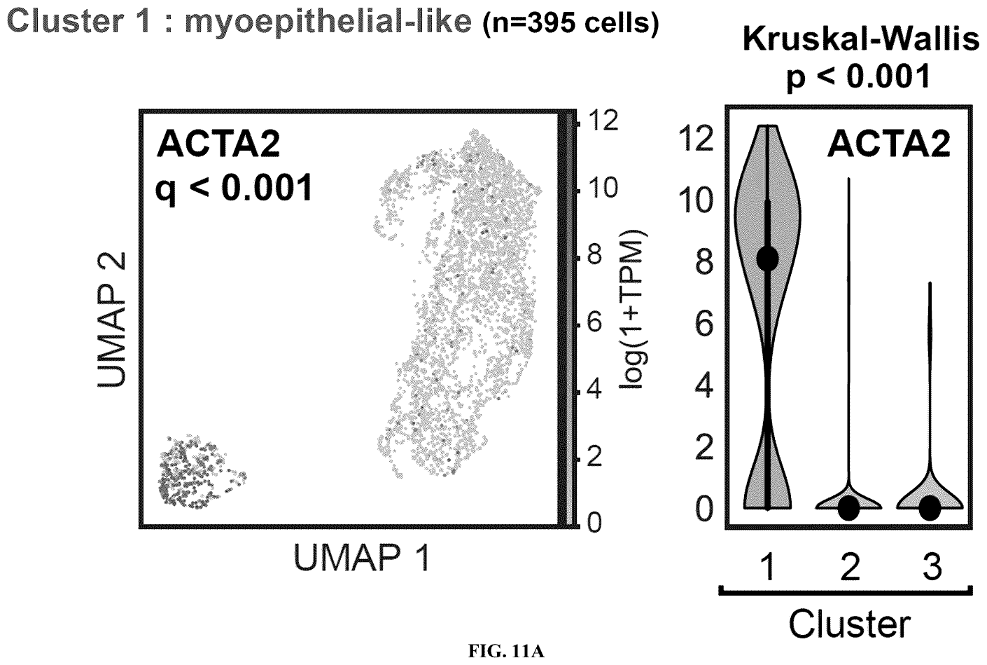

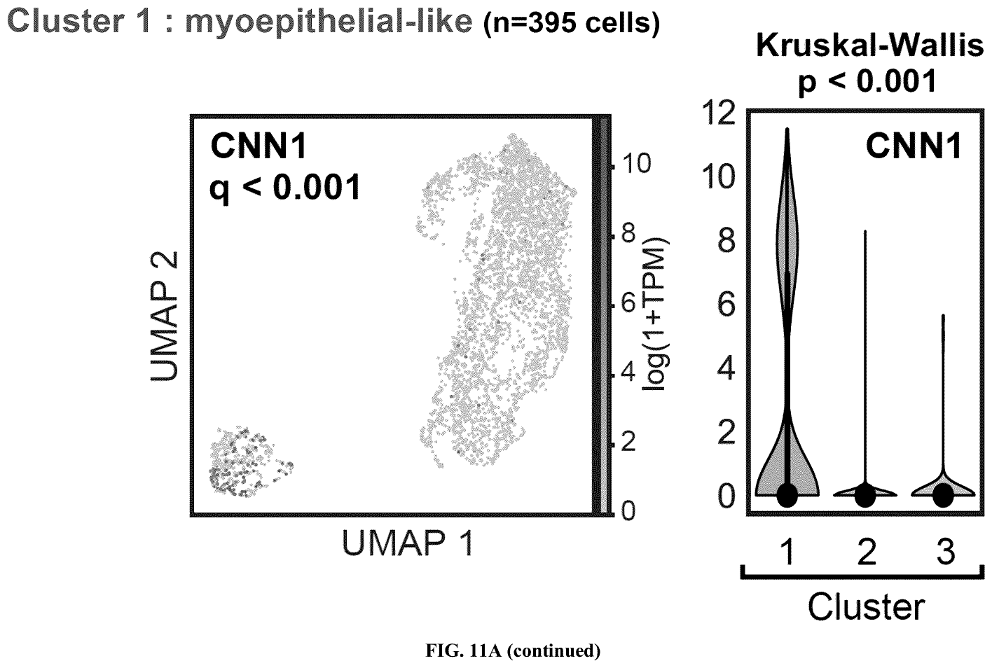

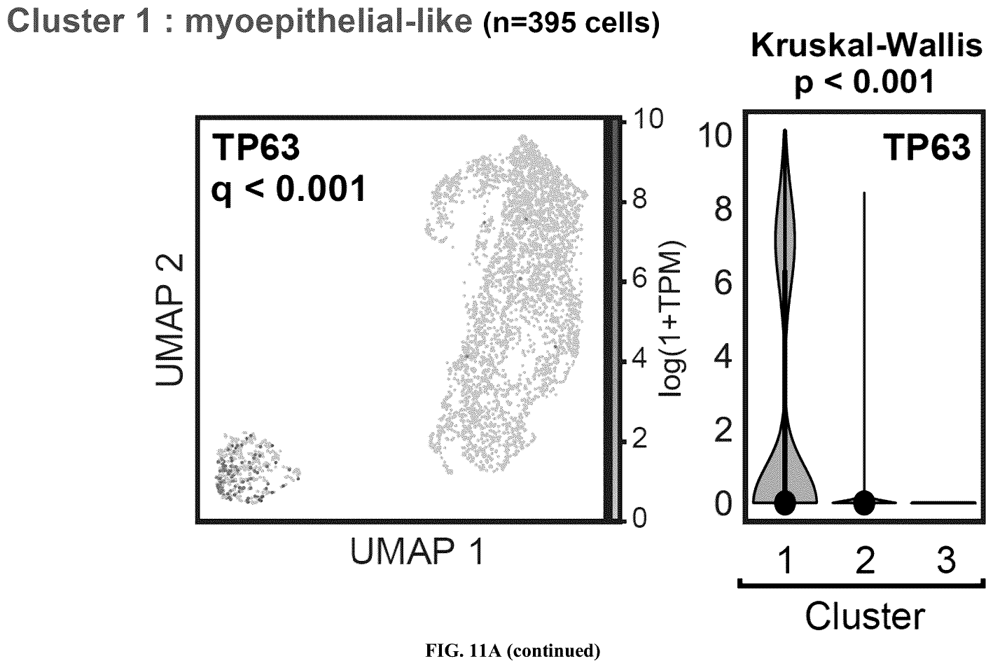

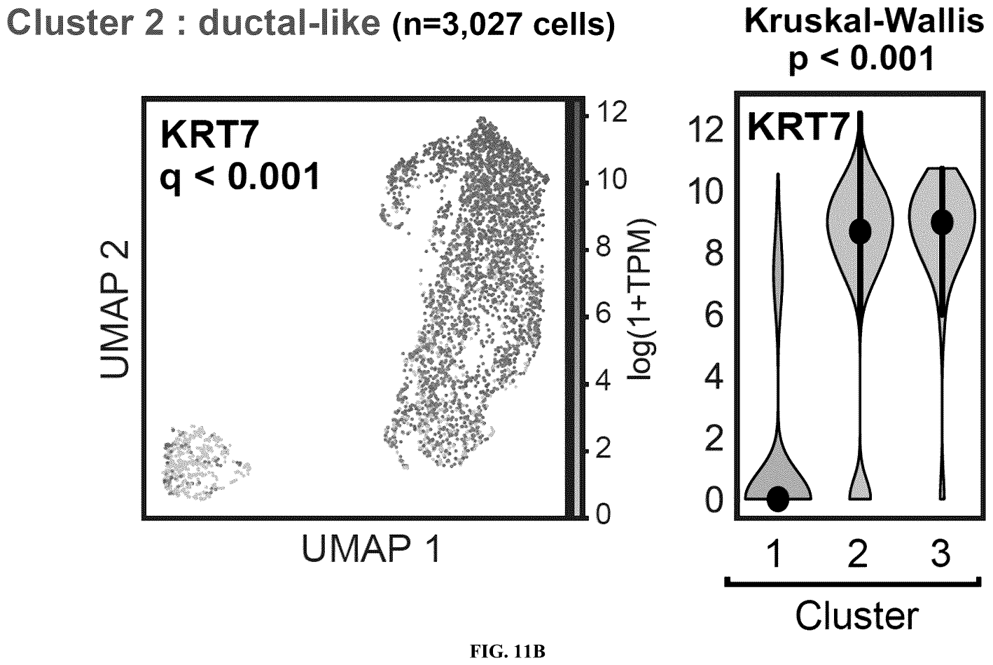

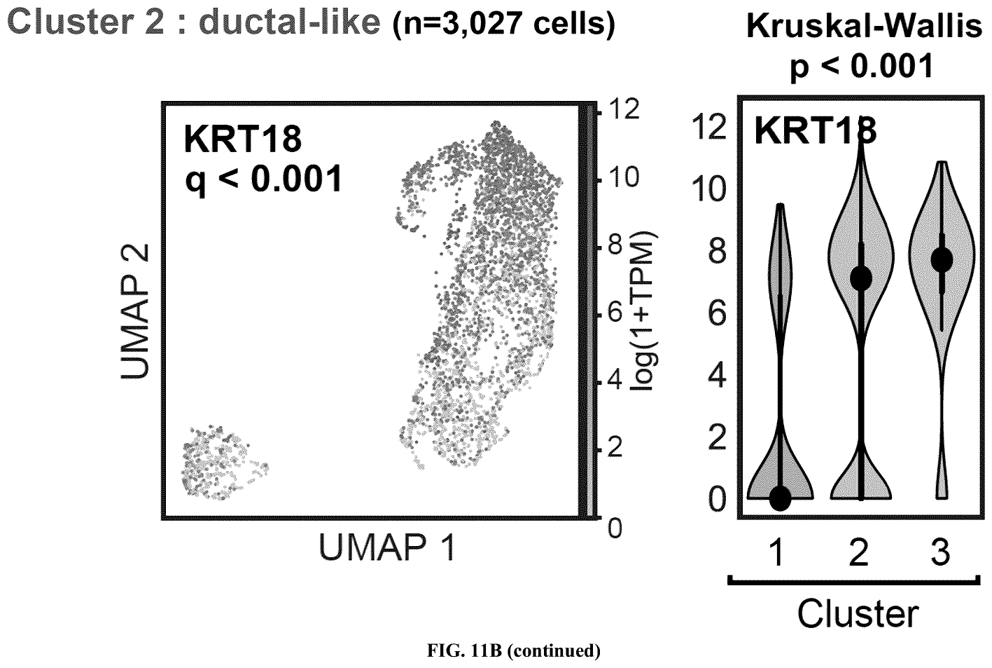

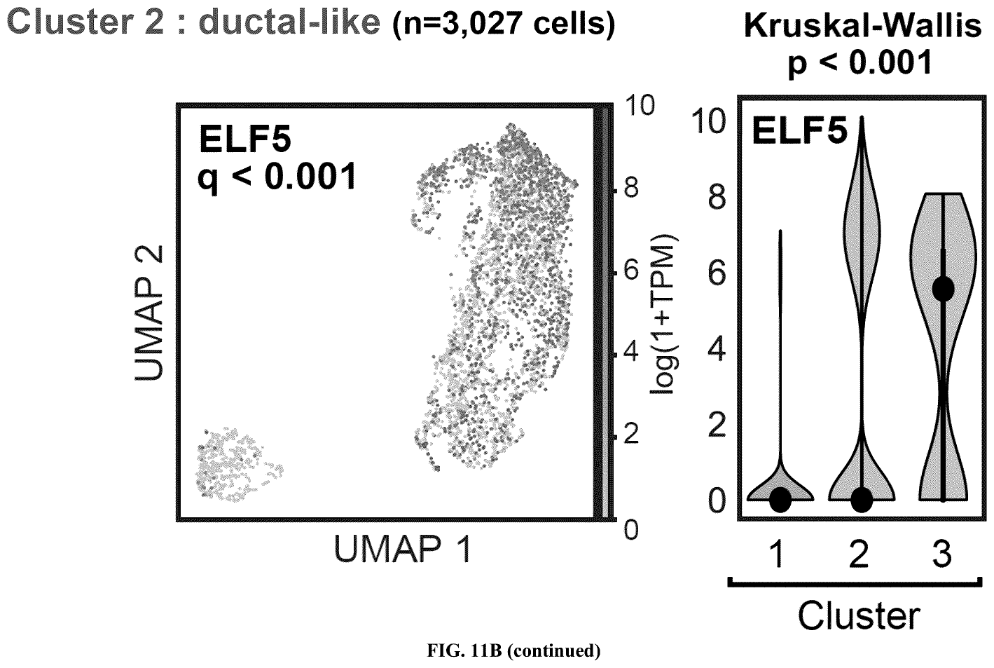

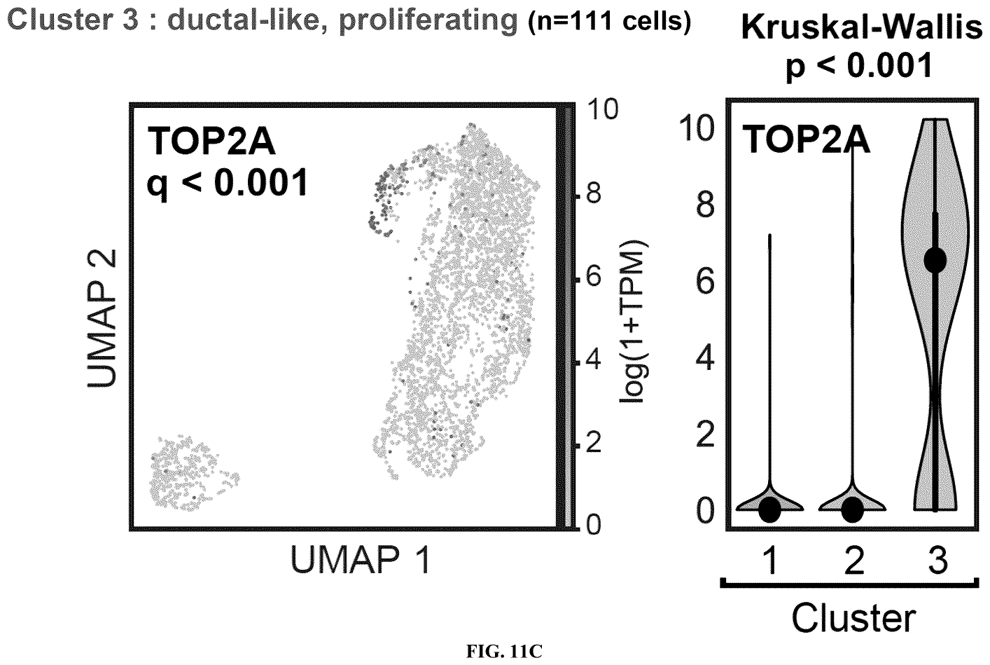

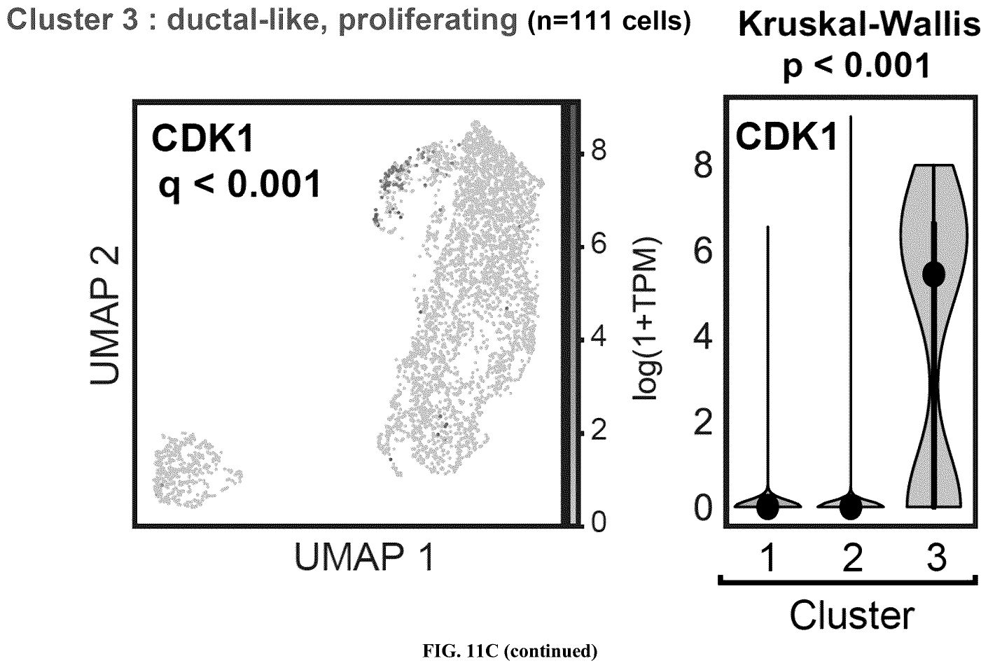

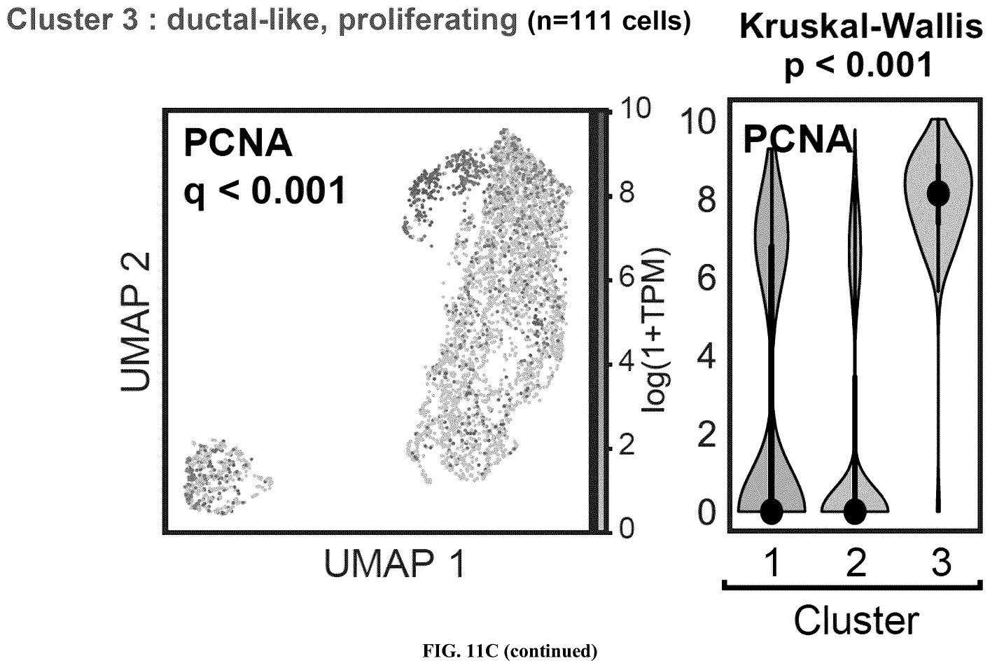

To identify surface markers differentially expressed between myoepithelial-like and ductal-like cells, a bulk preparation of epithelial cancer cells was analyzed by scRNA-seq (EpCAM⁺) and purified by FACS from a PDX line representative of a human ACC with classic “cribriform” histology (FIGS. 1A-B, and 9) [27]. The Randomly [28] algorithm was used to remove stochastic contributions to the transcriptional variability observed between cells, and then clustered cells based on systematic differences in transcriptional patterns, identifying an optimal clustering solution consisting of three sub-groups (FIG. 1C and FIG. 10). Of these three sub-groups, the largest two displayed mutually exclusive expression of known myoepithelial (ACTA2, CNN1, TP63) and ductal (KRT7, KRT18, ELF5) cell markers (FIG. 11), while a third appeared to represent a highly proliferating (MKI67^{high}) subset of ductal-like cells (FIGS. 1G, J, and 11C). Among the differentially expressed genes, those encoding for cell-surface markers CD49f (ITGA6) and KIT/CD117 (KIT) were identified, which associated with myoepithelial and ductal markers, respectively (FIGS. 1D-1I). Whether CD49f and KIT could be leveraged to visualize myoepithelial-like and ductal-like cells by FACS was then tested. Indeed, staining with fluorophore-conjugated antibodies directed against the two markers enabled clear discrimination of two cell populations (CD49f^{high}/KIT^{neg} vs. CD49f^{low}/KIT⁺) across 5 independent PDX lines representative of bi-phenotypic ACCs (FIG. 2A). Analysis of the same tumors by IHC also confirmed that KIT expression was restricted to ductal-like cells, and mutually exclusive to expression of TP63, a myoepithelial marker (FIG. 2B).

### Example 2. Transcriptional Profiling of CD49f^{high}/KIT^{neg} and CD49f^{low}/KIT⁺ Cells

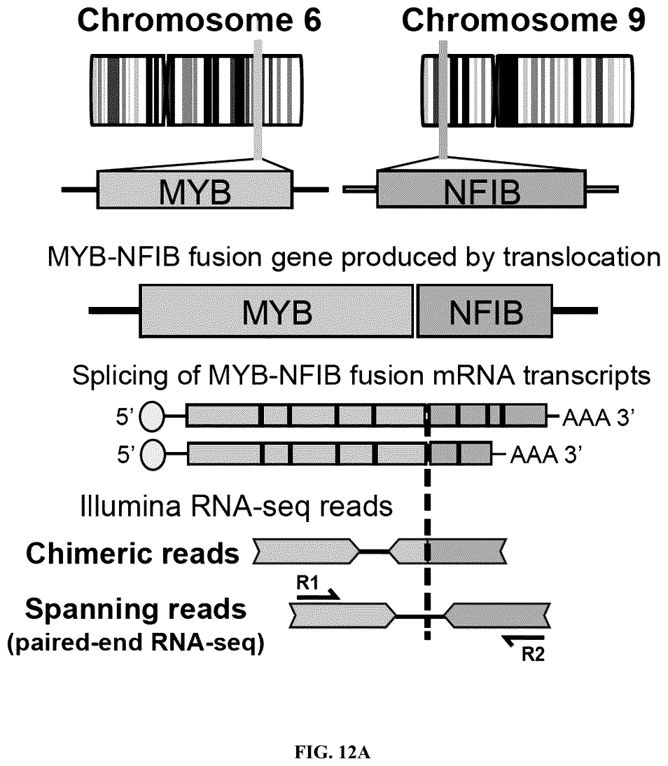

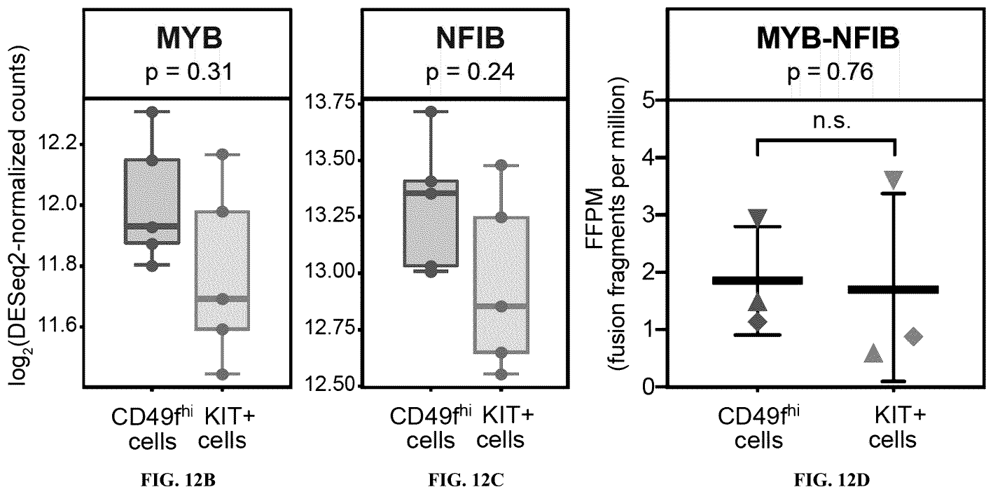

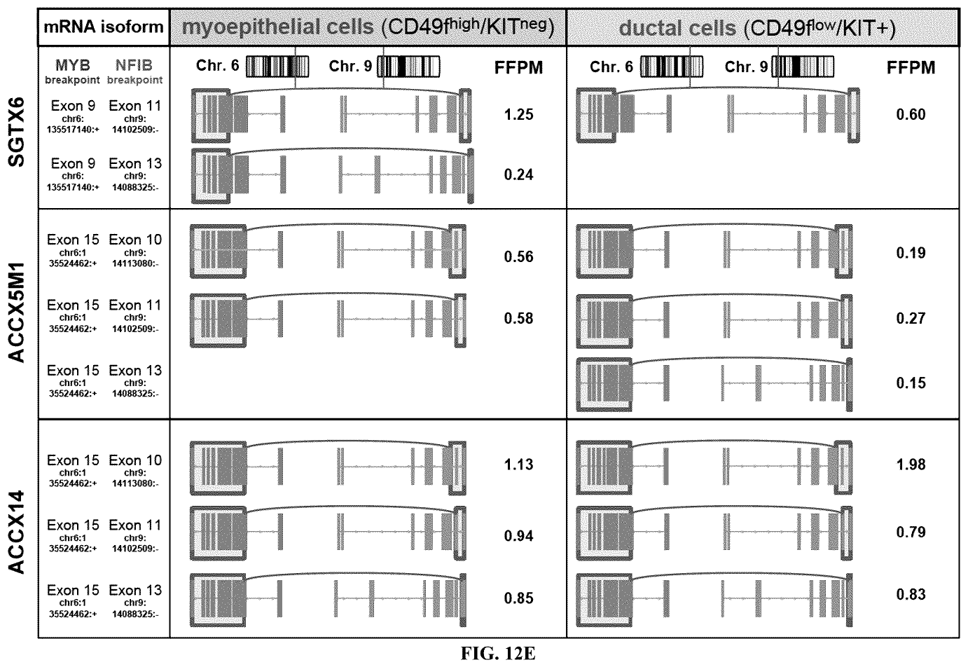

To understand whether CD49f^{high}/KIT^{neg} and CD49f^{low}/KIT⁺ cells isolated from different patients displayed similar gene-expression patterns, autologous pairs of the two cell-types were sorted from 5 bi-phenotypic PDX lines, and were analyzed by conventional RNA-seq. When analyzed by principal component analysis (PCA), the 10 samples segregated into two equal clusters (5 samples/cluster) that matched the original phenotypes of sorted cells (CD49f^{high}/KIT^{neg} vs. CD49f^{low}/KIT⁺). The two clusters separated along the first principal component (PC1), which accounted for a dominant fraction (58%) of the variability within the dataset (FIG. 2C). This observation revealed that the two cell-types were defined by systematic differences in transcriptional profiles, strongly conserved across different tumors irrespective of patient-specific variables (e.g., site of origin, sex, repertoire of genetic alterations) (Table 1) [27]. DESeq2 [29] was used to identify genes differentially-expressed between the two cell-types (Table 2), and it was observed that CD49f^{high}/KIT^{neg} cells expressed markers of myoepithelial cells (e.g., ACTA2, MYH11, PDPN, TP63) [15-20], while CD49f^{low}/KIT⁺ cells expressed markers of the ductal/luminal lineages of exocrine glands (e.g., ELF5, KIT, SLPI, ANXA8) [40-43] (FIG. 2D), thus confirming their myoepithelial-like and ductal-like identities. Finally, STAR-Fusion was used to test whether CD49f^{high}/KIT^{neg} and CD49f^{low}/KIT⁺ cells, which are both known to carry t (6;9) MYB-NFIB translocations [44], differed in expression of MYB-NFIB chimeric transcripts. Analysis revealed that, in ACCs that harbored such translocations, both cell types expressed MYB-NFIB chimeric transcripts, without evidence of meaningful differences in terms of absolute levels or alternative splicing (FIG. 12).

TABLE 1
Clinical, pathological and molecular characteristics of the Adenoid Cystic Carcinomas (ACCs) from
which the patient derived xenograft (PDX) models utilized in this study have been established.

| | | | | Primary | | | | | NOTCH1 |
|---|---|---|---|---|---|---|---|---|---|
| | Patient | Patient | Site of | vs. | Metastatic | Tumor | Tumor | MYB | activating |
| PDX line | Age | Sex | Origin | Metastasis | site | Grade | histology | rearrangement | mutation |
| ACCX5M1 | 54 | Male | Oral cavity | Metastasis | Lung | G2 | cribriform | MYB-NFIB | wt |
| ACCX6 | 33 | Male | Parotid gland | Metastasis | Lung | G2 | tubular/solid | MYB-TGFBR3 | wt |
| ACCX14 | 40 | Female | Trachea | Primary | n.a. | G1 | cribriform | MYB-NFIB | wt |
| ACCX22 | 36 | Female | Parotid gland | Primary | n.a. | G1 | cribriform | MYB-NFIB | wt |
| SGTX6 | 49 | Female | Oral cavity | Metastasis | Liver | G2 | cribriform | MYB-NFIB | wt |
| ACCX9 | 77 | Female | Parotid gland | Primary | n.a. | G3 | solid | MYB-NFIB | I1680Nmutation |
| ACCX11 | 55 | Female | Nasal sinus | Primary | n.a. | G3 | solid | MYB-NFIB | 3′UTR duplication |

TABLE 2
List of 643 genes identified as differentially expressed between myoepithelial-like
(CD49f^{high}/KIT^{neg}) and ductal-like (CD49f^{low}/KIT⁺) cells in human Adenoid Cystic Carcinomas (ACCs)

| Rank | Gene name | baseMean | log2FoldChange | lfcSE | stat | pvalue | padj | Population |
|---|---|---|---|---|---|---|---|---|
| 1 | ANXA8L1 | 901.3393 | 6.035 | 0.2428 | 20.7357 | 1.65E−95 | 2.83E−91 | KIT |
| 2 | CGB7 | 196.6317 | −4.274 | 0.2047 | −15.9946 | 1.39E−57 | 1.19E−53 | CD49f |
| 3 | ELF5 | 327.1784 | 5.464 | 0.2933 | 15.219 | 2.65E−52 | 1.51E−48 | KIT |
| 4 | KIT | 3296.8948 | 4.869 | 0.2718 | 14.2339 | 5.64E−46 | 2.41E−42 | KIT |
| 5 | JAG2 | 1958.0065 | −3.164 | 0.1548 | −13.9791 | 2.09E−44 | 7.15E−41 | CD49f |
| 6 | NTF4 | 174.9941 | −4.094 | 0.2252 | −13.7414 | 5.74E−43 | 1.64E−39 | CD49f |
| 7 | CEMIP | 112.951 | −4.797 | 0.2903 | −13.0784 | 4.38E−39 | 1.07E−35 | CD49f |
| 8 | TMPRSS2 | 558.6783 | 5.516 | 0.356 | 12.6865 | 7.03E−37 | 1.50E−33 | KIT |
| 9 | LFNG | 603.4045 | −3.474 | 0.2051 | −12.0638 | 1.64E−33 | 3.12E−30 | CD49f |
| 10 | PDGFA | 1520.0289 | −2.306 | 0.1091 | −11.973 | 4.92E−33 | 8.41E−30 | CD49f |
| 11 | UCN2 | 322.5907 | −4.38 | 0.2885 | −11.7169 | 1.04E−31 | 1.62E−28 | CD49f |
| 12 | BARX2 | 193.2961 | 4.378 | 0.2961 | 11.4078 | 3.82E−30 | 5.45E−27 | KIT |
| 13 | COL7A1 | 15802.7447 | −4.057 | 0.2682 | −11.4003 | 4.17E−30 | 5.48E−27 | CD49f |
| 14 | TP73 | 687.7793 | −4.199 | 0.2814 | −11.3706 | 5.86E−30 | 7.16E−27 | CD49f |
| 15 | PDGFRA | 2449.1635 | −2.786 | 0.1572 | −11.3611 | 6.53E−30 | 7.45E−27 | CD49f |
| 16 | PDZK1 | 2983.0554 | −5.295 | 0.395 | −10.873 | 1.55E−27 | 1.66E−24 | CD49f |
| 17 | SERPINF1 | 1045.0927 | −5.354 | 0.4027 | −10.8117 | 3.03E−27 | 3.05E−24 | CD49f |
| 18 | KLHL29 | 457.9386 | −3.9 | 0.2697 | −10.7519 | 5.81E−27 | 5.52E−24 | CD49f |
| 19 | NEBL | 580.1535 | 4.31 | 0.309 | 10.7089 | 9.24E−27 | 8.32E−24 | KIT |
| 20 | SLPI | 681.8024 | 4.544 | 0.334 | 10.6109 | 2.65E−26 | 2.27E−23 | KIT |
| 21 | MMP2 | 457.3375 | −4.56 | 0.3429 | −10.3826 | 2.97E−25 | 2.42E−22 | CD49f |
| 22 | HTRA1 | 4871.6349 | −5.634 | 0.4516 | −10.2621 | 1.04E−24 | 8.12E−22 | CD49f |
| 23 | CLDN8 | 138.1633 | 5.832 | 0.4713 | 10.2531 | 1.15E−24 | 8.53E−22 | KIT |
| 24 | PEG3 | 8830.8505 | −2.634 | 0.1605 | −10.1807 | 2.42E−24 | 1.72E−21 | CD49f |
| 25 | B3GALT5 | 344.1458 | 4.885 | 0.3827 | 10.1523 | 3.24E−24 | 2.21E−21 | KIT |
| 26 | COBL | 555.6633 | 4.152 | 0.3109 | 10.1389 | 3.71E−24 | 2.44E−21 | KIT |
| 27 | GUCY1A1 | 1913.8419 | 5.172 | 0.4144 | 10.0688 | 7.59E−24 | 4.67E−21 | KIT |
| 28 | NECTIN4 | 951.2089 | 4.768 | 0.3743 | 10.0682 | 7.64E−24 | 4.67E−21 | KIT |
| 29 | PRRX2 | 124.9847 | 4.487 | 0.3471 | 10.0458 | 9.58E−24 | 5.65E−21 | KIT |
| 30 | AIF1L | 1212.6531 | 2.475 | 0.1474 | 10.0077 | 1.41E−23 | 8.04E−21 | KIT |
| 31 | TPM2 | 4870.3372 | −3.306 | 0.2316 | −9.9598 | 2.29E−23 | 1.26E−20 | CD49f |
| 32 | RHOV | 1292.9564 | 5.07 | 0.4125 | 9.8672 | 5.77E−23 | 3.09E−20 | KIT |
| 33 | FBLN1 | 2329.8095 | −3.461 | 0.2507 | −9.815 | 9.70E−23 | 5.03E−20 | CD49f |
| 34 | CALML5 | 806.5001 | 4.956 | 0.4049 | 9.7723 | 1.48E−22 | 7.45E−20 | KIT |
| 35 | TMC6 | 264.4301 | 2.578 | 0.1616 | 9.7655 | 1.58E−22 | 7.74E−20 | KIT |
| 36 | ADGRV1 | 244.4375 | 3.359 | 0.2438 | 9.6781 | 3.74E−22 | 1.77E−19 | KIT |
| 37 | MYL9 | 4312.4982 | −4.281 | 0.3408 | −9.6287 | 6.05E−22 | 2.80E−19 | CD49f |
| 38 | CLDN3 | 757.9516 | 4.782 | 0.3958 | 9.5536 | 1.25E−21 | 5.64E−19 | KIT |
| 39 | AZGP1 | 10289.808 | 5.058 | 0.4258 | 9.5316 | 1.55E−21 | 6.79E−19 | KIT |
| 40 | LIMS2 | 292.3854 | −4.37 | 0.3542 | −9.5149 | 1.82E−21 | 7.78E−19 | CD49f |
| 41 | TGFB1I1 | 870.013 | −2.569 | 0.1652 | −9.5011 | 2.08E−21 | 8.67E−19 | CD49f |
| 42 | ENPP4 | 121.7435 | 3.105 | 0.2245 | 9.3783 | 6.70E−21 | 2.73E−18 | KIT |
| 43 | PDPN | 424.015 | −4.923 | 0.421 | −9.3175 | 1.19E−20 | 4.73E−18 | CD49f |
| 44 | PDZK1P1 | 240.4993 | −5.051 | 0.4348 | −9.3152 | 1.22E−20 | 4.73E−18 | CD49f |
| 45 | GABRP | 15379.1358 | 3.968 | 0.319 | 9.3059 | 1.33E−20 | 5.05E−18 | KIT |
| 46 | EDNRB | 864.5944 | −4.326 | 0.3575 | −9.3029 | 1.37E−20 | 5.08E−18 | CD49f |
| 47 | GAB2 | 843.2609 | 2.341 | 0.1447 | 9.2699 | 1.86E−20 | 6.78E−18 | KIT |
| 48 | ITGB4 | 16503.2802 | −2.317 | 0.1439 | −9.1502 | 5.68E−20 | 2.02E−17 | CD49f |
| 49 | IL17B | 118.6733 | −6.965 | 0.6547 | −9.1097 | 8.26E−20 | 2.82E−17 | CD49f |
| 50 | PPP1R14A | 194.3252 | −3.436 | 0.2674 | −9.1103 | 8.21E−20 | 2.82E−17 | CD49f |
| 51 | CSPG4 | 1891.1776 | −4 | 0.3294 | −9.1068 | 8.49E−20 | 2.85E−17 | CD49f |
| 52 | PKP1 | 3922.7787 | 5.028 | 0.4457 | 9.0377 | 1.60E−19 | 5.26E−17 | KIT |
| 53 | SLC28A3 | 172.9963 | 6.4 | 0.6031 | 8.9534 | 3.45E−19 | 1.11E−16 | KIT |
| 54 | TP63 | 1697.0218 | −4.299 | 0.3704 | −8.9074 | 5.22E−19 | 1.65E−16 | CD49f |
| 55 | ANXA8 | 901.4367 | 4.592 | 0.412 | 8.7198 | 2.79E−18 | 8.63E−16 | KIT |
| 56 | WLS | 719.0907 | −3.082 | 0.2388 | −8.7182 | 2.83E−18 | 8.63E−16 | CD49f |
| 57 | ADCY5 | 365.9682 | −5.828 | 0.5616 | −8.5973 | 8.16E−18 | 2.45E−15 | CD49f |
| 58 | PRR15L | 133.3958 | 6.392 | 0.6285 | 8.5786 | 9.60E−18 | 2.83E−15 | KIT |
| 59 | NGF | 443.9714 | −4.603 | 0.4224 | −8.5297 | 1.47E−17 | 4.25E−15 | CD49f |
| 60 | WNT3A | 63.6301 | −4.815 | 0.448 | −8.5158 | 1.65E−17 | 4.71E−15 | CD49f |
| 61 | ITPR2 | 2678.8676 | 2.885 | 0.2217 | 8.5037 | 1.84E−17 | 5.15E−15 | KIT |
| 62 | IGFBP5 | 7217.8304 | −3.825 | 0.3329 | −8.4844 | 2.17E−17 | 5.98E−15 | CD49f |
| 63 | SYT7 | 643.5542 | 3.668 | 0.316 | 8.4412 | 3.14E−17 | 8.52E−15 | KIT |
| 64 | ZNF423 | 81.1286 | −3.238 | 0.267 | −8.3822 | 5.19E−17 | 1.39E−14 | CD49f |
| 65 | SMOC2 | 947.5978 | −3.352 | 0.2815 | −8.3549 | 6.55E−17 | 1.72E−14 | CD49f |
| 66 | ANKRD65 | 263.0479 | −3.736 | 0.3278 | −8.3438 | 7.19E−17 | 1.86E−14 | CD49f |
| 67 | BICDL2 | 515.0125 | 4.217 | 0.3883 | 8.2847 | 1.18E−16 | 2.98E−14 | KIT |
| 68 | OSR1 | 1382.4957 | −2.347 | 0.1626 | −8.2861 | 1.17E−16 | 2.98E−14 | CD49f |
| 69 | TGFA | 742.8084 | 3.891 | 0.35 | 8.2613 | 1.44E−16 | 3.57E−14 | KIT |
| 70 | TNFSF10 | 136.9492 | 3.827 | 0.3429 | 8.2458 | 1.64E−16 | 4.01E−14 | KIT |
| 71 | COMP | 315.8074 | −5.216 | 0.512 | −8.2341 | 1.81E−16 | 4.36E−14 | CD49f |
| 72 | MATN2 | 5459.666 | −4.702 | 0.4513 | 8.2036 | 2.33E−16 | 5.43E−14 | CD49f |
| 73 | PDGFB | 655.8632 | −3.75 | 0.3353 | −8.2028 | 2.35E−16 | 5.43E−14 | CD49f |
| 74 | SEMA3A | 1076.78 | −4.463 | 0.4221 | 8.2048 | 2.31E−16 | 5.43E−14 | CD49f |
| 75 | SLC12A1 | 117.0236 | 3.792 | 0.3405 | 8.1993 | 2.42E−16 | 5.51E−14 | KIT |
| 76 | AZGP1P1 | 218.1797 | 4.189 | 0.3904 | 8.1699 | 3.09E−16 | 6.95E−14 | KIT |
| 77 | PRR36 | 428.811 | 2.874 | 0.2294 | 8.1663 | 3.18E−16 | 7.06E−14 | KIT |
| 78 | ITPR1 | 1808.4804 | −3.781 | 0.341 | 8.1553 | 3.48E−16 | 7.64E−14 | CD49f |
| 79 | SYT1 | 332.1406 | −3.722 | 0.3371 | −8.0751 | 6.74E−16 | 1.46E−13 | CD49f |
| 80 | MYH11 | 9263.9367 | −5.456 | 0.5575 | −7.9942 | 1.30E−15 | 2.79E−13 | CD49f |
| 81 | ABCG1 | 3129.6845 | −2.128 | 0.1422 | −7.9285 | 2.22E−15 | 4.68E−13 | CD49f |
| 82 | ARHGAP30 | 142.7318 | 5.404 | 0.5558 | 7.9239 | 2.30E−15 | 4.80E−13 | KIT |
| 83 | IKBKB | 3433.1264 | −1.858 | 0.1084 | −7.9159 | 2.46E−15 | 5.06E−13 | CD49f |
| 84 | IFITM10 | 290.6205 | −2.455 | 0.1849 | −7.8685 | 3.59E−15 | 7.31E−13 | CD49f |
| 85 | COL23A1 | 147.2281 | −4.424 | 0.4367 | −7.8398 | 4.51E−15 | 9.08E−13 | CD49f |
| 86 | BSPRY | 611.1863 | 3.857 | 0.3654 | 7.8195 | 5.30E−15 | 1.05E−12 | KIT |
| 87 | TNS4 | 4750.5667 | −3.203 | 0.2828 | −7.7882 | 6.80E−15 | 1.34E−12 | CD49f |
| 88 | CA6 | 100.2903 | 3.567 | 0.3307 | 7.7638 | 8.24E−15 | 1.60E−12 | KIT |
| 89 | GCHFR | 61.1717 | 4.063 | 0.3947 | 7.7614 | 8.40E−15 | 1.61E−12 | KIT |
| 90 | LOXL2 | 3855.6388 | −4.294 | 0.4249 | −7.7536 | 8.94E−15 | 1.70E−12 | CD49f |
| 91 | ESPN | 246.5415 | 3.161 | 0.2856 | 7.5641 | 3.91E−14 | 7.34E−12 | KIT |
| 92 | ZNF750 | 1556.0837 | 4.277 | 0.4334 | 7.5616 | 3.98E−14 | 7.40E−12 | KIT |
| 93 | LYN | 200.6719 | 4.079 | 0.4088 | 7.5324 | 4.98E−14 | 9.16E−12 | KIT |
| 94 | ANGPT2 | 55.2382 | −6.699 | 0.7585 | −7.5134 | 5.76E−14 | 1.05E−11 | CD49f |
| 95 | TMC4 | 460.0232 | 3.095 | 0.2797 | 7.4915 | 6.81E−14 | 1.23E−11 | KIT |
| 96 | SLC6A14 | 312.6531 | 7.792 | 0.9074 | 7.4856 | 7.12E−14 | 1.26E−11 | KIT |
| 97 | TRAM2 | 732.7912 | −2.191 | 0.1592 | −7.4855 | 7.13E−14 | 1.26E−11 | CD49f |
| 98 | ACTA2 | 10720.5426 | −4.235 | 0.4368 | −7.4047 | 1.31E−13 | 2.29E−11 | CD49f |
| 99 | IGFBP2 | 3661.053 | −2.764 | 0.2383 | −7.4017 | 1.34E−13 | 2.32E−11 | CD49f |
| 100 | GAS6 | 11898.4692 | −3.154 | 0.2915 | −7.3881 | 1.49E−13 | 2.55E−11 | CD49f |
| 101 | FERMT1 | 509.9414 | −2.368 | 0.1853 | −7.384 | 1.54E−13 | 2.60E−11 | CD49f |
| 102 | MMP1 | 51.723 | −3.697 | 0.3659 | −7.3715 | 1.69E−13 | 2.83E−11 | CD49f |
| 103 | DKK3 | 2633.3911 | −4.424 | 0.4672 | −7.3285 | 2.33E−13 | 3.86E−11 | CD49f |
| 104 | PRDM5 | 253.1344 | −2.118 | 0.1529 | −7.3086 | 2.70E−13 | 4.44E−11 | CD49f |
| 105 | PNMA8A | 1231.7511 | −2.003 | 0.1374 | −7.3028 | 2.82E−13 | 4.59E−11 | CD49f |
| 106 | PDLIM4 | 856.9996 | −2.091 | 0.1504 | −7.2515 | 4.12E−13 | 6.65E−11 | CD49f |
| 107 | DLK2 | 88.6658 | −5.991 | 0.6967 | −7.163 | 7.89E−13 | 1.26E−10 | CD49f |
| 108 | MAL2 | 522.7098 | 2.897 | 0.2679 | 7.0789 | 1.45E−12 | 2.30E−10 | KIT |
| 109 | MSRB3 | 764.4769 | −3.45 | 0.3471 | −7.0596 | 1.67E−12 | 2.62E−10 | CD49f |
| 110 | ISM1 | 77.2568 | −3.747 | 0.3893 | −7.0562 | 1.71E−12 | 2.66E−10 | CD49f |
| 111 | IRX4 | 1701.8632 | −2.175 | 0.167 | −7.0366 | 1.97E−12 | 3.04E−10 | CD49f |
| 112 | EHF | 5494.1239 | 3.48 | 0.3526 | 7.0321 | 2.03E−12 | 3.11E−10 | KIT |
| 113 | ATP13A5 | 44.7376 | 5.55 | 0.6478 | 7.0237 | 2.16E−12 | 3.27E−10 | KIT |
| 114 | NTRK3 | 5785.4515 | −3.702 | 0.3858 | −7.0039 | 2.49E−12 | 3.74E−10 | CD49f |
| 115 | CLDN7 | 442.7752 | 3.184 | 0.3121 | 6.9977 | 2.60E−12 | 3.87E−10 | KIT |
| 116 | LIMA1 | 4058.607 | −2.87 | 0.2693 | −6.945 | 3.78E−12 | 5.58E−10 | CD49f |
| 117 | POU2F3 | 110.8581 | 3.514 | 0.3624 | 6.9387 | 3.96E−12 | 5.79E−10 | KIT |
| 118 | GLIPR2 | 439.9039 | 2.043 | 0.1513 | 6.896 | 5.35E−12 | 7.75E−10 | KIT |
| 119 | C10orf90 | 37.7877 | 7.112 | 0.8913 | 6.8568 | 7.04E−12 | 1.01E−09 | KIT |
| 120 | RHPN2 | 1252.953 | 2.849 | 0.2706 | 6.8326 | 8.34E−12 | 1.19E−09 | KIT |
| 121 | PTGES | 537.5503 | 2.783 | 0.261 | 6.8313 | 8.41E−12 | 1.19E−09 | KIT |
| 122 | CCDC8 | 948.3262 | −1.68 | 0.0997 | −6.8212 | 9.03E−12 | 1.27E−09 | CD49f |
| 123 | HTR7 | 48.7858 | −4.869 | 0.5675 | −6.8188 | 9.18E−12 | 1.28E−09 | CD49f |
| 124 | EVA1A | 503.0518 | −2.344 | 0.1973 | −6.8141 | 9.48E−12 | 1.31E−09 | CD49f |
| 125 | MACC1 | 266.4891 | 3.474 | 0.3647 | 6.7824 | 1.18E−11 | 1.62E−09 | KIT |
| 126 | HSPG2 | 8211.0362 | −2.716 | 0.2536 | −6.7662 | 1.32E−11 | 1.79E−09 | CD49f |
| 127 | NCALD | 420.0531 | 2.664 | 0.2479 | 6.7117 | 1.92E−11 | 2.59E−09 | KIT |
| 128 | AC008132.2 | 20.3323 | 7.887 | 1.03 | 6.6864 | 2.29E−11 | 3.06E−09 | KIT |
| 129 | NAT8L | 30.1122 | 6.153 | 0.7743 | 6.6547 | 2.84E−11 | 3.76E−09 | KIT |
| 130 | LAMB1 | 44373.0317 | −2.794 | 0.2718 | −6.6023 | 4.05E−11 | 5.33E−09 | CD49f |
| 131 | TSPAN2 | 164.0281 | −3.133 | 0.3233 | −6.5991 | 4.14E−11 | 5.40E−09 | CD49f |
| 132 | LGALS9C | 29.8687 | −4.29 | 0.4989 | −6.594 | 4.28E−11 | 5.55E−09 | CD49f |
| 133 | ARFGEF3 | 363.1099 | 2.883 | 0.2864 | 6.5763 | 4.82E−11 | 6.20E−09 | KIT |
| 134 | DOK7 | 280.1069 | −2.374 | 0.2091 | −6.5687 | 5.08E−11 | 6.48E−09 | CD49f |
| 135 | COL4A1 | 16775.4624 | −3.238 | 0.3408 | −6.5659 | 5.17E−11 | 6.55E−09 | CD49f |
| 136 | WNT6 | 210.2219 | −2.474 | 0.2247 | −6.5601 | 5.38E−11 | 6.76E−09 | CD49f |
| 137 | THY1 | 39.5627 | −5.506 | 0.6871 | −6.5576 | 5.47E−11 | 6.83E−09 | CD49f |
| 138 | C6orf15 | 141.6523 | 6.524 | 0.8426 | 6.5552 | 5.56E−11 | 6.89E−09 | KIT |
| 139 | PLEKHB1 | 345.4407 | 2.682 | 0.2569 | 6.5487 | 5.80E−11 | 7.14E−09 | KIT |
| 140 | FBXL22 | 98.383 | −3.65 | 0.4055 | −6.5346 | 6.38E−11 | 7.74E−09 | CD49f |
| 141 | IL18R1 | 31.095 | 5.697 | 0.7187 | 6.5351 | 6.36E−11 | 7.74E−09 | KIT |
| 142 | ANGPTL2 | 228.7259 | −4.006 | 0.4604 | −6.5291 | 6.62E−11 | 7.97E−09 | CD49f |
| 143 | ELF3 | 5084.1013 | 3.469 | 0.3791 | 6.5128 | 7.37E−11 | 8.82E−09 | KIT |
| 144 | DLL1 | 1364.954 | −3.052 | 0.3163 | −6.4875 | 8.73E−11 | 1.04E−08 | CD49f |
| 145 | RERG | 698.8955 | 4.577 | 0.5521 | 6.4783 | 9.28E−11 | 1.09E−08 | KIT |
| 146 | TMEM63A | 3343.969 | 2.026 | 0.1586 | 6.4678 | 9.94E−11 | 1.16E−08 | KIT |
| 147 | IQCJ- | 901.3625 | −2.545 | 0.2396 | −6.4499 | 1.12E−10 | 1.30E−08 | CD49f |
| | SCHIP1 | | | | | | | |
| 148 | KIAA1324 | 1215.0732 | 3.69 | 0.4179 | 6.435 | 1.23E−10 | 1.43E−08 | KIT |
| 149 | RAB27B | 216.624 | 3.381 | 0.3703 | 6.4297 | 1.28E−10 | 1.47E−08 | KIT |
| 150 | LMOD1 | 361.6982 | −4.099 | 0.4822 | −6.4276 | 1.30E−10 | 1.48E−08 | CD49f |
| 151 | TPRG1 | 60.3668 | −3.114 | 0.3294 | −6.4168 | 1.39E−10 | 1.58E−08 | CD49f |
| 152 | ARHGEF10L | 1132.9484 | 2.228 | 0.1916 | 6.4109 | 1.45E−10 | 1.63E−08 | KIT |
| 153 | RGS16 | 617.9317 | −3.894 | 0.4522 | −6.4 | 1.55E−10 | 1.74E−08 | CD49f |
| 154 | MYLK | 7804.2955 | −3.591 | 0.406 | −6.3809 | 1.76E−10 | 1.96E−08 | CD49f |
| 155 | COL8A2 | 672.1866 | −2.613 | 0.2538 | −6.355 | 2.08E−10 | 2.30E−08 | CD49f |
| 156 | CCL28 | 338.9533 | 2.439 | 0.227 | 6.3407 | 2.29E−10 | 2.51E−08 | KIT |
| 157 | PDLIM7 | 2430.6586 | −2.439 | 0.2273 | −6.3278 | 2.49E−10 | 2.71E−08 | CD49f |
| 158 | KCNJ4 | 26.66 | 4.519 | 0.5585 | 6.3015 | 2.95E−10 | 3.19E−08 | KIT |
| 159 | MTSS1 | 1497.432 | −2.276 | 0.2036 | −6.2666 | 3.69E−10 | 3.97E−08 | CD49f |
| 160 | BEGAIN | 208.6656 | −2.932 | 0.3087 | −6.2585 | 3.89E−10 | 4.16E−08 | CD49f |
| 161 | TBC1D9 | 1121.9067 | −2.801 | 0.2883 | −6.2478 | 4.16E−10 | 4.42E−08 | CD49f |
| 162 | GRIN2C | 1013.049 | −2.674 | 0.268 | −6.2467 | 4.19E−10 | 4.43E−08 | CD49f |
| 163 | PRODH | 89.4462 | 3.991 | 0.4793 | 6.2406 | 4.36E−10 | 4.57E−08 | KIT |
| 164 | MFSD4A | 186.04 | 2.587 | 0.2543 | 6.239 | 4.40E−10 | 4.59E−08 | KIT |
| 165 | LRRC3B | 82.5272 | −4.369 | 0.5406 | −6.2329 | 4.58E−10 | 4.75E−08 | CD49f |
| 166 | TSHZ3 | 1005.1589 | −2.965 | 0.3155 | −6.2295 | 4.68E−10 | 4.82E−08 | CD49f |
| 167 | GSR | 374.5487 | 2.576 | 0.2533 | 6.2234 | 4.87E−10 | 4.98E−08 | KIT |
| 168 | ADAMTS2 | 745.7882 | −4.481 | 0.5604 | −6.2121 | 5.23E−10 | 5.32E−08 | CD49f |
| 169 | GPRC5A | 3612.5841 | 3.849 | 0.4588 | 6.2096 | 5.31E−10 | 5.38E−08 | KIT |
| 170 | PTCHD4 | 53.7187 | 4.632 | 0.5851 | 6.2073 | 5.39E−10 | 5.42E−08 | KIT |
| 171 | ERBB3 | 2682.6065 | 2.761 | 0.2839 | 6.2034 | 5.52E−10 | 5.53E−08 | KIT |
| 172 | RAP1GAP2 | 383.7273 | 3.963 | 0.4826 | 6.1402 | 8.24E−10 | 8.20E−08 | KIT |
| 173 | TINAGL1 | 1669.2674 | −2.575 | 0.2582 | −6.0992 | 1.07E−09 | 1.05E−07 | CD49f |
| 174 | JAM3 | 1674.7765 | −3.301 | 0.3773 | −6.0973 | 1.08E−09 | 1.06E−07 | CD49f |
| 175 | TGFB1 | 241.7437 | −2.282 | 0.2106 | −6.0886 | 1.14E−09 | 1.11E−07 | CD49f |
| 176 | TNNI2 | 883.6793 | −4.336 | 0.5484 | −6.0836 | 1.18E−09 | 1.14E−07 | CD49f |
| 177 | PAK5 | 92.4138 | −3.379 | 0.3926 | −6.0603 | 1.36E−09 | 1.31E−07 | CD49f |
| 178 | BCAM | 6511.5245 | −2.916 | 0.3171 | −6.0439 | 1.50E−09 | 1.45E−07 | CD49f |
| 179 | SNPH | 313.6635 | −2.775 | 0.2939 | −6.0392 | 1.55E−09 | 1.46E−07 | CD49f |
| 180 | THSD1 | 140.4014 | −2.116 | 0.1849 | −6.0394 | 1.55E−09 | 1.46E−07 | CD49f |
| 181 | ZBTB7B | 1090.3513 | 2.523 | 0.2521 | 6.0394 | 1.55E−09 | 1.46E−07 | KIT |
| 182 | TGM5 | 51.1962 | 3.972 | 0.493 | 6.0293 | 1.65E−09 | 1.55E−07 | KIT |
| 183 | ILDR1 | 57.4363 | 2.519 | 0.2527 | 6.013 | 1.82E−09 | 1.70E−07 | KIT |
| 184 | IGFBP4 | 2270.0582 | −2.28 | 0.2149 | −5.9572 | 2.57E−09 | 2.38E−07 | CD49f |
| 185 | HEY2 | 423.1618 | 2.886 | 0.3178 | 5.9359 | 2.92E−09 | 2.70E−07 | KIT |
| 186 | TRIL | 279.4709 | −2.362 | 0.2296 | −5.931 | 3.01E−09 | 2.77E−07 | CD49f |
| 187 | DBNDD2 | 449.9484 | 2.067 | 0.1803 | 5.9154 | 3.31E−09 | 3.03E−07 | KIT |
| 188 | COL9A2 | 15955.6162 | −3.545 | 0.4308 | 5.9085 | 3.45E−09 | 3.14E−07 | CD49f |
| 189 | VGLL1 | 120.9464 | 2.823 | 0.3088 | 5.9048 | 3.53E−09 | 3.20E−07 | KIT |
| 190 | GJC3 | 72.2072 | 3.141 | 0.3637 | 5.8877 | 3.92E−09 | 3.53E−07 | KIT |
| 191 | COL5A1 | 3217.5085 | −3.459 | 0.4191 | −5.8672 | 4.43E−09 | 3.97E−07 | CD49f |
| 192 | KRT12 | 45.014 | 7.438 | 1.1084 | 5.8084 | 6.31E−09 | 5.62E−07 | KIT |
| 193 | ITIH5 | 248.4262 | −3.502 | 0.4331 | 5.7776 | 7.58E−09 | 6.71E−07 | CD49f |
| 194 | KIRREL1 | 4252.7327 | −2.382 | 0.2393 | 5.7764 | 7.63E−09 | 6.73E−07 | CD49f |
| 195 | OASL | 95.8276 | 3.719 | 0.4721 | 5.7603 | 8.40E−09 | 7.36E−07 | KIT |
| 196 | TAGLN | 11588.5175 | −3.1 | 0.3665 | −5.7295 | 1.01E−08 | 8.79E−07 | CD49f |
| 197 | ATP8B4 | 394.2486 | −4.604 | 0.6298 | −5.7225 | 1.05E−08 | 9.12E−07 | CD49f |
| 198 | CPE | 253.9929 | −2.653 | 0.2899 | −5.7016 | 1.19E−08 | 1.03E−06 | CD49f |
| 199 | CLMP | 1037.3807 | −2.623 | 0.2855 | −5.6861 | 1.30E−08 | 1.12E−06 | CD49f |
| 200 | ITM2C | 5681.0934 | −2.267 | 0.2228 | −5.6841 | 1.31E−08 | 1.12E−06 | CD49f |
| 201 | LY6D | 53.593 | 7.344 | 1.1163 | 5.6826 | 1.33E−08 | 1.13E−06 | KIT |
| 202 | COL4A2 | 34762.9516 | −2.875 | 0.3302 | −5.6788 | 1.36E−08 | 1.14E−06 | CD49f |
| 203 | RBBP8NL | 149.0336 | 2.246 | 0.2194 | 5.6791 | 1.35E−08 | 1.14E−06 | KIT |
| 204 | C1QTNF12 | 67.206 | −3.245 | 0.3957 | −5.6732 | 1.40E−08 | 1.18E−06 | CD49f |
| 205 | CACNA1C | 323.8254 | −2.908 | 0.3367 | −5.6673 | 1.45E−08 | 1.21E−06 | CD49f |
| 206 | SYT8 | 3819.053 | −3.857 | 0.5043 | −5.6649 | 1.47E−08 | 1.22E−06 | CD49f |
| 207 | CARD9 | 99.5282 | 2.759 | 0.3109 | 5.6569 | 1.54E−08 | 1.27E−06 | KIT |
| 208 | SORBS1 | 1516.4581 | −2.836 | 0.3257 | 5.6377 | 1.72E−08 | 1.42E−06 | CD49f |
| 209 | SLC2A9 | 250.7817 | −2.135 | 0.2021 | −5.6162 | 1.95E−08 | 1.60E−06 | CD49f |
| 210 | PROM1 | 2106.8744 | 3.365 | 0.4219 | 5.6057 | 2.07E−08 | 1.69E−06 | KIT |
| 211 | ADAMTS9 | 2510.3693 | −2.562 | 0.2793 | −5.5936 | 2.22E−08 | 1.80E−06 | CD49f |
| 212 | SYTL4 | 31.3787 | 3.118 | 0.3821 | 5.5447 | 2.94E−08 | 2.38E−06 | KIT |
| 213 | LTBP2 | 2756.953 | −4.147 | 0.5676 | −5.5437 | 2.96E−08 | 2.38E−06 | CD49f |
| 214 | TPM1 | 7796.601 | −2.344 | 0.243 | −5.5335 | 3.14E−08 | 2.51E−06 | CD49f |
| 215 | FNDC1 | 3722.146 | −3.097 | 0.3791 | −5.5297 | 3.21E−08 | 2.55E−06 | CD49f |
| 216 | CHRM1 | 52.8707 | 2.995 | 0.3611 | 5.5257 | 3.28E−08 | 2.60E−06 | KIT |
| 217 | EHD2 | 1073.1608 | −2.43 | 0.2595 | −5.5119 | 3.55E−08 | 2.80E−06 | CD49f |
| 218 | AC007192.1 | 30.6791 | −6.011 | 0.9098 | −5.5073 | 3.64E−08 | 2.85E−06 | CD49f |
| 219 | SOX14 | 36.783 | 5.905 | 0.8906 | 5.5071 | 3.65E−08 | 2.85E−06 | KIT |
| 220 | HES4 | 714.2527 | 2.882 | 0.3438 | 5.4728 | 4.43E−08 | 3.44E−06 | KIT |
| 221 | LMTK3 | 90.7373 | 3.182 | 0.3988 | 5.472 | 4.45E−08 | 3.44E−06 | KIT |
| 222 | SLC45A3 | 451.3037 | −2.668 | 0.3051 | −5.4686 | 4.54E−08 | 3.49E−06 | CD49f |
| 223 | SIX3 | 72.4208 | 4.726 | 0.6844 | 5.4439 | 5.21E−08 | 4.00E−06 | KIT |
| 224 | NGFR | 820.6682 | −3.79 | 0.5128 | −5.4411 | 5.29E−08 | 4.04E−06 | CD49f |
| 225 | EMID1 | 161.2288 | −2.281 | 0.2365 | −5.4178 | 6.04E−08 | 4.57E−06 | CD49f |
| 226 | SPATA13 | 879.9748 | 2.842 | 0.3399 | 5.4181 | 6.02E−08 | 4.57E−06 | KIT |
| 227 | OLIG1 | 139.7232 | 3.705 | 0.5007 | 5.4019 | 6.59E−08 | 4.97E−06 | KIT |
| 228 | DEPP1 | 975.0208 | 2.784 | 0.3304 | 5.3992 | 6.70E−08 | 5.02E−06 | KIT |
| 229 | SQOR | 98.4853 | 2.468 | 0.272 | 5.398 | 6.74E−08 | 5.03E−06 | KIT |
| 230 | CFH | 240.6931 | −6.3 | 0.9839 | −5.3866 | 7.18E−08 | 5.34E−06 | CD49f |
| 231 | ATP8A1 | 186.8561 | 1.935 | 0.1738 | 5.3807 | 7.42E−08 | 5.49E−06 | KIT |
| 232 | CRHR1 | 391.0393 | 6.493 | 1.0228 | 5.3708 | 7.84E−08 | 5.78E−06 | KIT |
| 233 | ADAMTS12 | 82.3015 | −3.623 | 0.4886 | −5.3679 | 7.96E−08 | 5.85E−06 | CD49f |
| 234 | SCARF2 | 438.7794 | −2.099 | 0.2053 | −5.3524 | 8.68E−08 | 6.34E−06 | CD49f |
| 235 | TUSC1 | 371.8926 | −1.561 | 0.105 | −5.3383 | 9.38E−08 | 6.83E−06 | CD49f |
| 236 | THBS1 | 92666.1233 | −2.175 | 0.2202 | −5.3359 | 9.50E−08 | 6.89E−06 | CD49f |
| 237 | LTF | 470.2304 | 5.537 | 0.8536 | 5.3156 | 1.06E−07 | 7.67E−06 | KIT |
| 238 | GYPC | 376.6961 | −2.732 | 0.3265 | −5.3054 | 1.12E−07 | 8.06E−06 | CD49f |
| 239 | ITGA3 | 2619.1255 | −2.263 | 0.238 | −5.3051 | 1.13E−07 | 8.06E−06 | CD49f |
| 240 | DEPTOR | 18.7189 | 3.849 | 0.5384 | 5.2925 | 1.21E−07 | 8.57E−06 | KIT |
| 241 | EFEMP1 | 139.1878 | −3.101 | 0.3969 | −5.2924 | 1.21E−07 | 8.57E−06 | CD49f |
| 242 | IQGAP2 | 44.386 | 3.168 | 0.4105 | 5.282 | 1.28E−07 | 9.03E−06 | KIT |
| 243 | FLT4 | 141.929 | 3.047 | 0.3885 | 5.2685 | 1.38E−07 | 9.68E−06 | KIT |
| 244 | SLC5A1 | 167.4432 | 3.294 | 0.4357 | 5.266 | 1.39E−07 | 9.78E−06 | KIT |
| 245 | FGFR1 | 18946.1571 | −1.754 | 0.1433 | −5.262 | 1.42E−07 | 9.95E−06 | CD49f |
| 246 | XK | 14.4947 | 5.329 | 0.8237 | 5.2555 | 1.48E−07 | 1.03E−05 | KIT |
| 247 | AC008687.7 | 31.8481 | −4.007 | 0.5735 | −5.2435 | 1.58E−07 | 1.09E−05 | CD49f |
| 248 | VWA1 | 1220.1432 | −2.254 | 0.2395 | −5.2364 | 1.64E−07 | 1.13E−05 | CD49f |
| 249 | PLOD3 | 1206.4395 | −2.264 | 0.2416 | −5.2312 | 1.68E−07 | 1.16E−05 | CD49f |
| 250 | GSAP | 311.5681 | 2.121 | 0.2146 | 5.2224 | 1.77E−07 | 1.21E−05 | KIT |
| 251 | PTPRT | 2035.2518 | −3.363 | 0.4532 | −5.2145 | 1.84E−07 | 1.26E−05 | CD49f |
| 252 | PACSIN1 | 85.3394 | 4.188 | 0.6123 | 5.2071 | 1.92E−07 | 1.30E−05 | KIT |
| 253 | KCTD4 | 29.9571 | −3.755 | 0.5337 | 5.1627 | 2.43E−07 | 1.65E−05 | CD49f |
| 254 | PPM1H | 176.5077 | 2.184 | 0.2302 | 5.1427 | 2.71E−07 | 1.82E−05 | KIT |
| 255 | RASGEF1C | 17.0194 | 5.997 | 0.9719 | 5.1413 | 2.73E−07 | 1.83E−05 | KIT |
| 256 | KLK11 | 234.7249 | 3.963 | 0.5767 | 5.1381 | 2.78E−07 | 1.85E−05 | KIT |
| 257 | ASPG | 32.9458 | 6.987 | 1.1686 | 5.1231 | 3.01E−07 | 2.00E−05 | KIT |
| 258 | IL1R2 | 72.8028 | 4.747 | 0.7318 | 5.1202 | 3.05E−07 | 2.02E−05 | KIT |
| 259 | WDR81 | 937.2328 | −1.615 | 0.1202 | 5.1203 | 3.05E−07 | 2.02E−05 | CD49f |
| 260 | LOXL1 | 545.9287 | −2.241 | 0.2432 | −5.104 | 3.33E−07 | 2.19E−05 | CD49f |
| 261 | AC024940.2 | 219.693 | 2.846 | 0.3631 | 5.083 | 3.72E−07 | 2.44E−05 | KIT |
| 262 | GOLGA8F | 32.0993 | −6.217 | 1.028 | −5.0747 | 3.88E−07 | 2.53E−05 | CD49f |
| 263 | HES2 | 150.1238 | 3.404 | 0.4739 | 5.072 | 3.94E−07 | 2.56E−05 | KIT |
| 264 | AC007325.2 | 93.2822 | 3.324 | 0.4596 | 5.0571 | 4.26E−07 | 2.76E−05 | KIT |
| 265 | HAND2 | 61.1183 | 3.997 | 0.5928 | 5.0559 | 4.28E−07 | 2.77E−05 | KIT |
| 266 | MUC5B | 962.2404 | 3.517 | 0.4992 | 5.0434 | 4.57E−07 | 2.94E−05 | KIT |
| 267 | LRRC26 | 19.4478 | 5.072 | 0.8089 | 5.0341 | 4.80E−07 | 3.08E−05 | KIT |
| 268 | CCDC74A | 343.265 | −1.849 | 0.169 | 5.0253 | 5.03E−07 | 3.21E−05 | CD49f |
| 269 | NRG1 | 402.1622 | −4.181 | 0.6333 | −5.0228 | 5.09E−07 | 3.24E−05 | CD49f |
| 270 | GCSAM | 17.0419 | −5.733 | 0.9429 | −5.0199 | 5.17E−07 | 3.28E−05 | CD49f |
| 271 | CFD | 41.6696 | 4.509 | 0.7017 | 5.0007 | 5.71E−07 | 3.61E−05 | KIT |
| 272 | C11orf52 | 46.9087 | 3.644 | 0.5298 | 4.9901 | 6.03E−07 | 3.79E−05 | KIT |
| 273 | KCNK5 | 512.9469 | 2.21 | 0.2428 | 4.9833 | 6.25E−07 | 3.92E−05 | KIT |
| 274 | PTPRE | 495.4006 | −2.38 | 0.277 | −4.9811 | 6.32E−07 | 3.95E−05 | CD49f |
| 275 | ISG20 | 337.7975 | 1.918 | 0.1844 | 4.9772 | 6.45E−07 | 4.01E−05 | KIT |
| 276 | ALPL | 249.2299 | 4.477 | 0.7 | 4.9664 | 6.82E−07 | 4.23E−05 | KIT |
| 277 | RTL5 | 427.6659 | −2.487 | 0.3008 | −4.9432 | 7.69E−07 | 4.75E−05 | CD49f |
| 278 | GALNT5 | 34.1857 | −3.07 | 0.4195 | −4.9345 | 8.03E−07 | 4.94E−05 | CD49f |
| 279 | PHLDB1 | 3087.1589 | −2.321 | 0.2681 | −4.9278 | 8.32E−07 | 5.10E−05 | CD49f |
| 280 | PLEKHS1 | 199.4759 | 4.359 | 0.6829 | 4.9191 | 8.69E−07 | 5.31E−05 | KIT |
| 281 | PCDH1 | 467.2688 | 2.422 | 0.2892 | 4.9182 | 8.73E−07 | 5.32E−05 | KIT |
| 282 | MFAP4 | 118.8569 | −4.295 | 0.6705 | −4.9134 | 8.95E−07 | 5.43E−05 | CD49f |
| 283 | MOV10L1 | 58.4505 | −2.62 | 0.3299 | −4.9123 | 9.00E−07 | 5.44E−05 | CD49f |
| 284 | COL16A1 | 3041.4247 | −2.94 | 0.395 | −4.911 | 9.06E−07 | 5.46E−05 | CD49f |
| 285 | SYNPO2 | 281.6815 | −3.228 | 0.4539 | −4.9071 | 9.25E−07 | 5.55E−05 | CD49f |
| 286 | GAS1 | 812.578 | −2.719 | 0.3503 | −4.906 | 9.30E−07 | 5.56E−05 | CD49f |
| 287 | MB | 36.7469 | 4.707 | 0.7564 | 4.9001 | 9.58E−07 | 5.71E−05 | KIT |
| 288 | MYEOV | 107.7522 | 4.075 | 0.6298 | 4.8821 | 1.05E−06 | 6.23E−05 | KIT |
| 289 | INHBB | 1248.5895 | 2.776 | 0.3641 | 4.8771 | 1.08E−06 | 6.37E−05 | KIT |
| 290 | ANO1 | 3097.913 | −2.964 | 0.4028 | 4.8763 | 1.08E−06 | 6.37E−05 | CD49f |
| 291 | MMP10 | 97.5095 | −2.835 | 0.3768 | 4.8702 | 1.12E−06 | 6.55E−05 | CD49f |
| 292 | PTPN14 | 4551.1899 | −1.755 | 0.1554 | −4.8616 | 1.16E−06 | 6.82E−05 | CD49f |
| 293 | KIF13B | 1114.6002 | 1.968 | 0.1995 | 4.8514 | 1.23E−06 | 7.16E−05 | KIT |
| 294 | MBP | 319.7271 | 2.636 | 0.3376 | 4.8464 | 1.26E−06 | 7.31E−05 | KIT |
| 295 | TMEM211 | 14.5668 | 4.582 | 0.7399 | 4.8417 | 1.29E−06 | 7.46E−05 | KIT |
| 296 | BRINP1 | 111.1593 | −3.623 | 0.5421 | −4.8381 | 1.31E−06 | 7.57E−05 | CD49f |
| 297 | PLCH2 | 1444.6951 | −2.115 | 0.2306 | −4.8366 | 1.32E−06 | 7.61E−05 | CD49f |
| 298 | FSTL4 | 158.9567 | −2.918 | 0.3968 | −4.8342 | 1.34E−06 | 7.65E−05 | CD49f |
| 299 | OLFM4 | 150.2304 | 4.557 | 0.7357 | 4.8344 | 1.34E−06 | 7.65E−05 | KIT |
| 300 | SLC15A2 | 182.6362 | 2.747 | 0.3626 | 4.8165 | 1.46E−06 | 8.33E−05 | KIT |
| 301 | CDC42EP4 | 1361.243 | 2.274 | 0.2656 | 4.7968 | 1.61E−06 | 9.16E−05 | KIT |
| 302 | C9orf152 | 42.7535 | 3.5 | 0.5216 | 4.7925 | 1.65E−06 | 9.33E−05 | KIT |
| 303 | BOC | 6161.5026 | −2.112 | 0.2322 | −4.7889 | 1.68E−06 | 9.46E−05 | CD49f |
| 304 | SH3TC1 | 47.0147 | −2.88 | 0.3937 | −4.7769 | 1.78E−06 | 0.00010014 | CD49f |
| 305 | CD200 | 1260.5006 | −2.913 | 0.4015 | −4.7653 | 1.89E−06 | 0.000105775 | CD49f |
| 306 | COL5A2 | 779.6304 | −3.282 | 0.4794 | −4.7596 | 1.94E−06 | 0.000108404 | CD49f |
| 307 | GDPD5 | 99.9528 | 2.808 | 0.38 | 4.7576 | 1.96E−06 | 0.000109157 | KIT |
| 308 | MPPED1 | 12.7481 | −7.018 | 1.2682 | −4.7454 | 2.08E−06 | 0.000115554 | CD49f |
| 309 | AC068580.4 | 500.9154 | −2.575 | 0.3329 | −4.7323 | 2.22E−06 | 0.000122882 | CD49f |
| 310 | CFTR | 33.7884 | 6.276 | 1.116 | 4.7276 | 2.27E−06 | 0.000125379 | KIT |
| 311 | NET1 | 2819.8098 | 2.231 | 0.2619 | 4.7002 | 2.60E−06 | 0.000142931 | KIT |
| 312 | MTCL1 | 1006.5883 | −2.54 | 0.3278 | −4.6974 | 2.63E−06 | 0.000144448 | CD49f |
| 313 | IL34 | 41.0318 | 3.244 | 0.4798 | 4.677 | 2.91E−06 | 0.000159084 | KIT |
| 314 | CRLF1 | 377.9731 | −3.133 | 0.4564 | −4.6739 | 2.95E−06 | 0.000160955 | CD49f |
| 315 | MSMO1 | 1068.4691 | −1.825 | 0.177 | −4.6609 | 3.15E−06 | 0.000170966 | CD49f |
| 316 | SHC4 | 2033.1728 | 3.294 | 0.4925 | 4.6582 | 3.19E−06 | 0.000172617 | KIT |
| 317 | CMTM8 | 36.6138 | 3.026 | 0.4363 | 4.6448 | 3.40E−06 | 0.000183635 | KIT |
| 318 | ALDH3B2 | 273.4977 | 3.279 | 0.4912 | 4.6397 | 3.49E−06 | 0.000187628 | KIT |
| 319 | HGFAC | 115.5767 | −1.808 | 0.1742 | −4.6358 | 3.56E−06 | 0.000190624 | CD49f |
| 320 | KCNIP3 | 450.8885 | −2.229 | 0.2655 | −4.6298 | 3.66E−06 | 0.000195608 | CD49f |
| 321 | CFAP57 | 26.6506 | −3.647 | 0.572 | −4.6281 | 3.69E−06 | 0.000196652 | CD49f |
| 322 | LRP4 | 711.9052 | −1.981 | 0.2121 | −4.6237 | 3.77E−06 | 0.000200252 | CD49f |
| 323 | KRT80 | 549.403 | 3.01 | 0.4348 | 4.6221 | 3.80E−06 | 0.000201179 | KIT |
| 324 | SLC8A1 | 31.4355 | −5.646 | 1.0054 | −4.6211 | 3.82E−06 | 0.000201467 | CD49f |
| 325 | WTIP | 624.0178 | −1.667 | 0.1449 | −4.6065 | 4.10E−06 | 0.000215537 | CD49f |
| 326 | MISP | 42.1705 | 2.923 | 0.4187 | 4.5931 | 4.37E−06 | 0.000229072 | KIT |
| 327 | CDH13 | 1180.0653 | −3.444 | 0.5325 | 4.5898 | 4.44E−06 | 0.000232026 | CD49f |
| 328 | RAB7B | 141.7308 | −3.117 | 0.4629 | 4.5743 | 4.78E−06 | 0.00024921 | CD49f |
| 329 | PAMR1 | 12.9347 | −3.846 | 0.6238 | −4.5631 | 5.04E−06 | 0.000262038 | CD49f |
| 330 | TBX2 | 162.4363 | −2.329 | 0.2915 | −4.5609 | 5.09E−06 | 0.000263998 | CD49f |
| 331 | KCNN4 | 539.9205 | 3.131 | 0.4674 | 4.5593 | 5.13E−06 | 0.000264418 | KIT |
| 332 | PLCH1 | 403.7506 | 2.322 | 0.2899 | 4.5597 | 5.12E−06 | 0.000264418 | KIT |
| 333 | DZIP1L | 122.653 | −2.242 | 0.2727 | −4.5539 | 5.27E−06 | 0.000269662 | CD49f |
| 334 | PLEKHH1 | 593.6963 | 1.672 | 0.1476 | 4.5543 | 5.25E−06 | 0.000269662 | KIT |
| 335 | CDH11 | 2049.0008 | −2.381 | 0.3043 | 4.5382 | 5.67E−06 | 0.00028884 | CD49f |
| 336 | COL4A6 | 141.9402 | −3.128 | 0.4688 | −4.5383 | 5.67E−06 | 0.00028884 | CD49f |
| 337 | SYNE3 | 366.4454 | −2.824 | 0.402 | −4.5367 | 5.71E−06 | 0.000290038 | CD49f |
| 338 | HS6ST2 | 56.8621 | 4.364 | 0.742 | 4.5337 | 5.80E−06 | 0.0002933 | KIT |
| 339 | GREB1L | 64.3021 | 2.866 | 0.4118 | 4.5313 | 5.86E−06 | 0.000295732 | KIT |
| 340 | COL4A5 | 2041.2601 | −2.238 | 0.2737 | −4.5225 | 6.11E−06 | 0.000307405 | CD49f |
| 341 | ATP2A3 | 512.2445 | 3.921 | 0.6466 | 4.5167 | 6.28E−06 | 0.00031505 | KIT |
| 342 | FBXL16 | 60.0433 | 4.283 | 0.7273 | 4.5145 | 6.35E−06 | 0.000317455 | KIT |
| 343 | SPARC | 50651.6216 | −2.837 | 0.4072 | 4.5116 | 6.43E−06 | 0.000320545 | CD49f |
| 344 | TTYH1 | 2778.3378 | 3.271 | 0.5033 | 4.5112 | 6.45E−06 | 0.000320545 | KIT |
| 345 | KLK10 | 1195.683 | 3.681 | 0.5955 | 4.5019 | 6.74E−06 | 0.000333937 | KIT |
| 346 | TMEM59L | 197.2517 | −2.886 | 0.4194 | −4.4964 | 6.91E−06 | 0.000341587 | CD49f |
| 347 | C15orf62 | 88.1226 | 2.779 | 0.3963 | 4.4898 | 7.13E−06 | 0.000351327 | KIT |
| 348 | RASSF4 | 267.8517 | 2.545 | 0.3445 | 4.4857 | 7.27E−06 | 0.000357176 | KIT |
| 349 | B3GNT3 | 14.5736 | 6.24 | 1.1684 | 4.4848 | 7.30E−06 | 0.000357728 | KIT |
| 350 | ERICH5 | 11.5413 | 4.198 | 0.7152 | 4.4721 | 7.75E−06 | 0.00037852 | KIT |
| 351 | FEZ1 | 43.0128 | −3.127 | 0.4769 | −4.46 | 8.20E−06 | 0.00039938 | CD49f |
| 352 | KLK14 | 225.5139 | 3.665 | 0.5977 | 4.4583 | 8.26E−06 | 0.000401418 | KIT |
| 353 | CDH3 | 2812.5341 | −2.614 | 0.3628 | −4.4482 | 8.66E−06 | 0.000419483 | CD49f |
| 354 | WSCD2 | 98.5383 | −3.878 | 0.648 | −4.4403 | 8.98E−06 | 0.000433945 | CD49f |
| 355 | MESP1 | 12.5524 | 4.719 | 0.8386 | 4.4351 | 9.20E−06 | 0.000443474 | KIT |
| 356 | ARHGAP4 | 249.6623 | −1.864 | 0.1949 | −4.4326 | 9.31E−06 | 0.000447239 | CD49f |
| 357 | MFSD6L | 15.0921 | 3.997 | 0.6782 | 4.4186 | 9.93E−06 | 0.000475958 | KIT |
| 358 | SELENBP1 | 269.1599 | 2.547 | 0.3502 | 4.4174 | 9.99E−06 | 0.000477253 | KIT |
| 359 | FXYD3 | 548.0915 | 3.581 | 0.5845 | 4.4156 | 1.01E−05 | 0.000479942 | KIT |
| 360 | IL20RA | 118.6604 | 2.149 | 0.2606 | 4.4104 | 1.03E−05 | 0.000490157 | KIT |
| 361 | RGS9 | 75.1337 | −2.306 | 0.2966 | −4.4032 | 1.07E−05 | 0.000505279 | CD49f |
| 362 | PCSK9 | 43.2724 | −2.958 | 0.4449 | −4.402 | 1.07E−05 | 0.000506779 | CD49f |
| 363 | SORBS2 | 1267.8064 | 2.578 | 0.3593 | 4.3919 | 1.12E−05 | 0.000529534 | KIT |
| 364 | BMP6 | 180.0415 | 2.985 | 0.4522 | 4.3896 | 1.14E−05 | 0.000533487 | KIT |
| 365 | TCN1 | 35.6841 | 4.586 | 0.8171 | 4.3883 | 1.14E−05 | 0.000535244 | KIT |
| 366 | P3H1 | 846.5938 | −2.087 | 0.2494 | −4.3592 | 1.31E−05 | 0.000609911 | CD49f |
| 367 | SPNS2 | 118.9887 | 2.78 | 0.4087 | 4.3559 | 1.33E−05 | 0.000617667 | KIT |
| 368 | SQLE | 686.7909 | −1.727 | 0.1672 | −4.3475 | 1.38E−05 | 0.000639999 | CD49f |
| 369 | SPDEF | 65.6821 | 3.759 | 0.6357 | 4.3401 | 1.42E−05 | 0.000660058 | KIT |
| 370 | TBX22 | 513.5436 | −2.403 | 0.3238 | 4.3345 | 1.46E−05 | 0.000675478 | CD49f |
| 371 | MMP3 | 223.1797 | −3.708 | 0.6257 | −4.328 | 1.50E−05 | 0.000693552 | CD49f |
| 372 | AC022149.1 | 101.2681 | −8.202 | 1.6705 | −4.3109 | 1.63E−05 | 0.000747394 | CD49f |
| 373 | ARHGEF38 | 34.7332 | 4.035 | 0.7042 | 4.3104 | 1.63E−05 | 0.000747394 | KIT |
| 374 | SERPINB2 | 166.5659 | 3.257 | 0.525 | 4.2997 | 1.71E−05 | 0.000782283 | KIT |
| 375 | MMP7 | 783.1937 | 4.692 | 0.859 | 4.2977 | 1.73E−05 | 0.000787298 | KIT |
| 376 | CAPN6 | 552.5105 | −4.931 | 0.9149 | −4.2968 | 1.73E−05 | 0.000788084 | CD49f |
| 377 | MYOM3 | 48.5397 | −2.895 | 0.4432 | −4.2753 | 1.91E−05 | 0.000865864 | CD49f |
| 378 | COPZ2 | 21.6047 | −4.02 | 0.7069 | −4.2726 | 1.93E−05 | 0.000874267 | CD49f |
| 379 | ZNF385A | 339.8272 | −1.542 | 0.1274 | −4.2569 | 2.07E−05 | 0.000935449 | CD49f |
| 380 | CD160 | 31.0341 | −3.96 | 0.6982 | −4.2398 | 2.24E−05 | 0.001007146 | CD49f |
| 381 | CCDC74B | 144.2842 | −2.06 | 0.2501 | −4.2382 | 2.25E−05 | 0.001011704 | CD49f |
| 382 | IGSF5 | 22.1065 | 5.387 | 1.036 | 4.2344 | 2.29E−05 | 0.001025954 | KIT |
| 383 | THEMIS2 | 46.9252 | 2.314 | 0.3105 | 4.2319 | 2.32E−05 | 0.001034705 | KIT |
| 384 | CNN1 | 2223.53 | −3.326 | 0.5497 | 4.2307 | 2.33E−05 | 0.001037597 | CD49f |
| 385 | AAK1 | 1838.283 | −1.833 | 0.1975 | 4.2206 | 2.44E−05 | 0.001082601 | CD49f |
| 386 | E2F2 | 105.5198 | 2.358 | 0.322 | 4.2174 | 2.47E−05 | 0.001094895 | KIT |
| 387 | HIC1 | 242.665 | −2.289 | 0.3058 | −4.2156 | 2.49E−05 | 0.001100809 | CD49f |
| 388 | NDRG2 | 5930.7984 | 2.457 | 0.346 | 4.2126 | 2.52E−05 | 0.001112873 | KIT |
| 389 | DAPK1 | 4523.6967 | 1.741 | 0.176 | 4.2109 | 2.54E−05 | 0.001118486 | KIT |
| 390 | COL12A1 | 973.1815 | −2.857 | 0.4413 | −4.2086 | 2.57E−05 | 0.001126799 | CD49f |
| 391 | GPC1 | 1924.5856 | −1.682 | 0.1622 | −4.207 | 2.59E−05 | 0.001130641 | CD49f |
| 392 | PODXL | 596.0011 | 3.122 | 0.5045 | 4.2067 | 2.59E−05 | 0.001130641 | KIT |
| 393 | USP31 | 1116.8869 | −1.985 | 0.2343 | −4.2056 | 2.60E−05 | 0.001133191 | CD49f |
| 394 | COLEC12 | 3768.1463 | −2.019 | 0.2425 | −4.201 | 2.66E−05 | 0.001153507 | CD49f |
| 395 | INPP4B | 190.9927 | −2.421 | 0.3388 | −4.1955 | 2.72E−05 | 0.001178839 | CD49f |
| 396 | GULP1 | 184.2269 | 2.507 | 0.3611 | 4.1724 | 3.01E−05 | 0.001301708 | KIT |
| 397 | NOTUM | 26.4933 | 5.918 | 1.1801 | 4.1677 | 3.08E−05 | 0.001325899 | KIT |
| 398 | EFCAB1 | 100.1703 | −2.904 | 0.4583 | −4.1542 | 3.26E−05 | 0.00140257 | CD49f |
| 399 | SUSD4 | 385.1293 | 2.007 | 0.2425 | 4.1522 | 3.29E−05 | 0.001411813 | KIT |
| 400 | ADAMTS1 | 6463.6884 | −2.327 | 0.3198 | −4.1488 | 3.34E−05 | 0.001429139 | CD49f |
| 401 | SULT2B1 | 35.7297 | 3.478 | 0.5975 | 4.1466 | 3.37E−05 | 0.001439253 | KIT |
| 402 | C12orf54 | 17.8994 | −4.758 | 0.9068 | −4.1441 | 3.41E−05 | 0.001451316 | CD49f |
| 403 | LGR6 | 8501.0207 | −1.945 | 0.2287 | −4.1322 | 3.59E−05 | 0.00152497 | CD49f |
| 404 | MALL | 553.1104 | 2.824 | 0.4419 | 4.128 | 3.66E−05 | 0.00154928 | KIT |
| 405 | KRT19 | 1902.2321 | 3.313 | 0.5608 | 4.1249 | 3.71E−05 | 0.001566598 | KIT |
| 406 | EDARADD | 61.6097 | −2.218 | 0.2956 | −4.1205 | 3.78E−05 | 0.001592953 | CD49f |
| 407 | S100P | 607.8853 | 4.543 | 0.8608 | 4.1154 | 3.87E−05 | 0.001624551 | KIT |
| 408 | THSD4 | 119.5464 | 2.839 | 0.447 | 4.1143 | 3.88E−05 | 0.001628182 | KIT |
| 409 | CCND1 | 5645.4671 | 1.902 | 0.2193 | 4.111 | 3.94E−05 | 0.001644772 | KIT |
| 410 | CLIC3 | 33.0806 | 4.139 | 0.7636 | 4.1108 | 3.94E−05 | 0.001644772 | KIT |
| 411 | PXN | 2965.2848 | −1.586 | 0.1426 | −4.1073 | 4.00E−05 | 0.00166574 | CD49f |
| 412 | GNAZ | 206.9318 | −1.847 | 0.2075 | −4.0837 | 4.43E−05 | 0.001835995 | CD49f |
| 413 | RNF186 | 13.7117 | 7.327 | 1.5492 | 4.0841 | 4.43E−05 | 0.001835995 | KIT |
| 414 | AC027559.1 | 16.7947 | −4.665 | 0.898 | −4.0817 | 4.47E−05 | 0.001846649 | CD49f |
| 415 | RAI14 | 1175.8307 | −1.843 | 0.2065 | −4.0803 | 4.50E−05 | 0.00185204 | CD49f |
| 416 | TLR1 | 78.3341 | 2.427 | 0.3498 | 4.08 | 4.50E−05 | 0.00185204 | KIT |
| 417 | CDKN2A | 158.2714 | 2.467 | 0.3598 | 4.0766 | 4.57E−05 | 0.001867446 | KIT |
| 418 | CLIP3 | 319.2447 | −1.854 | 0.2096 | −4.0764 | 4.57E−05 | 0.001867446 | CD49f |
| 419 | EFNA5 | 676.657 | 2.521 | 0.3731 | 4.0774 | 4.55E−05 | 0.001867446 | KIT |
| 420 | IL20 | 51.9357 | −3.131 | 0.5237 | −4.0699 | 4.70E−05 | 0.001915714 | CD49f |
| 421 | STARD9 | 197.8257 | −1.898 | 0.221 | −4.0639 | 4.83E−05 | 0.001960485 | CD49f |
| 422 | MYZAP | 83.649 | 2.583 | 0.3911 | 4.0481 | 5.16E−05 | 0.002092867 | KIT |
| 423 | AEBP1 | 1799.0669 | −2.55 | 0.3835 | −4.0412 | 5.32E−05 | 0.002149859 | CD49f |
| 424 | DIRC3 | 30.1637 | −2.532 | 0.3793 | −4.0381 | 5.39E−05 | 0.002173689 | CD49f |
| 425 | HUNK | 569.1967 | −2.139 | 0.2822 | −4.0347 | 5.47E−05 | 0.002200715 | CD49f |
| 426 | BMP1 | 795.3271 | −1.675 | 0.1673 | −4.0326 | 5.52E−05 | 0.002214618 | CD49f |
| 427 | PPP1R9A | 426.7631 | 2.267 | 0.3146 | 4.0273 | 5.64E−05 | 0.002260445 | KIT |
| 428 | KCNQ5 | 311.6338 | −2.272 | 0.3159 | −4.0259 | 5.67E−05 | 0.002267015 | CD49f |
| 429 | PLAT | 7322.1824 | −2.576 | 0.3915 | 4.0255 | 5.69E−05 | 0.002267015 | CD49f |
| 430 | SCGB3A1 | 81.3496 | 4.577 | 0.8891 | 4.0235 | 5.73E−05 | 0.002280368 | KIT |
| 431 | GJA5 | 82.5457 | −4.769 | 0.9383 | −4.0173 | 5.89E−05 | 0.002335884 | CD49f |
| 432 | MED12L | 471.3542 | −2.271 | 0.3179 | −3.9988 | 6.37E−05 | 0.002521163 | CD49f |
| 433 | IL1RAP | 819.4736 | −2.336 | 0.3341 | −3.9979 | 6.39E−05 | 0.002524707 | CD49f |
| 434 | ZIM2 | 121.9578 | −2.324 | 0.3314 | 3.9951 | 6.47E−05 | 0.002548217 | CD49f |
| 435 | KRT16 | 4515.8286 | 2.537 | 0.3853 | 3.988 | 6.66E−05 | 0.002613657 | KIT |
| 436 | QPCT | 228.5486 | 2.367 | 0.3428 | 3.9882 | 6.66E−05 | 0.002613657 | KIT |
| 437 | MLIP | 12.4065 | 3.684 | 0.674 | 3.9814 | 6.85E−05 | 0.002680919 | KIT |
| 438 | KRT15 | 4406.4853 | 2.149 | 0.2887 | 3.9801 | 6.89E−05 | 0.002690116 | KIT |
| 439 | CD38 | 133.2537 | −2.574 | 0.396 | −3.9743 | 7.06E−05 | 0.002744191 | CD49f |
| 440 | IFI30 | 228.7474 | −1.929 | 0.2337 | 3.9748 | 7.04E−05 | 0.002744191 | CD49f |
| 441 | HAS3 | 534.4012 | −2.59 | 0.4003 | −3.9736 | 7.08E−05 | 0.002745605 | CD49f |
| 442 | FHOD3 | 2061.0756 | −2.283 | 0.323 | −3.9722 | 7.12E−05 | 0.002755457 | CD49f |
| 443 | ANKRD18B | 8.3421 | 4.25 | 0.8183 | 3.9709 | 7.16E−05 | 0.002764483 | KIT |
| 444 | AXL | 2391.755 | −2.883 | 0.4756 | −3.959 | 7.53E−05 | 0.002892544 | CD49f |
| 445 | BTC | 42.3254 | 2.397 | 0.353 | 3.9594 | 7.51E−05 | 0.002892544 | KIT |
| 446 | LAYN | 86.861 | −2.782 | 0.4504 | −3.9562 | 7.62E−05 | 0.002920996 | CD49f |
| 447 | LAMA3 | 1434.1452 | −2.624 | 0.4107 | −3.9549 | 7.66E−05 | 0.002930271 | CD49f |
| 448 | SRCIN1 | 281.6098 | 2.334 | 0.3373 | 3.9542 | 7.68E−05 | 0.002931683 | KIT |
| 449 | CMPK2 | 49.5154 | 2.049 | 0.2658 | 3.9475 | 7.90E−05 | 0.003008663 | KIT |
| 450 | HAS2 | 312.221 | −4.541 | 0.9006 | −3.9319 | 8.43E−05 | 0.00320292 | CD49f |
| 451 | MEF2C | 1693.4013 | −2.746 | 0.4445 | 3.9289 | 8.53E−05 | 0.003236397 | CD49f |
| 452 | NID1 | 5608.5996 | −3.032 | 0.5172 | −3.9281 | 8.56E−05 | 0.003239384 | CD49f |
| 453 | PCYT1B | 132.7159 | −2.468 | 0.3739 | −3.9272 | 8.59E−05 | 0.003239485 | CD49f |
| 454 | TP53I11 | 737.0575 | 2.11 | 0.2827 | 3.9271 | 8.60E−05 | 0.003239485 | KIT |
| 455 | ANPEP | 103.4471 | 3.862 | 0.7289 | 3.9265 | 8.62E−05 | 0.003239959 | KIT |
| 456 | TNRC18P1 | 61.6516 | −3.552 | 0.6517 | 3.9154 | 9.02E−05 | 0.003384834 | CD49f |
| 457 | EDN2 | 81.1771 | 2.649 | 0.4211 | 3.9148 | 9.05E−05 | 0.00338625 | KIT |
| 458 | SPTSSB | 17.5955 | 6.567 | 1.4229 | 3.9124 | 9.14E−05 | 0.003412466 | KIT |
| 459 | Z83844.3 | 18.0783 | −6.936 | 1.5221 | −3.9001 | 9.62E−05 | 0.003583601 | CD49f |
| 460 | MUC16 | 131.2314 | 5.082 | 1.0482 | 3.8945 | 9.84E−05 | 0.003658276 | KIT |
| 461 | PIEZO2 | 164.0481 | −2.197 | 0.3076 | 3.8931 | 9.90E−05 | 0.003672592 | CD49f |
| 462 | PGBD5 | 10.0328 | 4.436 | 0.884 | 3.8868 | 0.000101587 | 0.003760902 | KIT |
| 463 | FAM133A | 10.8613 | −5.616 | 1.1883 | −3.8843 | 0.000102607 | 0.003790462 | CD49f |
| 464 | SERPINF2 | 81.1146 | −2.369 | 0.3529 | −3.8789 | 0.000104941 | 0.003868357 | CD49f |
| 465 | SLC16A9 | 39.9455 | −4.756 | 0.9686 | −3.8775 | 0.000105517 | 0.003881222 | CD49f |
| 466 | JPH1 | 178.2247 | 2.133 | 0.2927 | 3.8707 | 0.00010851 | 0.003982726 | KIT |
| 467 | RSPO1 | 55.7695 | 4.025 | 0.7844 | 3.857 | 0.000114804 | 0.004204732 | KIT |
| 468 | KLHL6 | 66.5616 | −1.822 | 0.2134 | −3.8541 | 0.000116161 | 0.004245342 | CD49f |
| 469 | TMEM184A | 156.2349 | 2.672 | 0.434 | 3.8529 | 0.000116723 | 0.004256798 | KIT |
| 470 | CRIP3 | 33.5916 | −2.289 | 0.335 | −3.8475 | 0.000119346 | 0.004343186 | CD49f |
| 471 | MGST2 | 124.4079 | 2.175 | 0.3057 | 3.845 | 0.000120542 | 0.004377398 | KIT |
| 472 | KIAA1614 | 118.5731 | −2.524 | 0.3963 | −3.8441 | 0.000120972 | 0.004383684 | CD49f |
| 473 | SMIM22 | 10.1156 | 3.465 | 0.642 | 3.8395 | 0.000123274 | 0.004457682 | KIT |
| 474 | ANKRD62P1 | 11.0382 | 5.187 | 1.0909 | 3.8384 | 0.000123847 | 0.004468936 | KIT |
| 475 | AC008687.8 | 34.1056 | −3.464 | 0.6438 | 3.8277 | 0.000129353 | 0.004657802 | CD49f |
| 476 | EVPL | 942.3396 | 1.979 | 0.2559 | 3.8266 | 0.000129913 | 0.004668126 | KIT |
| 477 | NOTCH3 | 3494.6947 | 3.641 | 0.6921 | 3.8156 | 0.000135878 | 0.00487222 | KIT |
| 478 | TTC9 | 142.3829 | 2.051 | 0.2758 | 3.8103 | 0.000138805 | 0.00496678 | KIT |
| 479 | LYST | 4875.1521 | −2.111 | 0.292 | −3.806 | 0.000141252 | 0.005043775 | CD49f |
| 480 | NPR2 | 250.6488 | −1.769 | 0.2028 | −3.7924 | 0.000149171 | 0.00531546 | CD49f |
| 481 | LY6E | 1531.7981 | −1.78 | 0.206 | −3.7842 | 0.000154217 | 0.005483857 | CD49f |
| 482 | GLIS1 | 22.7065 | −3.105 | 0.5564 | −3.7822 | 0.000155445 | 0.005516032 | CD49f |
| 483 | ETV3L | 9.5752 | 5.672 | 1.2388 | 3.7712 | 0.000162494 | 0.005754247 | KIT |
| 484 | ACTN1 | 12837.7348 | −1.545 | 0.1445 | −3.7694 | 0.000163618 | 0.00577015 | CD49f |
| 485 | SEMA3E | 53.7024 | 2.614 | 0.4282 | 3.7699 | 0.000163302 | 0.00577015 | KIT |
| 486 | BAIAP2 | 1551.1792 | 1.691 | 0.1836 | 3.7669 | 0.000165283 | 0.005816872 | KIT |
| 487 | NTNG2 | 418.2681 | −2.912 | 0.5098 | −3.7512 | 0.000175968 | 0.0061802 | CD49f |
| 488 | CPQ | 941.7188 | −2.143 | 0.3048 | −3.7501 | 0.000176787 | 0.006196255 | CD49f |
| 489 | LRRC1 | 530.2945 | 2.157 | 0.3087 | 3.7474 | 0.00017865 | 0.006248717 | KIT |
| 490 | LIPH | 405.289 | 2.734 | 0.4634 | 3.742 | 0.000182528 | 0.006371329 | KIT |
| 491 | CHST7 | 29.4 | −2.474 | 0.3945 | −3.735 | 0.000187722 | 0.006539284 | CD49f |
| 492 | FHL1 | 144.594 | −2.892 | 0.5126 | −3.6915 | 0.00022293 | 0.007749986 | CD49f |
| 493 | ALS2CL | 223.5081 | 2.217 | 0.3305 | 3.6809 | 0.000232409 | 0.008063139 | KIT |
| 494 | SYBU | 31.648 | 2.785 | 0.4853 | 3.6781 | 0.000234948 | 0.008134712 | KIT |
| 495 | SDCBP2 | 85.6154 | 2.255 | 0.3413 | 3.677 | 0.000235962 | 0.008153312 | KIT |
| 496 | KIAA0040 | 1986.4147 | −2.086 | 0.2956 | −3.6726 | 0.000240116 | 0.008280119 | CD49f |
| 497 | SERPINH1 | 3354.5389 | −1.859 | 0.2343 | −3.6679 | 0.000244511 | 0.008414726 | CD49f |
| 498 | GPR143 | 12.1329 | 4.33 | 0.9079 | 3.6672 | 0.000245234 | 0.008422648 | KIT |
| 499 | TTYH2 | 147.1444 | 1.61 | 0.1667 | 3.658 | 0.000254231 | 0.008714179 | KIT |
| 500 | SMCO4 | 76.7477 | 1.911 | 0.2492 | 3.6546 | 0.000257571 | 0.008810986 | KIT |
| 501 | KLK7 | 713.1029 | 3.819 | 0.772 | 3.6513 | 0.000260904 | 0.008907204 | KIT |
| 502 | NUDT10 | 187.984 | −1.899 | 0.2465 | −3.6467 | 0.000265593 | 0.009049207 | CD49f |
| 503 | FHL2 | 3527.7189 | −1.901 | 0.2472 | −3.645 | 0.000267342 | 0.00908575 | CD49f |
| 504 | LIX1L | 337.7616 | −1.736 | 0.2021 | −3.6447 | 0.000267728 | 0.00908575 | CD49f |
| 505 | LCN1P1 | 5.2702 | 5.338 | 1.1915 | 3.6411 | 0.000271483 | 0.009180521 | KIT |
| 506 | MAP2 | 775.9916 | 2.158 | 0.3181 | 3.641 | 0.000271594 | 0.009180521 | KIT |
| 507 | ARNTL | 1079.3276 | −1.714 | 0.1962 | −3.639 | 0.000273676 | 0.009232644 | CD49f |
| 508 | OLIG2 | 28.4262 | 3.413 | 0.6668 | 3.619 | 0.000295777 | 0.009958595 | KIT |
| 509 | CISH | 279.7137 | −2.28 | 0.3541 | −3.6164 | 0.000298777 | 0.010039853 | CD49f |
| 510 | ATP2C2 | 288.4416 | 1.911 | 0.2522 | 3.6126 | 0.000303115 | 0.010165659 | KIT |
| 511 | ALCAM | 630.3827 | 2.124 | 0.3112 | 3.6109 | 0.000305194 | 0.010195371 | KIT |
| 512 | FBXO32 | 5508.9385 | −1.568 | 0.1572 | −3.6112 | 0.000304737 | 0.010195371 | CD49f |
| 513 | NOTCH4 | 155.7148 | −1.58 | 0.1608 | 3.6085 | 0.000307998 | 0.010268992 | CD49f |
| 514 | SNTB1 | 206.1967 | 2.752 | 0.4857 | 3.6079 | 0.00030873 | 0.010273395 | KIT |
| 515 | FST | 1273.2514 | −3.69 | 0.7467 | −3.6022 | 0.000315589 | 0.010481219 | CD49f |
| 516 | NOD2 | 36.4729 | 2.886 | 0.5269 | 3.5792 | 0.000344609 | 0.011422842 | KIT |
| 517 | GGTA1P | 38.2625 | 4.897 | 1.0901 | 3.5754 | 0.000349751 | 0.011570876 | KIT |
| 518 | LYPD3 | 1945.5962 | 2.596 | 0.4468 | 3.5726 | 0.000353511 | 0.011660874 | KIT |
| 519 | OSMR | 1335.2271 | −1.831 | 0.2327 | 3.5723 | 0.000353835 | 0.011660874 | CD49f |
| 520 | Clorf226 | 514.9094 | −1.709 | 0.1987 | −3.5666 | 0.000361644 | 0.011895318 | CD49f |
| 521 | ADGRL3 | 10.1635 | 4.354 | 0.9415 | 3.5626 | 0.000367262 | 0.012056907 | KIT |
| 522 | SSPN | 249.4232 | −1.679 | 0.1906 | 3.5616 | 0.000368647 | 0.012079205 | CD49f |
| 523 | GGT6 | 78.0126 | 2.084 | 0.3047 | 3.5569 | 0.000375254 | 0.012272171 | KIT |
| 524 | LRP1 | 2456.2752 | −2.296 | 0.3649 | 3.5525 | 0.00038155 | 0.012443255 | CD49f |
| 525 | RGS10 | 138.6058 | 1.989 | 0.2783 | 3.5523 | 0.00038194 | 0.012443255 | KIT |
| 526 | COCH | 23.6365 | 2.588 | 0.4472 | 3.551 | 0.000383745 | 0.012454999 | KIT |
| 527 | RAMP1 | 10.3044 | −3.735 | 0.7701 | −3.551 | 0.000383757 | 0.012454999 | CD49f |
| 528 | SCPEP1 | 4557.5388 | −1.981 | 0.2769 | −3.544 | 0.000394045 | 0.012764669 | CD49f |
| 529 | LURAPIL | 264.3306 | 4.037 | 0.8571 | 3.5433 | 0.000395187 | 0.012777462 | KIT |
| 530 | CHURC1- | 11.7052 | 3.97 | 0.8384 | 3.5419 | 0.000397282 | 0.012820954 | KIT |
| | FNTB | | | | | | | |
| 531 | NNAT | 97.8609 | −2.529 | 0.4328 | 3.5335 | 0.000410026 | 0.013207329 | CD49f |
| 532 | CAMK1D | 222.5141 | 2.44 | 0.4077 | 3.5322 | 0.000412124 | 0.013233445 | KIT |
| 533 | SOST | 14.4792 | −5.508 | 1.2764 | −3.532 | 0.000412385 | 0.013233445 | CD49f |
| 534 | STMN3 | 700.4213 | −1.909 | 0.2581 | −3.5241 | 0.000424876 | 0.013608753 | CD49f |
| 535 | IKBKE | 44.6592 | 2.099 | 0.312 | 3.5232 | 0.000426353 | 0.013630535 | KIT |
| 536 | TNNT1 | 12.4882 | 4.361 | 0.9565 | 3.5139 | 0.000441548 | 0.014089986 | KIT |
| 537 | RECK | 306.8298 | −1.785 | 0.2238 | −3.5087 | 0.000450287 | 0.014342108 | CD49f |
| 538 | CHPT1 | 1269.2875 | 1.601 | 0.1715 | 3.5062 | 0.000454528 | 0.01444814 | KIT |
| 539 | FUT2 | 64.9484 | 2.826 | 0.5209 | 3.5058 | 0.000455306 | 0.01444814 | KIT |
| 540 | SERPING1 | 334.5733 | −2.638 | 0.4692 | 3.4916 | 0.000480218 | 0.015210476 | CD49f |
| 541 | GUCY1B1 | 364.7751 | 1.824 | 0.2363 | 3.4879 | 0.000486883 | 0.01536633 | KIT |
| 542 | PPP1R12B | 1802.903 | −1.82 | 0.2351 | 3.4878 | 0.000486936 | 0.01536633 | CD49f |
| 543 | CDCP1 | 354.1224 | 1.857 | 0.2462 | 3.4825 | 0.000496754 | 0.015647309 | KIT |
| 544 | DOC2B | 67.7359 | −2.755 | 0.504 | −3.482 | 0.000497674 | 0.015647466 | CD49f |
| 545 | SLC2A3 | 1976.4177 | −2.253 | 0.3602 | −3.4786 | 0.000504118 | 0.015820974 | CD49f |
| 546 | KRT4 | 77.7793 | 4.087 | 0.8878 | 3.4777 | 0.000505674 | 0.015840748 | KIT |
| 547 | FBXL7 | 555.8869 | −1.991 | 0.2851 | −3.476 | 0.000508937 | 0.015913814 | CD49f |
| 548 | TACC1 | 1095.7398 | −1.96 | 0.2763 | −3.4751 | 0.000510568 | 0.015935699 | CD49f |
| 549 | TRABD2B | 102.6444 | −3.575 | 0.7434 | 3.4642 | 0.00053174 | 0.016566268 | CD49f |
| 550 | IL15RA | 25.1641 | 2.323 | 0.3821 | 3.4637 | 0.000532797 | 0.016569021 | KIT |
| 551 | SLC16A7 | 223.1216 | −1.518 | 0.1499 | −3.4531 | 0.000554194 | 0.017203143 | CD49f |
| 552 | CA3 | 95.6696 | 1.902 | 0.2623 | 3.4387 | 0.000584437 | 0.018109076 | KIT |
| 553 | GPX3 | 168.7184 | 2.669 | 0.4855 | 3.4371 | 0.000588045 | 0.018187917 | KIT |
| 554 | RASGRF1 | 89.0483 | 2.883 | 0.5488 | 3.432 | 0.0005992 | 0.018499474 | KIT |
| 555 | TUFT1 | 1509.538 | 1.897 | 0.2616 | 3.4284 | 0.000607188 | 0.018712343 | KIT |
| 556 | STAB1 | 15.591 | −3.469 | 0.7214 | −3.4225 | 0.000620408 | 0.019085372 | CD49f |
| 557 | RCN3 | 636.9197 | −2.025 | 0.2998 | −3.4187 | 0.000629213 | 0.019321476 | CD49f |
| 558 | TENM2 | 230.0989 | −3.206 | 0.6465 | 3.4131 | 0.000642245 | 0.0196863 | CD49f |
| 559 | SPIRE2 | 50.9859 | 1.979 | 0.287 | 3.4098 | 0.000649994 | 0.01988818 | KIT |
| 560 | AC000093.1 | 580.8663 | −1.648 | 0.1902 | −3.4048 | 0.000662109 | 0.020222715 | CD49f |
| 561 | ACTG2 | 6028.3104 | −2.197 | 0.352 | −3.4019 | 0.000669151 | 0.020401367 | CD49f |
| 562 | ITIH3 | 7.2091 | −5.598 | 1.357 | −3.3882 | 0.000703508 | 0.021410691 | CD49f |
| 563 | ADAMTS16 | 170.7772 | 2.384 | 0.4088 | 3.3858 | 0.000709804 | 0.021563924 | KIT |
| 564 | ART3 | 1122.6934 | 2.022 | 0.3026 | 3.3793 | 0.000726771 | 0.02204024 | KIT |
| 565 | RIMS2 | 10.3405 | 4.355 | 0.9944 | 3.3737 | 0.00074159 | 0.022449833 | KIT |
| 566 | PTPN22 | 9.3917 | 4.613 | 1.0716 | 3.3719 | 0.000746545 | 0.02255991 | KIT |
| 567 | AP000873.1 | 7.8141 | −4.135 | 0.9304 | −3.3695 | 0.000753006 | 0.022715024 | CD49f |
| 568 | AC079594.2 | 13.463 | 3.873 | 0.853 | 3.3685 | 0.000755865 | 0.022761116 | KIT |
| 569 | GPR157 | 214.3912 | 1.747 | 0.2226 | 3.3548 | 0.000794103 | 0.023870531 | KIT |
| 570 | LAMC1 | 8488.8513 | −2 | 0.2991 | −3.3427 | 0.000829653 | 0.024895426 | CD49f |
| 571 | OVCH2 | 87.4232 | −3.345 | 0.7022 | −3.3392 | 0.000840129 | 0.025165605 | CD49f |
| 572 | RPS27L | 627.2672 | 1.538 | 0.1613 | 3.3377 | 0.00084473 | 0.02525919 | KIT |
| 573 | RAB3IP | 670.8784 | 1.595 | 0.1786 | 3.3308 | 0.000865916 | 0.025847526 | KIT |
| 574 | ADAMTS5 | 17.6183 | −2.521 | 0.4569 | −3.3286 | 0.000872783 | 0.026007097 | CD49f |
| 575 | BTBD11 | 219.4451 | −2.398 | 0.4201 | −3.3273 | 0.000877028 | 0.026088162 | CD49f |
| 576 | MAOA | 219.8309 | 1.967 | 0.291 | 3.3229 | 0.000890845 | 0.026453159 | KIT |
| 577 | PDZK1IP1 | 26.0163 | 2.229 | 0.37 | 3.3224 | 0.000892421 | 0.026454004 | KIT |
| 578 | SNCG | 7.2332 | −4.017 | 0.9094 | 3.3174 | 0.000908615 | 0.026887445 | CD49f |
| 579 | PRKAR2B | 561.0789 | 1.86 | 0.2595 | 3.3141 | 0.000919462 | 0.027161459 | KIT |
| 580 | CRACR2B | 2164.5733 | 2.574 | 0.476 | 3.3075 | 0.000941187 | 0.02775528 | KIT |
| 581 | ACVR2A | 772.668 | −1.759 | 0.2298 | −3.3047 | 0.000950688 | 0.027987219 | CD49f |
| 582 | SERPINE1 | 2388.1619 | −2.58 | 0.4782 | −3.3039 | 0.000953586 | 0.02802429 | CD49f |
| 583 | CHST1 | 59.3797 | 2.506 | 0.4565 | 3.2996 | 0.000968295 | 0.028407757 | KIT |
| 584 | ARMH4 | 559.8646 | −1.587 | 0.1781 | −3.2943 | 0.000986792 | 0.028851444 | CD49f |
| 585 | MOXD1 | 69.3233 | −3.634 | 0.7995 | −3.2943 | 0.000986724 | 0.028851444 | CD49f |
| 586 | NAP1L3 | 184.2053 | −2.031 | 0.3133 | −3.2919 | 0.000995258 | 0.029049313 | CD49f |
| 587 | RASSF2 | 447.6381 | 2.235 | 0.3754 | 3.2891 | 0.001005043 | 0.029284926 | KIT |
| 588 | ITGA2 | 6195.4062 | −2.534 | 0.4668 | 3.2863 | 0.001015061 | 0.029526531 | CD49f |
| 589 | LAMA5 | 7470.6576 | −1.342 | 0.104 | 3.2836 | 0.001025003 | 0.029742366 | CD49f |
| 590 | TNPO1P3 | 6.8153 | −4.914 | 1.192 | 3.2833 | 0.001025959 | 0.029742366 | CD49f |
| 591 | SLC9A7P1 | 7.4957 | −3.699 | 0.8223 | −3.2818 | 0.001031527 | 0.029853208 | CD49f |
| 592 | ATP13A4 | 52.6738 | 2.629 | 0.497 | 3.2771 | 0.001048747 | 0.030300293 | KIT |
| 593 | COL6A6 | 10.5857 | −5.054 | 1.2385 | −3.2734 | 0.001062592 | 0.030648524 | CD49f |
| 594 | DSG3 | 1048.3057 | 3.783 | 0.8508 | 3.2713 | 0.001070377 | 0.030821104 | KIT |
| 595 | KRT18 | 2454.1012 | 1.924 | 0.2826 | 3.2689 | 0.001079592 | 0.031034182 | KIT |
| 596 | TPST2 | 225.6008 | −1.351 | 0.1075 | −3.2678 | 0.001084038 | 0.031109712 | CD49f |
| 597 | USP11 | 2454.7519 | −1.471 | 0.1445 | −3.2598 | 0.001115075 | 0.031946799 | CD49f |
| 598 | NNMT | 174.4923 | −2.921 | 0.5895 | 3.2592 | 0.001117088 | 0.031950965 | CD49f |
| 599 | NCS1 | 1880.3259 | −1.677 | 0.2082 | 3.2493 | 0.001156894 | 0.033034237 | CD49f |
| 600 | COL9A1 | 22052.093 | −2.363 | 0.4199 | 3.2466 | 0.001168028 | 0.033296587 | CD49f |
| 601 | NALCN | 71.0769 | 3.155 | 0.665 | 3.2411 | 0.001190491 | 0.033880449 | KIT |
| 602 | POSTN | 16.036 | −3.329 | 0.7199 | −3.2347 | 0.00121755 | 0.034592992 | CD49f |
| 603 | TENM1 | 55.1558 | −2.846 | 0.5716 | −3.2287 | 0.001243356 | 0.035267606 | CD49f |
| 604 | MISP3 | 62.3082 | 2.306 | 0.4049 | 3.2254 | 0.001258096 | 0.035626618 | KIT |
| 605 | CLDN9 | 10.287 | 4.287 | 1.0196 | 3.2242 | 0.001263191 | 0.035711765 | KIT |
| 606 | SPON2 | 167.8571 | 2.582 | 0.4912 | 3.22 | 0.001281751 | 0.036176669 | KIT |
| 607 | CD14 | 38.7359 | 2.311 | 0.4073 | 3.2183 | 0.001289651 | 0.036339688 | KIT |
| 608 | MANIC1 | 76.8408 | −2.171 | 0.3646 | −3.2109 | 0.001323126 | 0.037221617 | CD49f |
| 609 | CHST11 | 234.6937 | −2.953 | 0.6088 | 3.2085 | 0.001334103 | 0.037468794 | CD49f |
| 610 | RASSF5 | 141.2211 | 2.195 | 0.3733 | 3.2022 | 0.001363761 | 0.038238966 | KIT |
| 611 | LDLR | 13152.4255 | −1.814 | 0.2542 | −3.2013 | 0.001367956 | 0.038293801 | CD49f |
| 612 | CLVS2 | 14.0623 | −4.828 | 1.1973 | −3.1968 | 0.001389653 | 0.038837609 | CD49f |
| 613 | BMPER | 278.6907 | −2.666 | 0.5212 | −3.196 | 0.001393305 | 0.038876153 | CD49f |
| 614 | RAB17 | 168.7561 | −2.023 | 0.3202 | −3.1951 | 0.001397686 | 0.038934883 | CD49f |
| 615 | GPRASP1 | 477.827 | −1.933 | 0.2922 | −3.1928 | 0.001408896 | 0.039183337 | CD49f |
| 616 | CYP4F3 | 51.9857 | 3.361 | 0.7405 | 3.1888 | 0.001428771 | 0.039671597 | KIT |
| 617 | CYP21A1P | 13.0183 | −3.691 | 0.8442 | −3.1874 | 0.001435771 | 0.039801329 | CD49f |
| 618 | ZNF385C | 175.2435 | 1.682 | 0.2141 | 3.183 | 0.001457358 | 0.040334392 | KIT |
| 619 | FZD9 | 59.2912 | 2.05 | 0.33 | 3.1821 | 0.00146206 | 0.040399161 | KIT |
| 620 | TMOD1 | 16.3618 | 4.162 | 0.9943 | 3.1799 | 0.001473133 | 0.04063946 | KIT |
| 621 | HEY1 | 132.8223 | 2.062 | 0.3342 | 3.1784 | 0.001481107 | 0.040793642 | KIT |
| 622 | CCDC9B | 522.1492 | 1.88 | 0.2771 | 3.174 | 0.001503495 | 0.04132814 | KIT |
| 623 | TMEM71 | 6.1863 | 4.408 | 1.0739 | 3.1737 | 0.001505346 | 0.04132814 | KIT |
| 624 | C1R | 532.6668 | −1.828 | 0.2614 | −3.1694 | 0.001527648 | 0.041821374 | CD49f |
| 625 | TFAP2B | 34.7476 | 2.839 | 0.5802 | 3.1693 | 0.001528202 | 0.041821374 | KIT |
| 626 | SHANK2 | 2488.7981 | 1.894 | 0.2825 | 3.1658 | 0.001546589 | 0.042256965 | KIT |
| 627 | TNMD | 26.0455 | −7.466 | 2.0448 | −3.1624 | 0.00156478 | 0.042685808 | CD49f |
| 628 | ANGPT1 | 18.7201 | 3.198 | 0.6951 | 3.1619 | 0.001567418 | 0.042689666 | KIT |
| 629 | NDNF | 9.3048 | −4.376 | 1.0689 | 3.1585 | 0.001585742 | 0.042983403 | CD49f |
| 630 | PLXNA4 | 41.3796 | 2.538 | 0.4869 | 3.1588 | 0.001584398 | 0.042983403 | KIT |
| 631 | TMEM176B | 83.1694 | 3.564 | 0.8116 | 3.1588 | 0.001584376 | 0.042983403 | KIT |
| 632 | AL122013.1 | 10.5348 | −3.412 | 0.7645 | −3.1553 | 0.001603165 | 0.043386925 | CD49f |
| 633 | SNCA | 42.8323 | −2.745 | 0.5546 | −3.1464 | 0.00165295 | 0.044663608 | CD49f |
| 634 | SDK1 | 599.8234 | −1.797 | 0.2533 | −3.1452 | 0.001659613 | 0.044772898 | CD49f |
| 635 | KCNIP1 | 6.2452 | −4.763 | 1.2016 | −3.1319 | 0.001736761 | 0.046780419 | CD49f |
| 636 | NRXN2 | 68.0734 | −2.839 | 0.5876 | −3.1302 | 0.001747078 | 0.046984308 | CD49f |
| 637 | TPPP2 | 5.834 | 3.93 | 0.9368 | 3.1273 | 0.001763983 | 0.047364472 | KIT |
| 638 | ARL4D | 202.6761 | −1.998 | 0.3192 | −3.1262 | 0.001770662 | 0.047469297 | CD49f |
| 639 | OXGR1 | 9.4328 | 4.232 | 1.0346 | 3.1235 | 0.001787038 | 0.047833322 | KIT |
| 640 | NPTX2 | 350.4174 | −2.225 | 0.3934 | −3.1146 | 0.001842186 | 0.049079041 | CD49f |
| 641 | RHOJ | 290.0129 | −2.337 | 0.4292 | −3.1146 | 0.001841976 | 0.049079041 | CD49f |
| 642 | SLC52A1 | 335.8293 | −2.348 | 0.4326 | −3.1148 | 0.001840857 | 0.049079041 | CD49f |
| 643 | CWH43 | 8.7603 | 5.048 | 1.3012 | 3.1107 | 0.001866146 | 0.049640062 | KIT |

### Example 3. Developmental Relationship of CD49f^{high}/KIT^{neg} and CD49f^{low}/KIT⁺ Cells

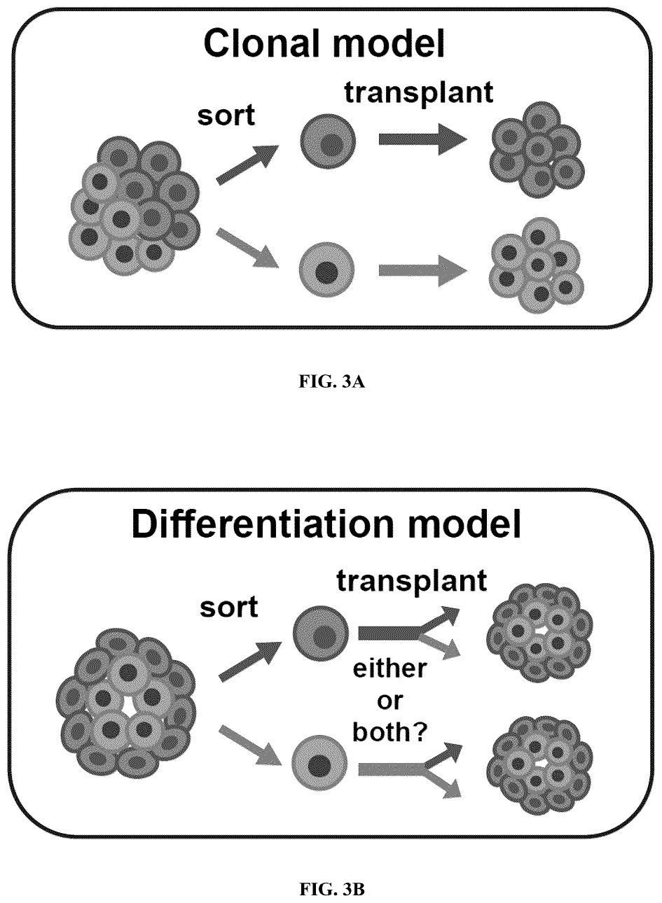

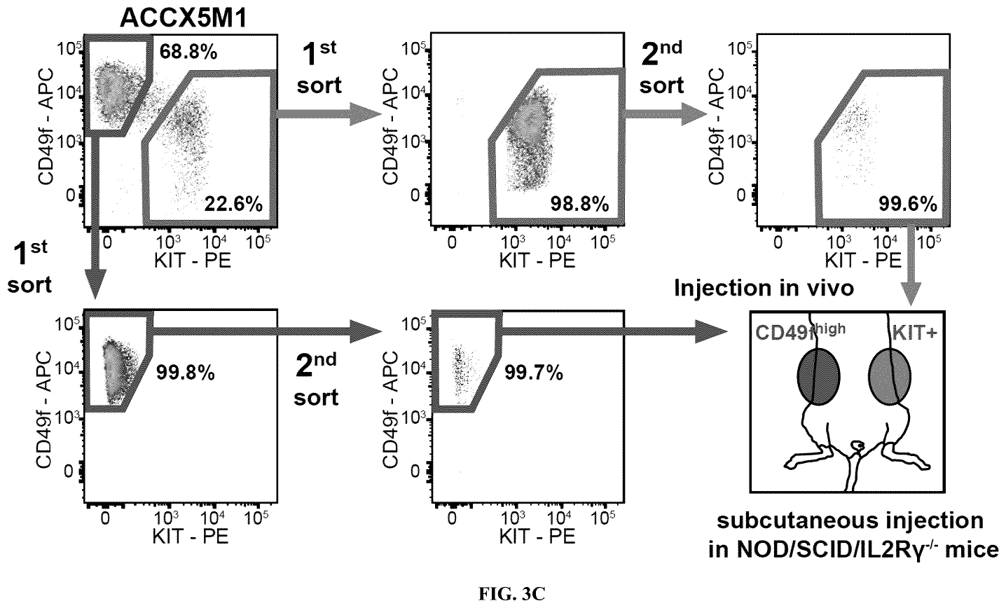

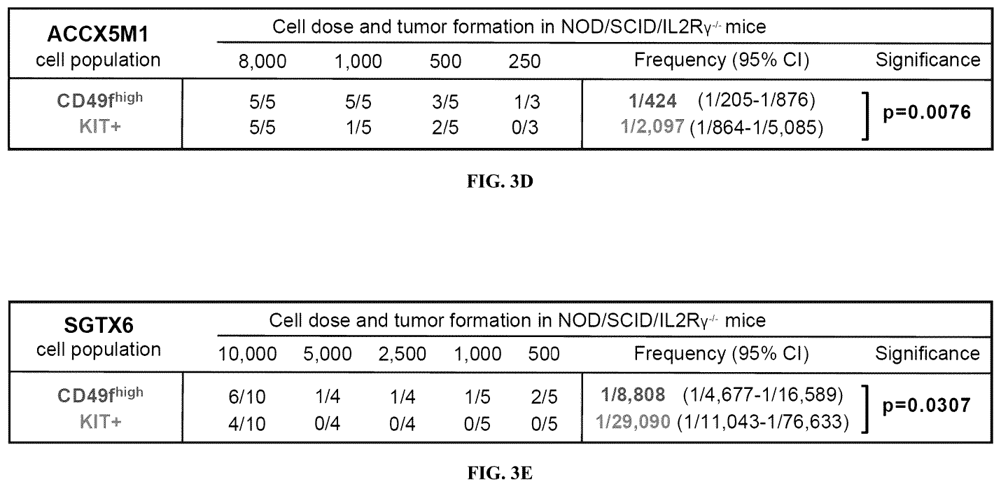

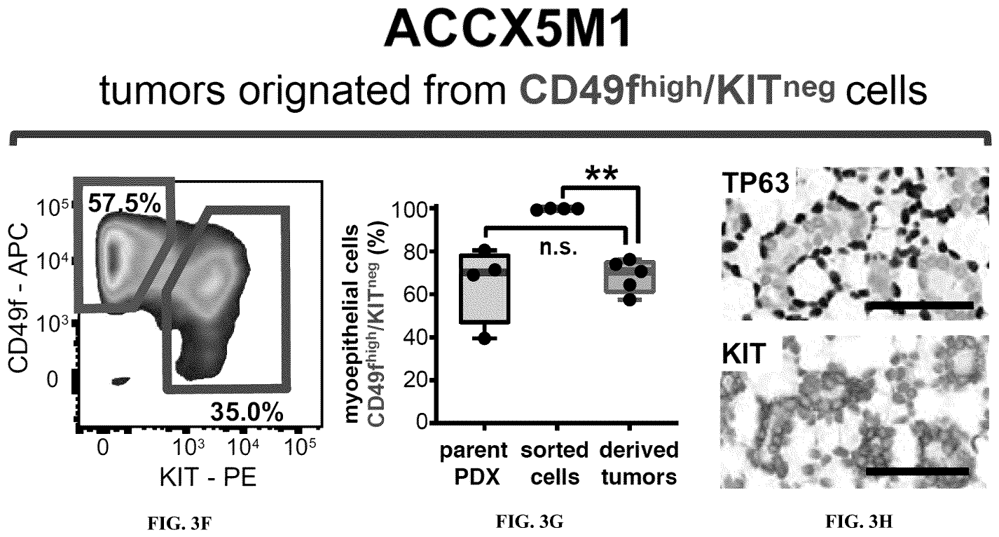

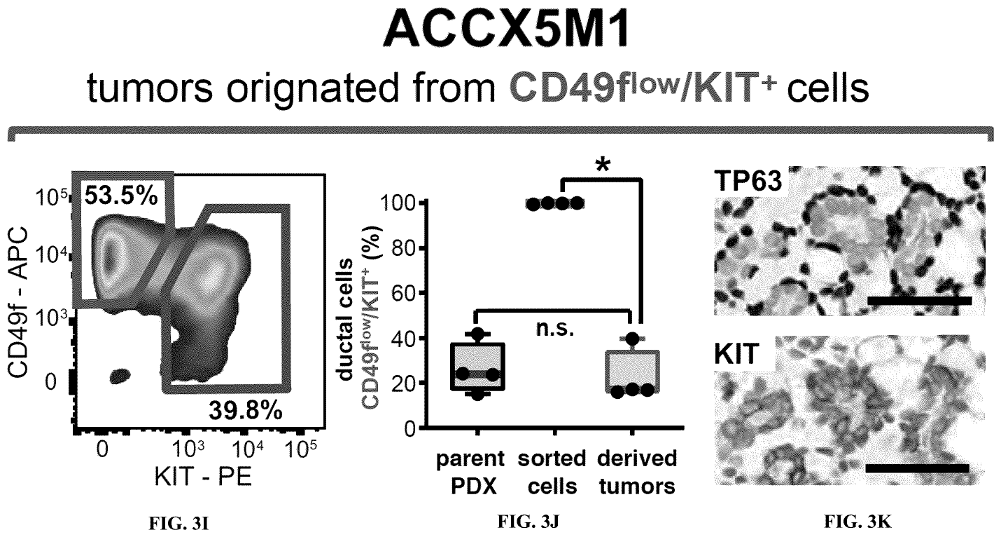

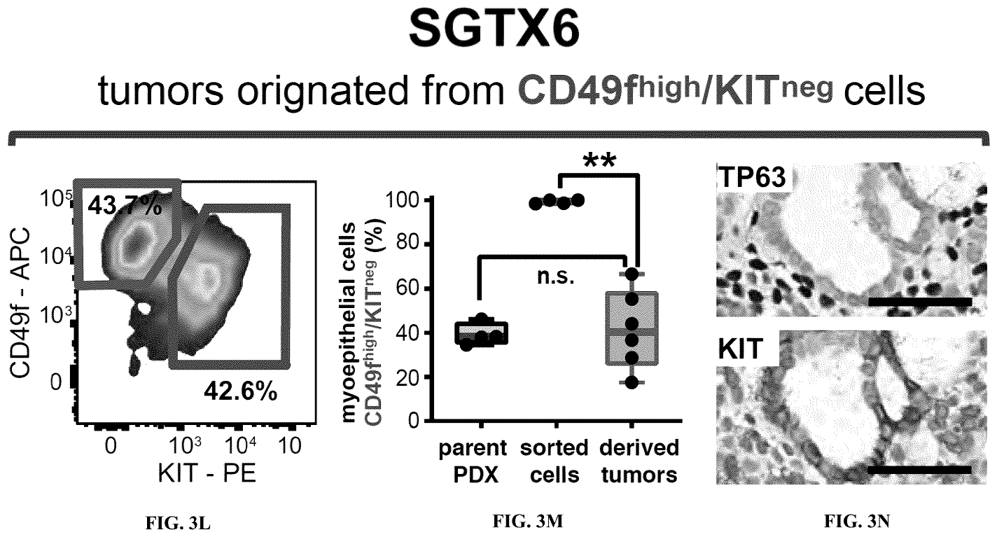

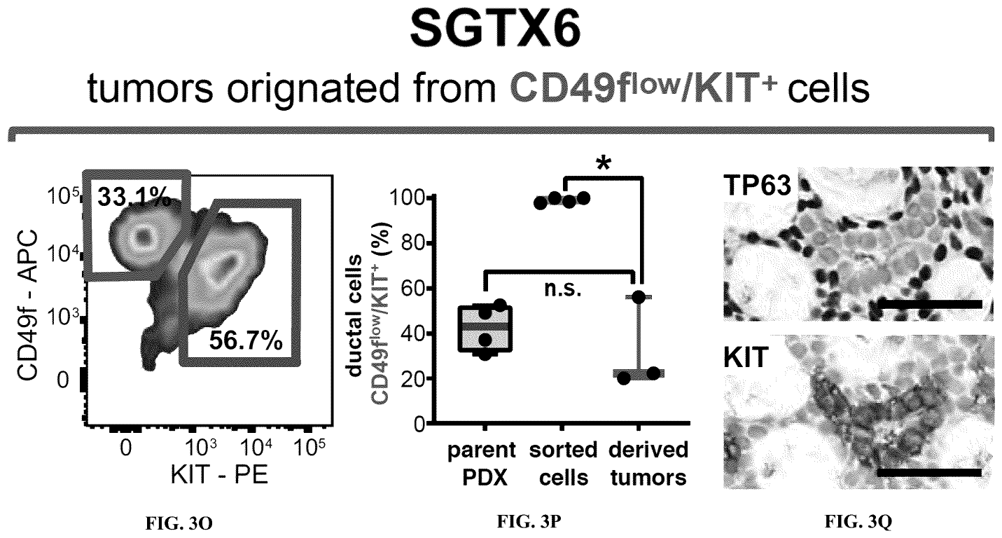

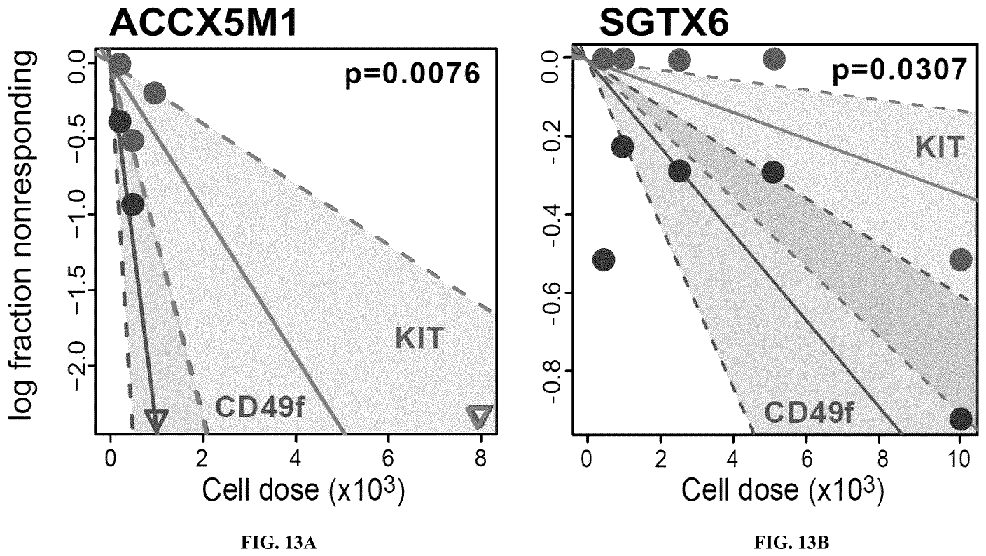

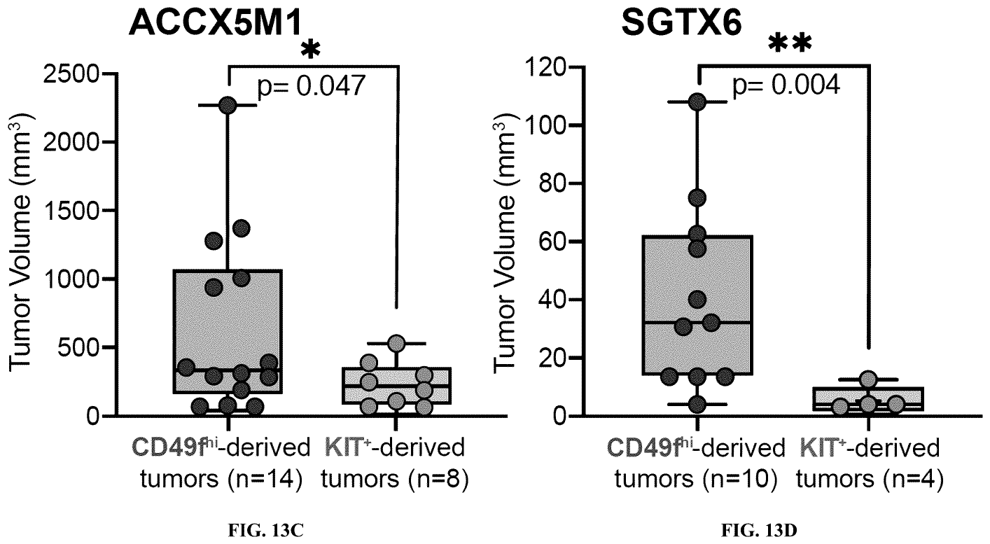

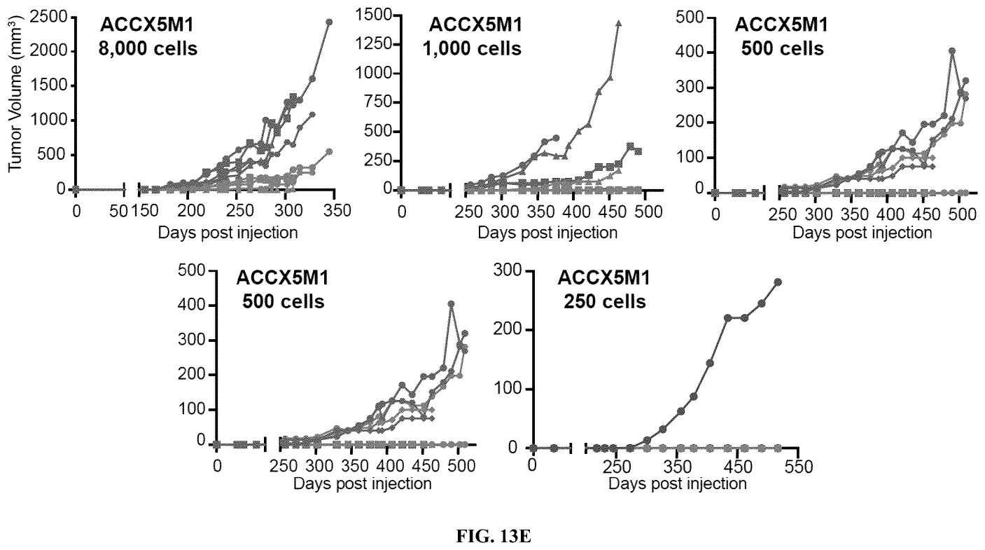

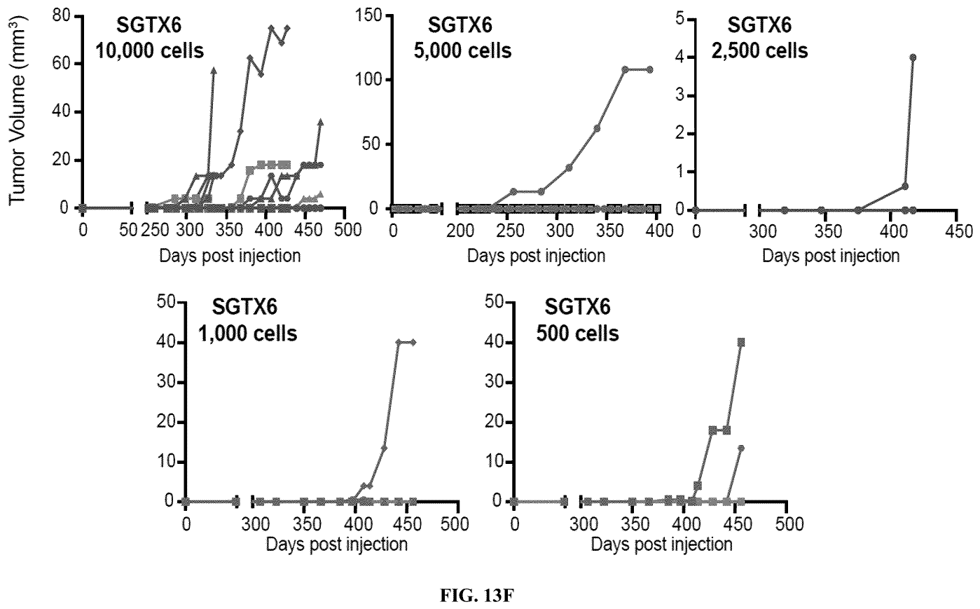

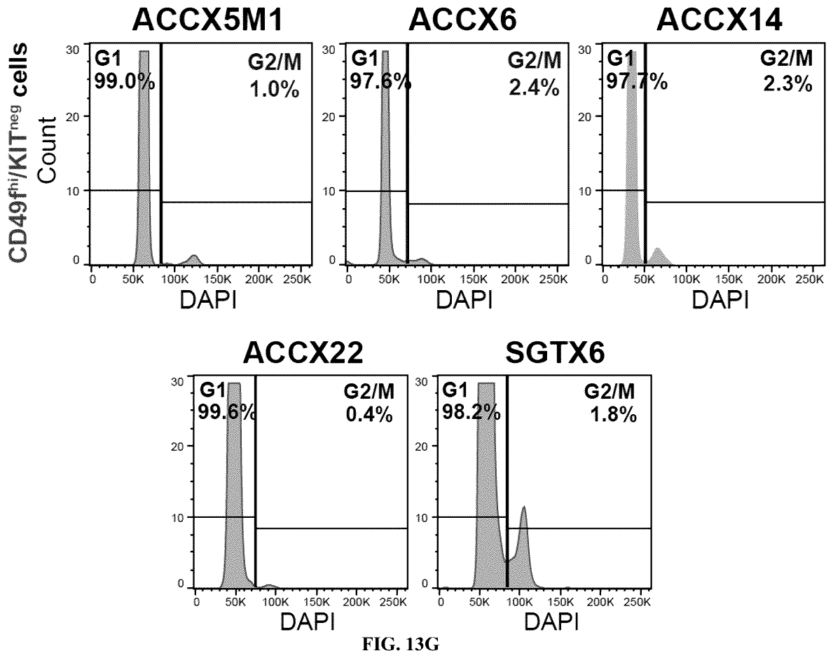

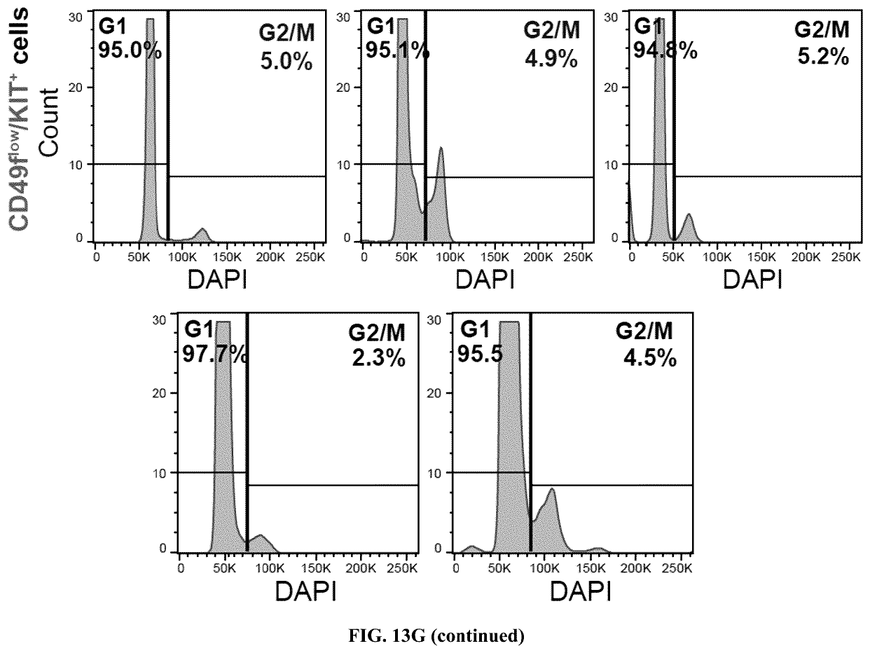

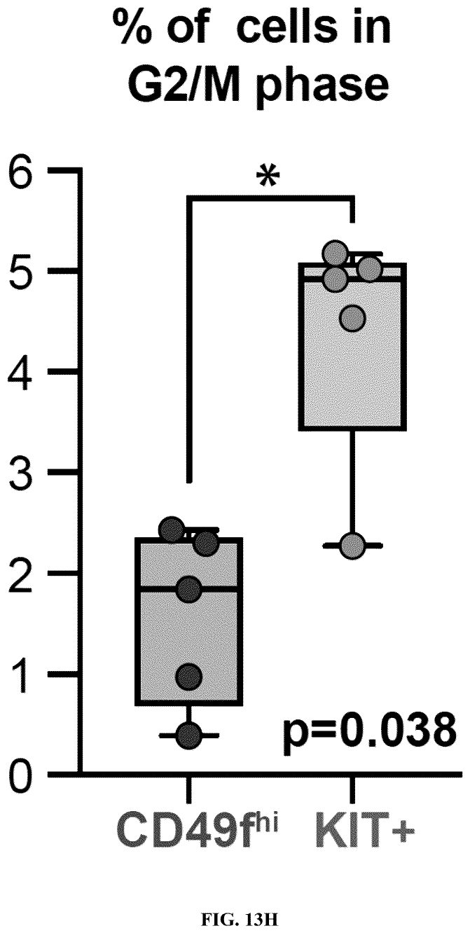

The next test was whether the two cell populations represented different genetic clones that co-existed within the same tissue (FIG. 3A), or whether they were linked by a developmental relationship, whereby one population could differentiate into the other, in a process akin to those sustaining the normal morphogenesis of epithelial tissues (FIG. 3B). To explore this concept, prospective xeno-transplantation studies was performed with purified preparations of the two cell populations, in order to evaluate their tumor-initiating and multi-lineage differentiation capacity. Autologous pairs of CD49f^{high}/KIT^{neg} and CD49f^{low}/KIT⁺ cells were double-sorted by FACS from two bi-phenotypic PDX lines (ACCX5M1, SGTX6) and injected, side-by-side, at progressively decreasing doses (10,000-250 cells/injection) in immune-deficient animals (FIG. 3C) [31]. It was observed that the frequency of tumor-initiating cells was higher in CD49f^{high}/KIT^{neg} as compared to CD49f^{low}/KIT⁺ cells (FIGS. 3D-3E, 13A-13B), resulting in larger and faster-growing tumors (FIGS. 13C-13F) despite CD49f^{high}/KIT^{neg} cells having a smaller fraction of actively proliferating cells (FIGS. 13G-13H). These results revealed that myoepithelial-like cells represent a biologically aggressive component of human ACCs, despite having a more quiescent phenotype. The cell composition of tumors originated from transplantation of sorted cells was then analyzed. The results showed that tumors originated from sorted CD49f^{high}/KIT^{neg} cells contained both cell types, at frequencies comparable to those observed in parent lines, irrespectively of the number of injected cells (FIGS. 3F-3H, 3L-3N). This observation showed that CD49f^{high}/KIT^{neg} cells can differentiate into CD49f^{low}/KIT⁺ cells, thus excluding the “clonal” hypothesis. When the few tumors originated from CD49f^{low}/KIT⁺ cells were analyzed, it was also found that they were indistinguishable from parent lines (FIGS. 3I-3K, 3O-3Q). In this specific case, however, given the high number of CD49f^{low}/KIT⁺ cells required for tumor-initiation, the possibility that such tumors arose from cross-contaminations of CD49f^{high}/KIT^{neg} cells could not be excluded, despite the high purity achieved by double-sorting.

### Example 4. Differential Expression of Mechanistic Regulators of Retinoic Acid (RA) Signaling

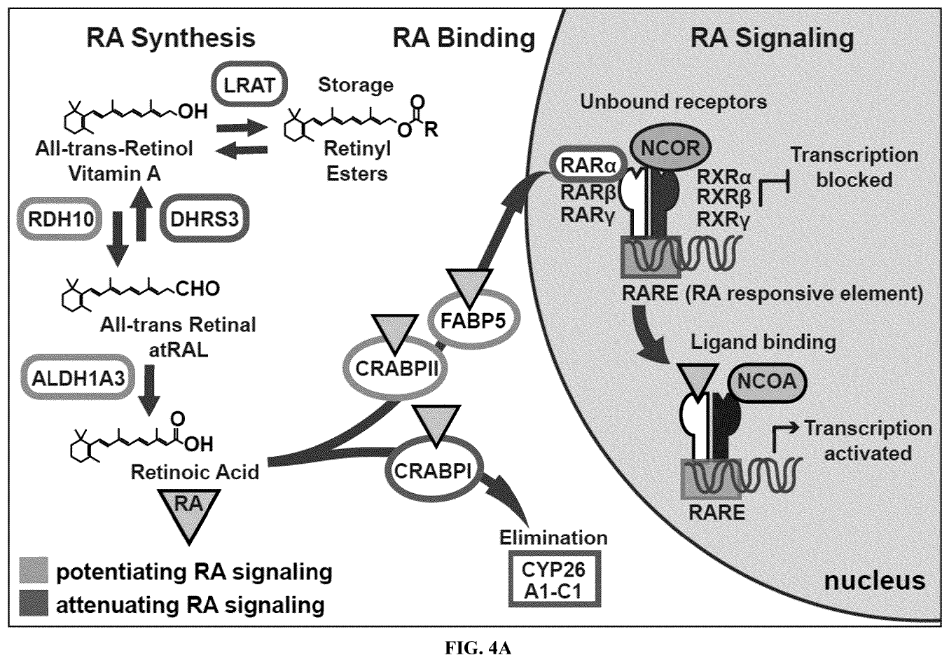

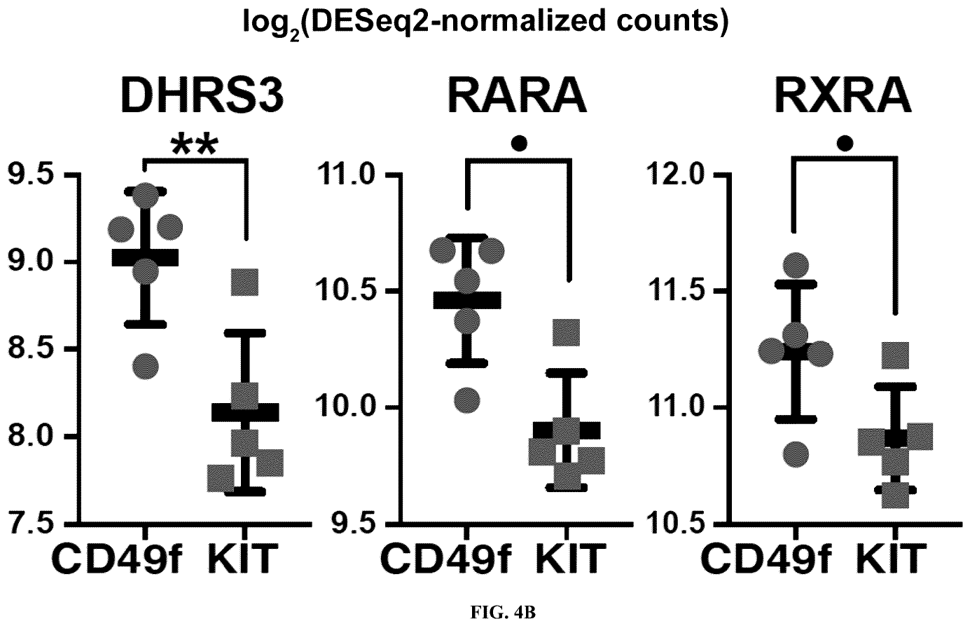

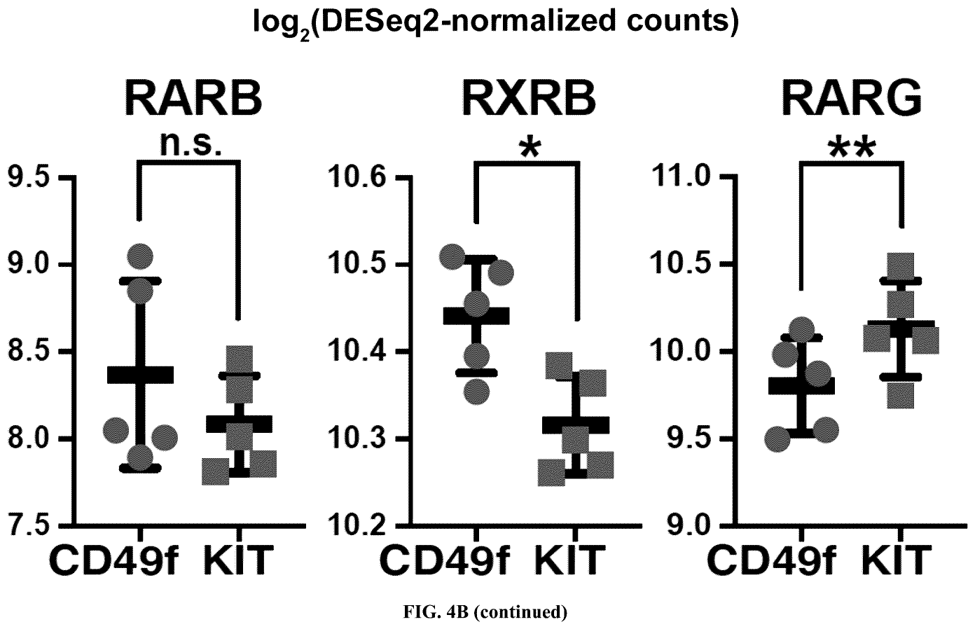

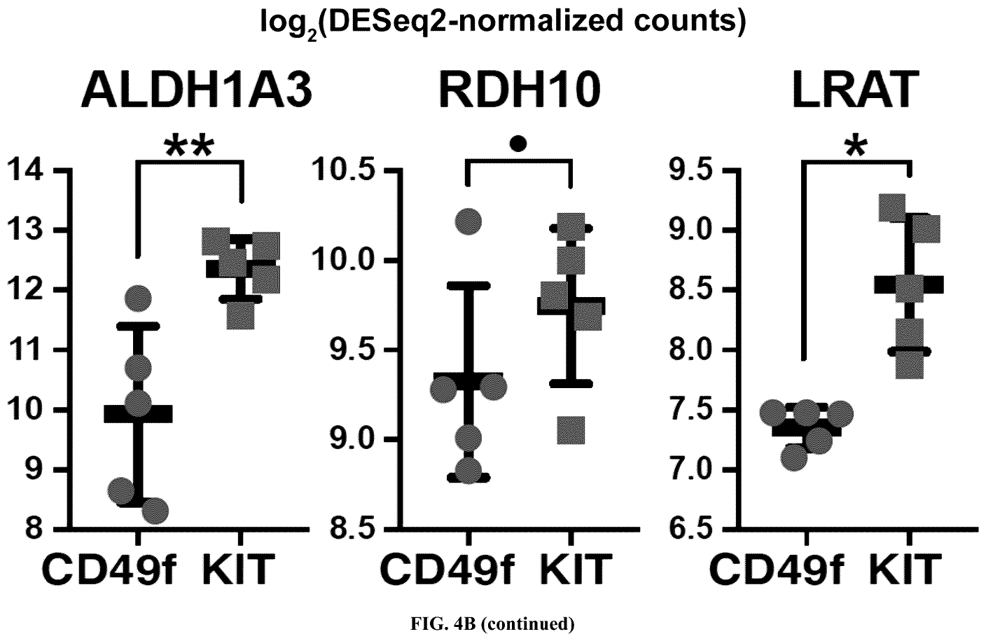

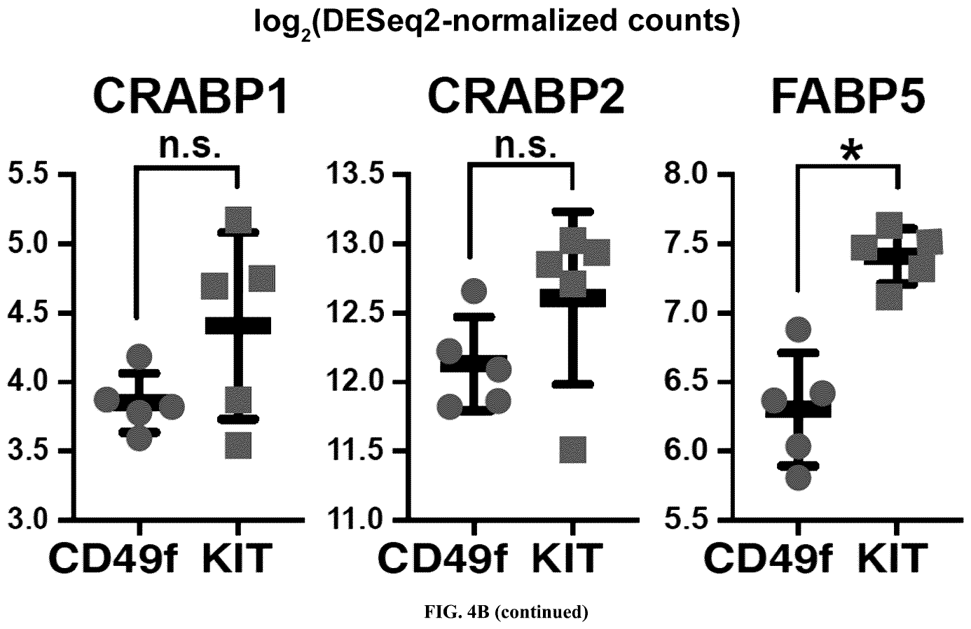

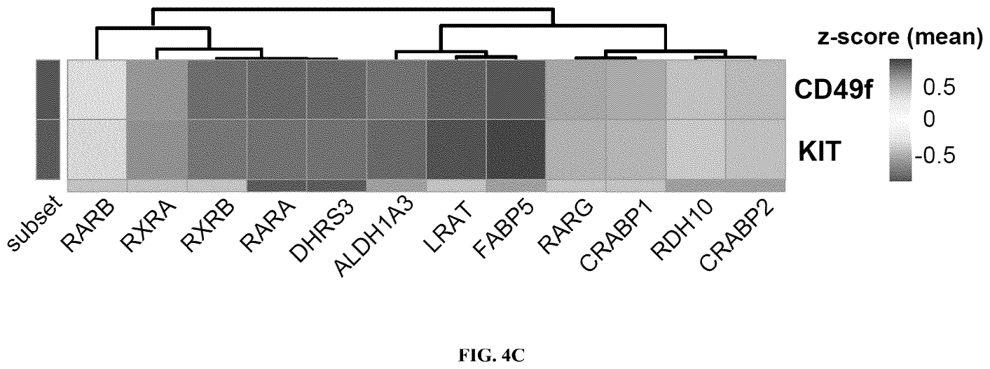

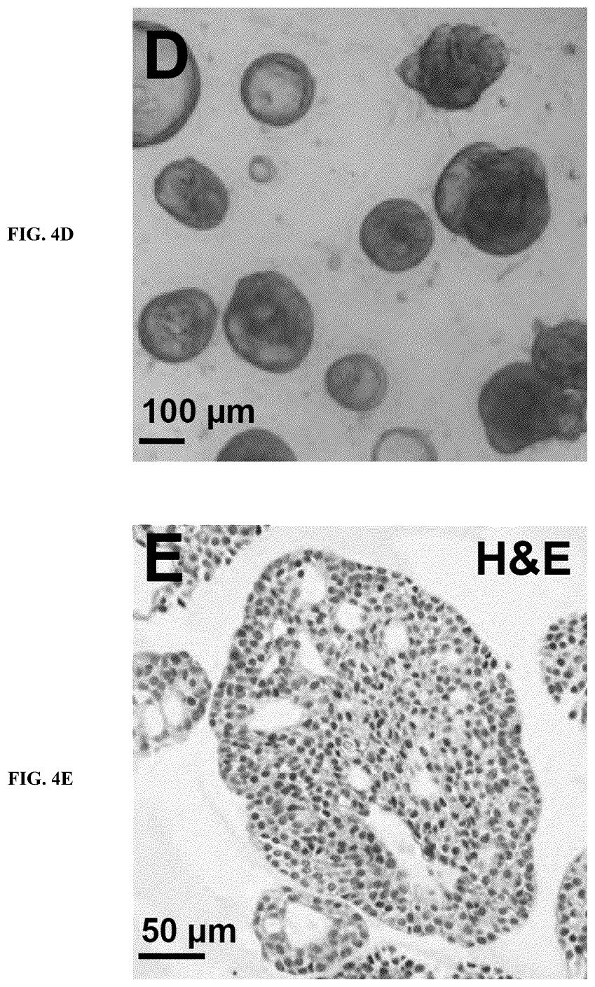

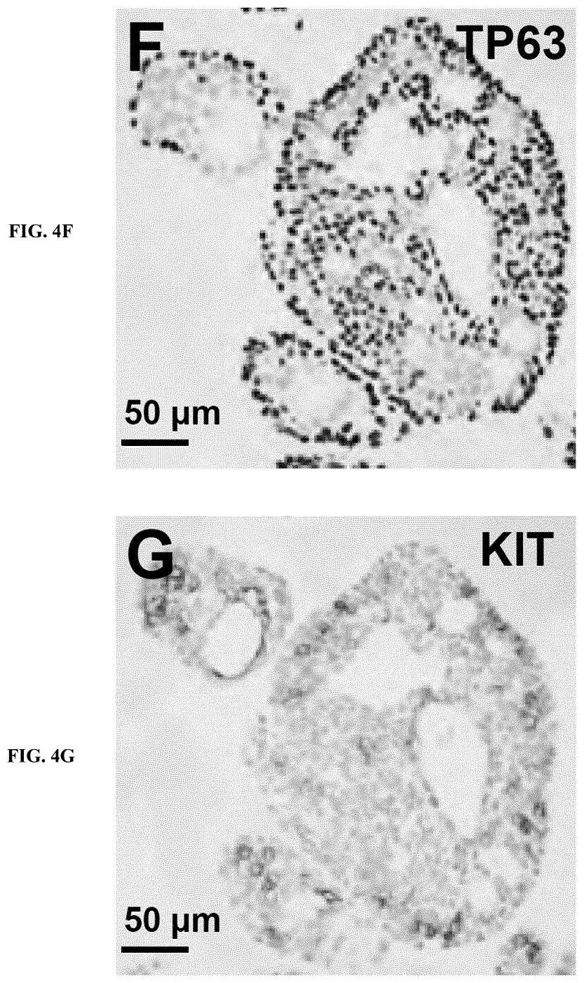

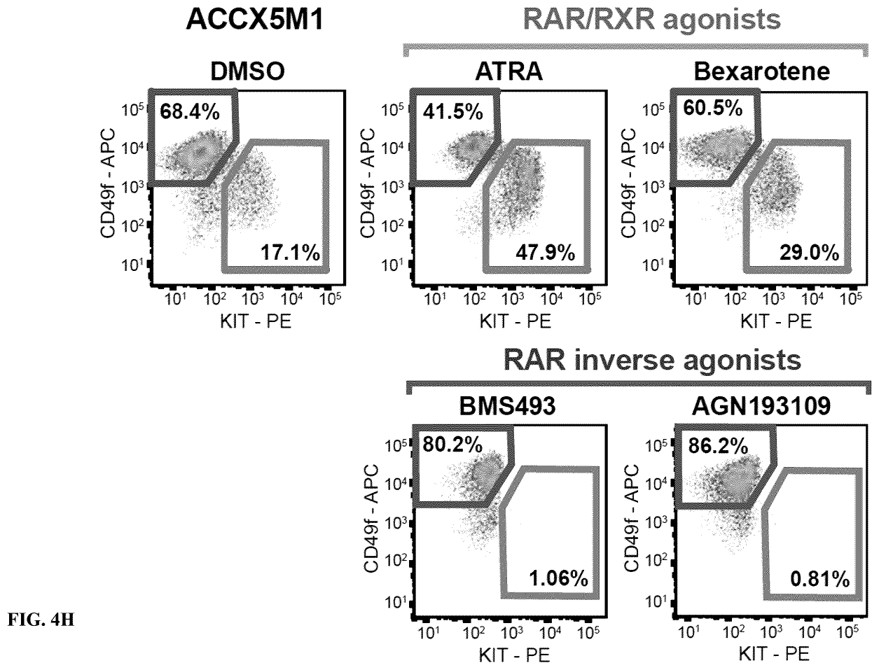

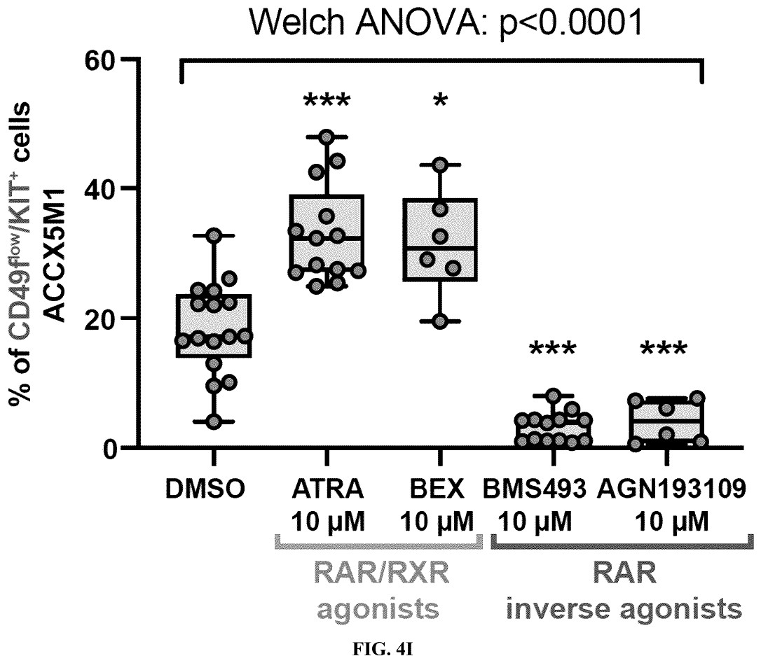

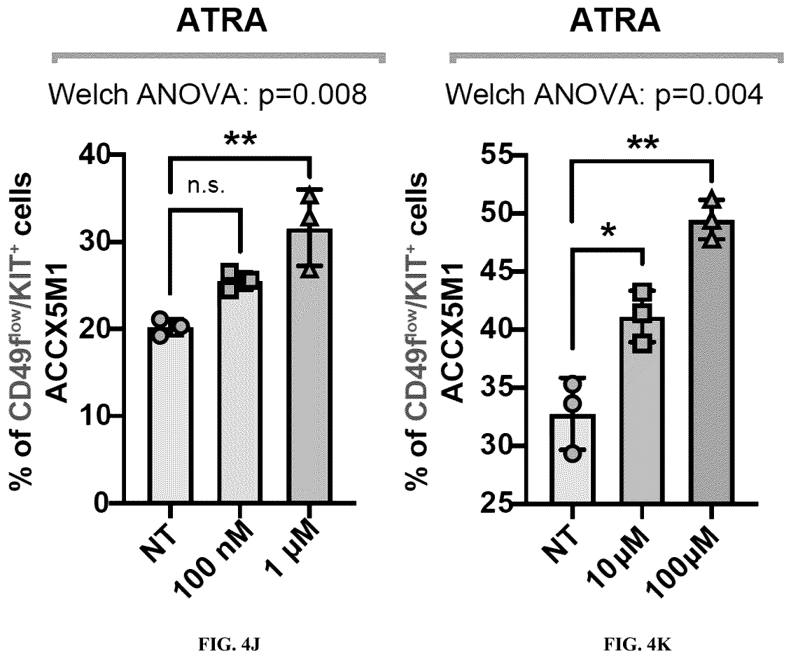

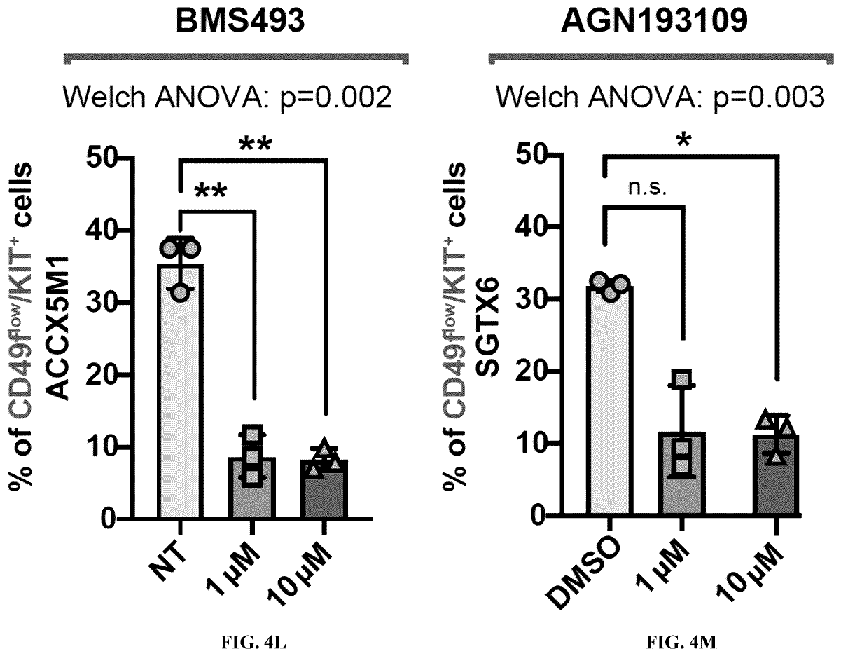

To elucidate the molecular mechanisms that control the differentiation of CD49f^{high}/KIT^{neg} cells into CD49f^{low}/KIT⁺ cells, signaling pathways were sought with differential activation in the two cell-types. It was tested whether CD49f^{high}/KIT^{neg} and CD49f^{low}/KIT⁺ cells differed in expression of genes encoding for mechanistic regulators of RA signaling, such as enzymes involved in RA biosynthesis [45-47], RA binding proteins [48-50] and RA receptors [51] (FIG. 4A), given that RA signaling plays a key role in the differentiation of SG epithelia [52-54] and antagonizes MYB signaling in human ACCs [55, 56]. It was found that activators of RA signaling were over-expressed in CD49f^{low}/KIT⁺ cells, whereas suppressors of RA signaling were over-expressed in CD49f^{high}/KIT^{neg} cells, in a coordinated fashion (FIGS. 4B-4C).

### Example 5. In Vitro Effects of RAR/RXR Activation and Inhibition

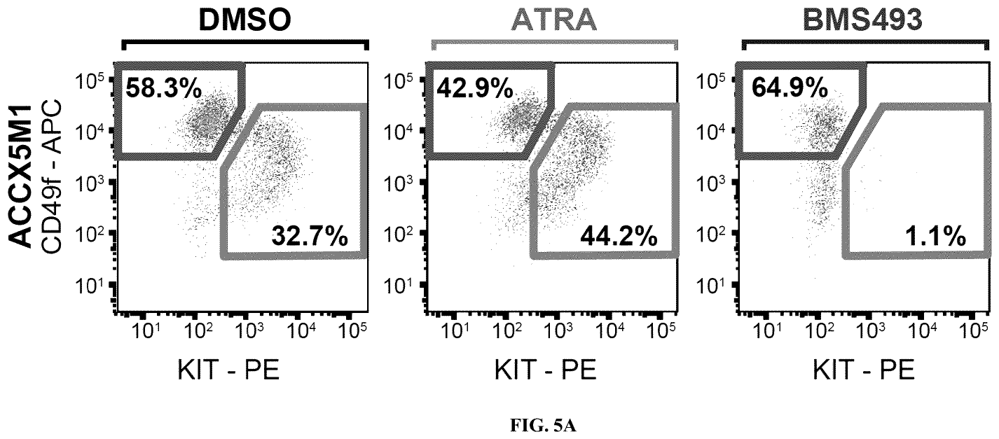

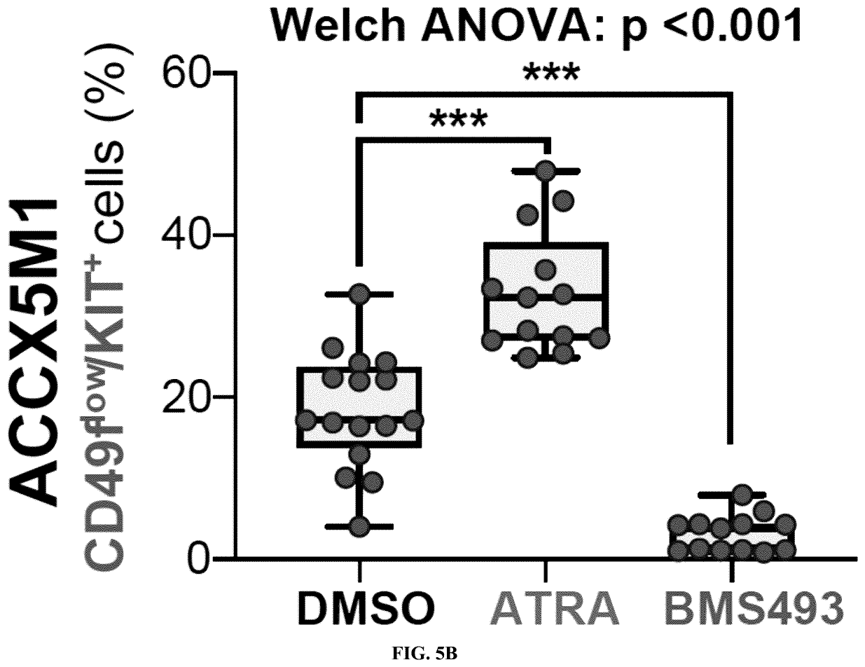

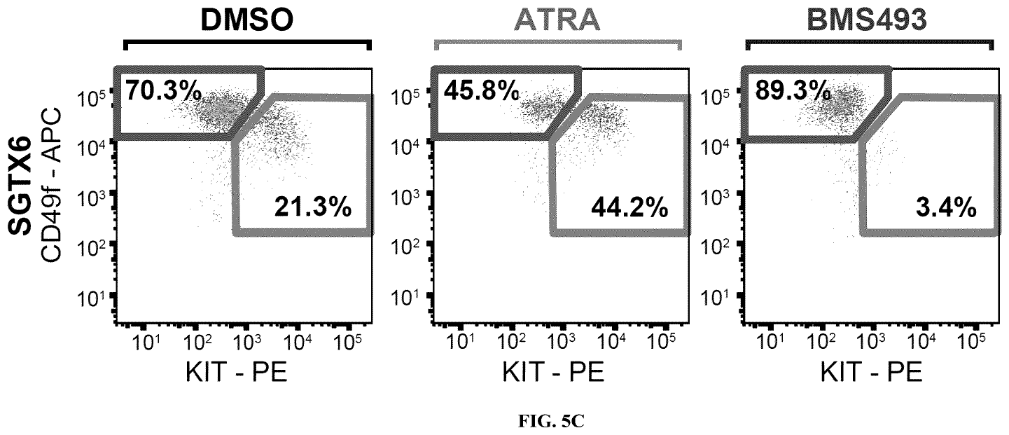

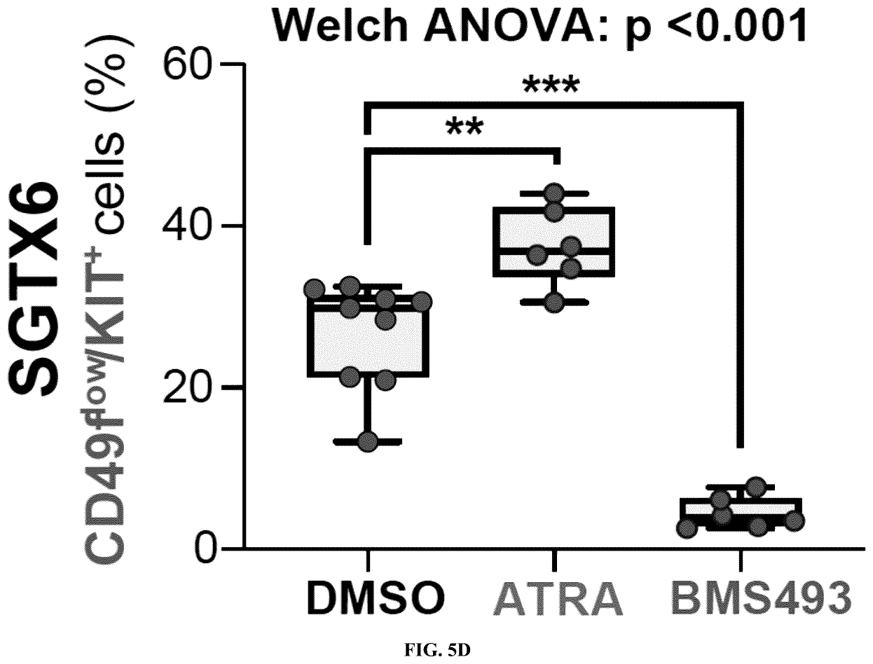

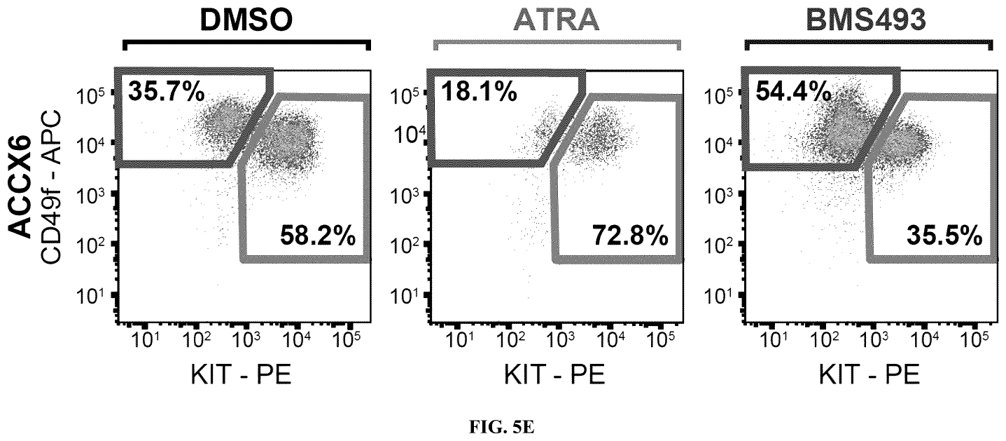

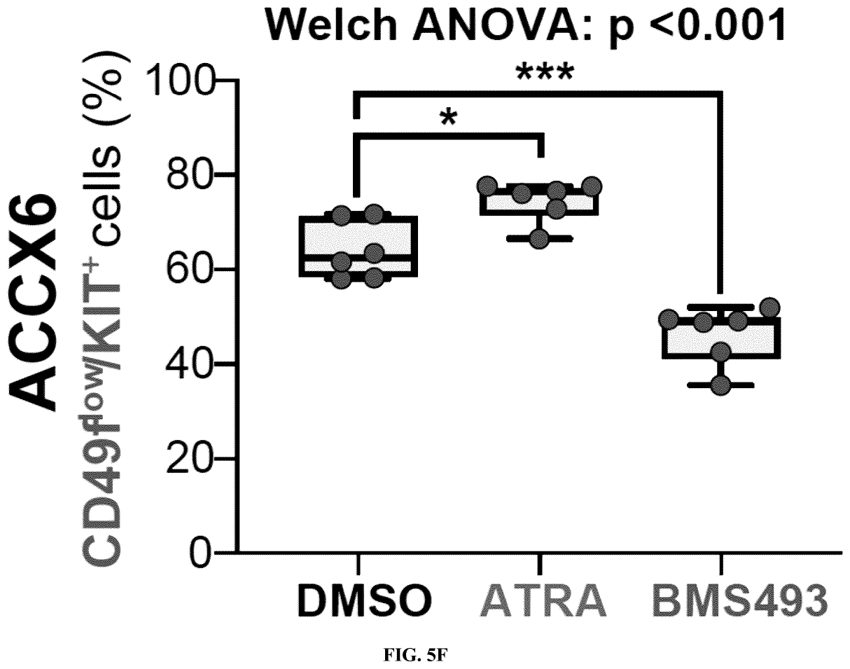

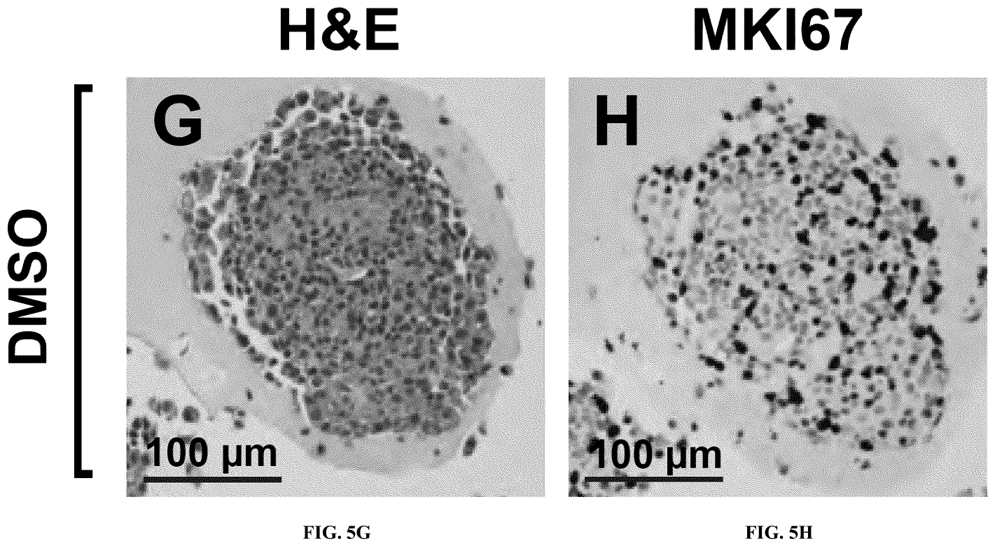

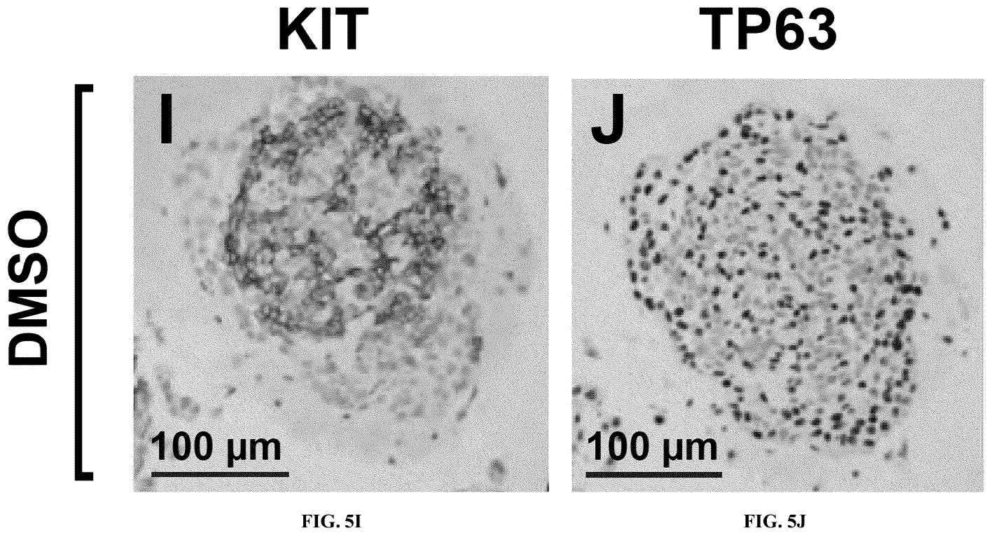

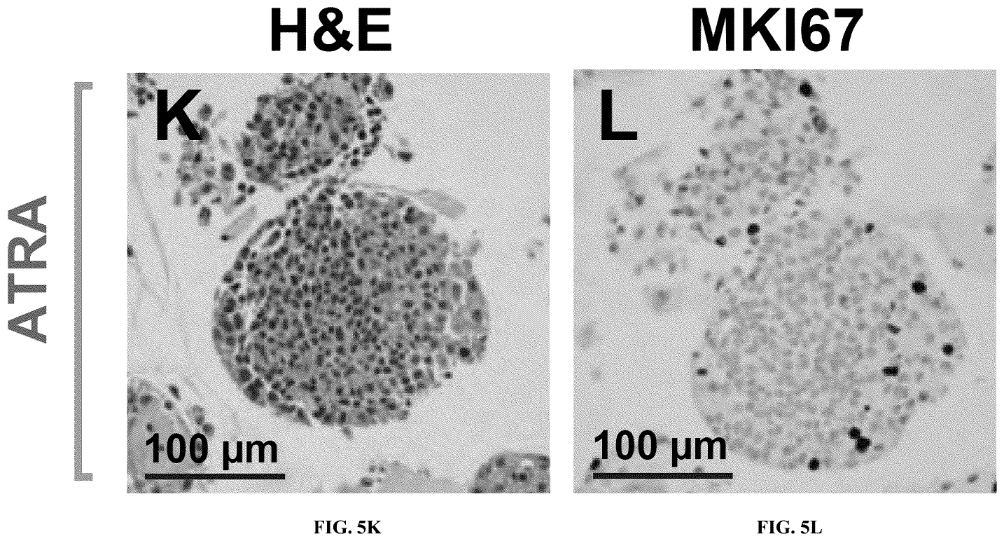

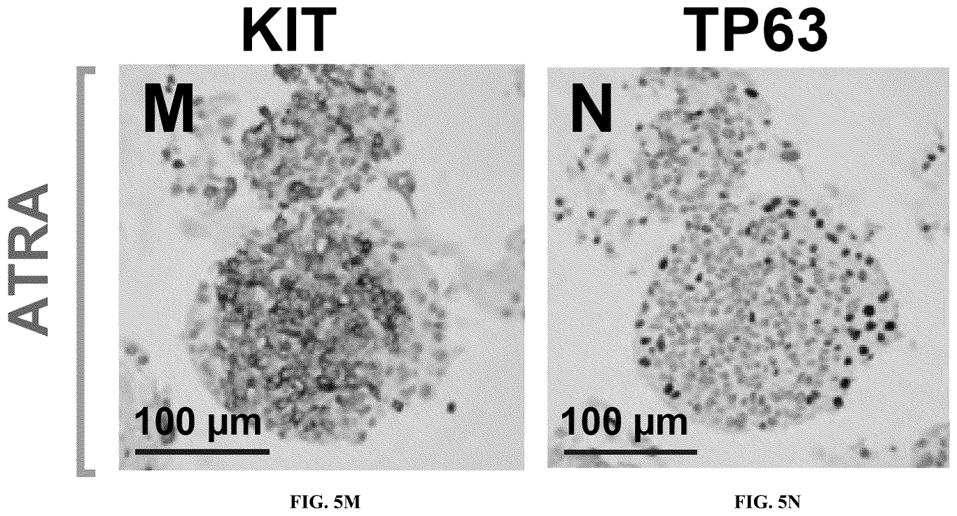

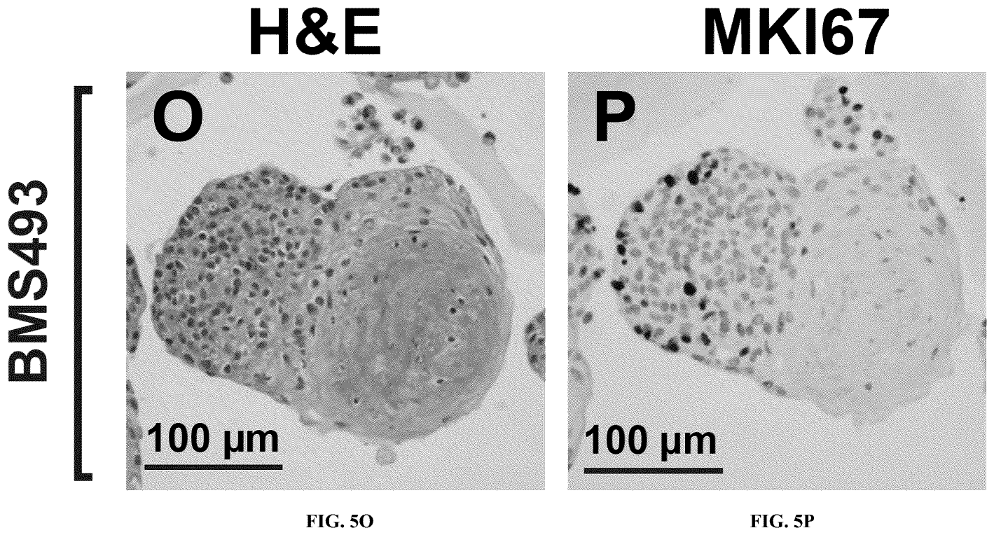

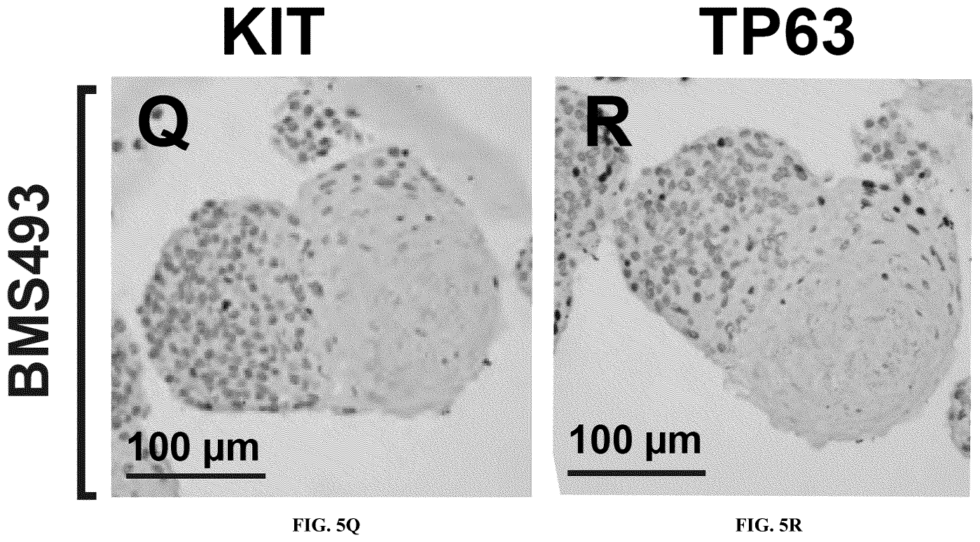

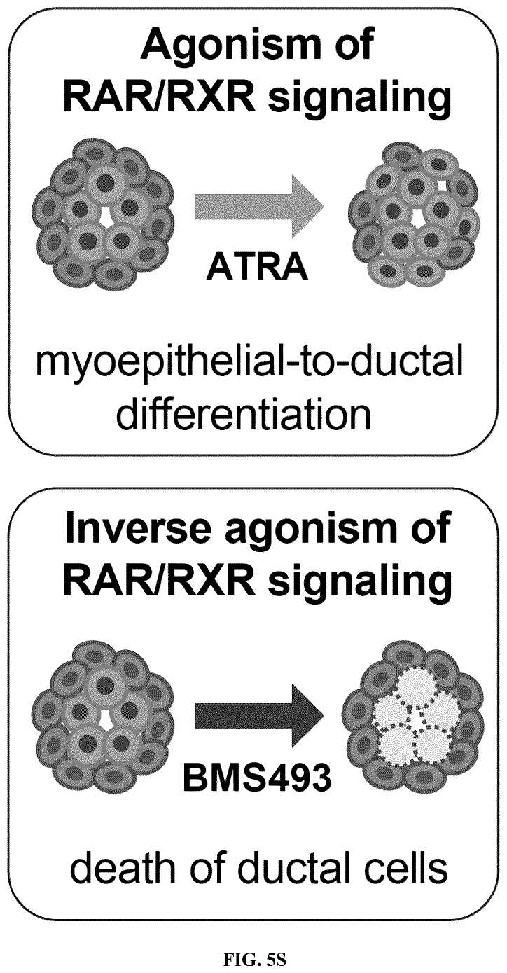

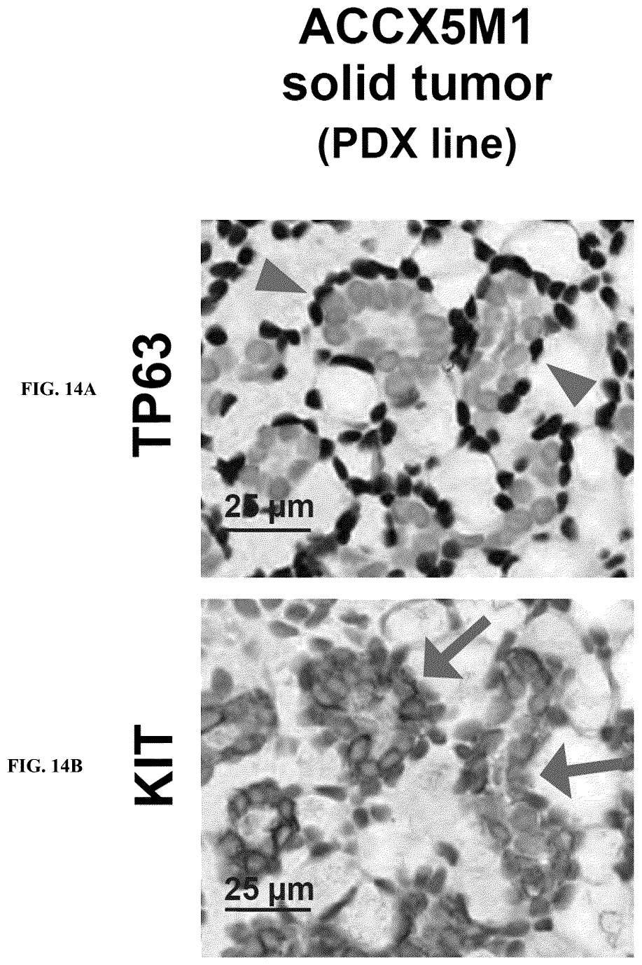

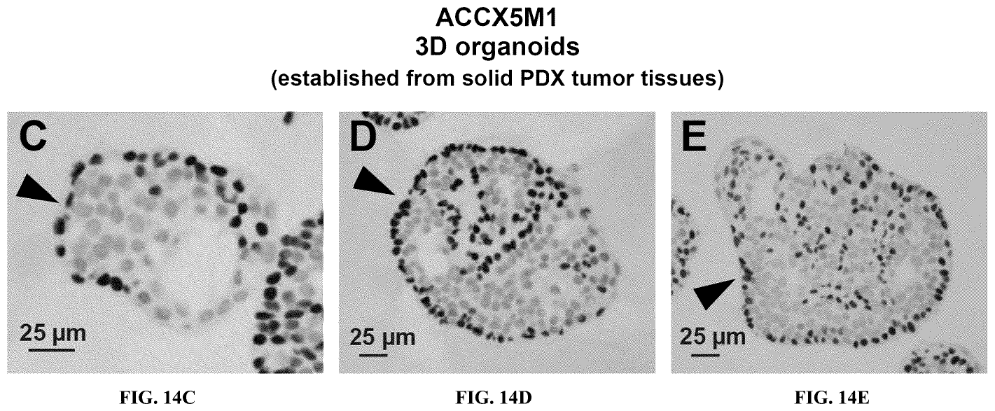

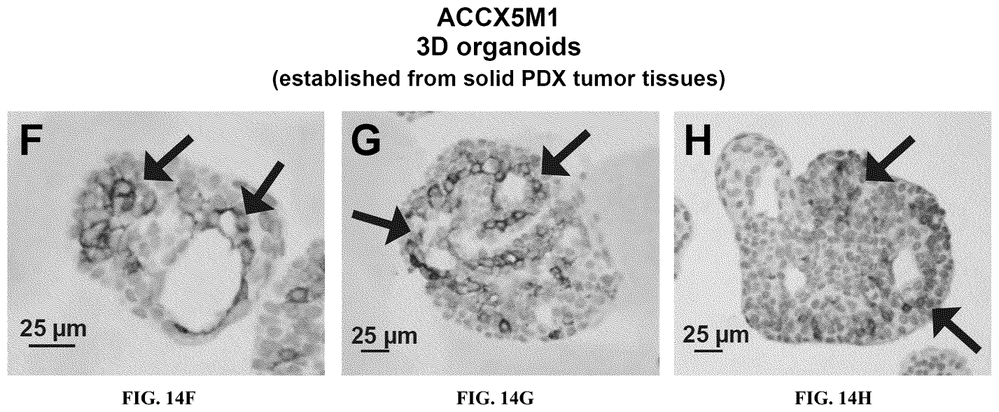

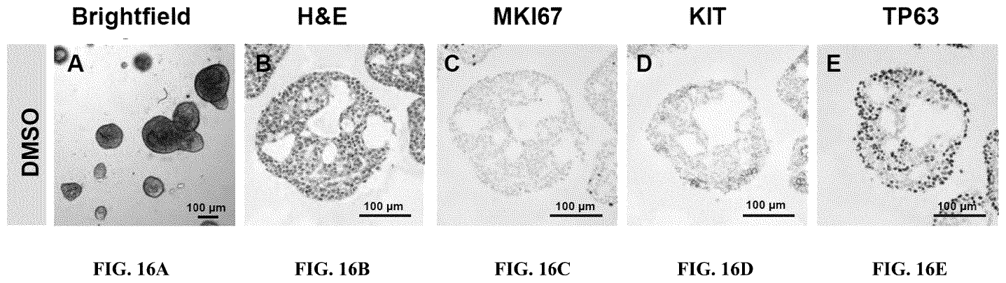

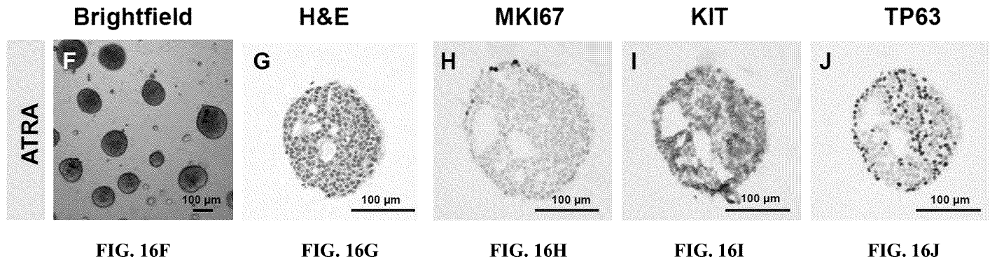

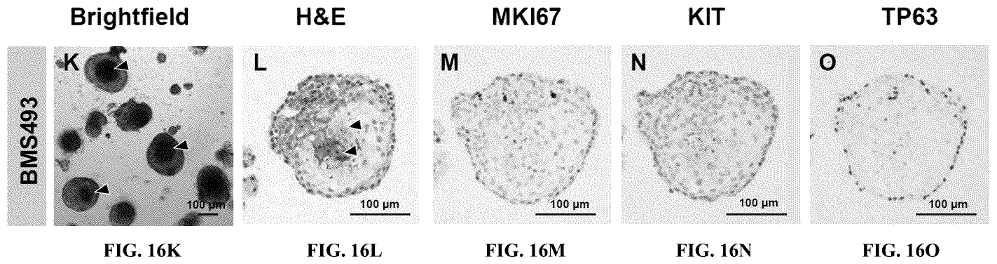

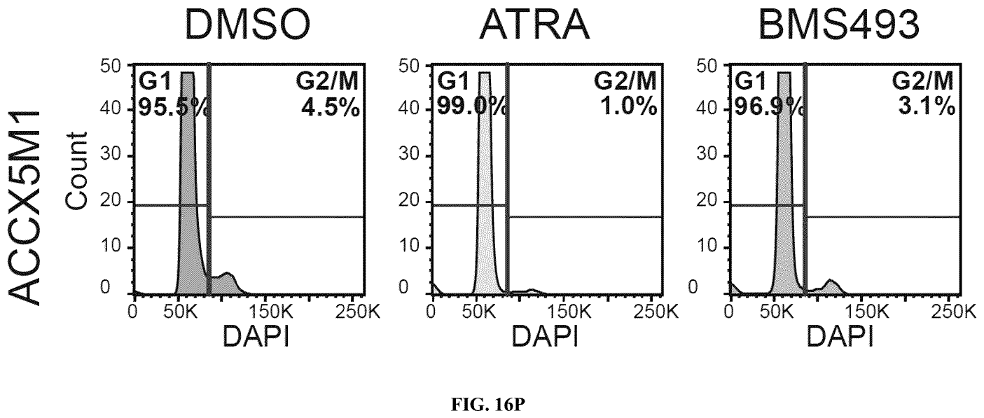

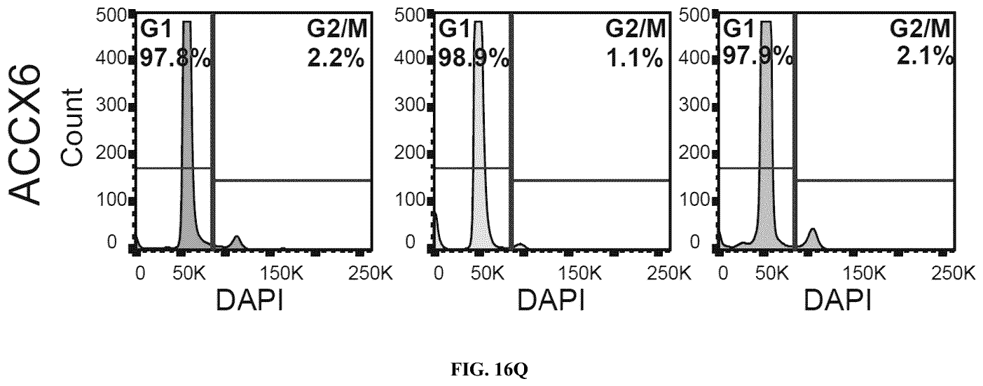

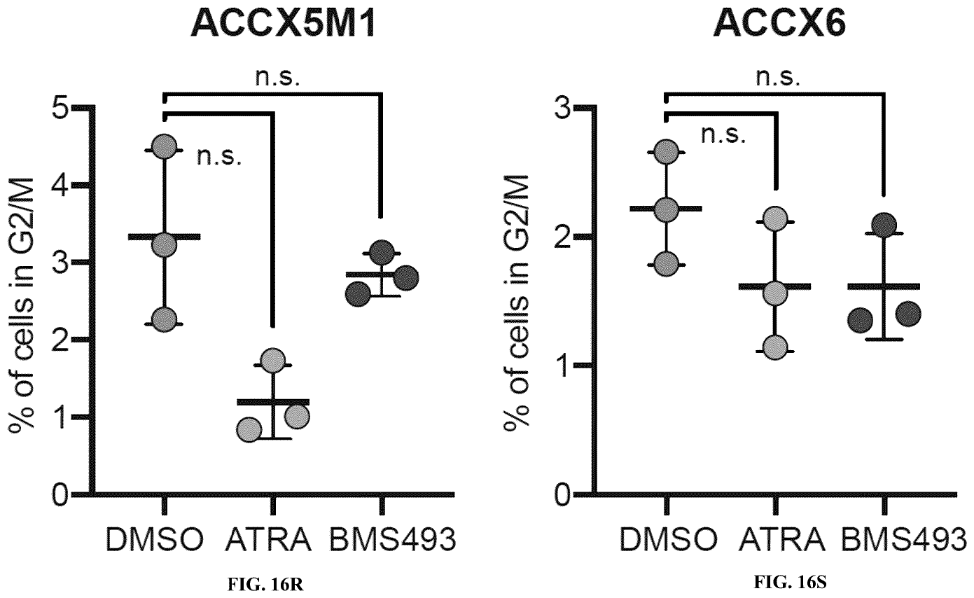

To elucidate the role played by RA signaling in regulating cell differentiation, a three-dimensional (3D) in vitro organoid tissue-culture system [32-34] was leveraged that recapitulated the bi-phenotypic composition of primary tissues (FIGS. 4D-4G), as well as key elements of their histological architecture (FIG. 14). It was observed that, upon stimulation of organoid cultures with agonists of RARs (ATRA) or RXRs (bexarotene), the percentage of CD49f^{low}/KIT⁺ cells increased, while suppression of RAR/RXR signaling with inverse agonists (BMS493, AGN193109) resulted in selective loss of CD49f^{low}/KIT⁺ cells (FIGS. 4H-4I). These effects were observed at concentrations that spanned the drugs' known ED₅₀ (0.1-10 μM) (FIGS. 4J-4M) and were reproduced across three bi-phenotypic PDX lines (ACCX5M1, SGTX6, ACCX6) (FIGS. 5A-5F). To clarify the mechanism causing such changes in cell composition, it was tested whether ATRA or BMS493 induced preferential proliferation of one cell-type. Analysis by IHC and FACS showed no increases in the frequency of MKI67⁺ cells (FIGS. 5G-5R, 16A-16O) or cells in the G2/M phase of the cell cycle (FIGS. 16P-16S) in either cell-type. The IHC analysis also confirmed an increase in KIT⁺/TP63^{neg} cells in ATRA-treated organoids and a stark loss of KIT⁺/TP63^{neg} cells after BMS493-treatment (FIGS. 5G-5R, 16A-16O). Remarkably, organoids treated with BMS493 displayed a striking change in morphology, with areas occupied by KIT⁺ cells undergoing nuclear fragmentation, suggesting selective cytotoxicity towards ductal-like cells (FIGS. 5O-5R, 16L). It was hypothesized that agonism and suppression of RAR/RXR signaling might have lineage-specific effects on the two cell populations (FIG. 5S). To formally test this hypothesis, CD49f^{high}/KIT^{neg} and CD49f^{low}/KIT⁺ cells were purified and treated individually with ATRA (10 μM) or BMS493 (10 μM) using 2D monolayer cultures [35] (FIGS. 6A-6F). The experiment revealed that stimulation with ATRA did not impact the viability of CD49f^{high}/KIT^{neg} cells (FIG. 6B), but changed their phenotype, with a majority of cells becoming CD49f^{low}/KIT⁺ (FIG. 6C-6D), suggesting myoepithelial-to-ductal differentiation. Conversely, treatment of purified CD49f^{low}/KIT⁺ cells with BMS493 resulted in a substantial decrease in cell viability, indicating selective toxicity against ductal-like cells (FIG. 6E-6F). To provide orthogonal evidence in support of RAR/RXR signaling as a key mediator of myoepithelial-to-ductal differentiation, it was tested whether the effects of RAR/RXR inhibitors could be phenocopied by over-expression of a dominant-negative version of human RARα (DNhRARα), known to suppress the transcriptional activity of all three members of the human RAR family (RARα, RARβ, RARγ) [37]. Indeed, infection of CD49f^{high}/KIT^{neg} cells with a lentivirus driving constitutive expression of DNhRARα resulted in complete abrogation of their spontaneous differentiation into CD49f^{low}/KIT⁺ cells (FIGS. 6G-6N).

### Example 6. In Vivo Anti-Tumor Activity of BMS493

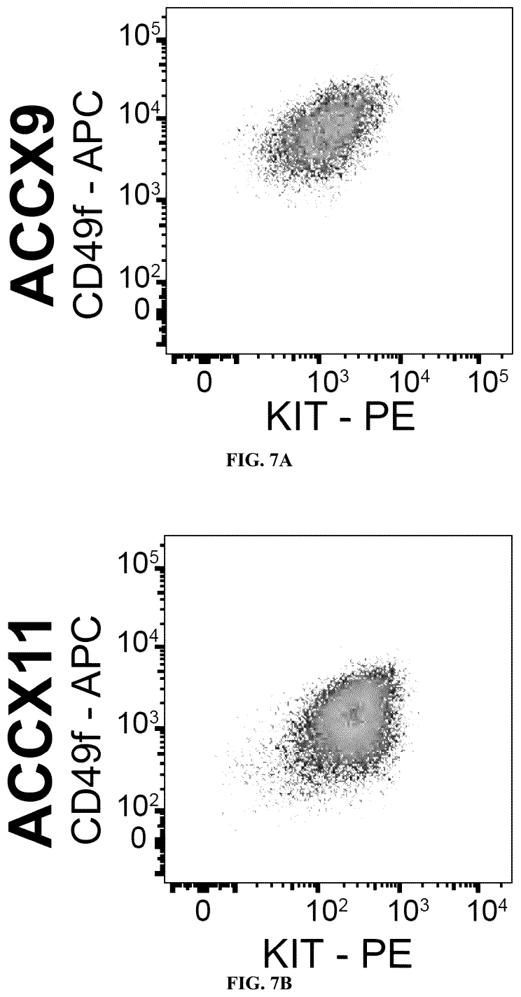

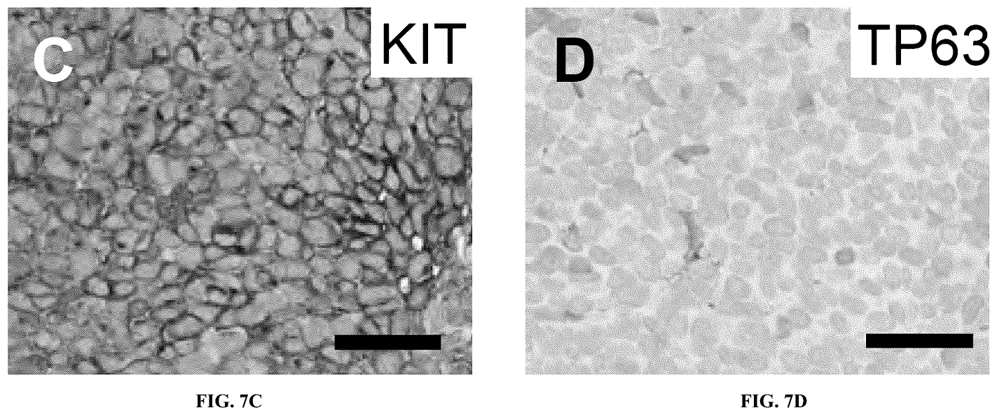

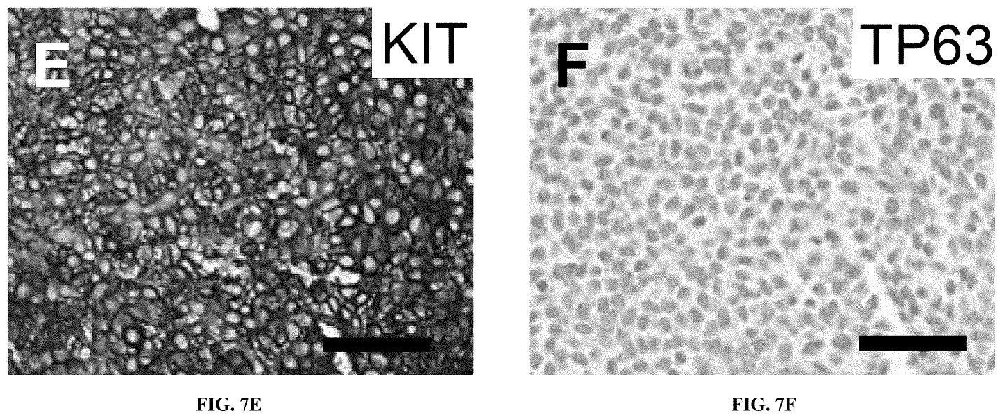

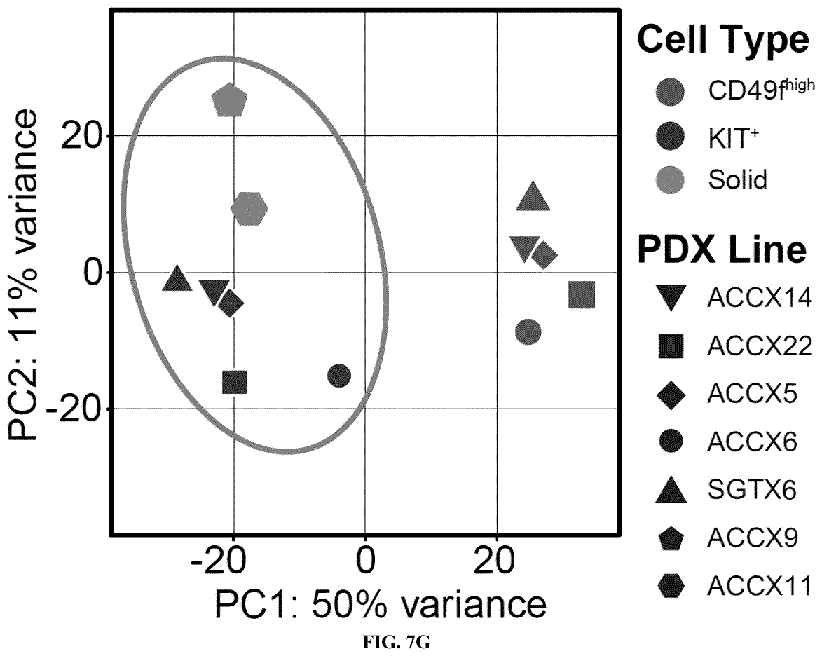

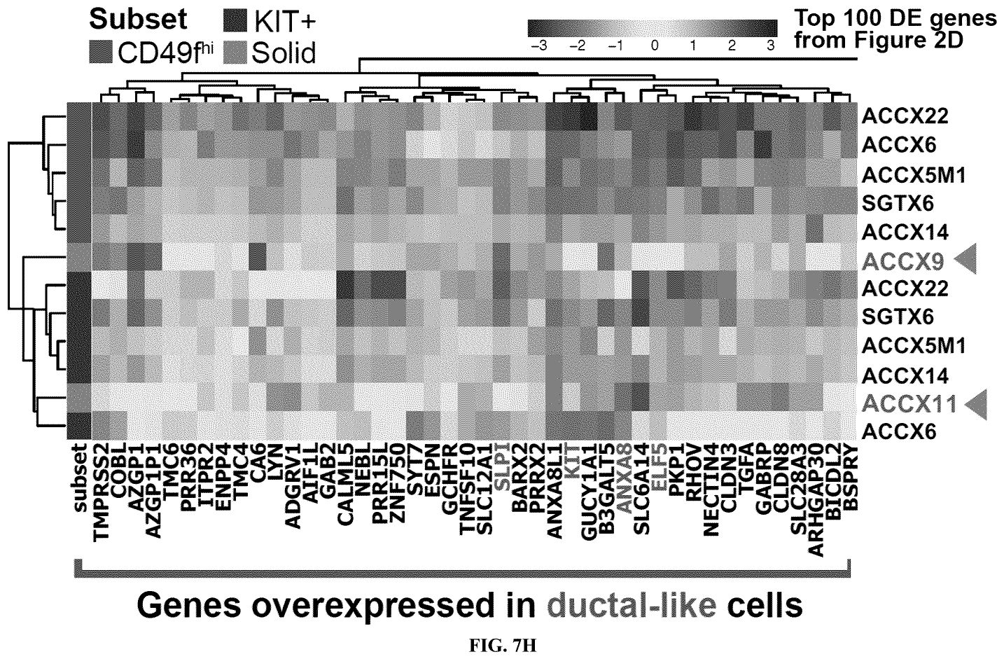

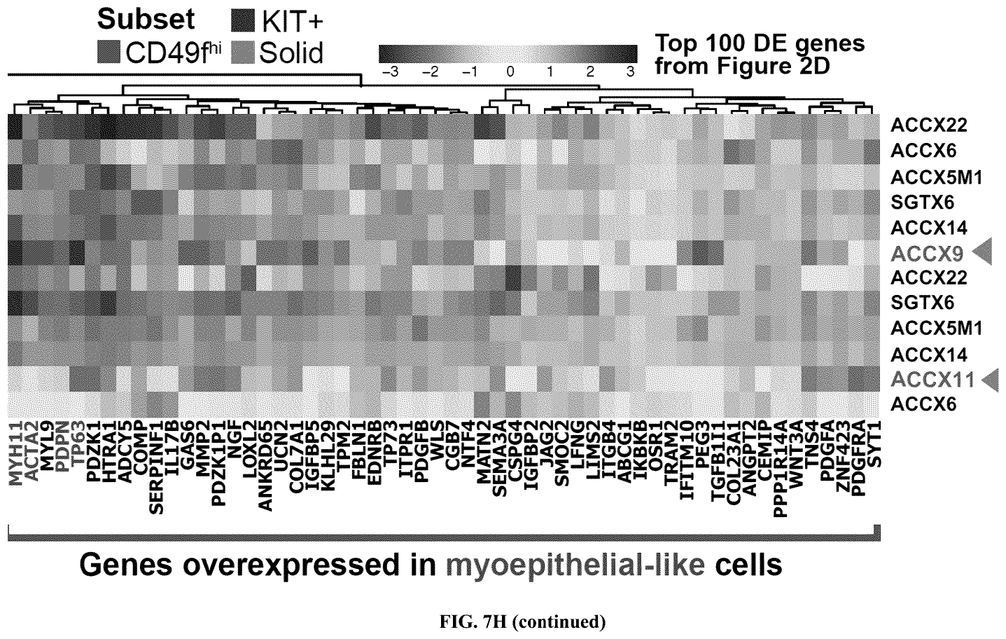

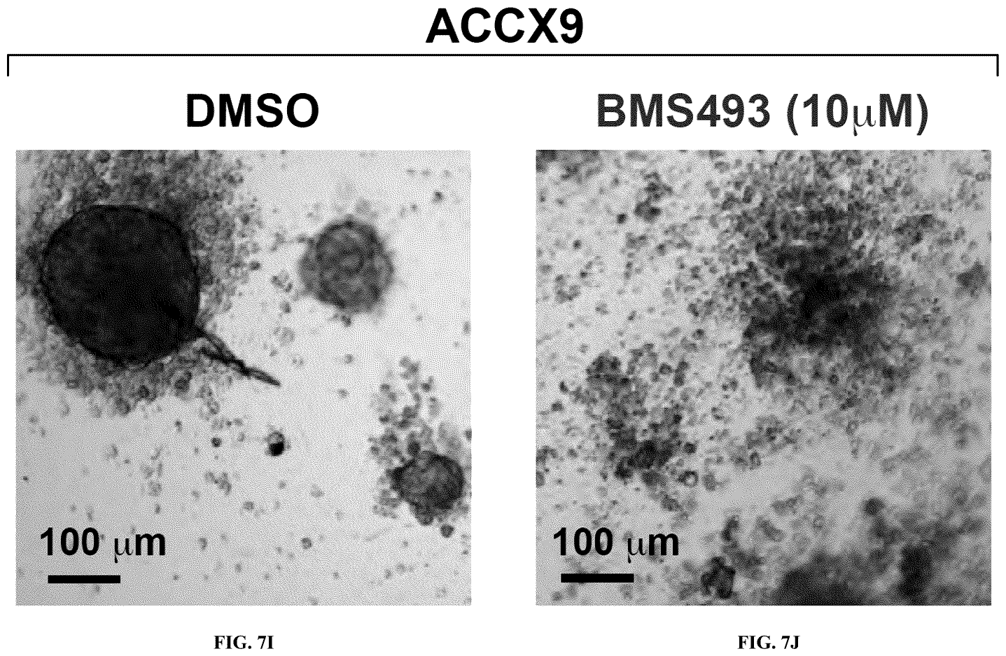

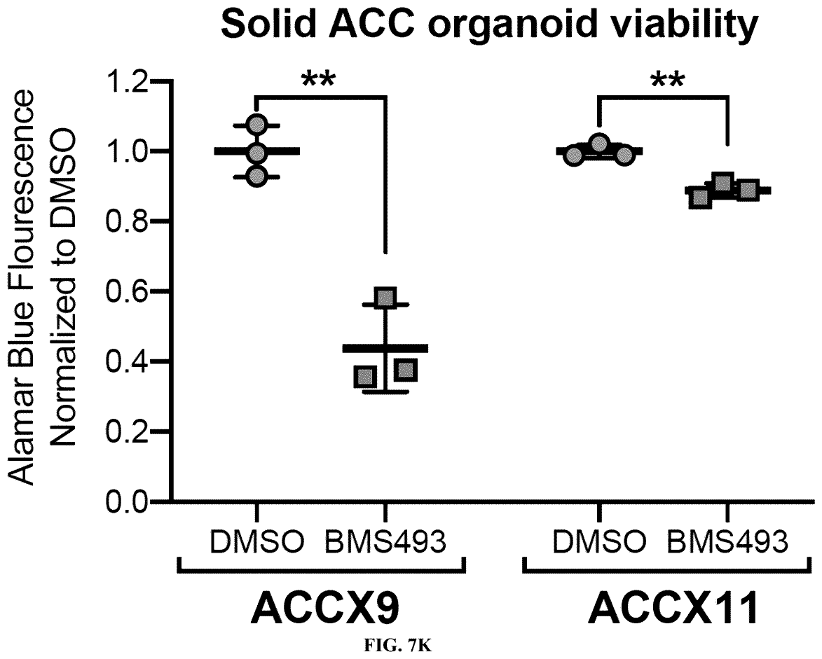

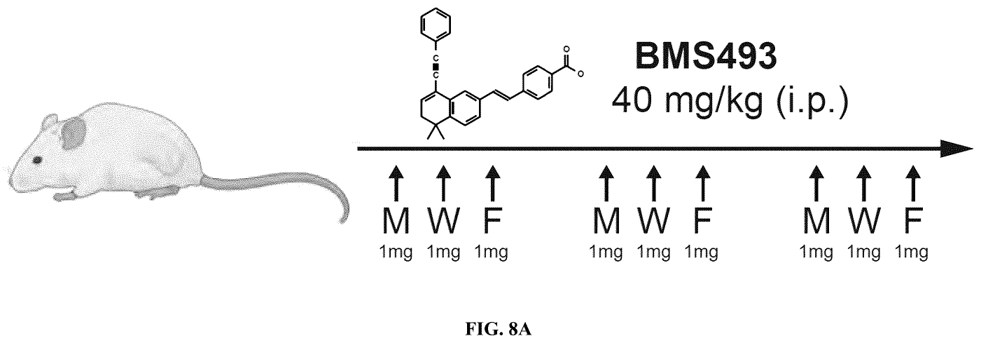

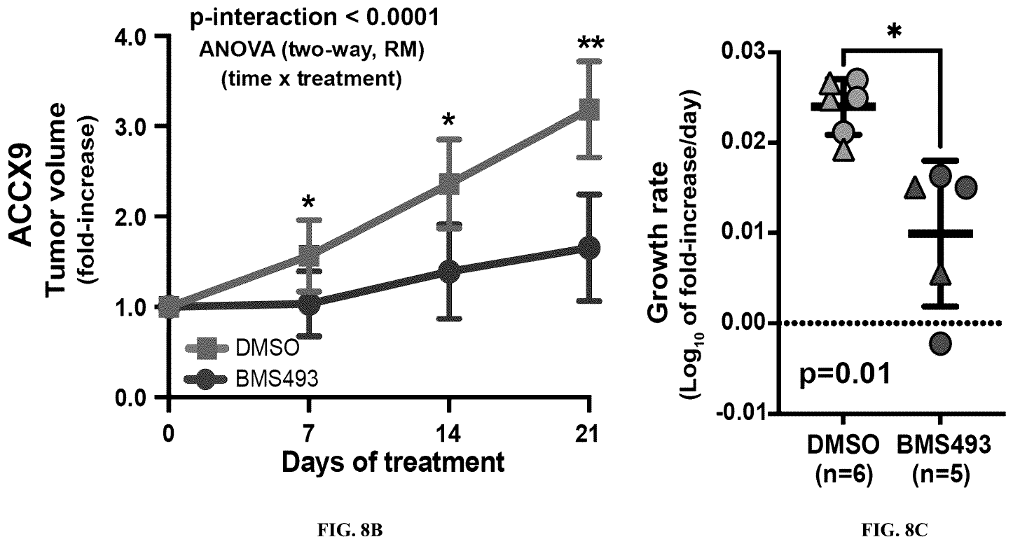

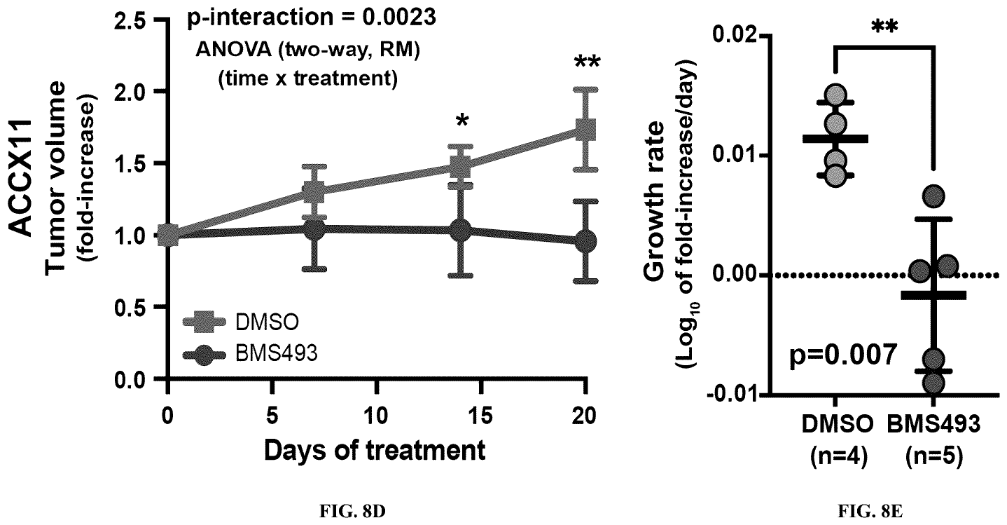

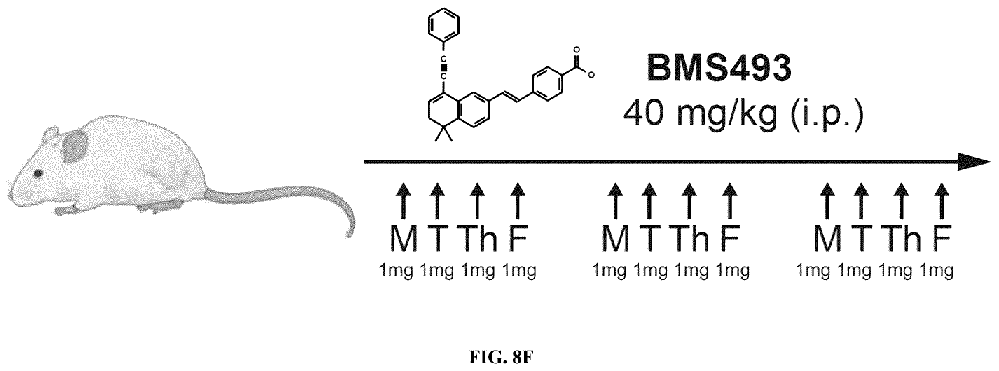

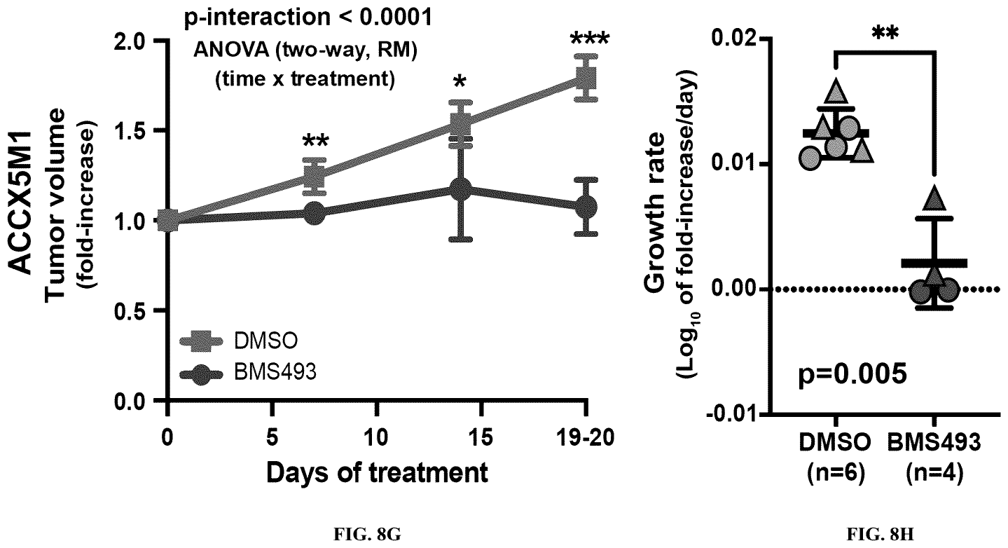

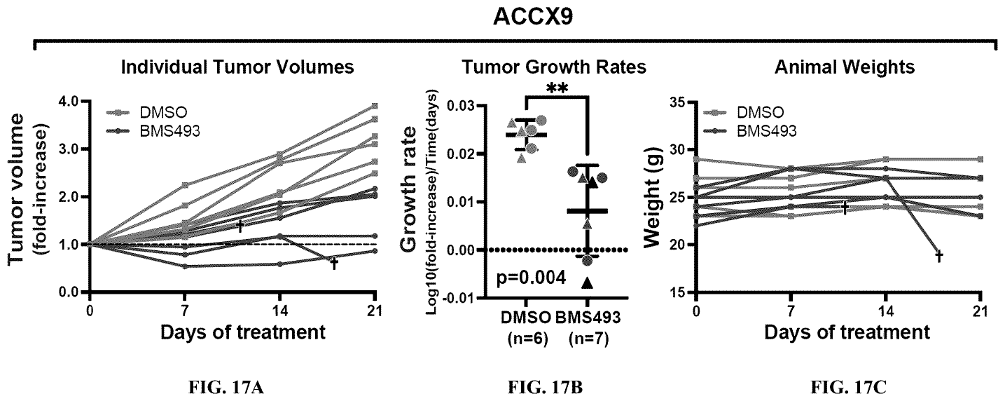

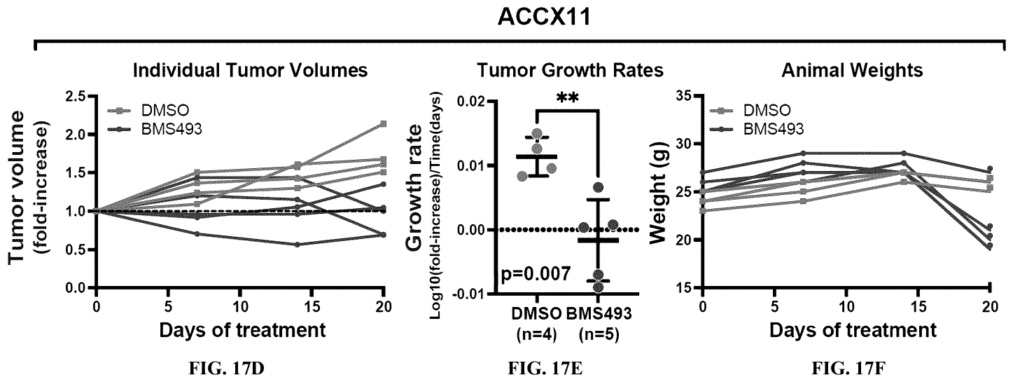

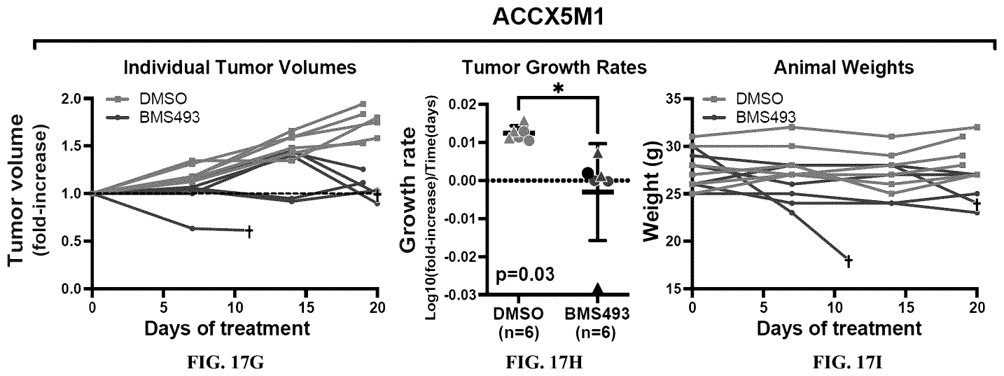

It was elucidated whether the selective toxicity displayed by BMS493 against ductal-like cells in vitro could be leveraged for the in vivo therapy of ACCs. It was hypothesized that, among ACCs, those enriched in ductal-like cells would represent the most susceptible targets. While most ACCs display bi-phenotypic histology, over the course of the disease, a subgroup progresses to a “solid” histological pattern, consisting predominantly of KIT⁺ cells [20]. Progression to solid histology associates with NOTCH1 activating mutations, increased proliferation kinetics and worse clinical outcomes [57-63]. To understand whether ACCs with solid histology represented mono-phenotypic expansions of ductal-like cells, two PDX models representative of this specific sub-type (ACCX9, ACCX11) [20] were analyzed and it was confirmed that they consisted of a single KIT⁺/TP63^{neg} population (FIGS. 7A-7F). RNA-seq was then performed on KIT⁺ cells purified by FACS from these two models, and repeated the PCA, combining the new data with those from purified pairs of myoepithelial-like and ductal-like cells from bi-phenotypic ACCs. Indeed, KIT⁺ cells from solid ACCs clustered with CD49f^{low}/KIT⁺ cells from bi-phenotypic ACCs (FIG. 7G), indicating retention of a ductal-like transcriptional profile (FIG. 7H). Furthermore, when treated with BMS493 (10 μM), organoids established from solid PDX lines displayed loss of structural integrity and decreased viability, indicating retention of sensitivity to suppression of RAR/RXR signaling (FIGS. 7I-7K). As a final step, it was tested whether in vivo administration of BMS493 (40 mg/kg, i.p.) could be leveraged for the treatment of PDX lines with either solid (ACCX9, ACCX11) or cribriform (ACCX5M1) histology (FIG. 8). A more intense regimen was utilized for the cribriform model (4 times/week×3 weeks, FIG. 8F) as compared to the solid models (3 times/week×3 weeks, FIG. 8A), assuming lower sensitivity. Treatment with BMS493 was associated with side-effects reminiscent of vitamin A deficiency (e.g., encrusted eyelids, rough coat, scaling of skin) [64]. Out of 18 tumor-bearing animals treated with BMS493, 33% (n=6/18) experienced tumor shrinkage (FIG. 17). Four animals (22%) were prematurely euthanized due to abrupt deterioration of general health conditions. In three of these animals, health deterioration occurred immediately following tumor shrinkage, suggesting acute toxicity due to tumor lysis (FIG. 17). Overall, treatment with BMS493 led to a statistically significant reduction in tumor growth across all three models, even after removal of animals undergoing premature euthanasia (FIGS. 8 B-8E, 8G-8H, FIG. 17).

### Example 7. Methods and Materials

### A. PDX Lines

PDX lines representative of human ACCs (Table 1) were obtained from the Adenoid Cystic Carcinoma Registry (ACCR) at the University of Virginia and propagated subcutaneously (s.c.) in female NOD·Cg-Prkdc^{scid} Il2rg^{tm1Wj1}/SzJ (NSG) mice (The Jackson Laboratory; stock #005557) [25].

PDX lines established from 7 independent human ACCs (ACCX5M1, ACCX14, ACCX22, SGTX6, ACCX6, ACCX9, ACCX11) were obtained from the Adenoid Cystic Carcinoma Registry (ACCR) at the University of Virginia [27]. PDX models were derived from donors of both sexes (females: n=5; males: n=2), with an age distribution of 33-77 years. Clinical and pathological characteristics of patient donors and corresponding primary tumors, as provided by the ACCR and previous publications [27], are described in Table 1. Tumor tissues were propagated in adult (>6 weeks of age), female, NOD·Cg-Prkdc^{scid} Il2rg^{tm1Wj1}/SzJ mice, also known as NOD/SCID/IL2Rγ^{-/-} (NSG) mice (The Jackson Laboratory; stock #005557), by sub-cutaneous xenotransplantation of solid fragments, following previously published procedures [25, 23].

### B. Animal Welfare

Animal experiments were approved by the Institutional Animal Care and Use Committee (IACUC) of Columbia University (research protocols: AC-AAAL7751, AC-AAAW1466, AC-AABM9553).

All animal experiments were performed with the approval of the Institutional Animal Care and Use Committee (IACUC) of Columbia University (research protocols: AC-AAAL7751, AC-AAAW1466, AC-AABM9553). Procedures involving the use of live animals were approved by the IACUC, and all researchers involved in animal studies completed required training on the use and care of research animals. Columbia University is accredited by the Association for Assessment and Accreditation of Laboratory Animal Care International (AAALAC; accreditation: #000687) and maintains Animal Welfare Assurance with the Public Health Service (PHS; assurance #D16-00003). Columbia University is also licensed to conduct animal experiments by the United States Department of Agriculture (USDA; license #21-R-0082) and the New York State Department of Health (NYSDOH; license #A141).

### C. Data and Software Availability

RNA-sequencing datasets were deposited in the database of Genotypes and Phenotypes (dbGAP), under accession number: phs002764. All software used in this study is either publicly or commercially available.

All computer software used in this study is either deposited in public repositories or commercially available, and listed in detail under the specific section of the present appendix that describes the experimental procedure involving its use.

The software used for the analysis of single-cell RNA-sequencing (scRNA-seq) datasets included:
cellranger (v3.1.0);
Randomly [28];
Scanpy;

The software used for the analysis of conventional RNA-sequencing (RNA-seq) datasets included:
bcl2fastq2 (v2.20);
kallisto (v0.44.0);
DESeq2 (v1.28.1) [29];
R (v4.0.1) and associated tidyverse (v1.3.0) software packages: ggplot2 (v3.3.3; RRID:SCR_014601), pheatmap (v1.0.12), RColorBrewer (v1.1-2), DEGreport (v1.24.1), dplyr (v1.0.5), tibble (v3.1.0), reshape2 (v1.4.4), GOstats (v2.54.0);
sva (v3.36.0) with ComBat-seq [76];
STAR-fusion (v1.7.0) [30];

The software used for Extreme Limiting Dilution Analysis (ELDA) [5] is publicly available. The acquisition and contrast-enhancement of microscopic images representative of tissues analyzed by immunohistochemistry (IHC) was performed using the QuPath software and Adobe Photoshop (v22.5.0; RRID:SCR_014199). Analysis of flow cytometry data was performed using FACSDiva (Becton Dickinson; RRID:SCR_001456) and FlowJo (version 10.7.1, Becton Dickinson; RRID:SCR_008520).

### D. Fluorescence-Activated Cell Sorting (FACS)

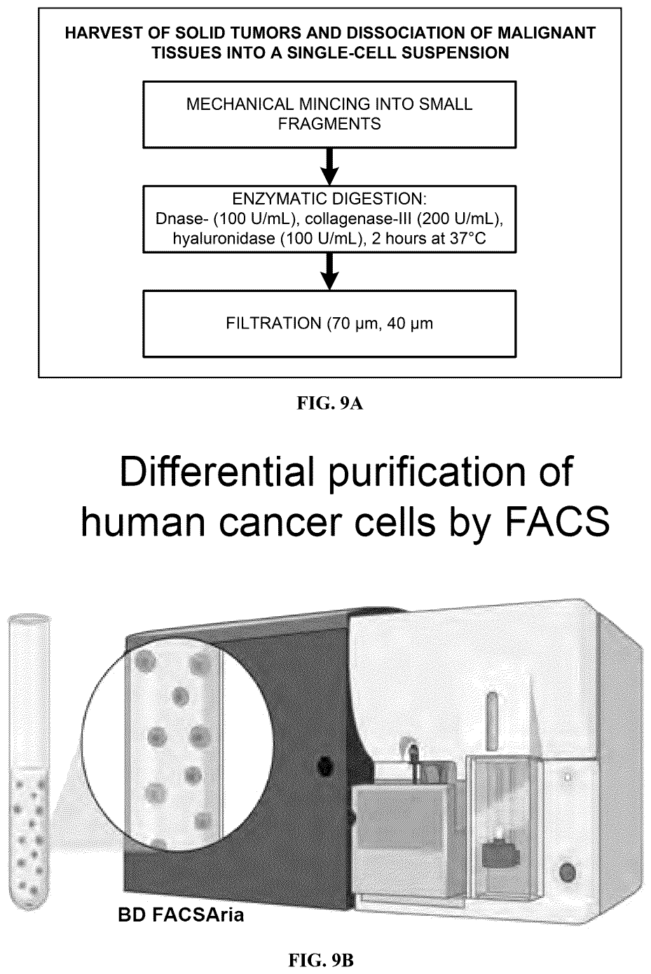

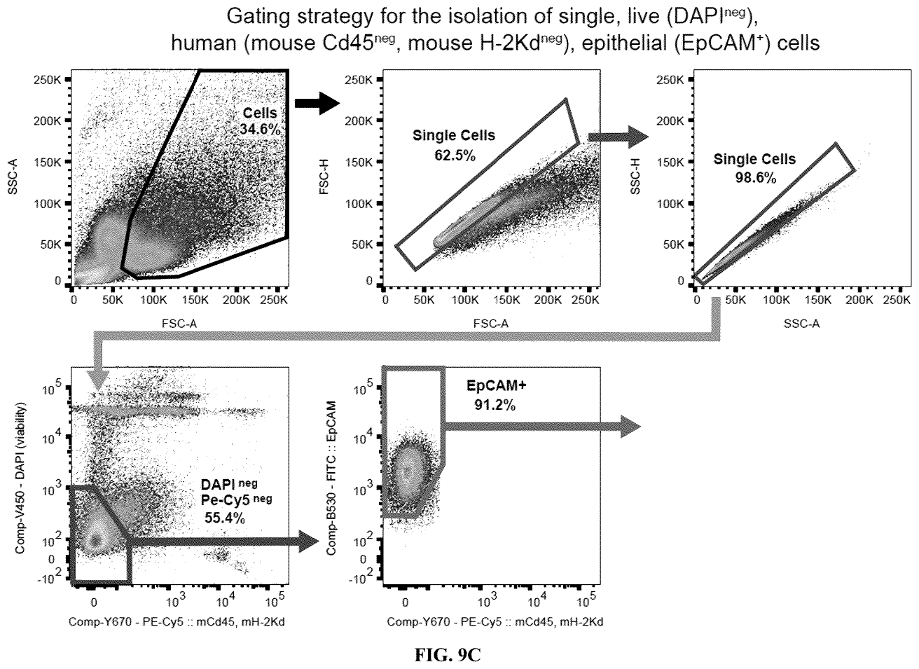

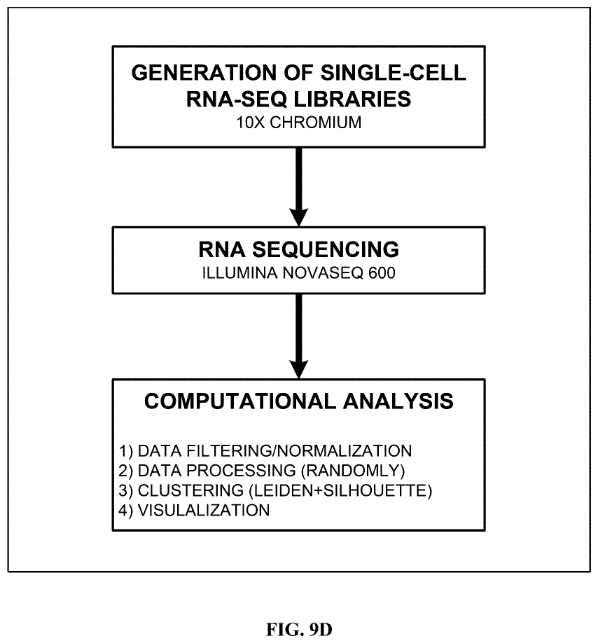

Solid tumors were dissociated into single-cell suspensions, and malignant cells isolated by FACS, following established protocols (FIG. 9) [23, 25]. Monoclonal antibodies used to visualize different sub-types of malignant cells included: mouse-anti-human-EpCAM-FITC (clone: 9C4), rat-anti-human/mouse-CD49f-APC (clone: GoH3) and mouse-anti-human-KIT-PE (clone: 104D2). Mouse cells were excluded using: mouse-anti-mouse-H-2Kd-biotin (clone: SF1.1), rat-anti-mouse-Cd45-PE/Cyanine5 (clone: 30-F11) and streptavidin-PE/Cyanine5 (BD Biosciences). Cell-cycle distribution of sorted cells was evaluated using DAPI, following permeabilization with BD Cytofix/Cytoperm (BD Biosciences).

Single-cell suspensions were either analyzed using a high-parameter f^{low} cytometer (LSRFortessa; Becton Dickinson) or used as starting material to purify selected sub-populations using a cell-sorter (FACSAria-III; Becton Dickinson), following previously established analytical pipelines [25, 23], with minor modifications (FIG. 9C). In experiments performed using the LSRFortessa, cell doublets were eliminated using a sequential gating strategy, based on forward-scatter area vs. forward-scatter width (FSC-A vs. FSC-W) and side-scatter area vs. side-scatter width (SSC-A vs. SSC-W) profiles. In experiments performed using the FACSAria-III, cell doublets were eliminated using a similar strategy, with sequential gating based on forward-scatter area vs. forward-scatter height (FSC-A vs. FSC-H) and side-scatter area vs. side-scatter height (SSC-A vs. SSC-H) profiles (FIG. 9C). Dead cells and cells of murine origin (i.e., cells expressing mouse stromal markers, such as H-2K^{d} and Cd45) were eliminated by exclusion of DAPI⁺ and PE/Cyanine5⁺ cells, respectively (FIG. 9C). Human epithelial cancer cells were differentially isolated from other cell-types by selective inclusion of EpCAM⁺ cells (FIG. 9C) and then sorted into myoepithelial-like (CD49f^{high}/KIT^{neg}) and ductal-like (CD49f^{low}/KIT⁺) sub-types using trapezoid gates designed to match the expression patterns of individual PDX lines (FIG. 2A). Data was acquired using the FACSDiva software (Becton Dickinson; RRID:SCR_001456) and analyzed using FlowJo (version 10.7.1, Becton Dickinson; RRID:SCR_008520).

### E. RNA Sequencing

ScRNA-seq experiments were performed using Chromium Single Cell 3′ Solution (10× Genomics) and NovaSeq-6000 (Illumina) platforms, and analyzed using cellranger (v3.1.0) and Randomly [28]. In conventional RNA-seq experiments, RNA was isolated using the NucleoSpin® RNA XS kit (Takara) and cDNA libraries prepared using the TruSeq Stranded mRNA kit (Illumina). Conventional RNA-seq reactions were run on either HiSeq-4000 or NovaSeq-6000 platforms (Illumina), and results analyzed using DESeq2 and STAR-fusion [30]. Differentially expressed genes were identified based on false-discovery rates (FDRs), calculated using the Benjamini-Hochberg method.

Live, human cancer cells (DAPI^{neg}, H-2Kd^{neg}, Cd45^{neg}, EpCAM⁺) were purified by FACS from a solid xenograft (ACCX22) and single-cell libraries were prepared using the Chromium Single Cell 3′ Solution (10× Genomics) with the Single Cell 3′ v3 chemistry, following the manufacturer's instructions. RNA-sequencing was performed on the NovaSeq-6000 platform (Illumina) at the JP Sulzberger Columbia Genome Center. Sequencing reads were mapped to human transcriptome GRCh38-3.0.0 and analyzed with the cellranger pipeline (version 3.1.0; 10× Genomics). The raw sequencing data (FASTQ) generated by this experiment have been deposited in the dbGAP repository (https://www.ncbi.nlm.nih.gov/gap) and are publicly available under accession number: phs002764.

Analysis of bulk RNA-seq data was performed in R (version 4.0.1). Data was normalized for batch effects using ComBat-seq [76] and gene expression values expressed using the r log function, which transforms data to the log 2 scale, after normalization of read counts with respect to library size. The presence of different subgroups of samples, defined by systematic differences in their gene-expression profiles, was visualized by Principal Component Analysis (PCA), performed using the plotPCA function with default parameters (i.e., using the 500 genes displaying the highest variance across the full dataset). Genes differentially expressed between CD49f^{high}/KIT^{neg} and CD49f^{low}/KIT⁺ cells across five PDX lines representative of by-phenotypic ACCs (ACCX5M1, ACCX6, ACCX14, ACCX22, SGTX6) were identified using the DESeq2 package (RRID:SCR_0156871) [2]. Differentially expressed genes were defined as those displaying a >2-fold difference in mean expression levels between the two populations (log₂ fold-change >1) that was considered statistically robust based on a two-tailed Wald test corrected for multiple comparisons (FDR<0.05; Benjamini-Hochberg method). The genes identified as differentially expressed were 643 and were ranked based on the p-value from the Wald test (Table 2). Variance in gene-expression levels across different samples was visualized using heatmaps, generated using the pheatmap function, with scaling performed by mean-centering expression values for each gene and calculating z-scores. Heatmaps were generated using the 100 genes identified as being the most significant for differential expression between the two populations, after ranking based on the p-value from the Wald test. Heatmaps were organized by hierarchical clustering of both genes and samples, and resulting clusters visualized using dendrograms. Differences in the expression level of genes encoding for mechanistic mediators of retinoic acid (RA) signaling, including both activators and suppressors (ALDH1A3, DHRS3, CRABP1, CRABP2, FABP5, RARA, RARB, RARG, RXRA, RXRB, RDH10, LRAT), were tested for statistical significance using Student's t-test (paired samples, two-tailed).

RNA-seq datasets were analyzed for the presence of MYB-NFIB chimeric transcripts, as well as for differences in the relative representation of splicing isoforms, using the STAR-fusion software (version 1.7.0) [30], after mapping raw sequencing results (FASTQ files) to the GRCh37 human reference genome. Differences in the aggregate expression levels of MYB-NFIB chimeric transcripts, expressed as fusion fragments per million (FFPM), were tested for statistical significance using a Student's t-test (paired samples, two-tailed).

### F. Immunohistochemistry (IHC)

Formalin-fixed, paraffin-embedded tissue-blocks were stained with the following antibodies: mouse-anti-human-TP63 (clone: 4A4), rabbit-anti-human-KIT (clone: YR145), rabbit-anti-human-MKI67 (clone: 30-9).

Freshly isolated tissue-specimens were washed in Dulbecco's Phosphate Buffer Solution (DPBS) and fixed overnight (12-18 hours) in a 10% formalin solution (Sigma, HT501320). Formalin-fixed paraffin-embedded (FFPE) tissue-blocks were stained either using conventional histochemical stains, such as hematoxylin and eosin (H&E), or by immunohistochemistry (IHC). IHC stains were performed on the BenchMark ULTRA automated platform (Ventana) and visualized with the UltraView DAB Detection Kit (Ventana), following heat-induced epitope retrieval (HIER) using the Cell Conditioning 1 (pH 7.3) solution, and staining (32 minutes) with one of the following primary antibodies: mouse-anti-human-TP63 (clone 4A4; Ventana), rabbit-anti-human-KIT (clone YR145; Cell Marque; RRID: AB_1159085) or rabbit-anti-human-MKI67 (clone 30-9; Ventana; RRID: AB_2631262). Stained slides were imaged using a digital scanner (Leica SCN400), and regions of interest were captured using the QuPath software (https://qupath.github.io/, version 0.2.3). Image brightness and contrast were adjusted using Adobe Photoshop (version 22.5.0; RRID: SCR_014199). Adjustments were applied uniformly to the entire image.

### G. Tissue Dissociation and Preparation of Single-Cell Suspensions

Solid ACC tumors were harvested from NSG mice, washed with cold (4° C.) DPBS and dissociated into-single-cell suspensions based on previously published protocols [25, 23], with minor modifications. Very briefly, tumor tissues were cut into small pieces (approximate volume: 1-2 mm³) with surgical scissors, followed by thorough mechanical mincing with a razor blade. The resulting tissue fragments were resuspended in a “disaggregation medium”, consisting of: RPMI-1640 medium (Sigma, R8758) supplemented with 2 mM L-alanyl-L-glutamine (Corning; 25-015-CI), 100 U/mL penicillin and 100 μg/mL streptomycin (Sigma, P4333), 1× Antibiotic Antimycotic Solution (Corning; 30-004-Cl), 20 mM HEPES (Corning, 25-060-CI), 1 mM sodium pyruvate (Gibco, 11360070), 100 units/ml hyaluronidase (Worthington, LS002592), 100 units/ml DNase-I (Worthington, LS002139), and 200 units/ml collagenase-III (Worthington, LS004183). Tissue fragments were then incubated at 37° C. for two hours, with pipetting every 10-15 minutes to promote cell dissociation. The resulting cell suspension was then serially filtered through 70-μm and 40-μm nylon meshes, in order to remove undigested tissue fragments and cell clumps. Red blood cells (RBCs) were removed by osmotic lysis, achieved by incubating the cell-suspension (5 minutes, on ice) in a hypotonic buffer (155 mM ammonium chloride, 0.01 M Tris-HCl; Red Blood Cell Lysing Buffer Hybri-Max; Sigma, R7757). Dissociated single cells were then spun at 1,500 rpm for 5 minutes, and re-suspended by gentle pipetting in a “flow cytometry buffer” (FCB) solution, consisting of: 1× Hank's Balanced Salt Solution (HBSS, Sigma H6648) with 2% heat-inactivated adult bovine serum (Sigma, B9433), 20 mM HEPES (Corning, 25-060-C1), 5 mM EDTA (Sigma, 3690), 1 mM sodium pyruvate (Gibco, 11360-070), 100 U/ml penicillin and 100 μg/ml streptomycin (Sigma, P4333), and 1× Antibiotic Antimycotic solution (Corning, 30-004-Cl).

To prevent unspecific binding of antibodies, cells were incubated with human IgGs (5 mg/ml; Innovative Research, VN00089472) in FCB, on ice (4° C.) for 15 minutes. Cells were then washed with FCB, and stained (15 minutes, 4° C.) with monoclonal antibodies, at a dilution determined by individual titration experiments. Antibodies used for removal of mouse stromal cells included: mouse-anti-mouse-H-2K^{d}-biotin (clone SF1-1.1, dilution 1:20; BioLegend; RRID: AB_313739) and rat-anti-mouse-Cd45-PE/Cyanine5 (clone 30-F11, dilution 1:100; BioLegend; RRID: AB_312975). Biotin-conjugated antibodies were visualized by secondary staining with streptavidin PE/Cyanine5 (dilution 1:200; BioLegend, 405205). Antibodies used for staining of human tumor cells included: mouse-anti-human-EpCAM-FITC (clone 9C4, dilution 1:30; BioLegend; RRID: AB_756078), rat-anti-human/mouse-CD49f-APC (clone GoH3, dilution 1:40; BioLegend; RRID: AB_1575047) and mouse-anti-human-KIT-PE (clone 104D2, dilution 1:50; BioLegend; RRID: AB_314983). After staining, cells were washed with 1 mL FCB to remove unbound antibodies and resuspended in FCB containing DAPI (dilution 1:10,000; Invitrogen D3571).

### H. In Vivo Tumorigenicity

Autologous pairs of CD49f^{high}/KIT^{neg} and CD49f^{low}/KIT⁺ cells were double-sorted by FACS, resuspended in High-Concentration Matrigel (Corning), and injected s.c., side-by-side, into opposite flanks (left/right) of NSG mice. The frequency of tumor-initiating cells was calculated by Extreme Limiting Dilution Analysis (ELDA) [31].

To understand whether myoepithelial-like (CD49f^{high}/KIT^{neg}) and ductal-like (CD49f^{low}/KIT⁺) cells differed in their tumorigenic capacity (i.e., the capacity to initiate and sustain the growth of new tumors upon xeno-transplantation), an Extreme Limiting Dilution Analysis (ELDA) of their tumorigenic cell frequencies was performed, following the procedure described by Yifang Hu and Gordon K. Smyth (Bioinformatics Division, Walter and Eliza Hall Institute) [31]. Very briefly, autologous pairs of CD49f^{high}/KIT^{neg} and CD49f^{low}/KIT⁺ cells were “double-sorted” by FACS starting from the same tumor specimens, representative of two bi-phenotypic PDX lines (ACCX5M1, SGTX6). The two populations were sorted in parallel, using a cell-sorter equipped for 2-way parallel purification (FACSAria-III, Becton Dickinson), as described above and in previous publications [25, 23]. Double-sorting consisted in two sequential rounds of sorting, whereby, after the first sort, cells were spun down, resuspended in 0.5 mL of fresh FCB with DAPI, and then sorted a second time, using identical gates (FIG. 3A). Cells were assessed for purity and viability after the second sort, resuspended in fresh FCB and counted using a hemocytometer. Cells were then aliquoted at various doses (range: 250-10,000 cells) in 100 μl of cold (4° C.) FCB and kept on ice. High-concentration (HC) Matrigel matrix (Corning, 354262), was thawed on ice, diluted (1:2) with ice cold FCB, and finally added at 1:1 ratio to the suspensions of sorted cells (100 μl of diluted HC Matrigel+100 μL of sorted cells in FCB) for a final volume of 200 μl/injection aliquot. Each aliquot of sorted cells admixed with HC Matrigel (200 μl) was then injected subcutaneously (s.c.) in an NSG mice using 23 G×1¼ needles. Autologous pairs of CD49f^{high}/KIT^{neg} and CD49f^{low}/KIT⁺ cells were injected in parallel in the s.c. tissue of the left and right flank of the same animals (NSG mice, adult females; The Jackson Laboratory; stock #005557) in order to exclude confounding effects from individual variabilities in each animal's immune-competence. Animals were assessed weekly for the presence or absence of tumors in either flank. Upon tumor formation, tumor volume was measured weekly using the following formula:

volume=width²×length/2

Animals were either euthanized when tumors reached a maximum diameter of 2.0 cm, or monitored for a minimum of 10 months, to exclude tumor engraftment. Upon euthanasia, animals were dissected and the s.c. tissues of both flanks examined, to exclude the presence of sub-palpable tumors. Finally, the tumorigenicity data obtained from each PDX line were aggregated and analyzed using an online calculator developed by the authors who first developed the ELDA procedure (Yifang Hu, Gordon K. Smyth) and publicly available on the website of their academic institution [31]. Very briefly, the first step of the ELDA procedure consists in performing a maximum likelihood estimation (MLE) of the frequency of tumor-initiating cells (and its 95% confidence interval) in each of the analyzed populations. The MLE is performed using linear regression, as enabled by Generalized Linear Models (GLM). The second step of the ELDA procedure consists in testing for inequality the frequencies of tumor-initiating cells observed in different populations (in this case: CD49f^{high}/KIT^{neg} vs. CD49f^{low}/KIT⁺ cells) by performing a Likelihood-Ratio Test (LRT), in which the significance of the test's statistic (a natural logarithm of the likelihood ratio) is estimated by approximation using the χ² distribution (Wilk's theorem). Finally, tumors originated from the injection of purified preparations of either CD49f^{high}/KIT^{neg} or CD49f^{low}/KIT⁺ cells were analyzed by f^{low}-cytometry and IHC, to evaluate their cell composition. Differences in the percentage of CD49f^{high}/KIT^{neg} cells and CD49f^{low}/KIT⁺ cells observed between parent tumors, purified preparations of each cell type, and tumors generated from in vivo injection of such purified populations were visualized using box-plots [17] and tested for statistical significance using a Mann-Whitney U-test (one-tailed), aimed at testing whether: a) tumors originated from sorted cells of a specific phenotype displayed a higher content of that same cell-type as compared to their parent tumors; and b) tumors originated from sorted cells of a specific phenotype displayed a lower content of that same cell-type as compared to the corresponding preparations of sorted cells.

a. In Vitro Tissue-Cultures

ACC cells were cultured either as three-dimensional (3D) organoids [32-34] or two-dimensional (2D) monolayers and treated with all-trans retinoic acid (ATRA; 0.1-10 μM), bexarotene (10 μM), BMS493 (1-10 μM) or AGN193109 (1-10 μM). Lentivirus vectors [36] were based on the pLL3.7 backbone (Addgene; #11795), re-engineered to drive constitutive expression of a dominant negative version of human RARα (Addgene; #15153) in tandem with a fluorescent reporter (EGFP). Cell viability was assessed using the alamarBlue HS Cell Viability Reagent [38].

Organoid cultures were initiated from dissociated primary tissues of human Adenoid Cystic Carcinoma (ACC) patient-derived xenograft (PDX) lines [27], and cultured in vitro using previously described 3D organoid tissue-culture protocols [32-34], with minor modifications. Very briefly, one day prior to organoid plating, irradiated (100 Gy) feeder cells, consisting of a 1:1 mixture of L-Wnt-3A mouse fibroblasts (ATCC, CRL-2647), and R-Spondin1-HEK-293T cells (Trevigen, 3710-001-K), were thawed and plated at a density of 400,000 cells/well in a 24-well plate, after resuspension in a “feeder medium”, consisting of DMEM (Corning, 10-013-CV) containing 10% FBS (VWR, 89510-194), 100 U/mL penicillin and 100 μg/mL streptomycin (Millipore Sigma, P4333), 2 mM L-alanyl-L-glutamine (Corning 25-015-CI), 1 mM sodium pyruvate (Gibco, 11360070), and 20 mM HEPES (Corning, 25-060-CI). Cell lines were purchased at the start of the study and authenticated by the manufactures. Both cell lines were tested for mycoplasma contamination [86] and resulted negative. After 24 hours, the feeder medium was replaced with an “organoid medium”, consisting of DMEM Nutrient Mixture F-12 HAM tissue-culture medium (Sigma, D8437), supplemented with 10% heat-inactivated FBS (VWR, 89510-194), 2 mM L-alanyl-L-glutamine (Corning 25-015-CI), 20 mM HEPES (Corning, 25-060-CI), 1 mM sodium pyruvate (Gibco, 11360070), 100 U/mL penicillin and 100 μg/mL streptomycin (Sigma, P4333), 1× Antibiotic Antimycotic Solution (Corning 30-004-Cl), 1×ITES media supplement (Lonza, 17-839Z), 10 mM Nicotinamide (Sigma, 72340), and 100 ug/ml Heparin (Millipore Sigma, H3393). On the day of organoid plating, solid tumor tissues were minced into small fragments, then serially filtered through a 100 μm and a 40 μm mesh strainer. After the second filtration step, fragments trapped by the 40 μm strainer (i.e. tissue fragments smaller than 100 μm, but larger than 40 μm), were gently washed from the mesh with cold disaggregation medium and pelleted by centrifugation (1500 RPM, 5 minutes). Fragments were then resuspended in “complete organoid medium”, i.e., organoid medium supplemented with 50 ng/ml hEGF (Stem Cell Technologies, 78006.2), 500 ng/ml hR-Spondin1 (R&D systems, 4645-RS), and 10 μM Y-27632 (R&D Systems, 1254), and plated in transwell inserts (Greiner Thincert, 24 well, 0.4 μM pore size, 662641) atop a polymerized layer (˜100 μL) of Matrigel (Corning, 354234). Finally, transwells were placed in 24-well plates atop feeder cells and cultures were incubated at 37° C. with 5% CO₂. In vitro organoid cultures were established from five independent PDX models (ACCX5M1, ACCX6, SGTX6, ACCX9, ACCX11). In all experiments, the minimum number of technical and/or biological replicates was 3 (range: 3-13), and all attempts at replication were successful. Upon histological and immunohistochemical (IHC) analysis, organoids established from PDX lines that were representative of bi-phenotypic ACCs, appeared to recapitulate many of the distinctive architectural features observed in primary tumors (FIG. 14), including: 1) bi-phenotypic composition: the organoids contained two clearly distinct cell-types, characterized by mutually exclusive expression of either TP63 (a marker characteristic of myoepithelial-like cells) or KIT (a marker characteristic of ductal-like cells); 2) adenoid organization: the organoids displayed a 3D architecture that recapitulated key elements of the histological organization of parental tissues, whereby ductal-like cells (KIT⁺) appeared to cluster at the center of the organoids and arrange in “ring-like” structures around a lumen, while myoepithelial-like cells (TP63⁺) appeared to form a “crown” around ductal-like cells, lining the outer surface of the organoid, and making direct contact with the 3D Matrigel scaffolding (which contains basement membrane proteins and proteo-glycans similar to those found in the pseudo-cysts of primary ACCs).

b. In Vitro Studies with Direct and Inverse Agonist of Retinoic Acid (RA) Signaling

Stock solutions of direct and inverse agonists of RA signaling, including all-trans retinoic acid (ATRA, 100 mM; Sigma, R2625), bexarotene (100 mM; Tocris, 5819), BMS493 (10 mM; Tocris, 3509), and AGN193109 (10 mM; Sigma, SML2034), were prepared in DMSO and stored at −20° C., protected from light. On the day of use, stock solutions were thawed and added to complete organoid medium, at appropriate concentrations (0.1-10 μM). Due to the short half-life of retinoids, medium with retinoid compounds was kept for a maximum of 3 days at 4° C. and changed daily for the duration of tissue-culture (7 days).

Organoid cultures established from human ACCs were dissociated from Matrigel by incubation in a solution of 2 mg/mL Dispase-II (Thermo Fisher, 17105041) and 200 U/mL collagenase-III (Worthington, LS004183) in DPBS at 37° C. for 15 minutes. Organoids were then transferred to 1.5 mL plastic tubes and pelleted by centrifugation (10,000 rpm, 2 minutes). Excess Matrigel and disaggregation solution were carefully aspirated. To dissolve remaining Matrigel, organoid pellets were briefly (3 minutes) resuspended in 0.25% Trypsin at 37° C., and then washed with cell culture medium containing 10% FBS. To prepare organoids for immunohistochemistry, organoids were pelleted by centrifugation and fixed in 10% formalin for 4-12 hours. Fixed organoids were embedded in paraffin blocks, from which 4 μm tissue-sections were cut and stained, following protocols identical to those used for tumor tissues (described above). In the case of FACS experiments, organoid pellets were resuspended in disaggregation medium containing DNase-I (100 U/mL), collagenase-III (200 U/ml), and hyaluronidase (100 U/ml) and incubated at 37° C. for 20-30 minutes. Disaggregated organoids were then pelleted and incubated in 0.25% trypsin (10-15 minutes) to generate single cell suspensions. Dissociated cells were washed with cell culture medium containing 10% FBS to inhibit trypsin activity, followed by blocking with human IgGs (5 mg/ml) and staining with antibodies. Differences in the percentage of CD49f^{high}/KIT^{neg} and CD49f^{low}/KIT⁺ cells between organoids treated with different compounds were tested for statistical significance using either Student's t-test for independent samples (two-tailed) or Welch's one-way ANOVA (i.e., assuming unequal variance) followed by Dunnett's T3 test for multiple pair-wise comparisons [87]. Brightfield images of organoid cultures (4× magnification) were acquired using a Cytation-5 Cell Imaging Reader (BioTek). Images of hematoxylin and eosin (H&E) or IHC-stained organoids were acquired using a Nikon Eclipse E600 microscope with NIS-Elements Software (version 5.21). Brightness and contrast were adjusted uniformly throughout whole images using Adobe Photoshop (version 22.5.0).

CD49f^{high}/KIT^{neg} and CD49f^{low}/KIT⁺ cell populations were sorted by FACS from ACC xenografts (ACCX5M1). Sorted cell populations were resuspended in 100 μl of complete organoid medium supplemented with either DMSO, ATRA (10 μM) or BMS493 (10 μM). Cells were plated in 96-well plates (30,000 cells/well) and medium was changed daily for the duration of treatment (1 week), as also described in previous studies [35].

Both 2D and 3D cultures of ACC cells from PDX lines were grown in 96-well black plates with optically clear bottoms (Thermo Scientific, 165305), and then treated with either retinoids (ATRA, 10 μM; BMS493, 10 μM) or DMSO alone (1:1000) for one week. On the final day of treatment, a 20% solution of alamarBlue HS cell viability reagent (Invitrogen, A50100) was prepared in complete organoid medium and added to each well (final concentration of alamarBlue reagent=10%). Samples were incubated overnight (18-24 hours) at 37° C. and protected from light [38]. Sample fluorescence was measured using a Cytation-5 Cell Imaging Reader (BioTek) and fluorescence readings (ex/em 530/590) normalized to their mean in DMSO-treated control samples. Differences in the mean value of normalized fluorescent readings were tested for statistical significance using a Student's t-test for independent samples (two-tailed).

c. Experiments with Lentivirus Vectors Encoding for a Dominant-Negative Variant of the Human Retinoic Acid Receptor Alpha (DNhRARα)

The cDNA encoding for a dominant negative form of the human retinoic acid receptor alpha (DNhRARα), consisting in a shortened version of the receptor, truncated at amino-acid 403 (i.e., lacking the C-terminal transcriptional activation domain) [37], was obtained from the Addgene public repository, where it is available as part of a lentivirus construct based on the RCAS backbone (Addgene catalog: #15153) [88]. Very briefly, the DNhRARα cDNA was subcloned into a modified version of the pLentiLox3.7 (pLL3.7) lentivirus backbone (Addgene catalog: #11795), in which: 1) the mouse U6 promoter used to express short-hairpin RNA (shRNA) constructs was removed; and 2) a multi-cloning site (mcs) and an internal ribosomal entry site (IRES) from the encephalomyocarditis virus (EMCV) [89, 90] were inserted immediately following a cytomegalovirus (CMV) promoter driving the expression of an enhanced green fluorescent protein (EGFP) fluorescent reporter. The resulting lentivirus construct (pLL3.7-DNhRARα-EGFP) was able to drive the constitutive and simultaneous expression of both DNhRARα and EGFP, as a result of a polycistronic mRNA that encoded the two cDNAs in tandem. Lentivirus infectious particles were produced following established protocols and procedures [36] for 3^{rd} generation lentivirus vectors [91, 92], with minor modifications [23]. Lentivirus infectious particles were produced by co-transfection in human embryonic kidney HEK293 cells (GenHunter; catalog: Q401) of four distinct plasmids, including three plasmids encoding for distinct structural and/or functional elements of the virion (pMDLg/pRRE, Addgene #12251; pCMV-VSV-G, Addgene #8454; pRSV-Rev, Addgene #12253) and one plasmid encoding the transgene of interest (pLL3.7-DNhRARα-EGFP). Plasmids were transfected into HEK293 cells using the JetPRIME transfection reagent (Polyplus Transfection), following the manufacturer recommendations. HEK293 cells were then incubated in tissue-culture media supplemented with caffeine (4 mM) to increase the yield of lentivirus infectious particles in cell supernatants [93], which were harvested 24-48 hours after the end of the transfection procedure, and immediately filtered to remove cellular debris (filter pore size: 0.45 μm). Lentivirus infectious particles were concentrated (100:1) by ultra-centrifugation (70,000 g, 2 hours at 4° C.) and then used to infect (1:2) previously established (1 week old) two-dimensional (2D) cultures of myoepithelial-like (CD49f^{high}/KIT^{neg}) cells. To increase infection efficiency, concentrated virus particles were “spinoculated” onto target cells (i.e., centrifugated at 1,200 g, 2 hours, 4° C.) in the presence of polybrene (8 μg/ml), and then left incubating with target cells at 37° C. for 12 hours [94, 95]. Infected 2D cultures were subsequently washed with fresh medium and cultured for an additional week, before final analysis by f^{low} cytometry. In all experiments designed to evaluate the capacity of DNhRARα to suppress the differentiation of myoepithelial-like cells into ductal-like cells (and/or the survival of ductal-like cells), analyses were restricted to lentivirus-infected cells, identified based on differential expression of the lentivirus-encoded fluorescent reporter (EGFP⁺). Differences in the mean percentage of cells with a ductal-like phenotype (CD49f^{low}/KIT⁺) among lentivirus-infected cells (EGFP⁺) across experimental replicates of the same culture (n=3) were tested for statistical significance using a Student's t-test for independent samples (two-tailed).

d. In Vivo Therapeutic Studies

Tumor-bearing animals were treated by intra-peritoneal (i.p.) injection of BMS493 (1 mg×3-4 days/week×3 weeks) resuspended in 0.15 M hydroxypropyl-β-cyclodextrin (HP-β-CD; Cayman Chemicals).

BMS493 (Tocris, 3509) was resuspended in DMSO (stock concentration: 50 mg/mL) and stored at −20° C. in single-use aliquots (20 μl). On the day of in vivo administration, single-use aliquots were thawed, and BMS493 was further diluted to a concentration of 2 mg/mL in DPBS supplemented with 0.15M hydroxypropyl β-cyclodextrin (HP-β-CD; Cayman Chemicals, 16169), for a total volume of 0.5 mL per dose (1 mg/dose). To facilitate compound dissolution, diluted BMS493 or DMSO was warmed at 37° C. for 10 minutes prior to injection. Mice were treated with either BMS493 or vehicle alone (DMSO, 0.15 M HP-β-CD) by intraperitoneal injection, according to two treatment regimens: Regimen 1 (for mono-phenotypic tumors), consisting in 3 doses/week (treatment on: Monday, Wednesday, Friday) for 3 weeks (total dose: 9 mg); or Regimen 2 (for bi-phenotypic tumors) consisting in 4 doses/week (treatment on: Monday, Tuesday, Thursday, Friday), for 3 weeks (total dose: 12 mg). Tumor volume was measured weekly, mice were weighted twice per week, and animals were monitored daily. Tumor volume was calculated using the following formula:

volume=width²×length/2

To enable robust comparisons across different treatment groups, tumor volumes were normalized to their starting values, and reported as fold-increases over time. Differences in mean normalized tumor volumes between treated and untreated mice were tested for statistical significance using two approaches: 1) at each time-point, using a Student's t-test (two-tailed); and 2) across the full experimental cohort, using a two-way (time-point×treatment) ANOVA for repeated measures (where measurements performed on the same mouse at different time-points are treated as repeated measures) [96]. Differences between growth rates (i.e., Log₁₀ of the fold-increase in tumor volume/time) were tested for statistical significance using a two-tailed Welch's t-test (i.e., assuming unequal variance). Tumor growth rates were calculated assuming exponential kinetics [96], following the procedure described by Hather et al. [37]. In vivo treatments were performed in three independent PDX models to ensure generalizability.

For in vivo BMS493 treatments, sample size was calculated so that the experiment would have sufficient statistical power to enable a test for the treatment's ability to alter a tumor's cell composition. Calculations were based under the assumption of aiming to test the ability of the treatment to alter cell the composition in the ACCX5M1 bi-phenotypic PDX line, where ductal-like cells, which were anticipated to be preferentially sensitive to BMS493 treatment, represented a minority. A sample size was calculated that would enable the experiment to have more than 80% probability (1−β=0.8) to measure a statistically significant difference (α=0.05) in the percentage of cells belonging to the minority phenotype when comparing treated and untreated cohorts using a t-test for continuous variables, and assuming 1) a mean baseline percentage of the minority population in untreated tumors of 23% (SD=4%) and 2) an effect of the treatment that would cause a reduction in their mean percentage to 15% (SD=3%). This corresponds to the percentage of cells that would be observed if the tested drug was able to kill one-third (35%) of the cells in the target minority population. The calculated sample size was 4.15 mice, and it was planned to have a minimum of 5 mice per experimental group for the in vivo experiments. Due to variability in starting tumor volumes associated with PDX engraftment, animals were assigned to BMS493 and DMSO-treated cohorts in such a way that the difference in the average tumor volume at the start of treatment was non-significant. Investigators were not blinded to group allocation during data collection or analysis due to the frequency of injections. In experiments shown in FIG. 8, four animals (ACCX5M1: n=2; ACCX9: n=2) had to be sacrificed in accordance with the animal protocol, prior to the completion of the full treatment regimen, due treatment-related toxicities. Data for these animals is excluded from analysis presented in FIG. 8. Individual curves for all animals are shown in FIG. 17. Outcomes measured included tumor volume and tumor growth rate.

e. Statistical Analyses

For each of the reported experiments, the mathematical and statistical approaches utilized to analyze and visualize the results are described in detail under the corresponding paragraph of this Supplementary Methods appendix and summarized in concise form within the legends of the corresponding figures. Very briefly, the distribution of experimental data was visualized using either box-plots [85], violin-plots [82], dot-plots [81], scatter-plots, heatmaps, UMAPs [81], histograms, QQ plots [98] or, more simply, error bars centered around mean values+/−standard deviations, all generated with the aid of graphical software, such as GraphPad Prism (version 8) or R (version 4.0.1). Where graphically feasible, all experimental replicates were reported as individual data-points. In the specific case of box-plots, boxes correspond to the range of values between the upper and lower quartiles of the data distribution, horizontal bars correspond to medians, and whiskers to minimum and maximum data-points. The statistical significance of observed differences was evaluated using a variety of tests, chosen on a case-by-case basis, depending on experimental assumptions and data distributions. The statistical tests used in this study included: Wald's test, Student's t-test (for either paired or unpaired samples, depending on experimental design), Welch's t-test (when sample variances were deemed to be unequal based on an F-test), Welch's one-way ANOVA with Dunnet's T3 multiple comparisons test, two-way ANOVA for repeated measures, Mann-Whitney's U-test and the Kruskal-Wallis H-test. In the specific case of high-throughput experiments involving the simultaneous measurement of thousands of genes (e.g., scRNA-seq, RNA-seq), the identification of differentially expressed genes was based on false-discovery rates (FDR) calculated using the Benjamini-Hochberg method, in order to adjust for multiple comparisons.

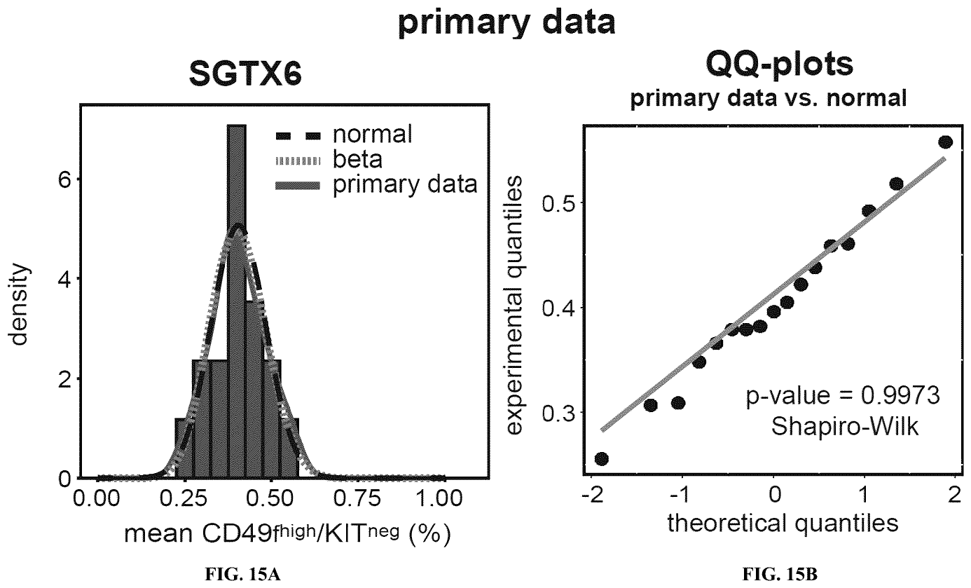

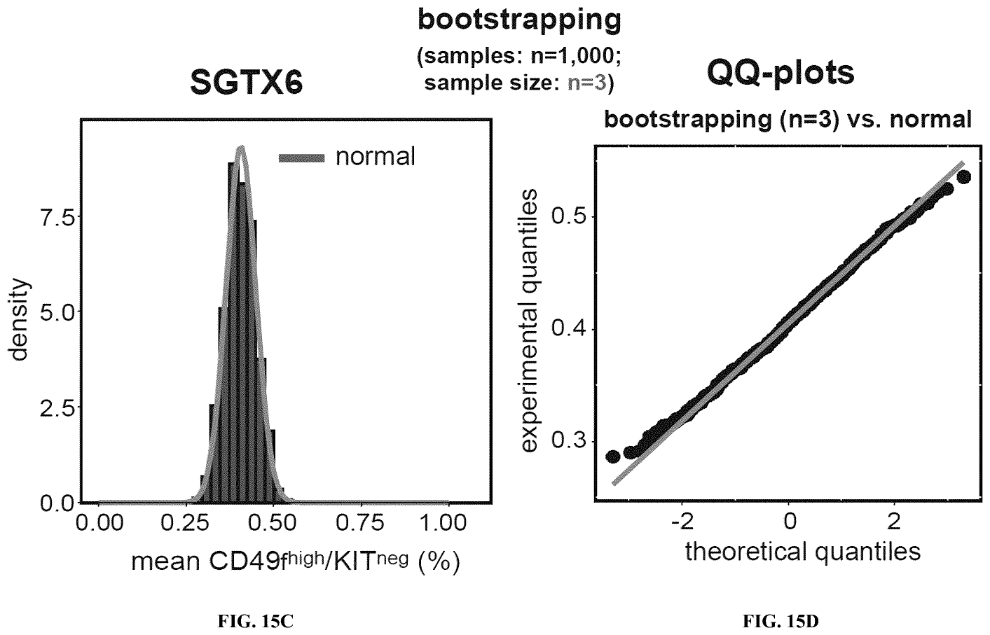

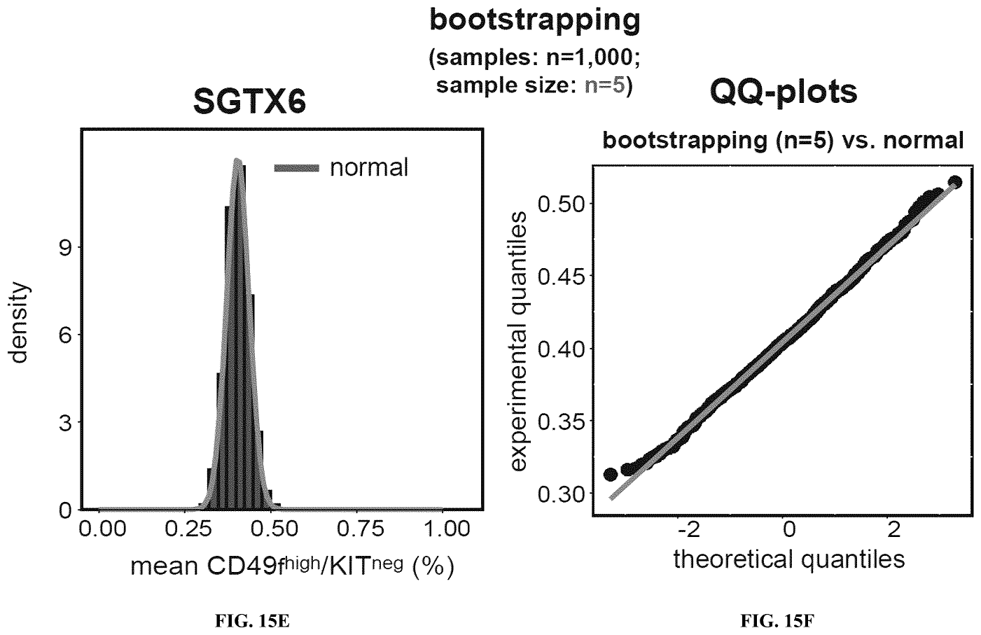

In experiments aimed at comparing different groups of tumors (or organoid cultures) in terms of their cell composition, the inferential approach consisted in using either a Student's t-test (two-tailed) or a Welch's ANOVA with Dunnett's T3 multiple comparisons test to evaluate the statistical significance of differences in the mean percentage of cancer cells belonging to a specific phenotype, either myoepithelial-like (CD49f^{high}/KIT^{neg}) or ductal-like (CD49f^{low}/KIT⁺), as calculated by averaging the percentages measured by FACS across independent tumor lesions (or organoid cultures) belonging to the same experimental group, with each tumor lesion (or organoid culture) representing an experimental replicate and an independent data-point. This approach was supported by an empirical study of the mathematical distribution of this type of primary variables in human ACCs (FIGS. 15A-15F). Very briefly, a historical series (n=17) of tumors were assembled that were established in the laboratory as independent replicates of the same bi-phenotypic PDX line (SGTX6) and that were analyzed by FACS in the absence of any perturbation or experimental manipulation (i.e., a homogeneous cohort of independent tumors representative of the baseline state of one of the PDX models), and then retrieved the corresponding data regarding the percentage of myoepithelial-like cells (CD49f^{high}/KIT^{neg}) and analyzed their distribution, using the “R” software (v4.1.2). The results of this study showed that, in the SGTX6 model, the distribution of the percentage of myoepithelial-like cells (CD49f^{high}/KIT^{neg}) was relatively well-approximated by the normal distribution, as revealed by: 1) a non-significant test for deviation from normality (Shapiro-Wilk; p=0.99); 2) a tight visual overlap between the curve describing the probability density of the primary data and the curve describing the probability density of a normal distribution with the same mean and standard deviation; and 3) a tight visual alignment of quantile data along the line of identity (x=y) in quantile-to-quantile (QQ) plots [98, 99]. The approximation to the normal distribution was further improved when a “bootstrapping” approach was used to model the distribution of the mean percentages expected to originate from the primary data, performed by iteratively re-sampling the primary dataset for sub-samples consisting of either three (n=3) or five (n=5) experimental replicates, picked randomly from the primary dataset and with replacements (number of re-sampling iterations: n=1,000). Because, in this experimental al setting, myoepithelial-like (CD49f^{high}/KIT^{neg}) or ductal-like (CD49f^{low}/KIT⁺) cells are mutually exclusive, and, together, form the entirety of the malignant cell population, the results observed for myoepithelial-like (CD49f^{high}/KIT^{neg}) cells are also expected to apply symmetrically to ductal-like (CD49f^{low}/KIT⁺) cells. From a theoretical point of view, these observations appeared coherent with a scenario in which the distribution of the primary variable could be described by aβ-distribution bound between 0 and 1, a distribution that is often used to model the distribution of variables consisting in a percentage (e.g., when a percentage is measured in each of a series of independent samples, each representing an experimental replicate) [100]. Indeed, in this experimental setting, the curve describing the probability density of the primary data also displayed a tight visual overlap with that of a β-distribution with the same mean and standard deviation of the primary data (Supplementary FIG. 7). Among the important mathematical features of the β-distribution is that, when the two parameters that control its shape (commonly referred to as α and β) are sufficiently similar and sufficiently high in value, it tends to rapidly approximate the normal distribution [101, 102]. In practical applications, this translates into the observation that, across many experimental settings, distributions of percentage values that are characterized by means that are not “extreme” (i.e., not close to the edges of the bounded range: 0-100%) can often be approximated by the normal distribution [101, 102]. Based on these experimental observations and theoretical considerations, it was concluded it would be acceptable for differences in the mean percentage of cancer cells with either a myoepithelial-like (CD49f^{high}/KIT^{neg}) or ductal-like (CD49f^{low}/KIT⁺) phenotype to be tested for statistical significance using parametric tests that assume a normal distribution of the data, especially when evaluating the statistical significance of differences in mean percentage values from samples containing 3-5 replicates.

### REFERENCES

1. Moskaluk C A. Adenoid cystic carcinoma: clinical and molecular features. Head Neck Pathol 2013; 7(1):17-22.
2. Lee R J, Tan A P, Tong E L, et al. Epidemiology, Prognostic Factors, and Treatment of Malignant Submandibular Gland Tumors: A Population-Based Cohort Analysis. JAMA Otolaryngol Head Neck Surg 2015; 141(10):905-12.
3. Gao M, Hao Y, Huang M X, et al. Clinicopathological study of distant metastases of salivary adenoid cystic carcinoma. Int J Oral Maxillofac Surg 2013; 42(8):923-8.
4. Bjorndal K, Krogdahl A, Therkildsen M H, et al. Salivary gland carcinoma in Denmark 1990-2005: outcome and prognostic factors. Results of the Danish Head and Neck Cancer Group (DAHANCA). Oral Oncol 2012; 48(2):179-85.
5. Lloyd S, Yu J B, Wilson L D, et al. Determinants and patterns of survival in adenoid cystic carcinoma of the head and neck, including an analysis of adjuvant radiation therapy. Am J Clin Oncol 2011; 34(1):76-81.
6. Laurie S A, Licitra L. Systemic therapy in the palliative management of advanced salivary gland cancers. J Clin Oncol 2006; 24(17):2673-8.
7. Dodd R L, Slevin N J. Salivary gland adenoid cystic carcinoma: a review of chemotherapy and molecular therapies. Oral Oncol 2006; 42(8):759-69.
8. Laurie S A, Ho A L, Fury M G, et al. Systemic therapy in the management of metastatic or locally recurrent adenoid cystic carcinoma of the salivary glands: a systematic review. Lancet Oncol 2011; 12(8):815-24.
9. Di Villeneuve L, Souza I L, Tolentino F D S, et al. Salivary Gland Carcinoma: Novel Targets to Overcome Treatment Resistance in Advanced Disease. Front Oncol 2020; 10:580141.
10. Persson M, Andrén Y, Mark J, et al. Recurrent fusion of MYB and NFIB transcription factor genes in carcinomas of the breast and head and neck. Proceedings of the National Academy of Sciences 2009; 106(44):18740-18744.
11. Mitani Y, Li J, Rao P H, et al. Comprehensive analysis of the MYB-NFIB gene fusion in salivary adenoid cystic carcinoma: Incidence, variability, and clinicopathologic significance. Clinical cancer research 2010; 16(19):4722-4731.
12. Ho A S, Kannan K, Roy D M, et al. The mutational landscape of adenoid cystic carcinoma. Nat Genet 2013; 45(7):791-8.
13. Rettig E M, Talbot C C, Jr., Sausen M, et al. Whole-Genome Sequencing of Salivary Gland Adenoid Cystic Carcinoma. Cancer Prev Res (Phila) 2016; 9(4):265-74.
14. Jiang Y, Gao R, Cao C, et al. MYB-activated models for testing therapeutic agents in adenoid cystic carcinoma. Oral Oncol 2019; 98:147-155.
15. Du F, Zhou C X, Gao Y. Myoepithelial differentiation in cribriform, tubular and solid pattern of adenoid cystic carcinoma: A potential involvement in histological grading and prognosis. Ann Diagn Pathol 2016; 22:12-7.
16. Jaso J, Malhotra R. Adenoid cystic carcinoma. Arch Pathol Lab Med 2011; 135(4):511-5.
17. Azumi N, Battifora H. The cellular composition of adenoid cystic carcinoma. An immunohistochemical study. Cancer 1987; 60(7):1589-98.
18. Prasad M L, Barbacioru C C, Rawal Y B, et al. Hierarchical cluster analysis of myoepithelial/basal cell markers in adenoid cystic carcinoma and polymorphous low-grade adenocarcinoma. Mod Pathol 2008; 21(2):105-14.
19. Wu H M, Ren G X, Wang L Z, et al. Expression of podoplanin in salivary gland adenoid cystic carcinoma and its association with distant metastasis and clinical outcomes. Mol Med Rep 2012; 6(2):271-4.
20. Drier Y, Cotton M J, Williamson K E, et al. An oncogenic MYB feedback loop drives alternate cell fates in adenoid cystic carcinoma. Nat Genet 2016; 48(3):265-72.
21. Parikh A S, Wizel A, Davis D, et al. Single-cell RNA sequencing identifies a paracrine interaction that may drive oncogenic notch signaling in human adenoid cystic carcinoma. Cell Rep 2022; 41(9):111743.
22. Dalerba P, Cho R W, Clarke M F. Cancer stem cells: models and concepts. Annu Rev Med 2007; 58:267-84.
23. Dalerba P, Kalisky T, Sahoo D, et al. Single-cell dissection of transcriptional heterogeneity in human colon tumors. Nat Biotechnol 2011; 29(12):1120-7.
24. Reya T, Morrison S J, Clarke M F, et al. Stem cells, cancer, and cancer stem cells. Nature 2001; 414(6859):105-11.
25. Dalerba P, Dylla S J, Park I K, et al. Phenotypic characterization of human colorectal cancer stem cells. Proc Natl Acad Sci USA 2007; 104(24):10158-63.
26. Wu A R, Neff N F, Kalisky T, et al. Quantitative assessment of single-cell RNA-sequencing methods. Nat Methods 2014; 11(1):41-6.
27. Moskaluk C A, Baras A S, Mancuso S A, et al. Development and characterization of xenograft model systems for adenoid cystic carcinoma. Lab Investigation 2011; 91(10):1480-90.
28. Aparicio L, Bordyuh M, Blumberg A J, et al. A random matrix theory approach to denoise single-cell data. Patterns 2020; 1(3):100035.
29. Love M I, Huber W, Anders S. Moderated estimation of fold change and dispersion for RNA-seq data with DESeq2. Genome biology 2014; 15(12):550.
30. Haas B J, Dobin A, Li B, et al. Accuracy assessment of fusion transcript detection via read-mapping and de novo fusion transcript assembly-based methods. Genome biology 2019; 20(1):1-16.
31. Hu Y, Smyth G K. ELDA: extreme limiting dilution analysis for comparing depleted and enriched populations in stem cell and other assays. Journal of Immunological Methods 2009; 347(1-2):70-78.
32. Shimono Y, Mukohyama J, Isobe T, et al. Organoid Culture of Human Cancer Stem Cells. Methods Mol Biol 2019; 1576:23-31.
33. Sato T, Stange D E, Ferrante M, et al. Long-term expansion of epithelial organoids from human colon, adenoma, adenocarcinoma, and Barrett's epithelium. Gastroenterology 2011; 141(5):1762-72.
34. Nanduri L S, Baanstra M, Faber H, et al. Purification and ex vivo expansion of fully functional salivary gland stem cells. Stem Cell Reports 2014; 3(6):957-64.
35. Panaccione A, Chang M T, Carbone B E, et al. NOTCH1 and SOX10 are Essential for Proliferation and Radiation Resistance of Cancer Stem-Like Cells in Adenoid Cystic Carcinoma. Clinical Cancer Research 2016; 22(8):2083.
36. Tiscornia G, Singer O, Verma I M. Production and purification of lentiviral vectors. Nat Protoc 2006; 1(1):241-5.
37. Damm K, Heyman R A, Umesono K, et al. Functional inhibition of retinoic acid response by dominant negative retinoic acid receptor mutants. Proc Natl Acad Sci USA 1993; 90(7):2989-93.
38. Eilenberger C, Kratz S R A, Rothbauer M, et al. Optimized alamarBlue assay protocol for drug dose-response determination of 3D tumor spheroids. MethodsX 2018; 5:781-787.
39. Bell D, Roberts D, Kies M, et al. Cell type-dependent biomarker expression in adenoid cystic carcinoma: biologic and therapeutic implications. Cancer 2010; 116(24):5749-56.
40. Iglesias J M, Cairney C J, Ferrier R K, et al. Annexin A8 identifies a subpopulation of transiently quiescent c-kit positive luminal progenitor cells of the ductal mammary epithelium. PLOS One 2015; 10(3):e0119718.
41. Lim E, Vaillant F, Wu D, et al. Aberrant luminal progenitors as the candidate target population for basal tumor development in BRCA1 mutation carriers. Nat Med 2009; 15(8):907-13.
42. Nguyen Q H, Pervolarakis N, Blake K, et al. Profiling human breast epithelial cells using single cell RNA sequencing identifies cell diversity. Nat Commun 2018; 9(1):2028.
43. Oakes S R, Naylor M J, Asselin-Labat M L, et al. The Ets transcription factor Elf5 specifies mammary alveolar cell fate. Genes Dev 2008; 22(5):581-6.
44. West R B, Kong C, Clarke N, et al. MYB expression and translocation in adenoid cystic carcinomas and other salivary gland tumors with clinicopathologic correlation. Am J Surg Pathol 2011; 35(1):92-9.
45. Ghyselinck N B, Duester G. Retinoic acid signaling pathways. Development 2019; 146(13).
46. Shannon S R, Moise A R, Trainor P A. New insights and changing paradigms in the regulation of vitamin A metabolism in development. Wiley Interdiscip Rev Dev Biol 2017; 6(3).
47. Metzler M A, Sandell L L. Enzymatic Metabolism of Vitamin A in Developing Vertebrate Embryos. Nutrients 2016; 8(12).
48. Dong D, Ruuska S E, Levinthal D J, et al. Distinct roles for cellular retinoic acid-binding proteins I and II in regulating signaling by retinoic acid. Journal of Biological Chemistry 1999; 274(34):23695-23698.
49. Schug T T, Berry D C, Shaw N S, et al. Dual transcriptional activities underlie opposing effects of retinoic acid on cell survival. Cell 2007; 129(4):723.
50. Napoli J L. Cellular retinoid binding-proteins, CRBP, CRABP, FABP5: Effects on retinoid metabolism, function and related diseases. Pharmacol Ther 2017; 173:19-33.
51. Bastien J, Rochette-Egly C. Nuclear retinoid receptors and the transcription of retinoid-target genes. Gene 2004; 328:1-16.
52. Wright D M, Buenger D E, Abashev T M, et al. Retinoic acid regulates embryonic development of mammalian submandibular salivary glands. Developmental Biology 2015; 407(1):57-67.
53. Abashev T M, Metzler M A, Wright D M, et al. Retinoic acid signaling regulates Krt5 and Krt14 independently of stem cell markers in submandibular salivary gland epithelium. Developmental Dynamics 2017; 246(2):135-147.
54. Metzler M A, Raja S, Elliott K H, et al. RDH10-mediated retinol metabolism and RARα-mediated retinoic acid signaling are required for submandibular salivary gland initiation. Development 2018; 145(15).
55. Mandelbaum J, Shestopalov I A, Henderson R E, et al. Zebrafish blastomere screen identifies retinoic acid suppression of <em>MYB</em> in adenoid cystic carcinoma. The Journal of Experimental Medicine 2018; 10.1084/jem.20180939.
56. Sun B, Wang Y, Sun J, et al. Establishment of patient-derived xenograft models of adenoid cystic carcinoma to assess pre-clinical efficacy of combination therapy of a PI3K inhibitor and retinoic acid. American journal of cancer research 2021; 11(3):773.
57. Ho A S, Ochoa A, Jayakumaran G, et al. Genetic hallmarks of recurrent/metastatic adenoid cystic carcinoma. J Clin Invest 2019; 129(10):4276-4289.
58. Stephens P J, Davies H R, Mitani Y, et al. Whole exome sequencing of adenoid cystic carcinoma. J Clin Invest 2013; 123(7):2965-8.
59. Ferrarotto R, Mitani Y, Diao L, et al. Activating NOTCH1 Mutations Define a Distinct Subgroup of Patients With Adenoid Cystic Carcinoma Who Have Poor Prognosis, Propensity to Bone and Liver Metastasis, and Potential Responsiveness to Notch1 Inhibitors. J Clin Oncol 2017; 35(3):352-360.
60. Ferrarotto R, Mitani Y, McGrail D J, et al. Proteogenomic Analysis of Salivary Adenoid Cystic Carcinomas Defines Molecular Subtypes and Identifies Therapeutic Targets. Clin Cancer Res 2021; 27(3):852-864.
61. Fordice J, Kershaw C, El-Naggar A, et al. Adenoid cystic carcinoma of the head and neck: predictors of morbidity and mortality. Arch Otolaryngol Head Neck Surg 1999; 125(2):149-52.
62. van Weert S, van der Waal I, Witte B I, et al. Histopathological grading of adenoid cystic carcinoma of the head and neck: analysis of currently used grading systems and proposal for a simplified grading scheme. Oral Oncol 2015; 51(1):71-6.
63. Romani C, Lorini L, Bozzola A, et al. Functional profiles of curatively treated adenoid cystic carcinoma unveil prognostic features and potentially targetable pathways. Sci Rep 2023; 13(1):1809.
64. Wolbach S B, Howe P R. Tissue changes following deprivation of fat-soluble A vitamin. The Journal of experimental medicine 1925; 42(6):753-777.
65. Adriance M C, Inman J L, Petersen O W, et al. Myoepithelial cells: good fences make good neighbors. Breast Cancer Research 2005; 7(5):1-8.
66. Sirka O K, Shamir E R, Ewald A J. Myoepithelial cells are a dynamic barrier to epithelial dissemination. The Journal of cell biology 2018; 217(10):3368-3381.
67. Hanna G J, ONeill A, Cutler J M, et al. A phase II trial of all-trans retinoic acid (ATRA) in advanced adenoid cystic carcinoma. Oral oncology 2021; 119:105366.
68. Dalerba P, Cho R W, Clarke M F. Cancer stem cells: models and concepts. Annu. Rev. Med. 2007; 58:267-284.
69. Jamieson C H, Ailles L E, Dylla S J, et al. Granulocyte-macrophage progenitors as candidate leukemic stem cells in blast-crisis CML. New England Journal of Medicine 2004; 351(7):657-667.
70. Akala O O, Park I K, Qian D, et al. Long-term haematopoietic reconstitution by Trp53−/−p16Ink4a−/−p19Arf−/− multipotent progenitors. Nature 2008; 453(7192):228-32.
71. Duvic M, Hymes K, Heald P, et al. Bexarotene is effective and safe for treatment of refractory advanced-stage cutaneous T-cell lymphoma: multinational phase II-III trial results. J Clin Oncol 2001; 19(9):2456-71.
72. Matthay K K, Villablanca J G, Seeger R C, et al. Treatment of high-risk neuroblastoma with intensive chemotherapy, radiotherapy, autologous bone marrow transplantation, and 13-cis-retinoic acid. Children's Cancer Group. N Engl J Med 1999; 341(16):1165-73.
73. Tallman M S, Andersen J W, Schiffer C A, et al. All-trans-retinoic acid in acute promyelocytic leukemia. N Engl J Med 1997; 337(15):1021-8.
74. Lo-Coco F, Avvisati G, Vignetti M, et al. Retinoic acid and arsenic trioxide for acute promyelocytic leukemia. N Engl J Med 2013; 369(2):111-21.
75. Walmsley S, Northfelt D W, Melosky B, et al. Treatment of AIDS-related cutaneous Kaposi's sarcoma with topical alitretinoin (9-cis-retinoic acid) gel. Panretin Gel North American Study Group. J Acquir Immune Defic Syndr 1999; 22(3):235-46.
76. Zhang Y, Parmigiani G, Johnson W E. ComBat-seq: batch effect adjustment for RNA-seq count data. NAR Genomics and Bioinformatics, 2:1qaa078(2020).
77. Crowley L, Cambuli F, Aparicio L, et al. A single-cell atlas of the mouse and human prostate reveals heterogeneity and conservation of epithelial progenitors. eLife, 9:e59465 (2020).
78. Traag V A, Waltman L, van Eck N J. From Louvain to Leiden: guaranteeing well-connected communities. Scientific Reports, 9:5233 (2019).
79. Wolf F A, Angerer P, Theis F J. SCANPY: large-scale single-cell gene expression data analysis. Genome Biology, 19:1-5 (2018).
80. Rousseeuw P J. Silhouettes: a graphical aid to the interpretation and validation of cluster analysis. Journal of Computational and Applied Mathematics, 20:53-65 (1987).
81. Becht E, McInnes L, Healy J, et al. Dimensionality reduction for visualizing single-cell data using UMAP. Nature Biotechnology, 37:38-44 (2018).
82. Hintze J L, Nelson R D. Violin plots: a box plot-density trace synergism. American Statistician, 52:181-184 (1998).
83. Leonard S, Lardenois A, Tarte K, et al. FlexDotPlot: a universal and modular dot plot visualization tool for complex multifaceted data. Bioinformatics Advances, 2:vbac019 (2022)
84. Schroeder A, Mueller O, Stocker S, et al. The RIN: an RNA integrity number for assigning integrity values to RNA measurements. BMC Molecular Biology, 7:3 (2006).
85. Williamson D F, Parker R A, Kendrick J S. The box plot: a simple visual method to interpret data. Annals of Internal Medicine, 110:916-921 (1989).
86. Uphoff C C, Drexler H G. Detection of mycoplasma contaminations. Methods in Molecular Biology, 946:1-13 (2013).
87. Dunnett C W. Pairwise multiple comparisons in the unequal variance case. Journal of the American Statistical Association, 75:796-800 (1980).
88. Sen J, Harpavat S, Peters M A, et al. Retinoic acid regulates the expression of dorsoventral topographic guidance molecules in the chick retina. Development, 132:5147-5159 (2005).
89. Bochkov Y A, Palmenberg A C. Translational efficiency of EMCV IRES in bicistronic vectors is dependent upon IRES sequence and gene location. Biotechniques, 41:283-292 (2006).
90. Kaminski A, Howell M T, Jackson R J. Initiation of encephalomyocarditis virus RNA translation: the authentic initiation site is not selected by a scanning mechanism. EMBO Journal, 9:3753-3759 (1990).
91. Ailles L E, Naldini L. HIV-1-derived lentiviral vectors. Current Topics in Microbiology and Immunology, 261:31-52 (2002).
92. Dull T, Zufferey R, Kelly M, et al. A third-generation lentivirus vector with a conditional packaging system. Journal of Virology, 72:8463-8471 (1998).
93. Ellis B L, Potts P R, Porteus M H. Creating higher titer lentivirus with caffeine. Human Gene Therapy, 22:93-100 (2011).
94. O'Doherty U, Swiggard W J, Malim M H. Human immunodeficiency virus type 1 spinoculation enhances infection through virus binding. Journal of Virology, 74:10074-10080 (2000).
95. Bondanza A, Valtolina V, Magnani Z, et al. Suicide gene therapy of graft-versus-host disease induced by central memory human T lymphocytes. Blood, 107:1828-1836 (2006).
96. Oberg A L, Heinzen E P, Hou X, et al. Statistical analysis of comparative tumor growth repeated measures experiments in the ovarian cancer patient derived xenograft (PDX) setting. Scientific Reports, 11:8076 (2021).
97. Hather G, Liu R, Bandi S, et al. Growth rate analysis and efficient experimental design for tumor xenograft studies. Cancer Informatics, 13 (S4):65-72 (2014).
98. Wilk M B, Gnanadesikan R. Probability plotting methods for the analysis of data. Biometrika, 55:1-17 (1968).
99. Das K R, Imon AHMR. A brief review of tests for normality. American Journal of Theoretical and Applied Statistics, 5:5-12 (2016).
100. Ferrari S L P, Cribari-Neto F. Beta regression for modelling rates and proportions. Journal of Applied Statistics, 31:799-815 (2004).
101. Ge H, Huang H Y, Li Y L, et al. Two approaches on accelerating Bayesian two-action learning automata. Intelligent Computing Methodologies, 9773:239-247 (2016).
102. Cook J D. Fast approximation of Beta inequalities. University of Texas, M D Anderson Cancer Center. Department of Biostatistics, Working Paper Series, Paper 76 (2012).

What is claimed is:

1. A method of reducing tumorigenicity and/or aggression of adenocarcinoma cells, the method comprising:
administering a therapeutic agent that activates retinoic acid receptor and/or retinoid-X receptor signaling to the adenocarcinoma cells.

2. The method of claim 1, further comprising:
detecting the expression of at least one cell surface marker in the adenocarcinoma cells, wherein the at least one cell surface marker is selected from the group consisting of: CD49f, TP63, and KIT/CD117,
wherein the adenocarcinoma cells are administered the therapeutic agent that activates retinoic acid receptor and/or retinoid-X receptor signaling to upon detection of:
more than 5% of the adenocarcinoma cells express TP63;
less than 95% of the adenocarcinoma cells express KIT/CD117; or
the adenocarcinoma cells have high expression of CD49f.

3. The method of claim 2, further comprising administering a therapeutic agent that inhibits retinoic acid receptor and/or retinoid-X receptor signaling to the adenocarcinoma cells after the administration of the therapeutic agent that activates retinoic acid receptor and/or retinoid-X receptor signaling.

4. A method of reducing viability of adenocarcinoma cells, the method comprising:
detecting the expression of at least one cell surface marker in the adenocarcinoma cells, wherein the at least one cell surface marker is selected from the group consisting of: CD49f, TP63, and KIT/CD117; and
administering a therapeutic agent that inhibits retinoic acid receptor and/or retinoid-X receptor signaling to the adenocarcinoma cells,
wherein the adenocarcinoma cells are administered the therapeutic agent that inhibits retinoic acid receptor and/or retinoid-X receptor signaling to upon detection of:
less than 5% of the adenocarcinoma cells express TP63;
more than 95% of the adenocarcinoma cells express KIT/CD117; or
the adenocarcinoma cells have low expression of CD49f.

5. The method of claim 4, further comprising:
administering a therapeutic agent that activates retinoic acid receptor and/or retinoid-X receptor signaling to the adenocarcinoma cells prior to administering the therapeutic agent that inhibits retinoic acid receptor and/or retinoid-X receptor signaling to the adenocarcinoma cells upon the detection of
more than 5% of the adenocarcinoma cells express TP63;
less than 95% of the adenocarcinoma cells express KIT/CD117; or
the adenocarcinoma cells have high expression of CD49f,
wherein the administration of the therapeutic agent that activates retinoic acid receptor and/or retinoid-X receptor signaling produces a population treated adenocarcinoma cells expressing KIT/CD117 without expression of TP63 or expressing KIT/CD117 with low expression of CD49f.

6. The method of claim 2, wherein the step of detecting the expression of the at least one cell surface marker in the adenocarcinoma cells comprises:
combining an antibody of CD49f, antibody of TP63, and/or an antibody of KIT/CD117 with the adenocarcinoma cells; and
sorting the adenocarcinoma cells based on binding of the antibody of CD49f, the antibody of TP63, and/or the antibody of KIT/CD117 to the adenocarcinoma cells.

7. The method of claim 6, wherein the antibody of CD49f and/or the antibody of KIT/CD11 are conjugated to a fluorescence marker, a magnetic particle, or microbubbles.

8. The method of claim 1, wherein the adenocarcinoma cells are adenoid cystic carcinoma (ACC).

9. A method of reducing the size of a tumor, the method comprising:
providing a tumor sample from a subject;
detecting the expression of at least one cell-surface marker in the tumor sample, wherein the at least one cell surface marker is selected from the group consisting of: CD49f, TP63, and KIT/CD117; and
administering a therapeutic agent that inhibits retinoic acid receptor and/or retinoid-X receptor signaling to the subject with a tumor sample comprising more than 95% of cells expressing KIT/CD117 or with a tumor sample comprising less than 5% of cells expressing TP63.

10. The method of claim 9, wherein the tumor sample of subject administered the therapeutic agent that inhibits retinoic acid receptor and/or retinoid-X receptor signaling has low expression level of CD49f.

11. The method of claim 9, further comprising administering a therapeutic agent that activates retinoic acid receptor and/or retinoid-X receptor signaling to the subject prior to administering the therapeutic agent that inhibits retinoic acid receptor and/or retinoid-X receptor signaling, wherein the tumor sample of the subject comprises:
more than 5% of the adenocarcinoma cells express TP63;
less than 95% of the adenocarcinoma cells express KIT/CD117; or
the adenocarcinoma cells have high expression of CD49f,
wherein the administration of the therapeutic agent that activates retinoic acid receptor and/or retinoid-X receptor signaling produces a population treated adenocarcinoma cells expressing KIT/CD117 without expression of TP63 or expressing KIT/CD117 with low expression of CD49f.

12. The method of claim 9, further comprising confirming the expression of at least a second cell-surface marker in the tumor sample selected from the group consisting of: ACTA2, MYH11, PDPN, ELF5, SLPI, and ANXA8, wherein the therapeutic agent that inhibits retinoic acid receptor and/or retinoid-X receptor signaling is administered to the subject with a tumor sample comprising more than 95% of cells expressing KIT/CD117 and at least a second cell-surface marker selected from the group consisting of ELF5, SLPI, and ANXA8.

13. The method of claim 9, wherein the step of detecting the expression of the at least one cell surface marker in the adenocarcinoma cells comprises:
combining an antibody of CD49f, antibody of TP63, and/or an antibody of KIT/CD117 with the adenocarcinoma cells; and
sorting the adenocarcinoma cells based on binding of the antibody of CD49f, the antibody of TP63, and/or the antibody of KIT/CD117 to the adenocarcinoma cells.

14. The method of claim 13, wherein the antibody of CD49f, the antibody of TP63, and the antibody of KIT/CD117 are conjugated to a fluorescence marker, a magnetic particle, or microbubbles.

15. The method of claim 9, wherein the tumor sample is from an adenoid cystic carcinoma (ACC).

16. The method of claim 1, wherein therapeutic agent that activates retinoic acid receptor/retinoid-X receptor signaling is selected from the group consisting of: all-trans retinoic acid (ATRA), bexarotene, or a combination thereof.

17. The method of claim 3, wherein the therapeutic agent that inhibits retinoic acid receptor/retinoid-X receptor signaling is selected from the group consisting of: BMS493, AGN193109, or a combination thereof.

18. The method of claim 3, wherein the therapeutic agent that inhibits retinoic acid receptor/retinoid-X receptor signaling is a gene construct encoding a dominant-negative version of RARα (DNRARα) that lacks its C-terminal transcriptional activation domain and/or is truncated at amino acid residue 403.

19. The method of claim 15, wherein the method comprises:
obtaining an ACC tumor sample from the subject;
sorting cells of the tumor sample based on the expression of CD49f and KIT/CD117 in the ACC tumor sample, wherein presence of cells positive for KIT/CD117 with low expression of CD49f indicates the presence of ductal-like ACC cells and cells negative for KIT/CD117 with high expression of CD49f indicates the presence myoepithelial-like ACC cells; and
administering a therapeutic agent to the subject that inhibits retinoic acid receptor and/or retinoid-X receptor signaling upon the indication of the presence of ductal-like ACC cells in the sample.

20. The method of claim 19, wherein the sorting step indicates the tumor sample comprises myoepithelial-like ACC cells, the method further comprising administering to the subject a therapeutic agent that activates retinoic acid receptor and/or retinoid-X receptor signaling before administering the therapeutic agent that inhibits retinoic acid receptor and/or retinoid-X receptor, thereby inducing the differentiation of myoepithelial-like tumor cells into ductal-like tumor cells.
